(19) United States
(12) Patent Application Publication
(10) Pub. No.: US 2020/0299282 A1
(43) Pub. Date: Sep. 24, 2020

(54) COMPOUNDS AND METHODS FOR TREATING CANCER

(71) Applicant: ANTIDOTE IP HOLDINGS, LLC, Fort Lauderdale, FL (US)

(72) Inventors: Michael S. Holzwarth, San Diego, CA (US); Elena V. Dneprovskaia, San Diego, CA (US)

(21) Appl. No.: 16/824,045

(22) Filed: Mar. 19, 2020

Related U.S. Application Data
(60) Provisional application No. 62/821,171, filed on Mar. 20, 2019.

(51) Int. Cl. C07D 413/04; C07C 311/21; C07D 235/14; C07D 271/06; C07D 277/28; C07D 213/40; C07D 285/08; C07D 277/82; C07D 215/06; C07D 239/42; C07D 401/04

(52) U.S. Cl. CPC C07D 413/04; C07C 311/21; C07D 235/14; C07D 271/06; C07D 277/28; C07D 401/04; C07D 285/08; C07D 277/82; C07D 215/06; C07D 239/42; C07D 213/40

(57) ABSTRACT

Substituted cinnamamide compounds and analogs, methods of making such compounds, pharmaceutical compositions and medicaments comprising such compounds, and methods of using such compounds to treat, prevent or ameliorate cancer are provided.

### INCORPORATION BY REFERENCE TO ANY PRIORITY APPLICATIONS

The present application claims the benefit of priority to U.S. Provisional Appl. No. 62/821,171, filed Mar. 20, 2019, which is incorporated by reference in its entirety.

### BACKGROUND

### Field

Substituted cinnamamide compounds and analogs, methods of making such compounds, pharmaceutical compositions and medicaments comprising such compounds, and methods of using such compounds to treat, prevent or ameliorate cancer are provided.

### DESCRIPTION

Over the past decade, cancer researchers have been primarily focusing on inhibiting the functions of various growth- and survival-promoting oncoproteins. While inhibition of various kinases involved in mitogenic signaling cascades initially proved to be very successful in treatment of cancer, the rapidly evolving cancer cells deploy various mechanisms to evade drug inhibition and eventually develop resistance to this targeted therapy. Such acquired resistance results in clinical relapse and resurgence of therapy-resistant tumors. The emergence of multi-drug resistant cancers requires development of novel approaches to their treatment.

p53 is a tumor suppressor protein that controls cell growth and tissue maintenance and plays a central role in preventing tumor suppression and development. The p53 pathway is activated in response to a broad variety of stress signals, such as gamma and UV irradiation, DNA damage, oncogene signaling, lack of nutrients, and oxidative damage. The level of the p53 response is carefully attenuated by post-translational modifications of the amino acid residues of the p53 protein, such as phosphorylation, acetylation, methylation, ubiquitination, sumoylation, neddylation and glutathionylation. These modifications affect p53 conformation, stability and its ability to form protein complexes with its various partners. The p53 response to stress signals can proceed via a transcription-dependent pathway and a transcription-independent pathway. The p53 transcription-dependent pathway relies on transcriptional up-regulation of genes involved in cell cycle arrest or apoptosis. The p53 transcription-independent pathway exerts its action in part via interactions with the Bcl-2 family of proteins affecting the polarization of the mitochondrial membrane. Other transcription-independent activities of the p53 protein are currently the focus of scientific investigations.

When assembled into a tetramer, or more specifically into a dimer of dimers, p53 shows sequence specific DNA-binding activity and activates expression of a number of genes involved in the DNA-repair mechanism, metabolism, cell cycle arrest, apoptosis and/or senescence of incipient cancer cells. In cancer cells the normal function of p53 is inactivated, which results in uncontrolled proliferation and genomic instability. In approximately 50% of cancers, p53 is inactivated by a missense mutation, a single base-pair substitution that results in translation of a different amino acid. 100+ different mutations have been identified in the p53 DNA-binding domain. p53 mutant proteins are broadly categorized into 3 main types—1) DNA-contact mutants, 2) structural mutants and 3) conformational mutants. The DNA-contact mutants seem to preserve the wild-type conformation, but lose the ability to form strong contacts with DNA, thus losing transcriptional activity either completely or partially. Structural mutants exhibit localized structural distortions of the amino acid residues, but mostly maintain native-like thermodynamic properties. Conformational mutants are thermodynamically unstable and are prone to rapid unfolding and aggregation. Both structural and conformational mutations are known to destabilize the active conformation of this highly flexible protein and disrupt its normal function. Furthermore, mutant p53 proteins exhibit oncogenic gain-of-function properties. Compounds disclosed in the present application may modulate both the p53 transcription-dependent and transcription-independent activities by stabilizing the active conformation of mutant p53 proteins, thereby restoring tumor suppression activity and preventing oncogenic gain-of-function activity.

### SUMMARY

Some embodiments of the present disclosure relate to compounds having the structure of Formula (I):

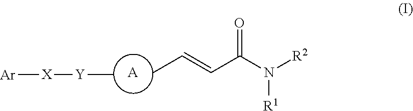

or a pharmaceutically acceptable salt thereof, wherein:

Ar is selected from the group consisting of C₆₋₁₀ aryl and 5 to 10 membered heteroaryl, each optionally substituted with one or more R¹⁰;

each of X and Y is independently selected from the group consisting of NR³, C(═O), and S(O)₂, provided that X and Y are not the same;

ring A is selected from the group consisting of optionally substituted C₆₋₁₀ aryl and optionally substituted 5 to 10 membered heteroaryl;

each of R¹ and R³ is independently selected from the group consisting of H, optionally substituted C₁₋₆ alkyl, optionally substituted C₆₋₁₀ aryl, optionally substituted 5 to 10 membered heteroaryl, optionally substituted 3 to 10 membered heterocyclyl, optionally substituted C₇₋₁₄ aralkyl, and optionally substituted C₃₋₇ carbocyclyl;

R² is selected from the group consisting of C₆₋₁₀ aryl and 5 to 10 membered heteroaryl, each optionally substituted with one or more R¹¹;

alternatively, R¹ and R² together with the nitrogen atom to which they are attached form a 3 to 10 membered heterocyclyl or a 5 to 10 membered heteroaryl, each optionally substituted with one or more R¹²;

each of R¹⁰, R¹¹ and R¹² is independently selected from the group consisting of C₁₋₆ alkyl, C₂₋₆ alkenyl, C₂₋₆ alkynyl, C₁₋₆ haloalkyl, C₁₋₆ alkoxy, C₁₋₆ haloalkoxy, (C₁₋₆ alkoxy)C₁₋₆ alkyl, C₁₋₆ alkylthiol, C₆₋₁₀ aryl, C₇₋₁₄ aralkyl, 5 to 10 membered heteroaryl, 3 to 7 membered heterocyclyl, C₃₋₇ carbocyclyl, halo, —CN, —NO₂, —NR^{5a}R^{6a}, —OR^{7a}, —C(O)R^{8a}, —C(O)OR^{7b}, —C(O)NR^{5b}R^{6b}, —NR^{5c}C(O)R^{8b}, —NR^{5d}S(O)₂R^{9a} and —S(O)₂R^{9b}, wherein each of C₁₋₆ alkyl, C₂₋₆ alkenyl, C₂₋₆ alkynyl, C₆₋₁₀ aryl, C₇₋₁₄ aralkyl, 5 to 10 membered heteroaryl, 3 to 7 membered heterocyclyl, and C₃₋₇ carbocyclyl is optionally substituted with one or more R¹¹; or two geminal R¹² form oxo;

each R¹³ is independently selected from the group consisting of C₁₋₆ alkyl, C₂₋₆ alkenyl, C₂₋₆ alkynyl, C₁₋₆ haloalkyl, C₁₋₆ alkoxy, C₁₋₆ haloalkoxy, (C₁₋₆ alkoxy)C₁₋₆ alkyl, C₁₋₆ alkylthiol, optionally substituted phenyl, optionally substituted 5 or 6 membered heteroaryl, optionally substituted 3 to 7 membered heterocyclyl, optionally substituted C₃₋₇ carbocyclyl, halo, —CN, —NO₂, SEM, —(C₀₋₆ alkylene)NR^{5a}R^{6a}, —OR^{7a}, —C(O)R^{8a}, —C(O)OR^{7b}, —C(O)NR^{5b}R^{6b}, —NR^{5c}C(O)R^{8b}, —NR^{5d}S(O)₂R^{9a} and —S(O)₂R^{9b}, or two geminal R¹³ form oxo;

each of R^{5a}, R^{5b}, R^{5c}, R^{5d}R^{6a}, and R^{6b} is independently selected from the group consisting of H, optionally substituted C₁₋₆ alkyl, optionally substituted (C₀₋₆ alkylene)C₆₋₁₀ aryl, optionally substituted (C₀₋₆ alkylene) 5 to 10 membered heteroaryl, optionally substituted (C₀₋₆ alkylene) 3 to 7 membered heterocyclyl, and optionally substituted (C₀₋₆ alkylene)C₃₋₇ carbocyclyl; or R^{5a} and R^{6a} together with the nitrogen atom to which they are attached form an optionally substituted 3 to 7 membered heterocyclyl; or R^{5b} and R^{6b} together with the nitrogen atom to which they are attached form an optionally substituted 3 to 7 membered heterocyclyl; and

each of R^{7a}, R^{7b}, R^{8a}, R^{8b}, R^{9a}, and R^{9b} is independently selected from the group consisting of H, optionally substituted C₁₋₆ alkyl, optionally substituted C₂₋₆ alkenyl, optionally substituted C₂₋₆ alkynyl, optionally substituted C₆₋₁₀ aryl, optionally substituted C₇₋₁₄ aralkyl, and optionally substituted C₃₋₇ carbocyclyl.

In some embodiments of the compound of Formula (I), ring A is

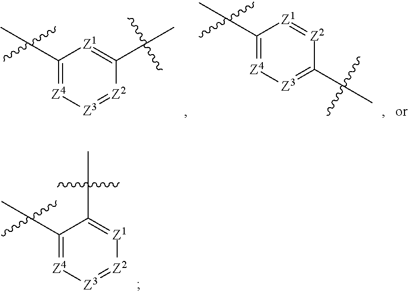

wherein Z¹ is CR^{4a} or N; Z² is CR^{4b} or N; Z³ is CR^{4c} or N; Z⁴ is CR^{4d} or N; each of R^{4a}, R^{4b}, R^{4c}, R^{4d} is independently selected from the group consisting of H, optionally substituted C₁₋₆ alkyl, optionally substituted C₂₋₆ alkenyl, optionally substituted C₂₋₆ alkynyl, C₁₋₆ haloalkyl, C₁₋₆ alkoxy, C₁₋₆ haloalkoxy, (C₁₋₆ alkoxy)C₁₋₆ alkyl, C₁₋₆ alkylthiol, halo, —CN, —NO₂, —NR^{5a}R^{6a}, —OR^{7a}, —C(O)R^{8a}, —C(O)OR^{7b}, —C(O)NR^{5b}R^{6b}, —NR^{5c}C(O)R^{8b}, —NR^{5d}S(O)₂R^{9a} and —SO₂R^{9b},

In some embodiments, the compounds of Formula (I) have the structure of Formula (II):

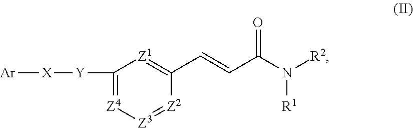

or pharmaceutically acceptable salts thereof. In some further embodiments, the compounds of Formula (II) have the structure Formula (II-A), (II-B), (II-C) or (II-D):

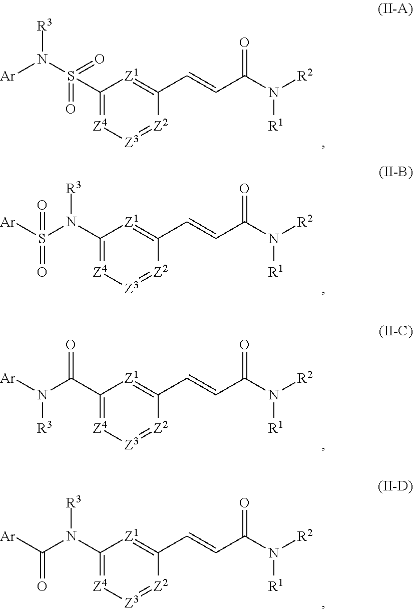

or pharmaceutically acceptable salts thereof.

In some embodiments of the compounds of Formula (I), (II), or (II-A)-(II-D), Z¹ is CR^{4a}, Z² is CR^{4b}, Z³ is CR^{4c} and Z⁴ is CR^{4d}. In some other embodiments, at least one of Z¹, Z², Z³, and Z⁴ is N. Further embodiments of the compounds have the structure of Formulas (III-A) through (III-D) or (IV-A) through (IV-D):

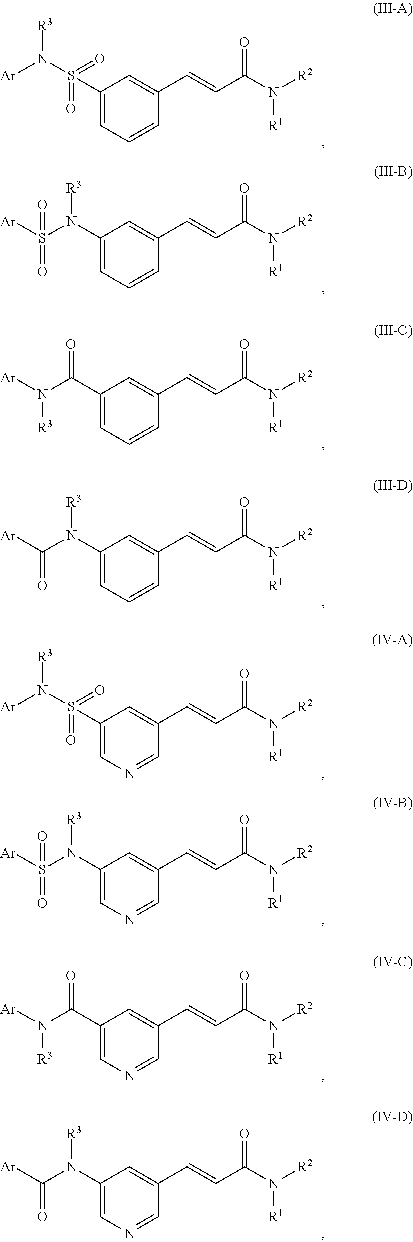

pharmaceutically acceptable salts thereof.

In some embodiments, the compounds of Formula (I) have the structure of Formula (V):

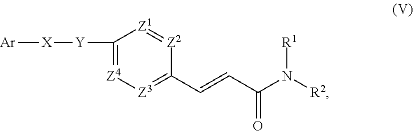

or pharmaceutically acceptable salts thereof. In some further embodiments, the compounds of Formula (V) have the structure Formula (V-A), (V-B), (V-C) or (V-D), or a pharmaceutically acceptable salt thereof:

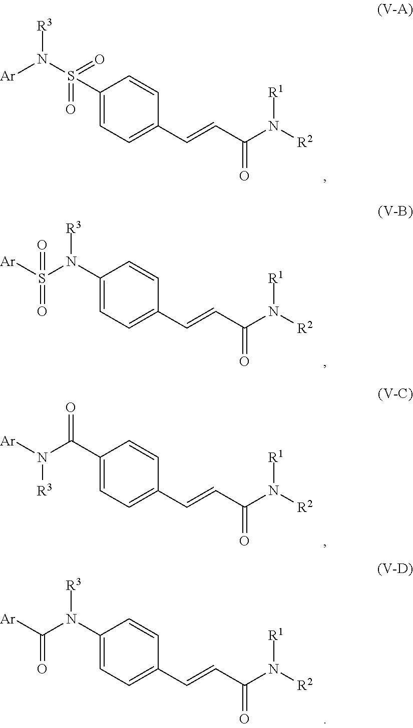

Some embodiments of the present disclosure relate to pharmaceutical compositions comprising a compound of Formula (I), (II), (II-A) through (II-D), (III-A) through (III-D), (IV-A) through (IV-D), (V), (V-A) through (V-D) as described herein, or a pharmaceutically acceptable salt thereof, and a pharmaceutically acceptable excipient or carrier.

Some embodiments of the present disclosure relate to methods of modulating or activating a p53 signaling pathway in a subject, comprising administering a therapeutically effective amount of a compound of Formula (I), (II), (II-A) through (II-D), (III-A) through (III-D), (IV-A) through (IV-D), (V), (V-A) through (V-D) as described herein, or a pharmaceutically acceptable salt thereof, or a pharmaceutical composition thereof, to a subject. In some embodiments, the subject possesses low levels of wild-type p53, a p53 mutation, or high levels of p53 protein having the p53 mutation.

Some other embodiments of the present disclosure relate to methods of inhibiting cancer cell growth, comprising contacting a cancer cell with an effective amount of a compound of Formula (I), (II), (II-A) through (II-D), (III-A) through (III-D), (IV-A) through (IV-D), (V), (V-A) through (V-D) as described herein, or a pharmaceutically acceptable salt thereof, or a pharmaceutical composition thereof. In some embodiments, the cancer cell has been identified as possessing wild-type p53, under-expressing p53, or a p53 mutation.

Some further embodiments of the present disclosure relate to methods of treating cancer, comprising: administering a therapeutically effective amount of a compound of Formula (I), (II), (II-A) through (II-D), (III-A) through (III-D), (IV-A) through (IV-D), (V), (V-A) through (V-D) as described herein, or a pharmaceutically acceptable salt thereof, or a pharmaceutical composition thereof to a subject. In some such embodiments, the subject has a p53 mutation in the DNA-binding domain.

In any of the methods disclosed herein, the p53 mutation may comprise R273H, R273C, R175H, R175L, G245S, G245C, C176F, R249S, R282W, C242W, R248Q, R248W, Y220C, or R280K, or combinations thereof. In some embodiments, the p53 mutation is selected from the group consisting of R273H, R273C, R175H, R175L, G245S, G245C, C176F, R249S, R282W, C242W, R248Q, R248W, Y220C, and R280K and combinations thereof. In some further embodiments, the p53 mutation is selected from the group consisting of R175H, G245S, and R248Q, and combinations thereof.

### BRIEF DESCRIPTION OF THE DRAWINGS

FIG. 1A is a bar chart illustrating the p21 gene reporter assay results with H1299 p53⁻/⁻ cells transfected with p53-R175H and p53-G245S plasmids for Compounds 12, 43, 45, 49, 79, 84 and 120, as compared to DMSO control at various concentrations.

FIG. 1B is a bar chart illustrating the p21 gene reporter assay results with H1299 p53⁻/⁻ cells transfected with p53-R175H and p53-G245S plasmids for Compounds 93, 104, 105, 123, 124, and 125, as compared to DMSO control at various concentrations.

FIG. 1C is a bar chart illustrating the p21 gene reporter assay results with H1299 p53⁻/⁻ cells transfected with p53-R175H and p53-G245S plasmids for Compounds 43, 49, 79, 84, 104, 105, 125, 76, 57, 25, and 56, as compared to DMSO control at various concentrations.

### DETAILED DESCRIPTION

The present application discloses novel cinnamamide analogs and derivatives that are useful therapeutic agents for treating, preventing or ameliorating cancers. In particular, these compounds may affect the overall conformation and stability of mutant p53 protein. As a result, the compounds disclosed therein may restore tumor suppression activity and preventing oncogenic gain-of-function activities of mutant p53 protein.

### Definitions

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, except for the claims, a group of items linked with the conjunction ‘and’ should not be read as requiring that each and every one of those items be present in the grouping, but rather should be read as ‘and/or’ unless the context indicates otherwise. Similarly, except for the claims, a group of items linked with the conjunction ‘or’ should not be read as requiring mutual exclusivity among that group, but rather should be read as ‘and/or’ unless the context indicates otherwise.

All references cited herein are incorporated by reference in their entirety unless stated otherwise. To the extent publications and patents or patent applications incorporated by reference contradict the disclosure contained in the specification, the specification is intended to supersede and/or take precedence over any such contradictory material. In the event that there are a plurality of definitions for a term herein, those in this section prevail unless stated otherwise.

As used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The section headings used herein are for organizational purposes only and are not to be construed as limiting the subject matter described.

As used herein, common organic abbreviations are defined as follows:

Ac Acetyl

aq. Aqueous

Bn Benzyl

Bz Benzoyl

BOC or Boc tert-Butoxycarbonyl

Bu n-Butyl

° C. Temperature in degrees Centigrade

DCM Methylene chloride

DMF N,N′-Dimethylformamide

DMSO Dimethylsulfoxide

ee % Enantiomeric excess

EtOH Ethanol

Et Ethyl

EtOAc Ethyl acetate

g Gram(s)

h or hr Hour(s)

iPr Isopropyl

m or min Minute(s)

MeOH MeOH

mL Milliliter(s)

PG Protecting group

Ph Phenyl

ppt Precipitate

rt Room temperature

SEM [2-(Trimethylsilyl)ethoxy]methyl acetal

Tert, t tertiary

TLC Thin-layer chromatography

μL Microliter(s)

As used herein, the phrase “p53 signaling pathway” refers to signal transduction cascades that include the p53 protein. These signal transduction cascades include, but not limited to, response to irradiation (for example, gamma or UV exposure), response to DNA damage (for example, missense mutations, nonsense mutation, oxidation, deamination, alkylation, single-strand breaks, and double-strand breaks), response to nutrient depletion, response to oxidative damage (for example, by reactive oxygen species), hypoxia, and response to oncogene signaling (for example, oncogenic Ras and oncogenic Myc signaling).

“Solvate” refers to the compound formed by the interaction of a solvent and a compound described herein or salt thereof. Suitable solvates are pharmaceutically acceptable solvates including hydrates.

The term “pharmaceutically acceptable salt” refers to salts that retain the biological effectiveness and properties of a compound and, which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, and hexyl.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy.

As used herein, “alkylthio” refers to the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, and tert-butylmercapto.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2-dienyl, and buta-1,2-dien-4-yl. Typical alkenyl groups include, but are in no way limited to, ethenyl, propenyl, butenyl, pentenyl, and hexenyl.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO- and RS-, in which R is an aryl as is defined above, such as “C₆₋₁₀ aryloxy” or “C₆₋₁₀ arylthio”, including but not limited to phenyloxy.

An “aralkyl” or “arylalkyl” is an aryl group connected, as a substituent, via an alkylene group, such as “C₇₋₁₄ aralkyl”, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, oxadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, and cycloheptylmethyl. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N and S, and in preferred five membered monocyclic heterocyclyls, the heteroatom(s) are selected from one or two heteroatoms selected from O, N, and S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes free amino (i.e., —NH₂).

As used herein, an “aminoalkyl” group refers to an amino group connected via an alkylene group.

As used herein, an “alkoxyalkyl” group refers to an alkoxy group connected via an alkylene group, such as a “C₂₋₈ alkoxyalkyl” and (C₁₋₆ alkoxy)C₁₋₆ alkyl and the like.

As used herein, SEM group has the structure:

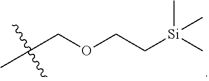 [SMILES: *COCC[Si](C)(C)C]

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C3-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C1-C6)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁₋₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁₋₆ alkyl, C₁-C₆ alkoxy, C₁-C₆ haloalkyl, and C₁-C₆ haloalkoxy), halo, cyano, hydroxy, C₁₋₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

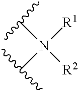

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

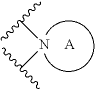

where ring A is a heteroaryl ring containing the depicted nitrogen.

Similarly, when two “adjacent” R groups are said to form a ring “together with the atom to which they are attached,” it is meant that the collective unit of the atoms, intervening bonds, and the two R groups are the recited ring. For example, when the following substructure is present:

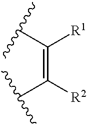

and R¹ and R² are defined as selected from the group consisting of hydrogen and alkyl, or R¹ and R² together with the atoms to which they are attached form an aryl or carbocylyl, it is meant that R¹ and R² can be selected from hydrogen or alkyl, or alternatively, the substructure has structure:

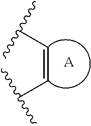

where A is an aryl ring or a carbocylyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

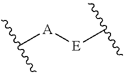

includes the substituent being oriented such that the A is attached at the leftmost attachment point of the molecule as well as the case in which A is attached at the rightmost attachment point of the molecule.

As used herein, “isosteres” of a chemical group are other chemical groups that exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated include —SO₃H, —SO₂HNR, —PO₂(R)₂, —PO₃(R)₂, —CONHNHSO₂R, —COHNSO₂R, and —CONRCN, where R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of carbocyclic and heterocyclic isosteres contemplated. The atoms of said ring structure may be optionally substituted at one or more positions with R as defined above.

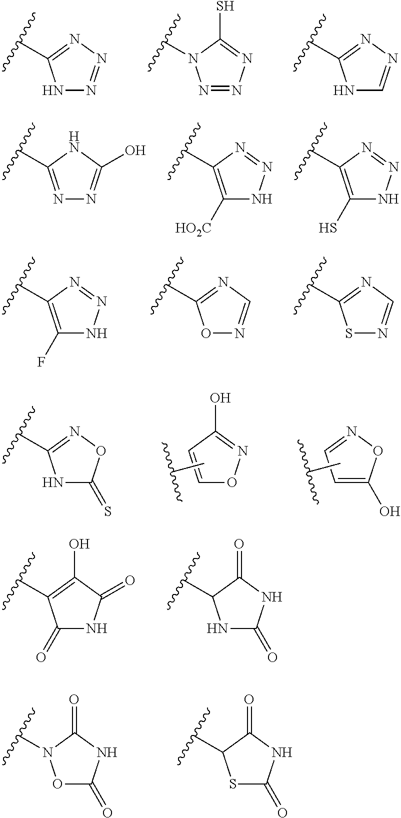

It is also contemplated that when chemical substituents are added to a carboxylic isostere, the compound retains the properties of a carboxylic isostere. It is contemplated that when a carboxylic isostere is optionally substituted with one or more moieties selected from R as defined above, then the substitution and substitution position is selected such that it does not eliminate the carboxylic acid isosteric properties of the compound. Similarly, it is also contemplated that the placement of one or more R substituents upon a carbocyclic or heterocyclic carboxylic acid isostere is not a substitution at one or more atom(s) that maintain(s) or is/are integral to the carboxylic acid isosteric properties of the compound, if such substituent(s) would destroy the carboxylic acid isosteric properties of the compound.

Other carboxylic acid isosteres not specifically exemplified in this specification are also contemplated.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A therapeutic effect relieves, to some extent, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes can be present in the compounds described. Each chemical element as represented in a compound structure can include any isotope of said element. For example, at any position of the compound that a hydrogen atom is be present, the hydrogen atom encompasses any isotope of hydrogen, including but not limited to hydrogen-1 (protium) and hydrogen-2 (deuterium). Thus, reference herein to a compound encompasses all potential isotopic forms unless the context clearly dictates otherwise. Deuteration replacement of a hydrogen-1 at a metabolically labile position of a compound may improve the pharmacokinetic properties of the compound.

### Compounds

Formula (I)

Some embodiments of present disclosure relate to compounds having the structure of Formula (I):

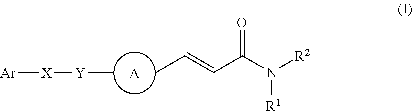

or a pharmaceutically acceptable salt thereof, wherein:

Ar is selected from the group consisting of C₆₋₁₀ aryl and 5 to 10 membered heteroaryl, each optionally substituted with one or more R¹⁰;

each of X and Y is independently selected from the group consisting of NR³, C(═O), and S(O)₂, provided that X and Y are not the same;

ring A is

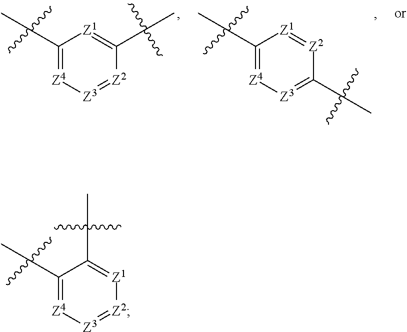

Z¹ is CR^{4a} or N;

Z² is CR^{4b} or N;

Z³ is CR^{4c} or N;

Z⁴ is CR^{4d} or N;

each of R¹ and R³ is independently selected from the group consisting of H, optionally substituted C₁₋₆ alkyl, optionally substituted C₆₋₁₀ aryl, optionally substituted 5 to 10 membered heteroaryl, optionally substituted 3 to 10 membered heterocyclyl, optionally substituted C₇₋₁₄ aralkyl, and optionally substituted C₃₋₇ carbocyclyl;

R² is selected from the group consisting of C₆₋₁₀ aryl and 5 to 10 membered heteroaryl, each optionally substituted with one or more R¹¹;

alternatively, R¹ and R² together with the nitrogen atom to which they are attached form a 3 to 10 membered heterocyclyl or a 5 to 10 membered heteroaryl, each optionally substituted with one or more R¹²;

each of R^{4a}, R^{4b}, R^{4c}, R^{4d} is independently selected from the group consisting of H, optionally substituted C₁₋₆ alkyl, optionally substituted C₂₋₆ alkenyl, optionally substituted C2-6 alkynyl, C₁₋₆ haloalkyl, C₁₋₆ alkoxy, C₁₋₆ haloalkoxy, (C₁₋₆ alkoxy)C₁₋₆ alkyl, C₁₋₆ alkylthiol, halo, —CN, —NO₂, —NR^{5a}R^{6a}, —OR^{7a}, —C(O)R^{8a}, —C(O)OR^{7b}, —C(O)NR^{5b}R^{6b}, —NR^{5c}C(O)R^{8b}, —NR^{5d}S(O)₂R^{9a} and —SO₂R^{9b};

each of R¹⁰, R¹¹ and R¹² is independently selected from the group consisting of C₁₋₆ alkyl, C₂₋₆ alkenyl, C₂₋₆ alkynyl, C₁₋₆ haloalkyl, C₁₋₆ alkoxy, C₁₋₆ haloalkoxy, (C₁₋₆ alkoxy)C₁₋₆ alkyl, C₁₋₆ alkylthiol, C₆₋₁₀ aryl, C₇₋₁₄ aralkyl, 5 to 10 membered heteroaryl, 3 to 7 membered heterocyclyl, C₃₋₇ carbocyclyl, halo, —CN, —NO₂, —NR^{5a}R^{6a}, —OR^{7a}, —C(O)R^{8a}, —C(O)OR^{7b}, —C(O)NR^{5b}R^{6b}, —NR^{5c}C(O)R^{8b}, —NR^{5d}S(O)₂R^{9a} and —S(O)₂R^{9b}, wherein each of C₁₋₆ alkyl, C₂₋₆ alkenyl, C26 alkynyl, C₆₋₁₀ aryl, C₇₋₁₄ aralkyl, 5 to 10 membered heteroaryl, 3 to 7 membered heterocyclyl, and C₃₋₇ carbocyclyl is optionally substituted with one or more R¹³; or two geminal R¹² form oxo;

each R¹³ is independently selected from the group consisting of C₁₋₆ alkyl, C₂₋₆ alkenyl, C₂₋₆ alkynyl, C₁₋₆ haloalkyl, C₁₋₆ alkoxy, C₁₋₆ haloalkoxy, (C₁₋₆ alkoxy)C₁₋₆ alkyl, C₁₋₆ alkylthiol, optionally substituted phenyl, optionally substituted 5 or 6 membered heteroaryl, optionally substituted 3 to 7 membered heterocyclyl, optionally substituted C₃₋₇ carbocyclyl, halo, —CN, —NO₂, SEM, —(C₀₋₆ alkylene)NR^{5a}R^{6a}, —OR^{7a}, —C(O)R^{8a}, —C(O)OR^{7b}, —C(O)NR^{5b}R^{6b},

—NR^{5c}C(O)R^{8b}, —NR^{5d}S(O)₂R^{9a} and —S(O)₂R^{9b}, or two geminal R¹³ form oxo;

each of R^{5a}, R^{5b}, R^{5c}, R^{5d,} R⁶, and R^{6b} is independently selected from the group consisting of H, optionally substituted C₁₋₆ alkyl, optionally substituted (C₀₋₆ alkylene)C₆₋₁₀ aryl, optionally substituted (C₀₋₆ alkylene) 5 to 10 membered heteroaryl, optionally substituted (C₀₋₆ alkylene) 3 to 7 membered heterocyclyl, and optionally substituted (C₀₋₆ alkylene)C₃₋₇ carbocyclyl; or R^{5s} and R6a together with the nitrogen atom to which they are attached form an optionally substituted 3 to 7 membered heterocyclyl; or R^{5b} and R^{6b} together with the nitrogen atom to which they are attached form an optionally substituted 3 to 7 membered heterocyclyl; and

each of R^{7a}, R^{7b}, R^{8a}, R^{8b}, R^{9a}, and R^{9b} is independently selected from the group consisting of H, optionally substituted C₁₋₆ alkyl, optionally substituted C₂₋₆ alkenyl, optionally substituted C₂₋₆ alkynyl, optionally substituted C₆₋₁₀ aryl, optionally substituted C₇₋₁₄ aralkyl, and optionally substituted C₃₋₇ carbocyclyl.

In some embodiments, the compounds of Formula (I) have the structure of Formula (II), or pharmaceutically acceptable salts thereof:

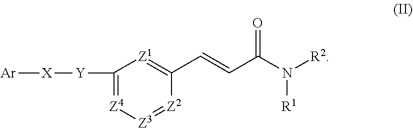

In some further embodiments, the compounds of Formula (II) also have the structure of Formula (II-A), (II-B), (II-C) or (II-D), or pharmaceutically acceptable salts thereof:

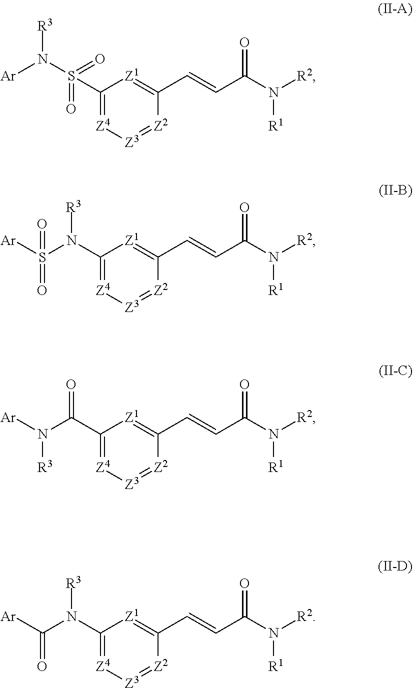

In some embodiments of the compounds of Formula (I), (II), (II-A), (II-A), (II-C) or (II-D), Z¹ is CR⁴. In some further embodiments, Z² is CR^{4b}. In some further embodiments, Z³ is CR^{4c}. In some further embodiments, Z⁴ is CR^{4d}. In one embodiment, Z¹ is CR^{4a}, Z² is CR^{4b}, Z³ is CR^{4c} and Z⁴ is CR^{4d}. In some such embodiments, the compounds have the structure of Formula (III-A), (III-B), (III-C) or (III-D):

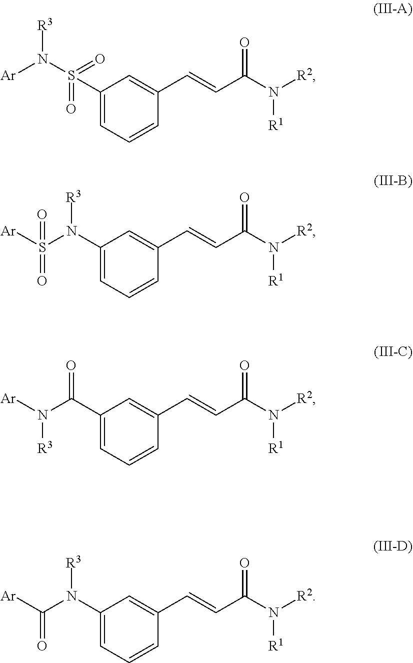

In some other embodiments of the compounds of Formula (I), (II), (II-A), (II-A), (II-C) or (II-D), at least one of Z¹, Z², Z³, and Z⁴ is N. In some such embodiments, Z¹ is N. In some such embodiments, Z² is N. In other such embodiments, Z³ is N. In other such embodiments, Z⁴ is N. In some such embodiments, the compounds have the structure of Formula (IV-A), (IV-B), (IV-C) or (IV-D):

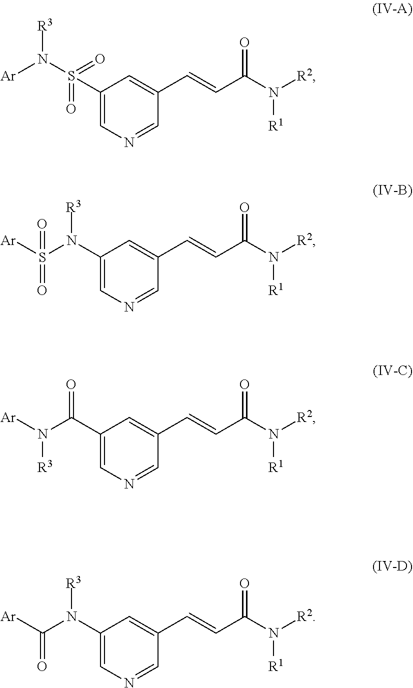

In some other embodiments, the compounds of Formula (I) have the structure of Formula (V):

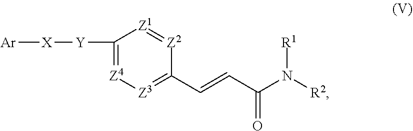

or pharmaceutically acceptable salts thereof. In some further embodiments, the compounds of Formula (V) have the structure Formula (V-A), (V-B), (V-C) or (V-D), or a pharmaceutically acceptable salt thereof. In some other embodiments of the compound of Formula (V), at least one of Z¹, Z², Z³, and Z⁴ is N.

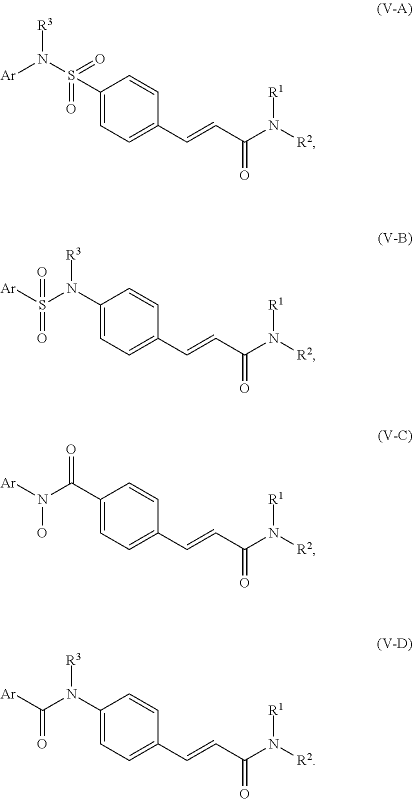

In some embodiments of the compounds of any one of Formula (I), (II), (II-A) through (II-D), (III-A) through (III-D), (IV-A) through (IV-D), (V), and (V-A) through (V-D), R¹ is H or C₁₋₆ alkyl. In some such embodiments, R¹ is methyl.

In some embodiments of the compounds of any one of Formula (I), (II), (II-A) through (II-D), (III-A) through (III-D), (IV-A) through (IV-D), (V), and (V-A) through (V-D), R³ is H or C₁₋₆ alkyl. In some such embodiments, R³ is methyl.

In some embodiments of the compounds of any one of Formula (I), (II), (II-A) through (II-D), (III-A) through (III-D), (IV-A) through (IV-D), (V), and (V-A) through (V-D), Ar is phenyl. In some such embodiment, Ar is unsubstituted. In some other embodiments, Ar is substituted with one or more R¹⁰. In some such embodiments, R¹⁰ is selected from the group consisting of halo, C₁₋₆ alkyl, C₁₋₆ haloalkyl, C₁₋₆ alkoxy, and C₁₋₆ haloalkoxy. In some further embodiments, R¹⁰ is F, Cl, —OCF₃, —CF₃, or methyl. In one embodiment, R¹⁰ is —CF₃.

In some embodiments of the compounds of any one of Formula (I), (II), (II-A) through (II-D), (III-A) through (III-D), (IV-A) through (IV-D), (V), and (V-A) through (V-D), R² is a 5, 6, 9 or 10 membered heteroaryl, for example, R² is selected from the group consisting of phenyl, oxadiazolyl, thiadiazolyl, thiazolyl, isothiazoyl, imidazolyl, oxazolyl, isoxazolyl, pyridyl, pyrimidyl, benzothiazolyl, benzoisothiazolyl, benzoxazolyl, benzoisoxazolyl, benzoimidazolyl, and quinolyl, each optionally substituted with one or more R¹¹. In some such embodiments, R² is optionally substituted phenyl. In some other embodiments, R² is optionally substituted pyridyl, for example,

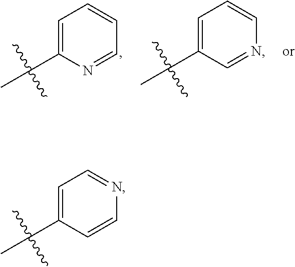 [SMILES: *c1ccccn1 ; *c1cccnc1 ; "or" ; *c1ccncc1]

each optionally substituted. In some other embodiments, R² is optionally substituted pyrimidyl, for example

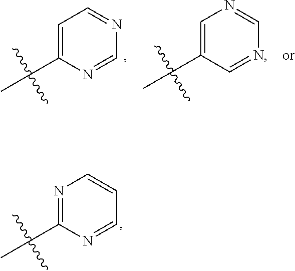 [SMILES: *c1ccncn1 ; *c1cncnc1 ; "or" ; *c1ncccn1]

each optionally substituted. In some embodiments, R² is optionally substituted pyridazinyl, for example,

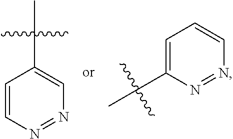 [SMILES: *c1ccnnc1 ; *c1cccnn1]

each optionally substituted. In some other embodiments, R² is optionally substituted quinolyl, for example optionally substituted

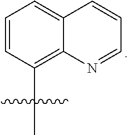 [SMILES: *c1cccc2cccnc12]

In some other embodiments, R² is oxadiazolyl or thiadiazolyl, for example,

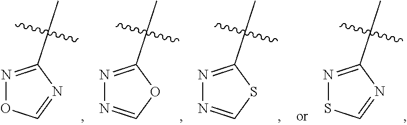 [SMILES: *c1ncon1 ; *c1nnco1 ; *c1nncs1 ; *c1ncsn1]

each optionally substituted. In some other embodiments, R² is thiazolyl or oxazolyl, for example

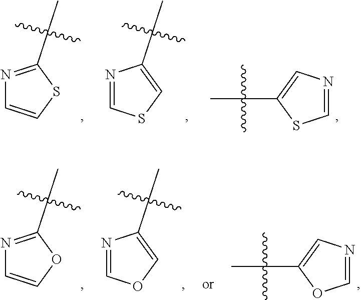 [SMILES: *c1nccs1 ; *c1cscn1 ; *c1cncs1 ; *c1ncco1 ; *c1cocn1 ; *c1cnco1]

each optionally substituted. In some such embodiments, R² is optionally substituted benzothiazolyl, for example

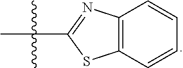 [SMILES: *c1nc2ccccc2s1]

In some embodiments, R² is unsubstituted. In some other embodiments, R² is substituted with one or more R¹¹, where R¹¹ is selected from the group consisting of halo, —OH, —CN, C₁₋₆ alkyl, C₂₋₆ alkynyl, C₁₋₆ haloalkyl, C₁₋₆ haloalkoxy, C₁₋₆ alkoxy, —NR^{5a}R—, —C(O)OR^{7b}, —C(O)NR^{5b}R^{6b}, 5 membered heteroaryl, 6 membered heteroaryl, 9 membered heteroaryl, and 6 membered heterocyclyl, wherein each of the C₁₋₆ alkyl, C₂₋₆ alkynyl, 5 membered heteroaryl, 6 membered heteroaryl, 9 membered heteroaryl, and 6 membered heterocyclyl is optionally substituted with one or more R¹³.

In some such embodiments, R¹¹ is 5, 6 or 9 membered heteroaryl, or 6 membered heterocyclyl, each optionally substituted with one or more R¹³. In some such embodiments, R¹¹ is optionally substituted imidazolyl, for example

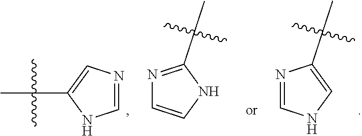

In some embodiments, R¹¹ is optionally substituted triazolyl, for example,

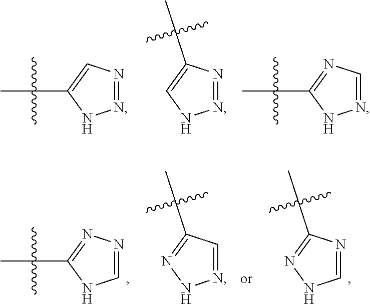 [SMILES: *c1cnn[nH]1 ; *c1c[nH]nn1 ; *c1ncn[nH]1 ; *c1nnc[nH]1 ; *c1cn[nH]n1 ; *c1nc[nH]n1]

each optionally substituted. In some such embodiments, R¹¹ is optionally substituted oxadiazolyl or thiadiazolyl, for example,

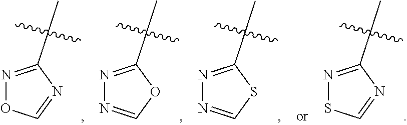 [SMILES: *c1ncon1 ; *c1nnco1 ; *c1nncs1 ; *c1ncsn1]

In some such embodiments, R¹¹ is optionally substituted thiazolyl, for example,

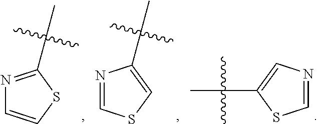

In some such embodiments, R¹¹ is optionally substituted pyridyl, for example

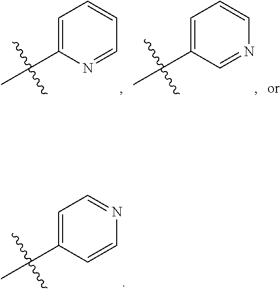 [SMILES: *c1ccccn1 ; *c1cccnc1 ; *c1ccncc1]

In some such embodiments, R¹¹ is optionally substituted benzoimidazolyl or indolyl, for example

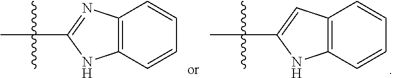 [SMILES: *c1nc2ccccc2[nH]1 ; *c1cc2ccccc2[nH]1]

In some other embodiments, R¹¹ is optionally substituted 6 membered heterocyclyl, for example morpholinyl, piperazinyl or piperidyl, such as

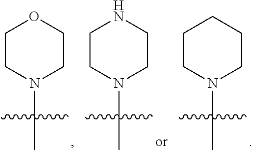

In some such embodiments, R¹³ is selected from the group consisting of C₁₋₆ alkyl, (C₁₋₆ alkoxy)C₁₋₆ alkyl, optionally substituted pyridyl, optionally substituted phenyl, optionally substituted cyclopropyl, SEM, and —(C₀₋₆ alkylene)NR^{5a}R^{6a}. The pyridyl or phenyl may be substituted with one or more substituents as defined herein, including but not limited to C₁₋₆ alkyl, C₁₋₆ haloalkyl, C₁₋₆ alkoxy, C₁₋₆ haloalkoxy, (C₁₋₆ alkoxy)C₁₋₆ alkyl, halo, or —CN. In some further embodiments, R¹¹ is a triazolyl substituted with —(C₀₋₆ alkylene)NR^{5a}R^{6a}, for example, —(CH₂)₁₋₃ NR^{5a}R^{6a}, wherein R^{5a} and R^{6a} are independently H or C₁₋₆ alkyl, or wherein R^{5a} and R^{6a} together with the nitrogen atom to which they are attached form an optionally substituted 5 or 6 membered heterocyclyl, for example, pyrrolidyl, piperazinyl, piperidyl, or morpholinyl. The 5 or 6 membered heterocyclyl may be substituted with one or more substituents as defined herein, including but not limited to C₁₋₆ alkyl, C₁₋₆ haloalkyl, C₁₋₆ alkoxy, C₁₋₆ haloalkoxy, (C₁₋₆ alkoxy)C₁₋₆ alkyl, halo, oxo, or —CN. In one embodiment, R¹¹ is piperizinyl, optionally substituted with (C₁₋₆ alkoxy)C₁₋₆ alkyl, for example, —(CH₂)₂OCH₃.

In some other embodiments, R¹¹ is C₁₋₆ alkyl, for example, methyl.

In some other embodiments, R¹¹ is C₂₋₆ alkynyl, for example, ethynyl.

In some other embodiments, R¹¹ is —NR^{5a}R^{6a}. In one embodiment, both R^{5a} and R^{6a} are H. In another embodiment, one of R^{5a} and R^{6a} is H. In another embodiment, R^{5a} and R^{6a} together with the nitrogen atom to which they are attached form an optionally substituted 5 or 6 membered heterocyclyl. In some such embodiments, the 5 membered heterocyclyl is pyrrolidyl. In some such embodiment, the 6 membered heterocyclyl is selected from the group consisting of piperazinyl, piperidyl, and morpholinyl. The 5 or 6 membered heterocyclyl may be substituted with one or more substituents as defined herein, including but not limited to C₁₋₆ alkyl, C₁₋₆ haloalkyl, C₁₋₆ alkoxy, C₁₋₆ haloalkoxy, (C₁₋₆ alkoxy)C₁₋₆ alkyl, halo, oxo, or —CN. In one embodiment, R¹¹ is piperizinyl, optionally substituted with (C₁₋₆ alkoxy)C₁₋₆ alkyl, for example, —(CH₂)₂OCH₃.

In some other embodiments, R¹¹ is —C(O)OR^{7b}, and wherein R^{7b} is H or C₁₋₆ alkyl.

In some other embodiments, R₁₁ is —C(O)NR^{5b}R^{6b}. In one embodiment, both R^{5a} and R^{6a} are H. In another embodiment, at least one of R^{5b} and R^{6b} is H, for example, R^{5b} is H, and R^{6b} is optionally substituted (C₀₋₆ alkylene) 6 membered heterocyclyl. In another embodiment, R^{5b} and R^{6b} together with the nitrogen atom to which they are attached form an optionally substituted 6 membered heterocyclyl. In some such embodiment, the 6 membered heterocyclyl is selected from the group consisting of piperazinyl, piperidyl, and morpholinyl. The 6 membered heterocyclyl may be substituted with one or more substituents as defined herein, including but not limited to C₁₋₆ alkyl, C₁₋₆ haloalkyl, C₁₋₆ alkoxy, C₁₋₆ haloalkoxy, (C₁₋₆ alkoxy)C₁₋₆ alkyl, halo, oxo, or —CN.

In some other embodiments of the compounds of any one of Formula (I), (II), (II-A) through (II-D), (III-A) through (III-D), (IV-A) through (IV-D), (V), and (V-A) through (V-D), R¹ and R² together with the nitrogen atom to which they are attached form a 6 membered heterocyclyl optionally substituted with one or more R¹². In some such embodiments, R¹² is C₂₋₆ alkynyl optionally substituted with one or more R¹³. In another embodiment, two geminal R¹² form oxo. In some such embodiments, R¹³ is selected from the group consisting of pyridyl, phenyl, and cyclopropyl, each may be optionally substituted with one or more substituents as defined herein, including but not limited to C₁₋₆ alkyl, C₁₋₆ haloalkyl, C₁₋₆ alkoxy, C₁₋₆ haloalkoxy, (C₁₋₆ alkoxy)C₁₋₆ alkyl, halo, oxo, or —CN.

Additional embodiments of the compounds described herein are illustrated in Table 1, or pharmaceutically acceptable salts thereof. In some embodiments, the compounds of Formula (I) are selected from the group consisting of Compounds 1-125 of Table 1, and pharmaceutically acceptable salts thereof.

TABLE 1
Exemplary Compounds of Formula (I)

| NO | Structure |
|---|---|
| 1 | 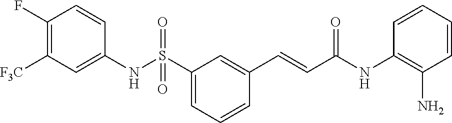 [SMILES: Nc1ccccc1NC(=O)/C=C/c1cccc(S(=O)(=O)Nc2ccc(F)c(C(F)(F)F)c2)c1] |
| 2 | 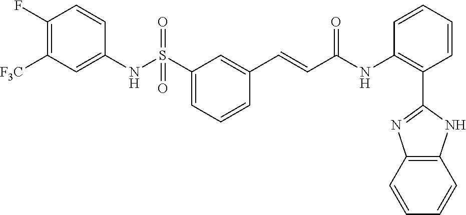 [SMILES: O=C(/C=C/c1cccc(S(=O)(=O)Nc2ccc(F)c(C(F)(F)F)c2)c1)Nc1ccccc1-c1nc2ccccc2[nH]1] |
| 3 | 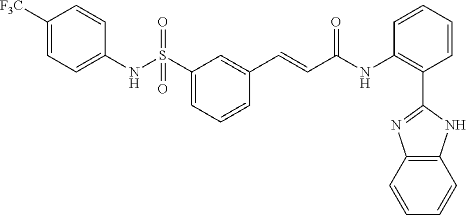 [SMILES: O=C(/C=C/c1cccc(S(=O)(=O)Nc2ccc(C(F)(F)F)cc2)c1)Nc1ccccc1-c1nc2ccccc2[nH]1] |
| 4 | 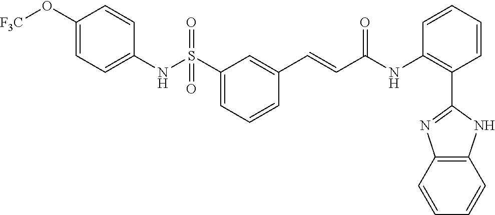 [SMILES: O=C(/C=C/c1cccc(S(=O)(=O)Nc2ccc(OC(F)(F)F)cc2)c1)Nc1ccccc1-c1nc2ccccc2[nH]1] |
| 5 | 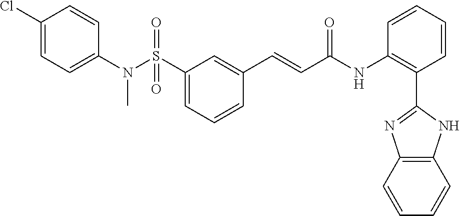 [SMILES: CN(c1ccc(Cl)cc1)S(=O)(=O)c1cccc(/C=C/C(=O)Nc2ccccc2-c2nc3ccccc3[nH]2)c1] |
| 6 | 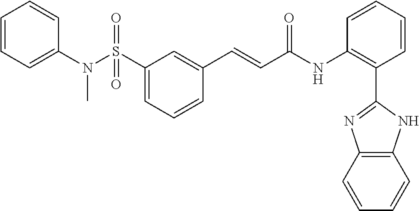 [SMILES: CN(c1ccccc1)S(=O)(=O)c1cccc(/C=C/C(=O)Nc2ccccc2-c2nc3ccccc3[nH]2)c1] |
| 7 | 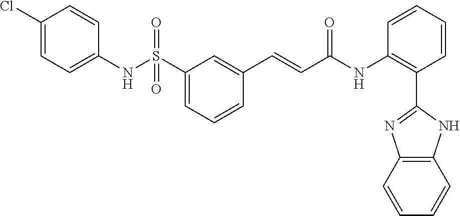 [SMILES: O=C(/C=C/c1cccc(S(=O)(=O)Nc2ccc(Cl)cc2)c1)Nc1ccccc1-c1nc2ccccc2[nH]1] |
| 8 | 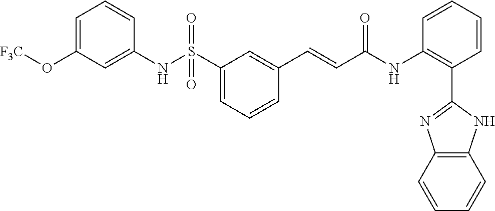 [SMILES: O=C(/C=C/c1cccc(S(=O)(=O)Nc2cccc(OC(F)(F)F)c2)c1)Nc1ccccc1-c1nc2ccccc2[nH]1] |
| 9 | 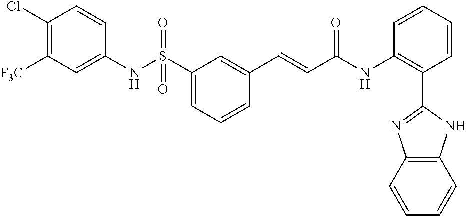 [SMILES: O=C(/C=C/c1cccc(S(=O)(=O)Nc2ccc(Cl)c(C(F)(F)F)c2)c1)Nc1ccccc1-c1nc2ccccc2[nH]1] |
| 10 | 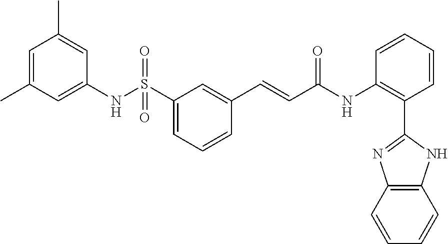 [SMILES: Cc1cc(C)cc(NS(=O)(=O)c2cccc(/C=C/C(=O)Nc3ccccc3-c3nc4ccccc4[nH]3)c2)c1] |
| 11 | 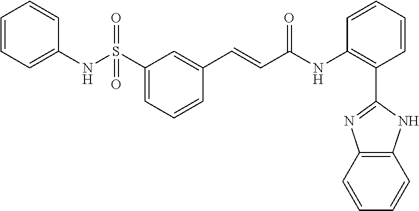 [SMILES: O=C(/C=C/c1cccc(S(=O)(=O)Nc2ccccc2)c1)Nc1ccccc1-c1nc2ccccc2[nH]1] |
| 12 | 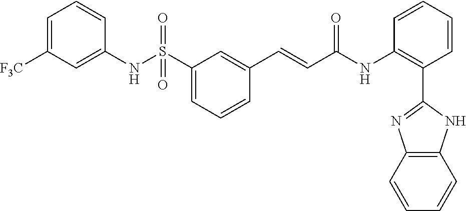 [SMILES: O=C(/C=C/c1cccc(S(=O)(=O)Nc2cccc(C(F)(F)F)c2)c1)Nc1ccccc1-c1nc2ccccc2[nH]1] |
| 13 | 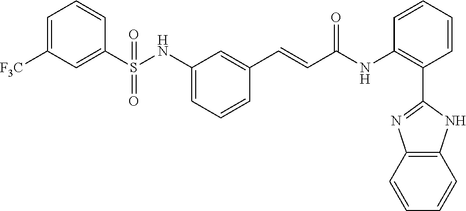 [SMILES: O=C(/C=C/c1cccc(NS(=O)(=O)c2cccc(C(F)(F)F)c2)c1)Nc1ccccc1-c1nc2ccccc2[nH]1] |
| 14 | 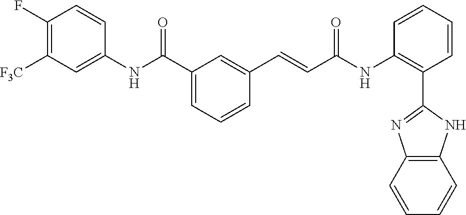 [SMILES: O=C(/C=C/c1cccc(C(=O)Nc2ccc(F)c(C(F)(F)F)c2)c1)Nc1ccccc1-c1nc2ccccc2[nH]1] |
| 15 | 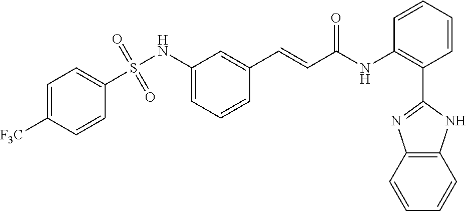 [SMILES: O=C(/C=C/c1cccc(NS(=O)(=O)c2ccc(C(F)(F)F)cc2)c1)Nc1ccccc1-c1nc2ccccc2[nH]1] |
| 16 | 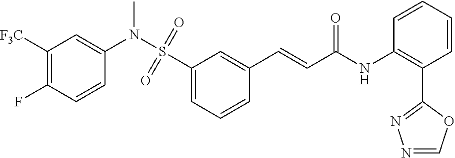 [SMILES: CN(c1ccc(F)c(C(F)(F)F)c1)S(=O)(=O)c1cccc(/C=C/C(=O)Nc2ccccc2-c2nnco2)c1] |
| 17 | 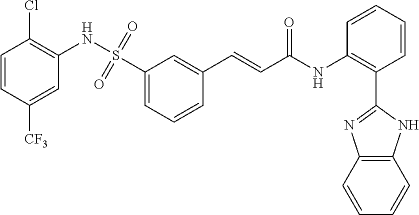 [SMILES: O=C(/C=C/c1cccc(S(=O)(=O)Nc2cc(C(F)(F)F)ccc2Cl)c1)Nc1ccccc1-c1nc2ccccc2[nH]1] |
| 18 | 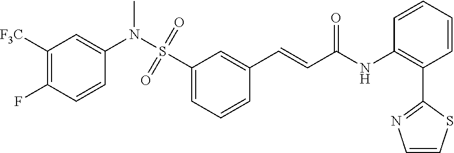 [SMILES: CN(c1ccc(F)c(C(F)(F)F)c1)S(=O)(=O)c1cccc(/C=C/C(=O)Nc2ccccc2-c2nccs2)c1] |
| 19 | 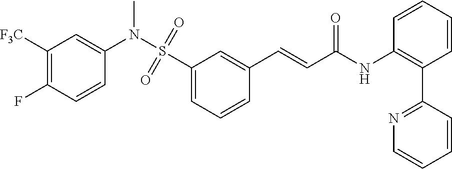 [SMILES: CN(c1ccc(F)c(C(F)(F)F)c1)S(=O)(=O)c1cccc(/C=C/C(=O)Nc2ccccc2-c2ccccn2)c1] |
| 20 | 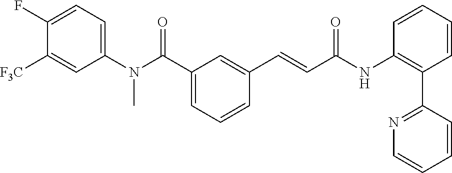 [SMILES: CN(C(=O)c1cccc(/C=C/C(=O)Nc2ccccc2-c2ccccn2)c1)c1ccc(F)c(C(F)(F)F)c1] |
| 21 | 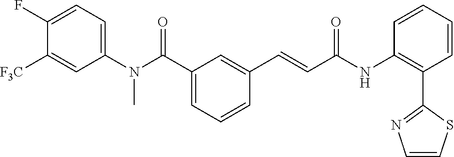 [SMILES: CN(C(=O)c1cccc(/C=C/C(=O)Nc2ccccc2-c2nccs2)c1)c1ccc(F)c(C(F)(F)F)c1] |
| 22 | 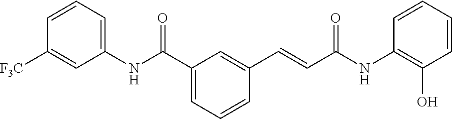 [SMILES: O=C(/C=C/c1cccc(C(=O)Nc2cccc(C(F)(F)F)c2)c1)Nc1ccccc1O] |
| 23 | 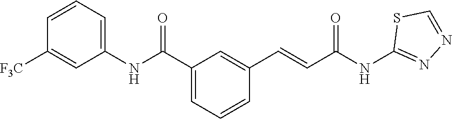 [SMILES: O=C(/C=C/c1cccc(C(=O)Nc2cccc(C(F)(F)F)c2)c1)Nc1nncs1] |
| 24 | 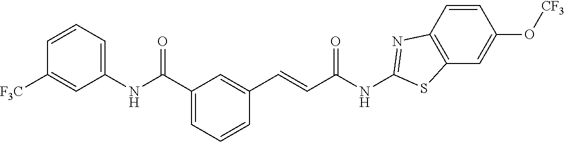 [SMILES: O=C(/C=C/c1cccc(C(=O)Nc2cccc(C(F)(F)F)c2)c1)Nc1nc2ccc(OC(F)(F)F)cc2s1] |
| 25 | 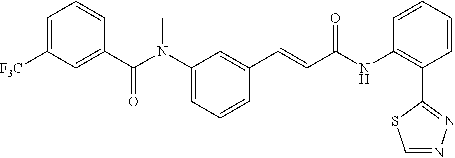 [SMILES: CN(C(=O)c1cccc(C(F)(F)F)c1)c1cccc(/C=C/C(=O)Nc2ccccc2-c2nncs2)c1] |
| 26 | 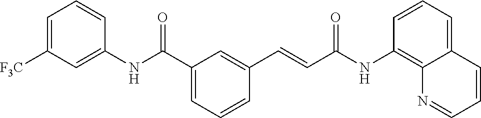 [SMILES: O=C(/C=C/c1cccc(C(=O)Nc2cccc(C(F)(F)F)c2)c1)Nc1cccc2cccnc12] |
| 27 | 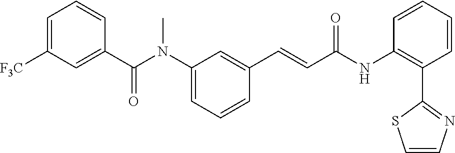 [SMILES: CN(C(=O)c1cccc(C(F)(F)F)c1)c1cccc(/C=C/C(=O)Nc2ccccc2-c2nccs2)c1] |
| 28 | 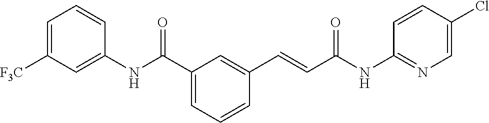 [SMILES: O=C(/C=C/c1cccc(C(=O)Nc2cccc(C(F)(F)F)c2)c1)Nc1ccc(Cl)cn1] |
| 29 | 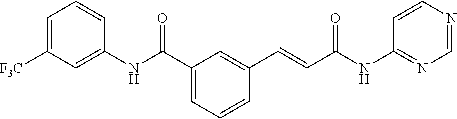 [SMILES: O=C(/C=C/c1cccc(C(=O)Nc2cccc(C(F)(F)F)c2)c1)Nc1ccncn1] |
| 30 | 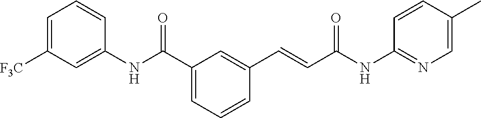 [SMILES: Cc1ccc(NC(=O)/C=C/c2cccc(C(=O)Nc3cccc(C(F)(F)F)c3)c2)nc1] |
| 31 | 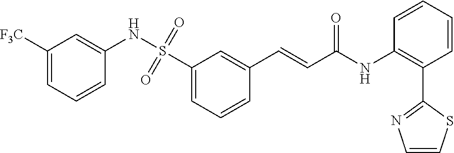 [SMILES: O=C(/C=C/c1cccc(S(=O)(=O)Nc2cccc(C(F)(F)F)c2)c1)Nc1ccccc1-c1nccs1] |
| 32 | 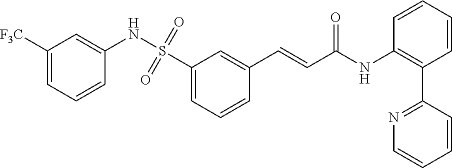 [SMILES: O=C(/C=C/c1cccc(S(=O)(=O)Nc2cccc(C(F)(F)F)c2)c1)Nc1ccccc1-c1ccccn1] |
| 33 | 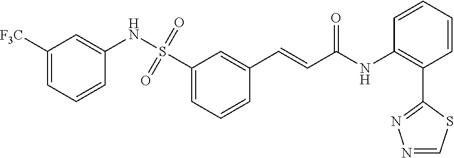 [SMILES: O=C(/C=C/c1cccc(S(=O)(=O)Nc2cccc(C(F)(F)F)c2)c1)Nc1ccccc1-c1nncs1] |
| 34 | 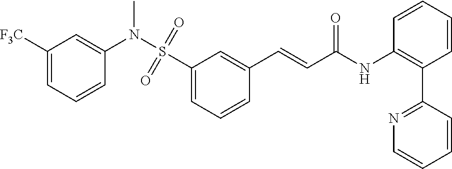 [SMILES: CN(c1cccc(C(F)(F)F)c1)S(=O)(=O)c1cccc(/C=C/C(=O)Nc2ccccc2-c2ccccn2)c1] |
| 35 | 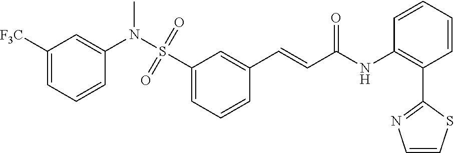 [SMILES: CN(c1cccc(C(F)(F)F)c1)S(=O)(=O)c1cccc(/C=C/C(=O)Nc2ccccc2-c2nccs2)c1] |
| 36 | 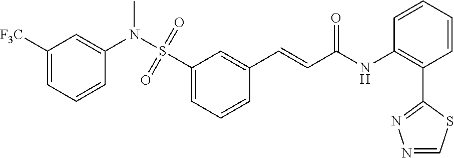 [SMILES: CN(c1cccc(C(F)(F)F)c1)S(=O)(=O)c1cccc(/C=C/C(=O)Nc2ccccc2-c2nncs2)c1] |
| 37 | 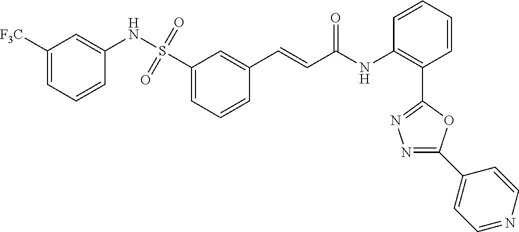 [SMILES: O=C(/C=C/c1cccc(S(=O)(=O)Nc2cccc(C(F)(F)F)c2)c1)Nc1ccccc1-c1nnc(-c2ccncc2)o1] |
| 38 | 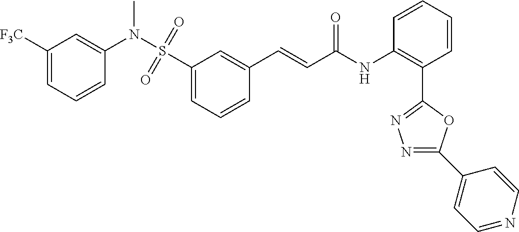 [SMILES: CN(c1cccc(C(F)(F)F)c1)S(=O)(=O)c1cccc(/C=C/C(=O)Nc2ccccc2-c2nnc(-c3ccncc3)o2)c1] |
| 39 | 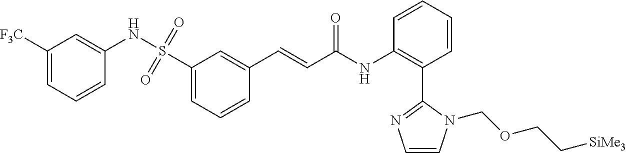 [SMILES: C[Si](C)(C)CCOCn1ccnc1-c1ccccc1NC(=O)/C=C/c1cccc(S(=O)(=O)Nc2cccc(C(F)(F)F)c2)c1] |
| 40 | 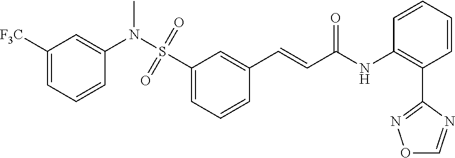 [SMILES: CN(c1cccc(C(F)(F)F)c1)S(=O)(=O)c1cccc(/C=C/C(=O)Nc2ccccc2-c2ncon2)c1] |
| 41 | 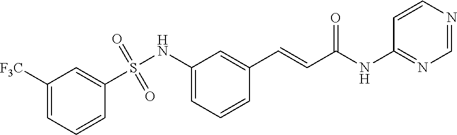 [SMILES: O=C(/C=C/c1cccc(NS(=O)(=O)c2cccc(C(F)(F)F)c2)c1)Nc1ccncn1] |
| 42 | 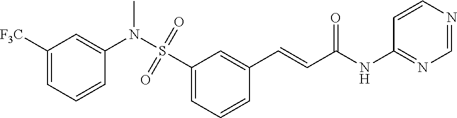 [SMILES: CN(c1cccc(C(F)(F)F)c1)S(=O)(=O)c1cccc(/C=C/C(=O)Nc2ccncn2)c1] |
| 43 | 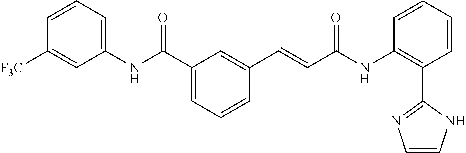 [SMILES: O=C(/C=C/c1cccc(C(=O)Nc2cccc(C(F)(F)F)c2)c1)Nc1ccccc1-c1ncc[nH]1] |
| 44 | 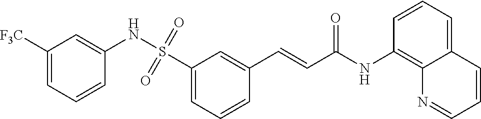 [SMILES: O=C(/C=C/c1cccc(S(=O)(=O)Nc2cccc(C(F)(F)F)c2)c1)Nc1cccc2cccnc12] |
| 45 | 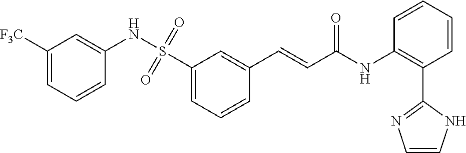 [SMILES: O=C(/C=C/c1cccc(S(=O)(=O)Nc2cccc(C(F)(F)F)c2)c1)Nc1ccccc1-c1ncc[nH]1] |
| 46 | 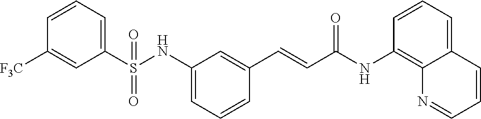 [SMILES: O=C(/C=C/c1cccc(NS(=O)(=O)c2cccc(C(F)(F)F)c2)c1)Nc1cccc2cccnc12] |
| 47 | 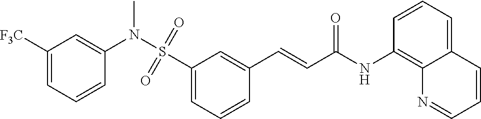 [SMILES: CN(c1cccc(C(F)(F)F)c1)S(=O)(=O)c1cccc(/C=C/C(=O)Nc2cccc3cccnc23)c1] |
| 48 | 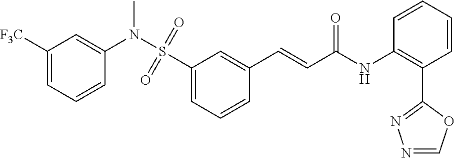 [SMILES: CN(c1cccc(C(F)(F)F)c1)S(=O)(=O)c1cccc(/C=C/C(=O)Nc2ccccc2-c2nnco2)c1] |
| 49 | 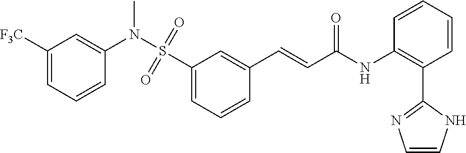 [SMILES: CN(c1cccc(C(F)(F)F)c1)S(=O)(=O)c1cccc(/C=C/C(=O)Nc2ccccc2-c2ncc[nH]2)c1] |
| 50 | 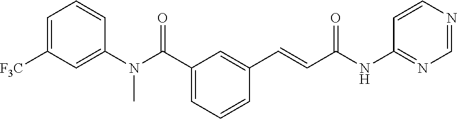 [SMILES: CN(C(=O)c1cccc(/C=C/C(=O)Nc2ccncn2)c1)c1cccc(C(F)(F)F)c1] |
| 51 | 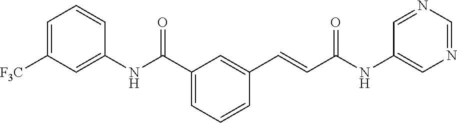 [SMILES: O=C(/C=C/c1cccc(C(=O)Nc2cccc(C(F)(F)F)c2)c1)Nc1cncnc1] |
| 52 | 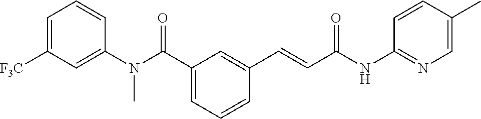 [SMILES: Cc1ccc(NC(=O)/C=C/c2cccc(C(=O)N(C)c3cccc(C(F)(F)F)c3)c2)nc1] |
| 53 | 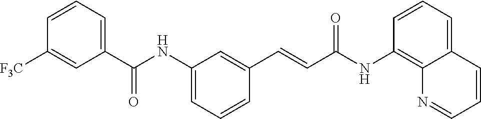 [SMILES: O=C(/C=C/c1cccc(NC(=O)c2cccc(C(F)(F)F)c2)c1)Nc1cccc2cccnc12] |
| 54 | 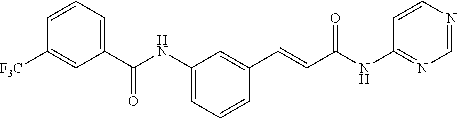 [SMILES: O=C(/C=C/c1cccc(NC(=O)c2cccc(C(F)(F)F)c2)c1)Nc1ccncn1] |
| 55 | 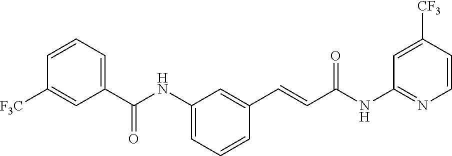 [SMILES: O=C(/C=C/c1cccc(NC(=O)c2cccc(C(F)(F)F)c2)c1)Nc1cc(C(F)(F)F)ccn1] |
| 56 | 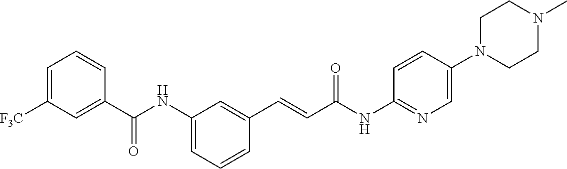 [SMILES: CN1CCN(c2ccc(NC(=O)/C=C/c3cccc(NC(=O)c4cccc(C(F)(F)F)c4)c3)nc2)CC1] |
| 57 | 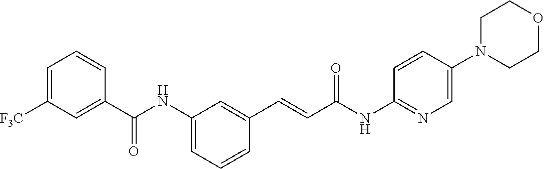 [SMILES: O=C(/C=C/c1cccc(NC(=O)c2cccc(C(F)(F)F)c2)c1)Nc1ccc(N2CCOCC2)cn1] |
| 58 | 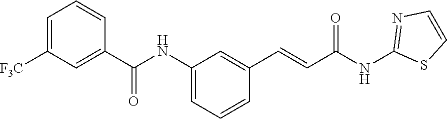 [SMILES: O=C(/C=C/c1cccc(NC(=O)c2cccc(C(F)(F)F)c2)c1)Nc1nccs1] |
| 59 | 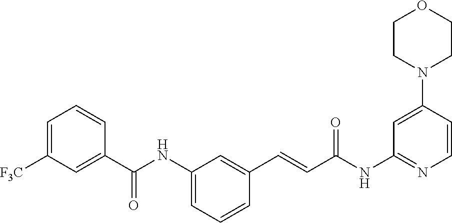 [SMILES: O=C(/C=C/c1cccc(NC(=O)c2cccc(C(F)(F)F)c2)c1)Nc1cc(N2CCOCC2)ccn1] |
| 60 | 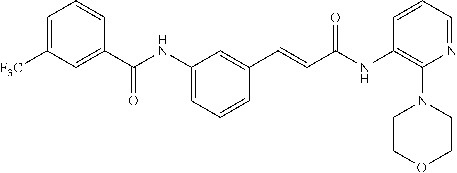 [SMILES: O=C(/C=C/c1cccc(NC(=O)c2cccc(C(F)(F)F)c2)c1)Nc1cccnc1N1CCOCC1] |
| 61 | 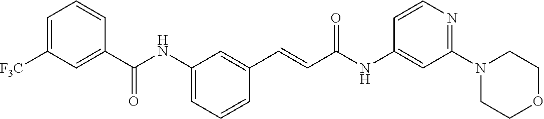 [SMILES: O=C(/C=C/c1cccc(NC(=O)c2cccc(C(F)(F)F)c2)c1)Nc1ccnc(N2CCOCC2)c1] |
| 62 | 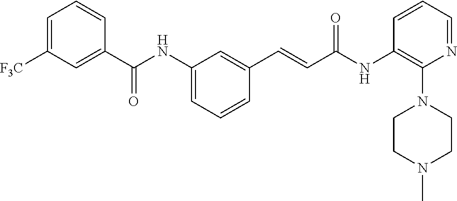 [SMILES: CN1CCN(c2ncccc2NC(=O)/C=C/c2cccc(NC(=O)c3cccc(C(F)(F)F)c3)c2)CC1] |
| 63 | 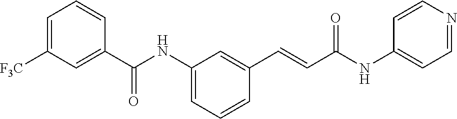 [SMILES: O=C(/C=C/c1cccc(NC(=O)c2cccc(C(F)(F)F)c2)c1)Nc1ccncc1] |
| 64 | 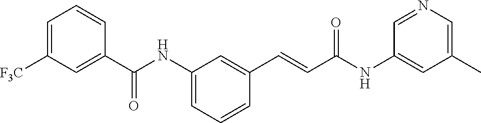 [SMILES: Cc1cncc(NC(=O)/C=C/c2cccc(NC(=O)c3cccc(C(F)(F)F)c3)c2)c1] |
| 65 | 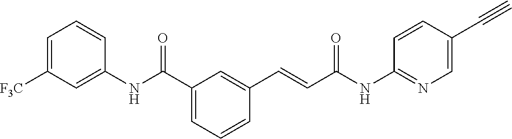 [SMILES: C#Cc1ccc(NC(=O)/C=C/c2cccc(C(=O)Nc3cccc(C(F)(F)F)c3)c2)nc1] |
| 66 | 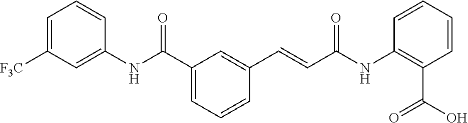 [SMILES: O=C(/C=C/c1cccc(C(=O)Nc2cccc(C(F)(F)F)c2)c1)Nc1ccccc1C(=O)O] |
| 67 | 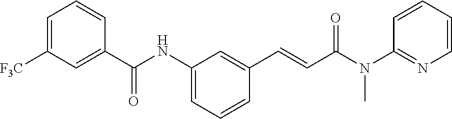 [SMILES: CN(C(=O)/C=C/c1cccc(NC(=O)c2cccc(C(F)(F)F)c2)c1)c1ccccn1] |
| 68 | 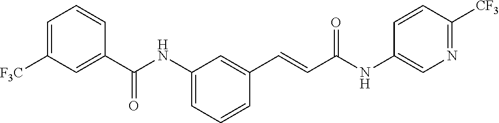 [SMILES: O=C(/C=C/c1cccc(NC(=O)c2cccc(C(F)(F)F)c2)c1)Nc1ccc(C(F)(F)F)nc1] |
| 69 | 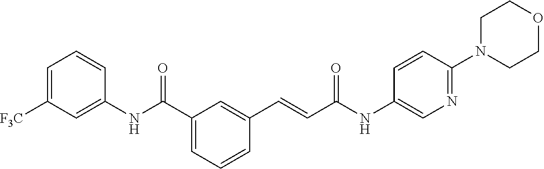 [SMILES: O=C(/C=C/c1cccc(C(=O)Nc2cccc(C(F)(F)F)c2)c1)Nc1ccc(N2CCOCC2)nc1] |
| 70 | 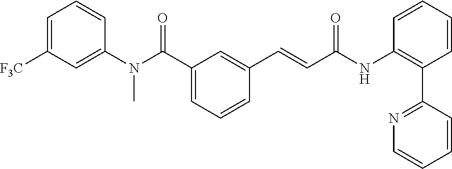 [SMILES: CN(C(=O)c1cccc(/C=C/C(=O)Nc2ccccc2-c2ccccn2)c1)c1cccc(C(F)(F)F)c1] |
| 71 | 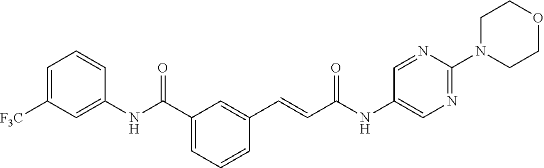 [SMILES: O=C(/C=C/c1cccc(C(=O)Nc2cccc(C(F)(F)F)c2)c1)Nc1cnc(N2CCOCC2)nc1] |
| 72 | 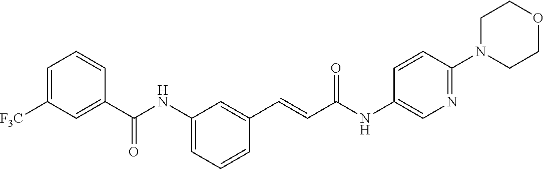 [SMILES: O=C(/C=C/c1cccc(NC(=O)c2cccc(C(F)(F)F)c2)c1)Nc1ccc(N2CCOCC2)nc1] |
| 73 | 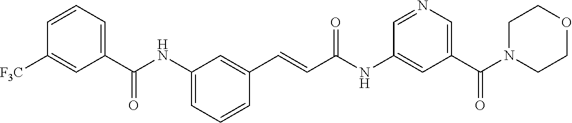 [SMILES: O=C(/C=C/c1cccc(NC(=O)c2cccc(C(F)(F)F)c2)c1)Nc1cncc(C(=O)N2CCOCC2)c1] |
| 74 | 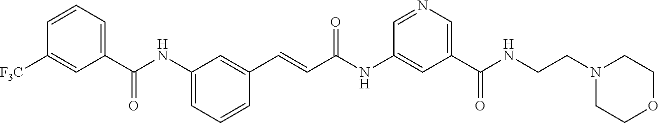 [SMILES: O=C(/C=C/c1cccc(NC(=O)c2cccc(C(F)(F)F)c2)c1)Nc1cncc(C(=O)NCCN2CCOCC2)c1] |
| 75 | 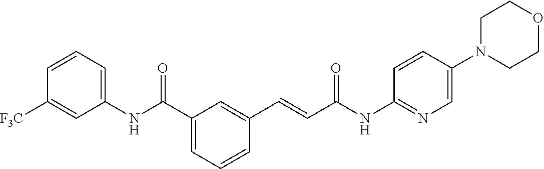 [SMILES: O=C(/C=C/c1cccc(C(=O)Nc2cccc(C(F)(F)F)c2)c1)Nc1ccc(N2CCOCC2)cn1] |
| 76 | 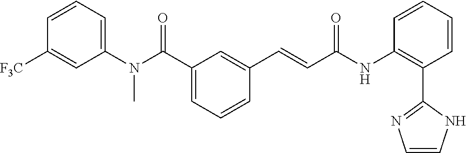 [SMILES: CN(C(=O)c1cccc(/C=C/C(=O)Nc2ccccc2-c2ncc[nH]2)c1)c1cccc(C(F)(F)F)c1] |
| 77 | 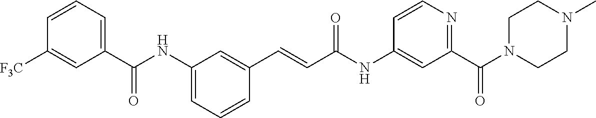 [SMILES: CN1CCN(C(=O)c2cc(NC(=O)/C=C/c3cccc(NC(=O)c4cccc(C(F)(F)F)c4)c3)ccn2)CC1] |
| 78 | 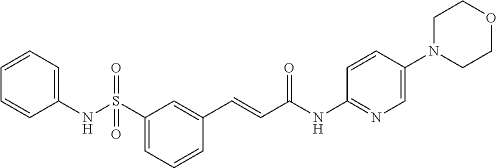 [SMILES: O=C(/C=C/c1cccc(S(=O)(=O)Nc2ccccc2)c1)Nc1ccc(N2CCOCC2)cn1] |
| 79 | 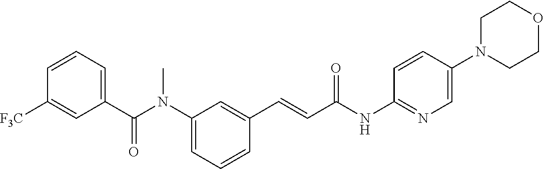 [SMILES: CN(C(=O)c1cccc(C(F)(F)F)c1)c1cccc(/C=C/C(=O)Nc2ccc(N3CCOCC3)cn2)c1] |
| 80 | 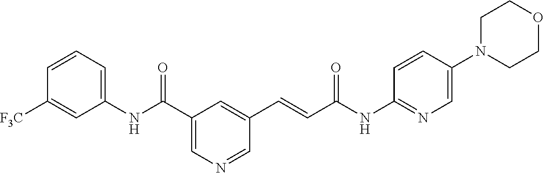 [SMILES: O=C(/C=C/c1cncc(C(=O)Nc2cccc(C(F)(F)F)c2)c1)Nc1ccc(N2CCOCC2)cn1] |
| 81 | 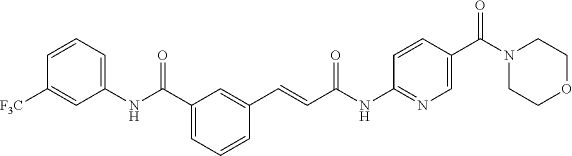 [SMILES: O=C(/C=C/c1cccc(C(=O)Nc2cccc(C(F)(F)F)c2)c1)Nc1ccc(C(=O)N2CCOCC2)cn1] |
| 82 | 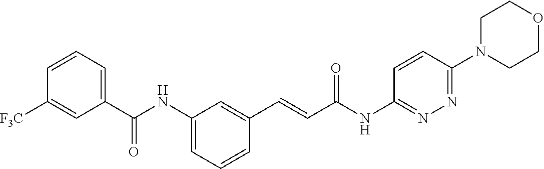 [SMILES: O=C(/C=C/c1cccc(NC(=O)c2cccc(C(F)(F)F)c2)c1)Nc1ccc(N2CCOCC2)nn1] |
| 83 | 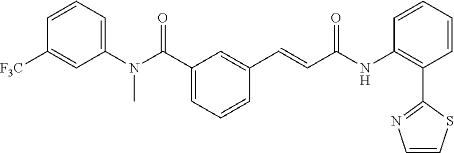 [SMILES: CN(C(=O)c1cccc(/C=C/C(=O)Nc2ccccc2-c2nccs2)c1)c1cccc(C(F)(F)F)c1] |
| 84 | 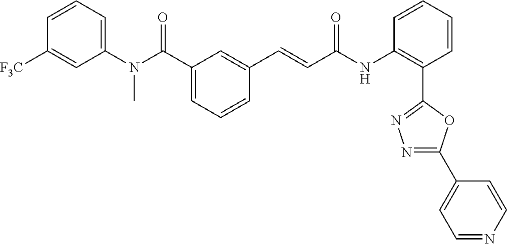 [SMILES: CN(C(=O)c1cccc(/C=C/C(=O)Nc2ccccc2-c2nnc(-c3ccncc3)o2)c1)c1cccc(C(F)(F)F)c1] |
| 85 | 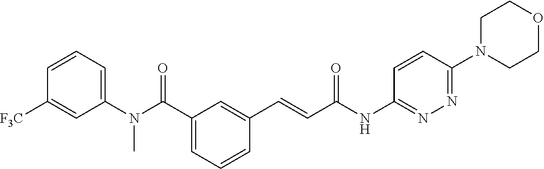 [SMILES: CN(C(=O)c1cccc(/C=C/C(=O)Nc2ccc(N3CCOCC3)nn2)c1)c1cccc(C(F)(F)F)c1] |
| 86 | 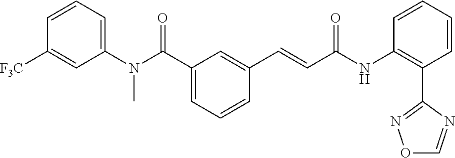 [SMILES: CN(C(=O)c1cccc(/C=C/C(=O)Nc2ccccc2-c2ncon2)c1)c1cccc(C(F)(F)F)c1] |
| 87 | 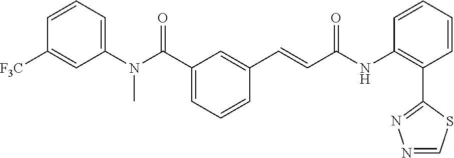 [SMILES: CN(C(=O)c1cccc(/C=C/C(=O)Nc2ccccc2-c2nncs2)c1)c1cccc(C(F)(F)F)c1] |
| 88 | 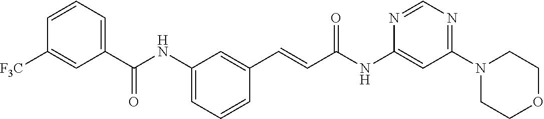 [SMILES: O=C(/C=C/c1cccc(NC(=O)c2cccc(C(F)(F)F)c2)c1)Nc1cc(N2CCOCC2)ncn1] |
| 89 | 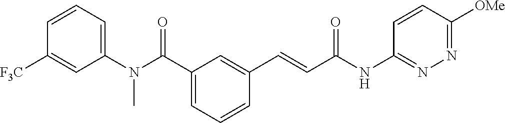 [SMILES: COc1ccc(NC(=O)/C=C/c2cccc(C(=O)N(C)c3cccc(C(F)(F)F)c3)c2)nn1] |
| 90 | 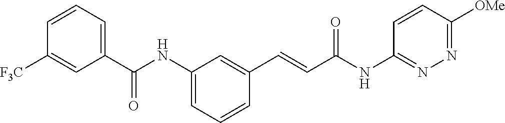 [SMILES: COc1ccc(NC(=O)/C=C/c2cccc(NC(=O)c3cccc(C(F)(F)F)c3)c2)nn1] |
| 91 | 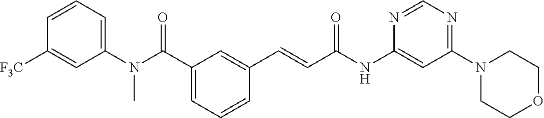 [SMILES: CN(C(=O)c1cccc(/C=C/C(=O)Nc2cc(N3CCOCC3)ncn2)c1)c1cccc(C(F)(F)F)c1] |
| 92 | 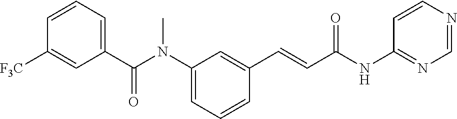 [SMILES: CN(C(=O)c1cccc(C(F)(F)F)c1)c1cccc(/C=C/C(=O)Nc2ccncn2)c1] |
| 93 | 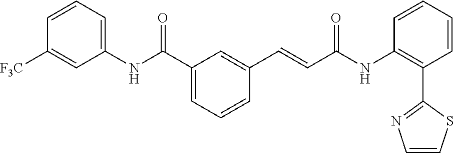 [SMILES: O=C(/C=C/c1cccc(C(=O)Nc2cccc(C(F)(F)F)c2)c1)Nc1ccccc1-c1nccs1] |
| 94 | 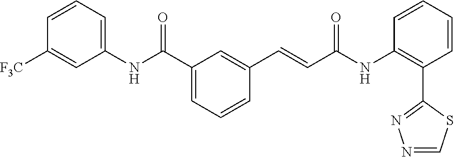 [SMILES: O=C(/C=C/c1cccc(C(=O)Nc2cccc(C(F)(F)F)c2)c1)Nc1ccccc1-c1nncs1] |
| 95 | 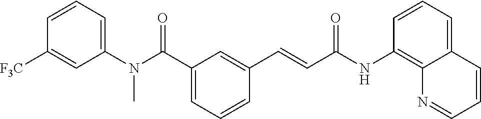 [SMILES: CN(C(=O)c1cccc(/C=C/C(=O)Nc2cccc3cccnc23)c1)c1cccc(C(F)(F)F)c1] |
| 96 | 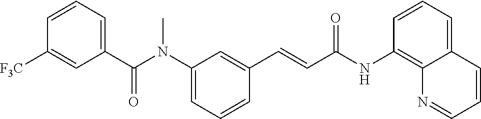 [SMILES: CN(C(=O)c1cccc(C(F)(F)F)c1)c1cccc(/C=C/C(=O)Nc2cccc3cccnc23)c1] |
| 97 | 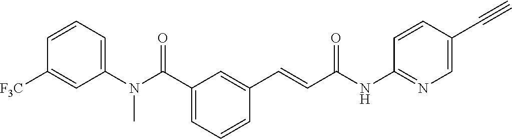 [SMILES: C#Cc1ccc(NC(=O)/C=C/c2cccc(C(=O)N(C)c3cccc(C(F)(F)F)c3)c2)nc1] |
| 98 | 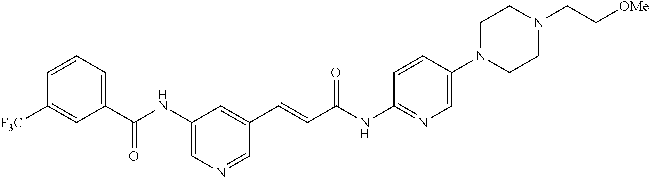 [SMILES: COCCN1CCN(c2ccc(NC(=O)/C=C/c3cncc(NC(=O)c4cccc(C(F)(F)F)c4)c3)nc2)CC1] |
| 99 | 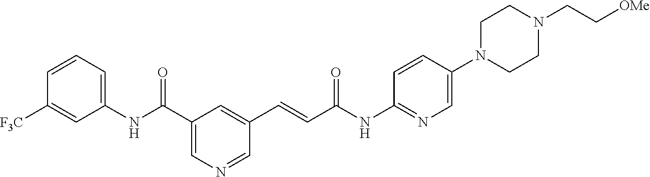 [SMILES: COCCN1CCN(c2ccc(NC(=O)/C=C/c3cncc(C(=O)Nc4cccc(C(F)(F)F)c4)c3)nc2)CC1] |
| 100 | 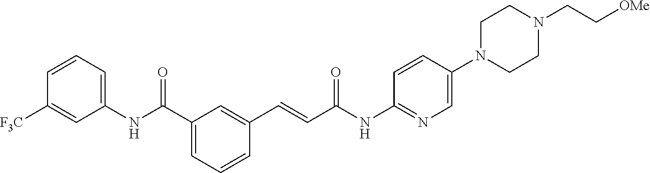 [SMILES: COCCN1CCN(c2ccc(NC(=O)/C=C/c3cccc(C(=O)Nc4cccc(C(F)(F)F)c4)c3)nc2)CC1] |
| 101 | 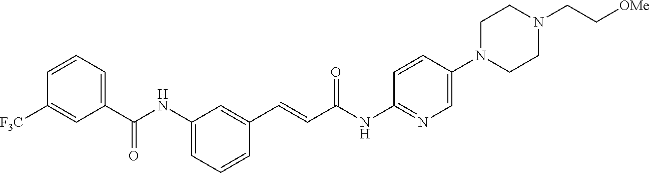 [SMILES: COCCN1CCN(c2ccc(NC(=O)/C=C/c3cccc(NC(=O)c4cccc(C(F)(F)F)c4)c3)nc2)CC1] |
| 102 | 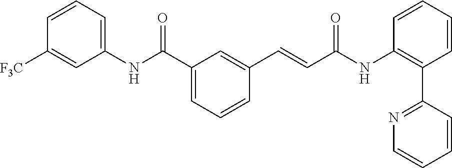 [SMILES: O=C(/C=C/c1cccc(C(=O)Nc2cccc(C(F)(F)F)c2)c1)Nc1ccccc1-c1ccccn1] |
| 103 | 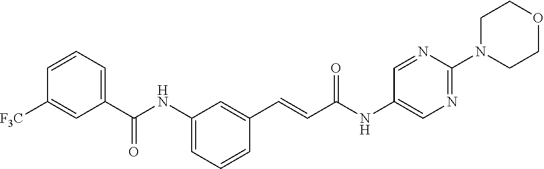 [SMILES: O=C(/C=C/c1cccc(NC(=O)c2cccc(C(F)(F)F)c2)c1)Nc1cnc(N2CCOCC2)nc1] |
| 104 | 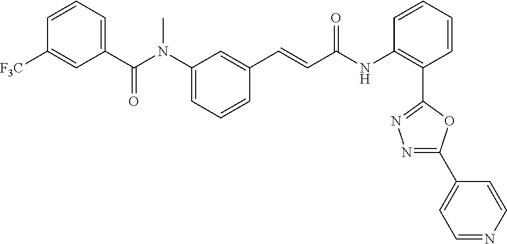 [SMILES: CN(C(=O)c1cccc(C(F)(F)F)c1)c1cccc(/C=C/C(=O)Nc2ccccc2-c2nnc(-c3ccncc3)o2)c1] |
| 105 | 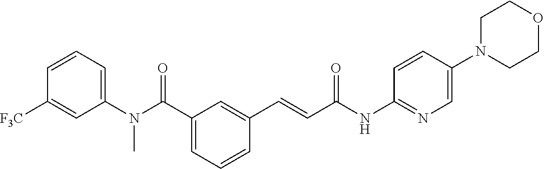 [SMILES: CN(C(=O)c1cccc(/C=C/C(=O)Nc2ccc(N3CCOCC3)cn2)c1)c1cccc(C(F)(F)F)c1] |
| 106 | 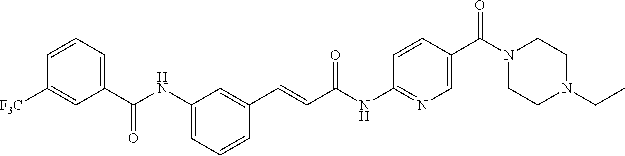 [SMILES: CCN1CCN(C(=O)c2ccc(NC(=O)/C=C/c3cccc(NC(=O)c4cccc(C(F)(F)F)c4)c3)nc2)CC1] |
| 107 | 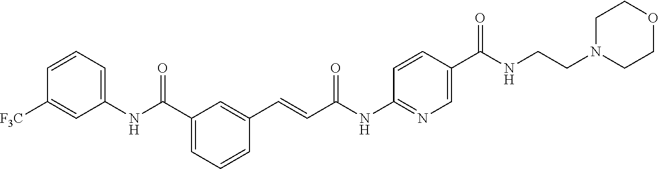 [SMILES: O=C(/C=C/c1cccc(C(=O)Nc2cccc(C(F)(F)F)c2)c1)Nc1ccc(C(=O)NCCN2CCOCC2)cn1] |
| 108 | 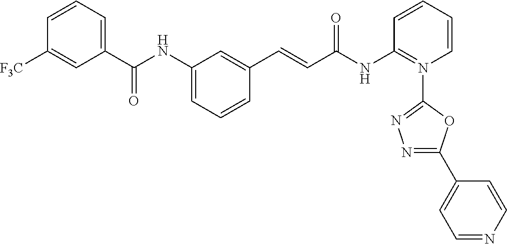 |
| 109 | 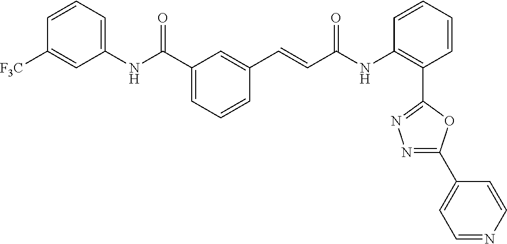 [SMILES: O=C(/C=C/c1cccc(C(=O)Nc2cccc(C(F)(F)F)c2)c1)Nc1ccccc1-c1nnc(-c2ccncc2)o1] |
| 110 | 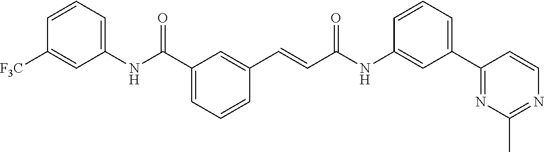 [SMILES: Cc1nccc(-c2cccc(NC(=O)/C=C/c3cccc(C(=O)Nc4cccc(C(F)(F)F)c4)c3)c2)n1] |
| 111 | 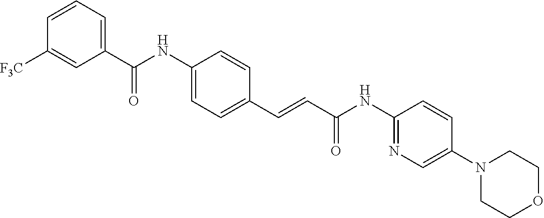 [SMILES: O=C(/C=C/c1ccc(NC(=O)c2cccc(C(F)(F)F)c2)cc1)Nc1ccc(N2CCOCC2)cn1] |
| 112 | 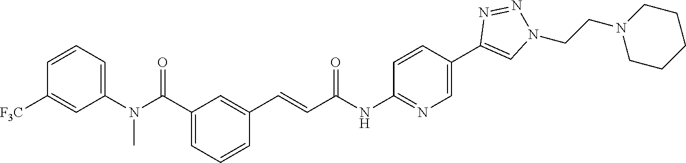 [SMILES: CN(C(=O)c1cccc(/C=C/C(=O)Nc2ccc(-c3cn(CCN4CCCCC4)nn3)cn2)c1)c1cccc(C(F)(F)F)c1] |
| 113 | 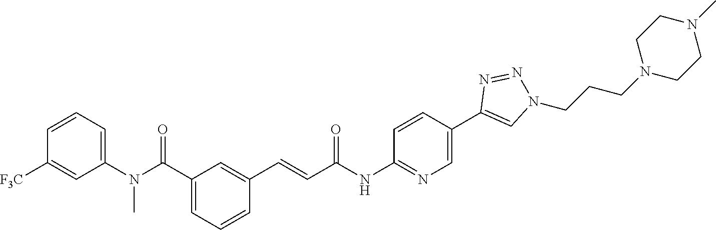 [SMILES: CN1CCN(CCCn2cc(-c3ccc(NC(=O)/C=C/c4cccc(C(=O)N(C)c5cccc(C(F)(F)F)c5)c4)nc3)nn2)CC1] |
| 114 | 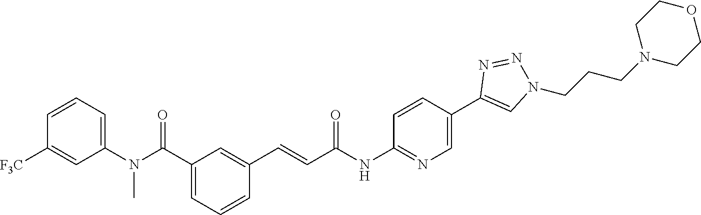 [SMILES: CN(C(=O)c1cccc(/C=C/C(=O)Nc2ccc(-c3cn(CCCN4CCOCC4)nn3)cn2)c1)c1cccc(C(F)(F)F)c1] |
| 115 | 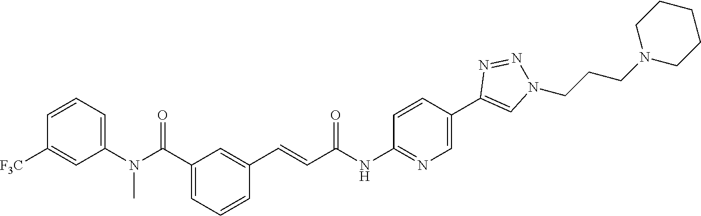 [SMILES: CN(C(=O)c1cccc(/C=C/C(=O)Nc2ccc(-c3cn(CCCN4CCCCC4)nn3)cn2)c1)c1cccc(C(F)(F)F)c1] |
| 116 | 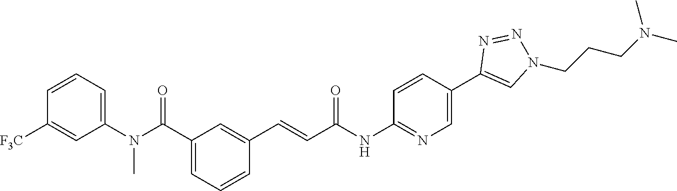 [SMILES: CN(C)CCCn1cc(-c2ccc(NC(=O)/C=C/c3cccc(C(=O)N(C)c4cccc(C(F)(F)F)c4)c3)nc2)nn1] |
| 117 | 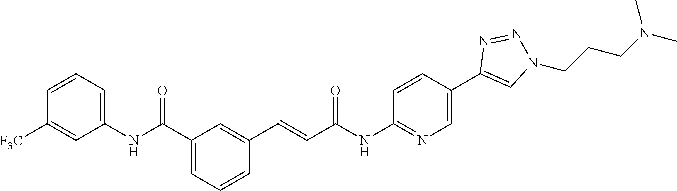 [SMILES: CN(C)CCCn1cc(-c2ccc(NC(=O)/C=C/c3cccc(C(=O)Nc4cccc(C(F)(F)F)c4)c3)nc2)nn1] |
| 118 | 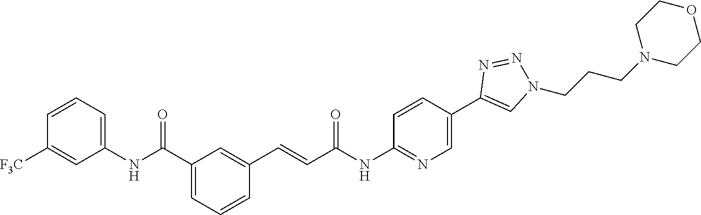 [SMILES: O=C(/C=C/c1cccc(C(=O)Nc2cccc(C(F)(F)F)c2)c1)Nc1ccc(-c2cn(CCCN3CCOCC3)nn2)cn1] |
| 119 | 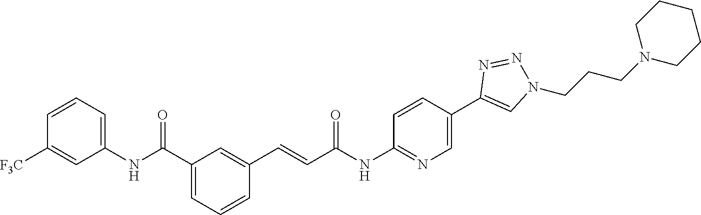 [SMILES: O=C(/C=C/c1cccc(C(=O)Nc2cccc(C(F)(F)F)c2)c1)Nc1ccc(-c2cn(CCCN3CCCCC3)nn2)cn1] |
| 120 | 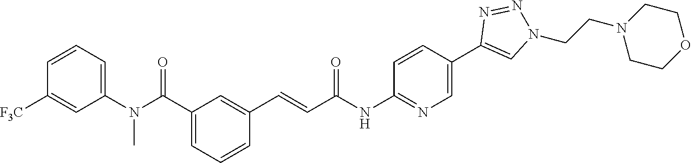 [SMILES: CN(C(=O)c1cccc(/C=C/C(=O)Nc2ccc(-c3cn(CCN4CCOCC4)nn3)cn2)c1)c1cccc(C(F)(F)F)c1] |
| 121 | 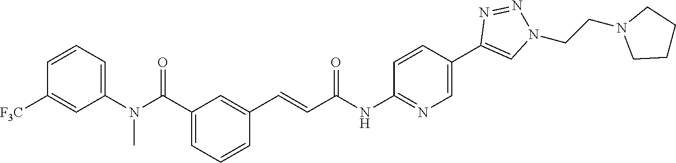 [SMILES: CN(C(=O)c1cccc(/C=C/C(=O)Nc2ccc(-c3cn(CCN4CCCC4)nn3)cn2)c1)c1cccc(C(F)(F)F)c1] |
| 122 | 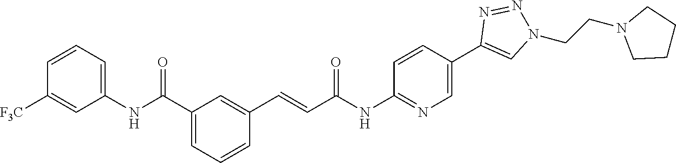 [SMILES: O=C(/C=C/c1cccc(C(=O)Nc2cccc(C(F)(F)F)c2)c1)Nc1ccc(-c2cn(CCN3CCCC3)nn2)cn1] |
| 123 | 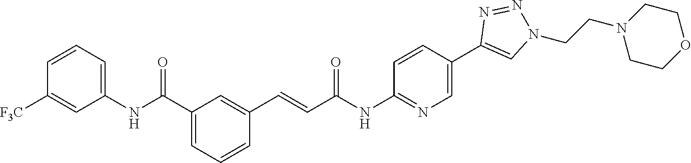 [SMILES: O=C(/C=C/c1cccc(C(=O)Nc2cccc(C(F)(F)F)c2)c1)Nc1ccc(-c2cn(CCN3CCOCC3)nn2)cn1] |
| 124 | 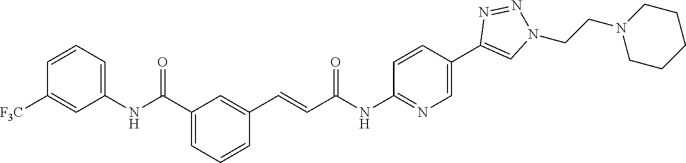 [SMILES: O=C(/C=C/c1cccc(C(=O)Nc2cccc(C(F)(F)F)c2)c1)Nc1ccc(-c2cn(CCN3CCCCC3)nn2)cn1] |
| 125 | 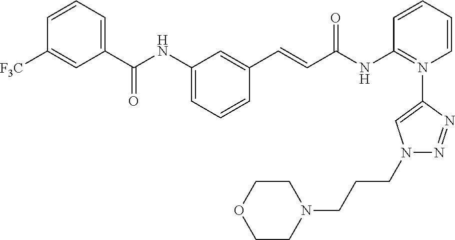 |

### Administration and Pharmaceutical Compositions

Some embodiments include pharmaceutical compositions comprising: (a) a therapeutically effective amount of a compound described herein (including salts, enantiomers, diastereoisomers, tautomers, polymorphs, and solvates thereof), or pharmaceutically acceptable salts thereof; or a metal complex comprising a compound described herein and (b) a pharmaceutically acceptable carrier, diluent, excipient or combination thereof.

The compounds are administered at a therapeutically effective dosage, e.g., a dosage sufficient to provide treatment for the disease states previously described. While human dosage levels have yet to be optimized for the compounds of the preferred embodiments, generally, a daily dose for most of the compounds described herein is from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety.

In addition to the selected compound useful as described above, come embodiments include compositions containing a pharmaceutically-acceptable carrier. The term “pharmaceutically-acceptable carrier”, as used herein, means one or more compatible solid or liquid filler diluents or encapsulating substances, which are suitable for administration to a mammal. The term “compatible”, as used herein, means that the components of the composition are capable of being commingled with the subject compound, and with each other, in a manner such that there is no interaction, which would substantially reduce the pharmaceutical efficacy of the composition under ordinary use situations. Pharmaceutically-acceptable carriers must, of course, be of sufficiently high purity and sufficiently low toxicity to render them suitable for administration preferably to an animal, preferably mammal being treated.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aq. solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include EtOH, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 5 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The actual dose of the active compounds described herein depends on the specific compound, and on the condition to be treated; the selection of the appropriate dose is well within the knowledge of the skilled artisan.

### Methods of Treatment

The mutations of p53 tumor suppressor proteins that lead to loss of wild-type p53 activity are frequently detected in many different tumor types. Perturbations in p53 signaling pathways are believed to be required for the development of most cancers, and therefore restoration or reactivation of p53 function will have significant therapeutic benefit. See Muller et al., Cancer Cell, 2014; 24:304-317.

Some embodiments of the present disclosure relate to a method of treating cancer, comprising administering an therapeutically effective amount of a compound of Formula (I), (II), (II-A) through (II-D), (I-A) through (III-D), (IV-A) through (IV-D), (V), or (V-A) through (V-D) as described herein, or a pharmaceutically acceptable salt thereof, a specific compound selected from Table 1, a pharmaceutically acceptable salt thereof, or a pharmaceutical composition thereof to a subject in need thereof. In some embodiments, the subject being treated has a p53 mutation in the DNA-binding domain.

Some embodiments of the present disclosure relate to a method of treating cancer, comprising selecting a subject having a p53 mutation in the DNA-binding domain; and administering an therapeutically effective amount of a compound of Formula (I), (II), (II-A) through (II-D), (III-A) through (III-D), (IV-A) through (IV-D), (V), or (V-A) through (V-D) as described herein, or a pharmaceutically acceptable salt thereof, a specific compound selected from Table 1, a pharmaceutically acceptable salt thereof, or a pharmaceutical composition thereof to a subject in need thereof.

Some embodiments of the present disclosure relate to methods of modulating or activating a p53 signaling pathway in a subject, comprising administering a therapeutically effective amount of a compound of Formula (I), (II), (II-A) through (II-D), (III-A) through (III-D), (IV-A) through (IV-D), (V), or (V-A) through (V-D) as described herein, or a pharmaceutically acceptable salt thereof, a specific compound selected from Table 1, a pharmaceutically acceptable salt thereof, or a pharmaceutical composition thereof to a subject in need thereof.

Some embodiments of the present disclosure relate to methods of inhibiting cancer cell growth, comprising contacting a cancer cell with an effective amount of a compound of Formula (I), (II), (II-A) through (II-D), (III-A) through (III-D), (IV-A) through (IV-D), (V), or (V-A) through (V-D) as described herein, or a pharmaceutically acceptable salt thereof, a specific compound selected from Table 1, a pharmaceutically acceptable salt thereof, or a pharmaceutical composition thereof.

Non-limiting examples of cancer that may be treated include breast cancer, lung cancer, colon cancer, prostate cancer, liver cancer, cervical cancer, ovarian cancer, bladder cancer, brain cancer, esophageal cancer, kidney cancer, leukemia, melanoma, non-hodgkin lymphoma, pancreatic cancer, skin cancer, thyroid cancer, and endometrial cancer.

Non-limiting examples of cancer cells that may have their growth inhibited include a breast cancer cell, a lung cancer cell, a colon cancer cell, a prostate cancer cell, a liver cancer cell, a cervical cancer cell, an ovarian cancer cell, a bladder cancer cell, a brain cancer cell, an esophageal cancer cell, a kidney cancer cell, a leukemia cell, a melanoma cell, a non-hodgkin lymphoma cell, a pancreatic cancer cell, a skin cancer cell, a thyroid cancer cell, and an endometrial cancer cell.

In some embodiments, the cancer cell has been identified as possessing wild-type p53. In some embodiments, the cancer cell has been identified as underexpressing p53. In some embodiments, the cancer cell has been identified as possessing a p53 mutation.

In some embodiments, the subject is a human. In some embodiments, the subject has been identified as possessing a p53 mutation. In some embodiments, the p53 mutation is in the p53 DNA-binding domain. Non-limiting examples of p53 mutations include, but are not limited to, mutations in amino acid residues 175, 176, 179, 220, 238, 242, 245, 248, 249, 273, 280, and/or 282, for example, R273H, R273C, R175H, R175L, G245S, G245C, C176F, R249S, R282W, C242W, R248Q, R248W, Y220C, and/or R280K, or combinations thereof. In some further embodiments, the p53 mutation comprises or is selected from R175H, G245S, or R248Q, or combinations thereof.

The methods described herein may include identifying a subject in need of treatment. In a preferred embodiment, the methods include identifying a mammal in need of treatment. In a highly preferred embodiment, the methods include identifying a human in need of treatment, where the human has p53 mutation in the DNA-binding domain. Identifying a subject in need of treatment may be accomplished by any means that indicates a subject who may benefit from treatment. For example, identifying a subject in need of treatment may occur by clinical diagnosis, laboratory testing such as genomic sequencing, or any other means known to one of skill in the art, including any combination of means for identification. In some embodiments, a subject in need of treatment has been identified as possessing a p53 mutation. In some embodiments, p53 mutations include, but are not limited to, R273H, R273C, R175H, R175L, G245S, G245C, C176F, R249S, R282W, C242W, R248Q, R248W, Y220C, and/or R280K, or combinations thereof. In some further embodiments, the p53 mutation comprises or is selected from R175H, G245S, or R248Q, or combinations thereof.

In some embodiments, the cancer cell has been identified as possessing low levels of wild-type p53. In some embodiments, the subject has been identified as possessing a p53 mutation. In some embodiments, the subject has been identified as possessing high levels of p53 protein having the p53 mutation. The terms “low levels” and “high levels” of p53, as used herein, refers to a lower than normal amount of p53 protein and a higher than normal amount of p53 protein, respectively. The normal amount of p53 protein refers to the p53 levels found in a normal cell of the same cell type, a normal tissue of the same tissue type, and/or a normal human subject. Thus, a low level of p53 refers to a lower level of p53 relative to the normal amount of p53. Similarly, a high level of p53 refers to a higher level of p53 relative to the normal amount of p53.

The terms “therapeutically effective amount,” as used herein, refer to an amount of a compound sufficient to cure, ameliorate, slow progression of, prevent, or reduce the likelihood of onset of the identified disease or condition, or to exhibit a detectable therapeutic, prophylactic, or inhibitory effect. The effect can be detected by, for example, the assays disclosed in the following examples. The precise effective amount for a subject will depend upon the subject's body weight, size, and health; the nature and extent of the condition; and the therapeutic or combination of therapeutics selected for administration. Therapeutically and prophylactically effective amounts for a given situation can be determined by routine experimentation that is within the skill and judgment of the clinician.

For any compound, the therapeutically or prophylactically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans.

Therapeutic/prophylactic efficacy and toxicity may be determined by standard pharmaceutical procedures in cell cultures or experimental animals, e.g., IC₅₀ is a measure of how effective a drug is. It indicates how much of a particular drug compound is needed to inhibit a given biological process (e.g., a cancer cell line) by half. It is commonly used as a measure of antagonist drug potency in pharmacological research. ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅/LD₅. Pharmaceutical compositions that exhibit large therapeutic indices are preferred. However, pharmaceutical compositions that exhibit narrow therapeutic indices are also within the scope of the invention. The data obtained from cell culture assays and animal studies may be used in formulating a range of dosage for human use. The dosage contained in such compositions is preferably within a range of circulating concentrations that include an ED₅₀ with little or no toxicity. The dosage may vary within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

In another aspect, treating a condition described herein results in a reduction in the rate of cellular proliferation. Preferably, after treatment, the rate of cellular proliferation is reduced by at least about 5%; more preferably, by at least about 10%; more preferably, by at least about 20%; more preferably, by at least about 30%; more preferably, by at least about 40%; more preferably, by at least about 50%; even more preferably, by at least about 60%; and most preferably, by at least about 75%. The rate of cellular proliferation may be measured by any reproducible means of measurement. In a preferred aspect, the rate of cellular proliferation is measured, for example, by measuring the number of dividing cells in a tissue sample per unit time.

In another aspect, treating a condition described herein results in a reduction in the proportion of proliferating cells. Preferably, after treatment, the proportion of proliferating cells is reduced by at least about 5%; more preferably, by at least about 10%; more preferably, by at least about 20%; more preferably, by at least about 30%; more preferably, by at least about 40%; more preferably, by at least about 50%; even more preferably, by at least about 60%; and most preferably, by at least about 75%. The proportion of proliferating cells may be measured by any reproducible means of measurement. In a preferred aspect, the proportion of proliferating cells is measured, for example, by quantifying the number of dividing cells relative to the number of non-dividing cells in a tissue sample. In another preferred aspect, the proportion of proliferating cells is equivalent to the mitotic index.

In another aspect, treating a condition described herein results in a decrease in size of an area or zone of cellular proliferation. Preferably, after treatment, size of an area or zone of cellular proliferation is reduced by at least 5% relative to its size prior to treatment; more preferably, reduced by at least about 10%; more preferably, reduced by at least about 20%; more preferably, reduced by at least about 30%; more preferably, reduced by at least about 40%; more preferably, reduced by at least about 50%; even more preferably, reduced by at least about 60%; and most preferably, reduced by at least about 75%. Size of an area or zone of cellular proliferation may be measured by any reproducible means of measurement. In a preferred aspect, size of an area or zone of cellular proliferation may be measured as a diameter or width of an area or zone of cellular proliferation.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In some embodiments, the agents are administered simultaneously. In some such embodiments, administration in combination is accomplished by combining the agents in a single dosage form. In some embodiments, the agents are administered sequentially. In some embodiments the agents are administered through the same route, such as orally. In some other embodiments, the agents are administered through different routes, such as one being administered orally and another being administered i.v. Thus, for example, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

### Synthesis

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### EXAMPLES

Additional embodiments are disclosed in further detail in the following examples, which are not in any way intended to limit the scope of the claims.

Example 1 provides synthetic schemes and procedures for the preparation of various intermediates and compounds described herein.

### Synthesis of (E)-3-(3-(3-(trifluoromethyl)benzamido)phenyl)acrylic acid Int-5

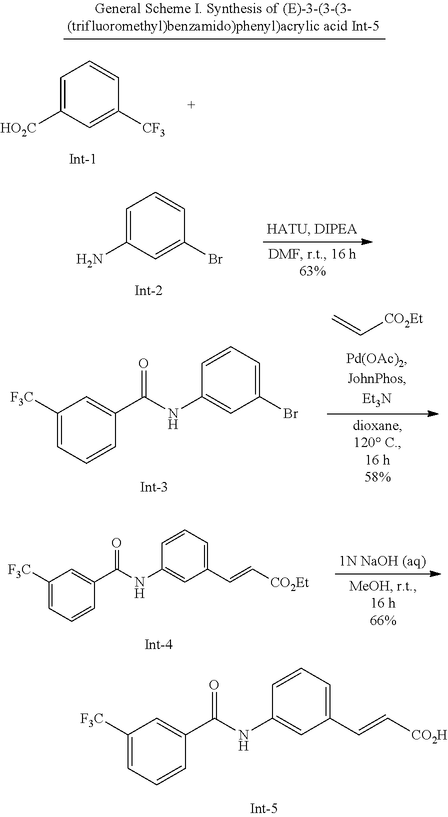

### N-(3-bromophenyl)-3-(trifluoromethyl)benzamide (Int-3)

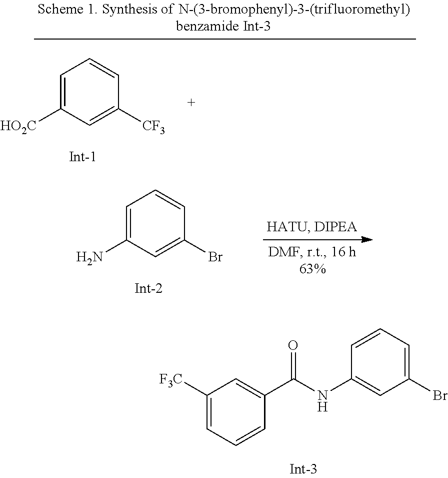 [SMILES: O=C(O)c1cccc(C(F)(F)F)c1 ; Nc1cccc(Br)c1 ; O=C(Nc1cccc(Br)c1)c1cccc(C(F)(F)F)c1]

### Experimental Procedure:

DIPEA (45.9 mL 263.0 mmol) was added to a stirred solution of 3-(trifluoromethyl)benzoic acid Int-1 (10.0 g, 52.60 mmol) in DMF (100 mL) at 0° C., followed by HATU (30.0 g, 78.90 mmol) at 0° C. and the reaction mixture was stirred for 15 min. Then 3-bromoaniline Int-2 (6.9 mL, 63.12 mmol) was added dropwise at 0° C. The reaction mixture was stirred at room temperature for 16 hrs. Then the reaction mixture was diluted with water (100 mL) and extracted with Ethyl Acetate (2×300 mL). The combined organic layers were washed with water (3×180 mL), brine (180 mL), dried over anhydrous sodium sulfate and concentrated under reduced pressure. The resulting crude compound was purified by column chromatography on silica gel (100-200 mesh) using 20% Ethyl Acetate in Hexane as eluent to afford 12.0 g of N-(3-bromophenyl)-3-(trifluoromethyl)benzamide Int-3 as a pale yellow solid in 63% yield. ¹H-NMR (300 MHz, DMSO-d₆): δ 10.60 (s, 1H), 8.30 (s, 1H), 8.27 (d, J=8.1 Hz, 1H), 8.10 (s, 1H), 7.99 (d, J=7.8 Hz, 1H), 7.85-7.73 (m, 2H), 7.40-7.30 (m, 2H); MS (ESI) m/z 343.99 [M+H]⁺.

### Ethyl (E)-3-(3-(3-(trifluoromethyl)benzamido)phenyl)acrylate (Int-4)

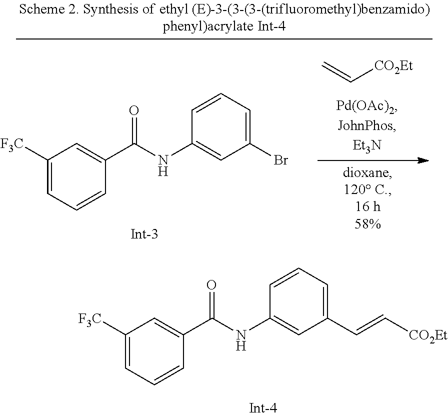

### Experimental Procedure:

Triethylamine (6.08 mL, 43.60 mmol) and Ethyl Acrylate (1.86 mL, 15.78 mmol) were added to a degassed solution of N-(3-bromophenyl)-3-(trifluoromethyl)benzamide Int-3 (3.0 g, 8.72 mmol) in 1,4-Dioxane (45 mL) in a sealed tube at room temperature. Then JohnPhos catalyst (0.47 g, 1.58 mmol) was added, followed by Pd(OAc)₂ (0.58 g, 0.87 mmol) and the reaction mixture was degassed for additional 10 min. Then the reaction mixture was heated at 120° C. for 16 hrs. After cooling down to room temperature, the reaction mixture was diluted with Ethyl Acetate (100 mL), filtered through a pad of Celite and washed with Ethyl Acetate. The filtrate was concentrated under reduced pressure, and the resulting crude compound was purified by column chromatography on silica gel (100-200 mesh) using 20% Ethyl Acetate in Hexane as eluent to afford 1.8 g of ethyl (E)-3-(3-(3-(trifluoromethyl)benzamido)phenyl)acrylate Int-4 as a pale brown liquid in 58% yield. ¹H-NMR (300 MHz, DMSO-d₆): δ 10.58 (s, 1H), 8.31 (s, 1H), 8.28 (d, J=8.1 Hz, 1H), 8.06 (s, 1H), 7.99 (d, J=7.8 Hz, 1H), 7.85-7.77 (m, 2H), 7.64 (d, J=16.2 Hz, 1H), 7.52 (d, J=8.1 Hz, 1H), 7.45 (t, J=7.5 Hz, 1H), 6.57 (d, J=15.6 Hz, 1H), 4.20 (q, J=6.9 Hz, 2H), 1.27 (t, J=6.9 Hz, 3H); MS (ESI) m/z 364.06 [M+H]⁺.

### (E)-3-(3-(3-(trifluoromethyl)benzamido)phenyl)acrylic acid (Int-5)

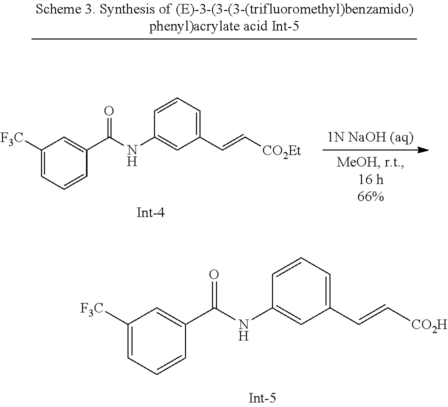 [SMILES: CCOC(=O)/C=C/c1cccc(NC(=O)c2cccc(C(F)(F)F)c2)c1 ; O=C(O)/C=C/c1cccc(NC(=O)c2cccc(C(F)(F)F)c2)c1]

### Experimental Procedure:

1 M NaOH solution (22 mL, 22.03 mmol) was added to a stirred solution of ethyl (E)-3-(3-(3-(trifluoromethyl)benzamido)phenyl)acrylate Int-4 (2.0 g, 5.51 mmol) in methanol (40 mL) at 0° C., and the reaction mixture was left to stir at room temperature for 12 hrs. Then methanol was removed under reduced pressure, and the resulting crude residue was diluted with water (10 mL) before the pH was adjusted to approx. 3-4 with 1 N HCl. The resulting precipitate was filtered off, washed with water (20 mL) and dried in vacuo to afford 1.2 g of (E)-3-(3-(3-(trifluoromethyl)benzamido)phenyl)acrylic acid Int-5 as an off-white solid in 66% yield. ¹H-NMR (400 MHz, DMSO-d₆): δ 12.42 (bs, 1H), 10.55 (s, 1H), 8.31 (s, 1H), 8.28 (d, J=8.0 Hz, 1H), 8.03 (s, 1H), 7.98 (d, J=8.0 Hz, 1H), 7.85-7.76 (m, 2H), 7.58 (d, J=16.0 Hz, 1H), 7.49-7.40 (m, 2H), 6.47 (d, J=15.6 Hz, 1H); MS (ESI) m/z 336.09 [M+H]⁺.

### Synthesis of (E)-3-(3-(N-methyl-3-(trifluoromethyl)benzamido)phenyl)acrylic acid Int-7

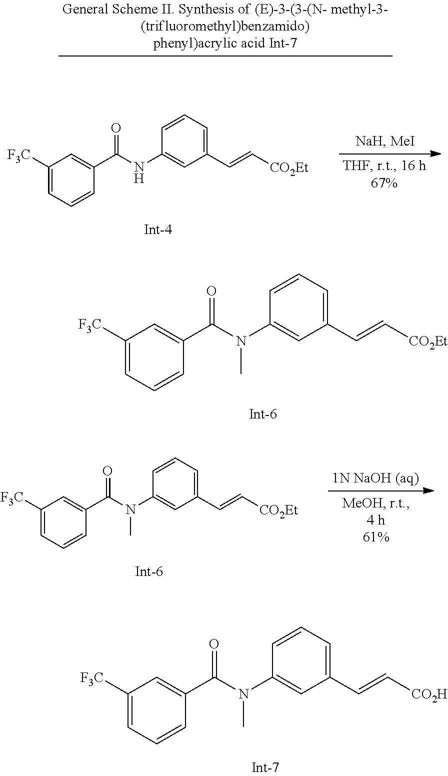 [SMILES: CCOC(=O)/C=C/c1cccc(NC(=O)c2cccc(C(F)(F)F)c2)c1 ; CCOC(=O)/C=C/c1cccc(N(C)C(=O)c2cccc(C(F)(F)F)c2)c1 ; CCOC(=O)/C=C/c1cccc(N(C)C(=O)c2cccc(C(F)(F)F)c2)c1 ; CN(C(=O)c1cccc(C(F)(F)F)c1)c1cccc(/C=C/C(=O)O)c1]

### (E)-3-(3-(N-methyl-3-(trifluoromethyl)benzamido) phenyl)acrylate (Int-6)

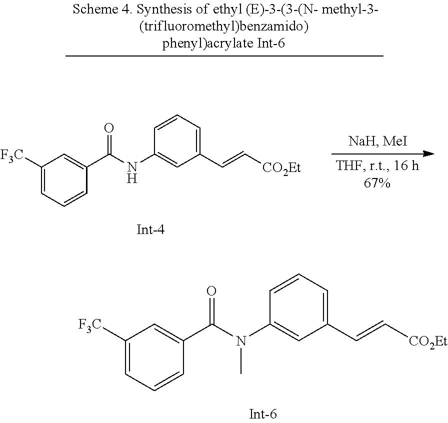 [SMILES: CCOC(=O)/C=C/c1cccc(NC(=O)c2cccc(C(F)(F)F)c2)c1 ; CCOC(=O)/C=C/c1cccc(N(C)C(=O)c2cccc(C(F)(F)F)c2)c1]

### Experimental Procedure:

Sodium hydride (0.66 g, 16.52 mmol) was added to a stirred solution of ethyl (E)-3-(3-(3-(trifluoromethyl)benzamido)phenyl)acrylate Int-4 (3.0 g, 8.26 mmol) in THF (60 mL) at 0° C., and the reaction mixture was stirred for 10 min. Then iodomethane (1.5 mL, 24.79 mmol) was added to the reaction mixture at 0° C., and it was left to stir at room temperature for 16 hrs. Then the reaction mixture was diluted with water (100 mL) and extracted with Ethyl Acetate (2×150 mL). Combined organic layers were washed with water (2×100 mL), brine (200 mL), dried over anhydrous sodium sulfate and concentrated under reduced pressure. The resulting crude compound was purified by column chromatography on silica gel (100-200 mesh) using 15% Ethyl Acetate in Hexane as eluent to afford 2.1 g of ethyl (E)-3-(3-(N-methyl-3-(trifluoromethyl) benzamido)phenyl)acrylate Int-6 as a pale yellow solid in 67% yield. ¹H-NMR (400 MHz, DMSO-d₆): δ 7.73-7.42 (m, 7H), 7.29 (t, J=10.4 Hz, 1H), 7.23 (d, J=10.4 Hz, 1H), 6.58 (d, J=20.8 Hz, 1H), 4.17 (q, J=9.2 Hz, 2H), 3.41 (s, 3H), 1.25 (t, J=9.2 Hz, 3H); MS (ESI) m/z 378.40 [M+H]⁺.

### (E)-3-(3-(N-methyl-3-trifluoromethyl)benzamido) phenyl)acrylic acid (Int-7)

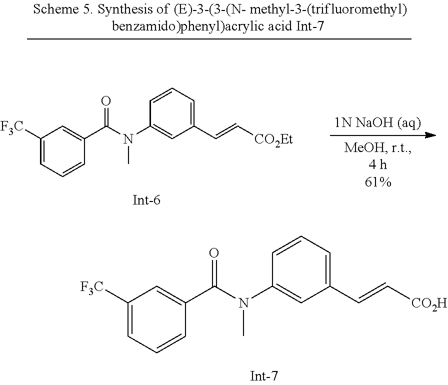 [SMILES: CCOC(=O)/C=C/c1cccc(N(C)C(=O)c2cccc(C(F)(F)F)c2)c1 ; CN(C(=O)c1cccc(C(F)(F)F)c1)c1cccc(/C=C/C(=O)O)c1]

### Experimental Procedure:

1 M NaOH solution (22 mL, 22.28 mmol) was added to a stirred solution of ethyl (E)-3-(3-(N-methyl-3-(trifluoromethyl)benzamido) phenyl)acrylate Int-6 (2.1 g, 5.57 mmol) in methanol (40 mL) at 0° C., and the reaction mixture was left to stir at room temperature for 4 hrs. Then methanol was removed under reduced pressure and the resulting crude residue was diluted with water (10 mL), before the pH was adjusted with 1 N HCl to approx. 3-4. The precipitate was filtered off, washed with water (20 mL) and dried in vacuo to afford 1.2 g of (E)-3-(3-(N-methyl-3-(trifluoromethyl)benzamido) phenyl)acrylic acid Int-7 in 61% yield as an off-white solid. ¹H-NMR (400 MHz, DMSO-d₆): δ 12.40 (bs, 1H), 7.63 (s, 1H), 7.61 (d, J=5.2 Hz, 2H), 7.56 (d, J=7.2 Hz, 1H), 7.51-7.41 (m, 3H), 7.29 (t, J=7.6 Hz, 1H), 7.22 (d, J=8.0 Hz, 1H), 6.46 (d, J=16.0 Hz, 1H), 3.41 (s, 3H); MS (ESI) m/z 350.08 [M+H].

### Synthesis of (E)-3-(3-((3-(trifluoromethyl)phenyl)carbamoyl)phenyl)acrylic acid Int-12

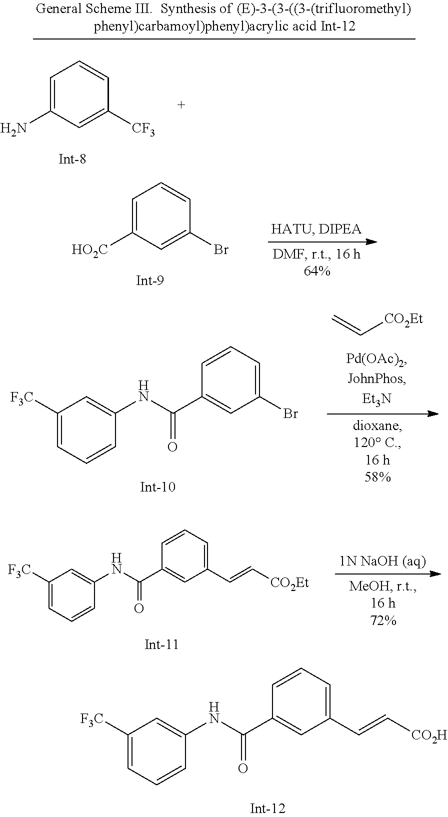

### 3-Bromo-N-(3-(trifluoromethyl)phenyl)benzamide (Int-10)

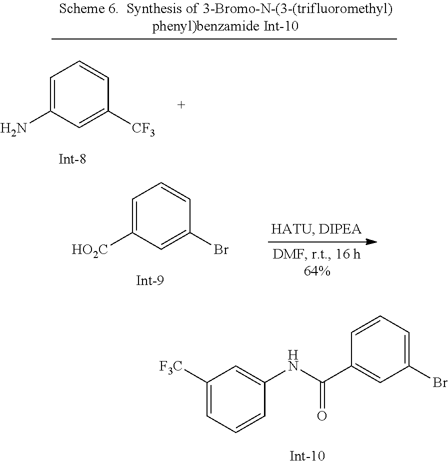 [SMILES: Nc1cccc(C(F)(F)F)c1 ; O=C(O)c1cccc(Br)c1 ; O=C(Nc1cccc(C(F)(F)F)c1)c1cccc(Br)c1]

### Experimental Procedure:

DIPEA (43.4 mL 248.80 mmol) was added to a stirred solution of 3-bromobenzoic acid Int-9 (10.0 g, 49.67 mmol) in DMF (100 mL) at 0° C., followed by HATU (28.3 g, 28.31 mmol). The solution was stirred for 15 min before 3-(trifluoromethyl)aniline Int-8 (7.4 mL, 59.61 mmol) was added dropwise at 0° C. The reaction mixture was left to stir at room temperature for 16 hrs. Then it was diluted with water (500 mL) and extracted with Ethyl Acetate (2×300 mL). Combined organic layers were washed with water (3×120 mL), brine (120 mL), dried over anhydrous sodium sulfate and concentrated under reduced pressure. The resulting crude compound was purified by column chromatography on silica gel (100-200 mesh) using 20% Ethyl Acetate in Hexane as eluent to afford 11.0 g of 3-bromo-N-(3-(trifluoromethyl)phenyl)benzamide Int-10 as a pale yellow solid in 64% yield. ¹H-NMR (400 MHz, MeOD): δ 8.16 (bs, 1H), 8.13 (t, J=1.7 Hz, 1H), 7.93 (ddd, J=7.8, 1.7, 1.0 Hz, 2H), 7.76 (ddd, J=8.0, 2.0, 1.0 Hz, 1H), 7.56 (t, J=8.0 Hz, 1H), 7.48-7.43 (m, 2H); MS (ESI) m/z 343.99 [M+H]⁺.

### Ethyl (E)-3-(3-((3-(trifluoromethyl)phenyl)carbamoyl) phenyl)acrylate (Int-1)

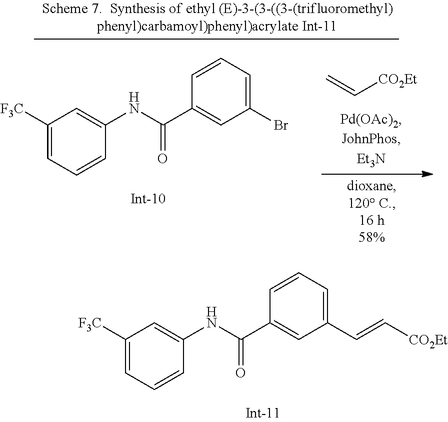

### Experimental Procedure:

Triethylamine (6.1 mL 43.60 mmol) and Ethyl Acrylate (1.86 mL 15.78 mmol) were added to a degassed solution of 3-bromo-N-(3-(trifluoromethyl)phenyl)benzamide Int-10 (3.0 g, 8.72 mmol) in 1,4-Dioxane (45 mL) in a sealed tube at room temperature. Then JohnPhos (0.47 g, 1.58 mmol) was added, followed by Pd(OAc)₂ (0.58 g, 0.87 mmol) and the reaction mixture was degassed for additional 10 min. Then the reaction mixture was heated at 120° C. for 16 hrs. Then it was cooled down to room temperature, diluted with Ethyl Acetate (100 mL), filtered through a pad of Celite and washed with Ethyl Acetate. The filtrate was concentrated under reduced pressure and the resulting crude compound was purified by column chromatography on silica gel (100-200 mesh) using 20% Ethyl Acetate in Hexane as eluent to afford 1.8 g of ethyl (E)-3-(3-(3-(trifluoromethyl)-benzamido)phenyl)acrylate Int-11 as pale brown liquid in 58% yield. ¹H-NMR (300 MHz, DMSO-d₆): δ 10.59 (s, 1H), 8.32 (s, 1H), 8.24 (s, 1H), 8.07 (d, J=8.4 Hz, 1H), 7.98 (t, J=8.1 Hz, 2H), 7.75 (d, J=16.2 Hz, 1H), 7.67-7.57 (m, 2H), 7.48 (d, J=7.8 Hz, 1H), 6.79 (d, J=15.9 Hz, 1H), 4.22 (q, J=7.2 Hz, 2H), 1.28 (t, J=7.5 Hz, 3H); MS (ESI) m/z 364.35 [M+H]⁺.

### (E)-3-(3-((3-(trifluoromethyl)phenyl)carbamoyl)phenyl)acrylic acid (Int-12)

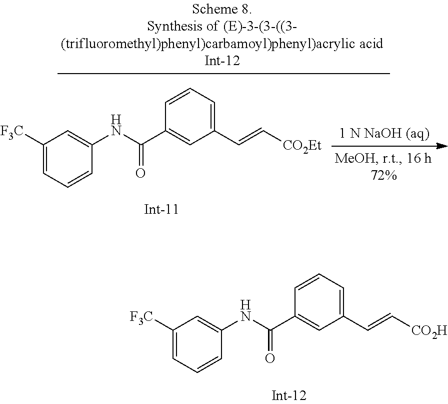

### Experimental Procedure:

1 M NaOH solution (22 mL, 22.03 mmol) was added to a stirred solution of ethyl (E)-3-(3-(3-(trifluoromethyl)benzamido)phenyl)acrylate Int-11 (2.0 g, 5.51 mmol) in methanol (40 mL) at 0° C. and the reaction mixture was left to stir at room temperature for 12 hrs. Then methanol was removed under reduced pressure, the residue was diluted with water (10 mL) and pH was adjusted to approx. 3-4 with 1N HCl solution. The resulting precipitate was filtered off, washed with water (20 mL) and dried in vacuo to afford 1.3 g of (E)-3-(3-((3-(trifluoromethyl)phenyl)carbamoyl)phenyl)acrylic acid Int-12 as an off-white solid in 72% yield. ¹H-NMR (400 MHz, DMSO-d₆): δ 12.50 (bs, 1H), 10.58 (s, 1H), 8.28 (s, 1H), 8.24 (s, 1H), 8.07 (d, J=8.4 Hz, 1H), 7.98 (d, J=8.0 Hz, 1H), 7.92 (d, J=7.6 Hz, 1H), 7.67 (d, J=16.0 Hz, 1H), 7.61 (app. q, J=8.4 Hz, 2H), 7.48 (d, J=7.6 Hz, 1H), 6.68 (d, J=16.0 Hz, 1H); MS (ESI) m/z 336.09 [M+H]⁺.

### Synthesis of (E)-3-(3-(methyl(3-(trifluoromethyl)phenyl)carbamoyl)phenyl)acrylic acid Int-14

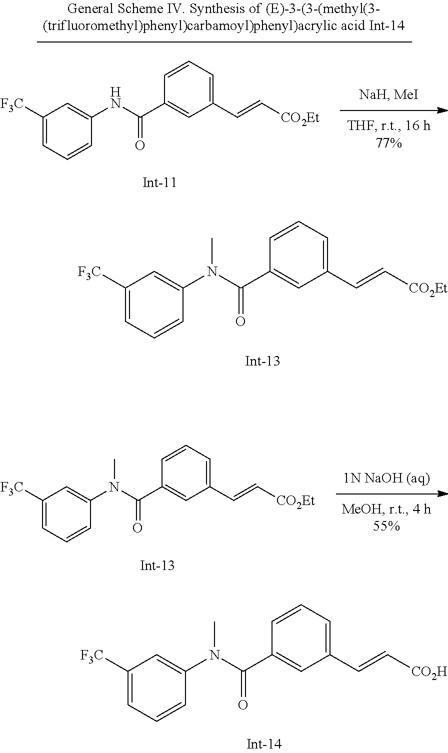 [SMILES: CCOC(=O)/C=C/c1cccc(C(=O)Nc2cccc(C(F)(F)F)c2)c1 ; CCOC(=O)/C=C/c1cccc(C(=O)N(C)c2cccc(C(F)(F)F)c2)c1 ; CCOC(=O)/C=C/c1cccc(C(=O)N(C)c2cccc(C(F)(F)F)c2)c1 ; CN(C(=O)c1cccc(/C=C/C(=O)O)c1)c1cccc(C(F)(F)F)c1]

### Ethyl (E)-3-(3-(methyl(3-(trifluoromethyl)phenyl) carbamoyl)phenyl)acrylate (Int-13)

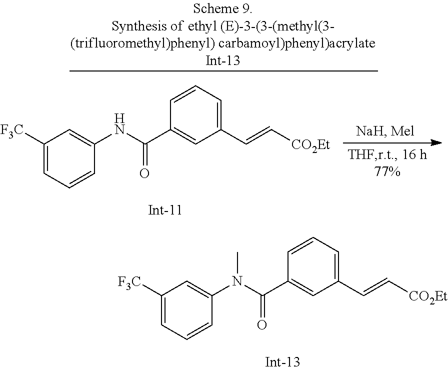

### Experimental Procedure:

Sodium hydride (0.66 g, 16.52 mmol) was added to a stirred solution of ethyl (E)-3-(3-(3-(trifluoromethyl)benzamido)phenyl)acrylate Int-11 (3.0 g, 8.26 mmol) in THF (60 mL) at 0° C. and the reaction mixture was stirred at 0° C. for 10 min. Then iodomethane (1.5 mL, 24.79 mmol) was added and the reaction mixture was stirred at room temperature for 16 hrs. Then it was diluted with water (100 mL) and extracted with Ethyl Acetate (2×150 mL). Combined organic layers were washed with water (2×100 mL), brine (200 mL), dried over anhydrous sodium sulfate and concentrated under reduced pressure. The resulting crude compound was purified by column chromatography on silica gel (100-200 mesh) using 15% Ethyl Acetate in Hexane as eluent to afford 2.4 g of ethyl (E)-3-(3-(N-methyl-3-(trifluoromethyl)benzamido) phenyl)acrylate Int-13 as a pale yellow solid in 77% yield. ¹H-NMR (300 MHz, CDCl₃): δ 7.53 (d, J=16.2 Hz, 1H), 7.47 (s, 1H), 7.45-7.31 (m, 4H), 7.25-7.18 (m, 3H), 6.28 (d, J=16.2 Hz, 1H), 4.25 (q, J=7.2 Hz, 2H), 3.51 (s, 3H), 1.33 (t, J=6.9 Hz, 3H); MS (ESI) m/z 378.40 [M+H]⁺.

### (E)-3-(3-(methyl(3-(trifluoromethyl)phenyl)carbamoyl)phenyl)acrylic acid (Int-14)

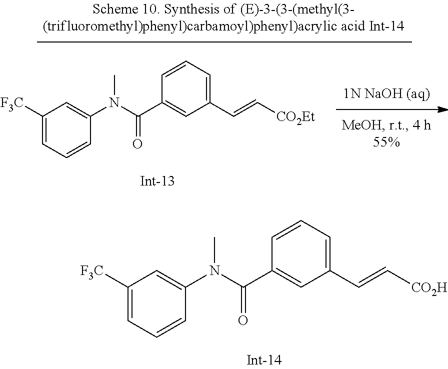 [SMILES: CCOC(=O)/C=C/c1cccc(C(=O)N(C)c2cccc(C(F)(F)F)c2)c1 ; CN(C(=O)c1cccc(/C=C/C(=O)O)c1)c1cccc(C(F)(F)F)c1]

### Experimental Procedure:

1 M NaOH solution (31.8 mL, 31.83 mmol) was added to a stirred solution of ethyl (E)-3-(3-(N-methyl-3-(trifluoromethyl)benzamido)-phenyl)acrylate Int-13 (3.0 g, 7.96 mmol) in methanol (60 mL) at 0° C., and the reaction mixture was stirred at room temperature for 4 hrs. Then methanol was removed under reduced pressure, the residue was diluted with water (10 mL), and pH was adjusted to approx. 3-4 with 1N HCl solution. The resulting precipitate was filtered off, washed with water (20 mL) and dried in vacuo to afford 1.5 g of (E)-3-(3-(methyl(3-(trifluoromethyl)phenyl)carbamoyl)phenyl)acrylic acid Int-14 as off-white solid in 55% yield. ¹H-NMR (400 MHz, DMSO-d₆): δ 12.40 (bs, 1H), 7.67-7.56 (m, 3H), 7.55-7.48 (m, 3H), 7.45 (d, J=16.0 Hz, 1H), 7.30 (d, J=4.4 Hz, 2H), 6.39 (d, J=16.0 Hz, 1H), 3.41 (s, 3H); MS (ESI) m/z 350.08 [M+H]⁺.

### Synthesis of (E)-3-(3-((3-(trifluoromethyl)phenyl)sulfonamido)phenyl)acrylic acid Int-18

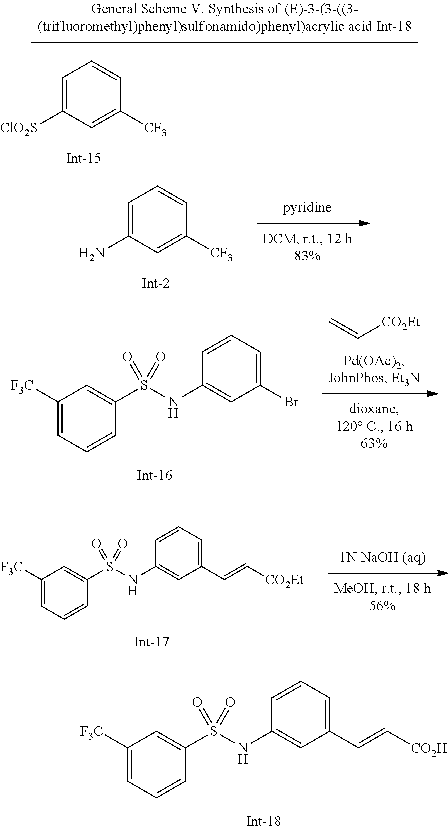

### N-(3-Bromophenyl)-3-(trifluoromethyl)benzenesulfonamide (Int-16)

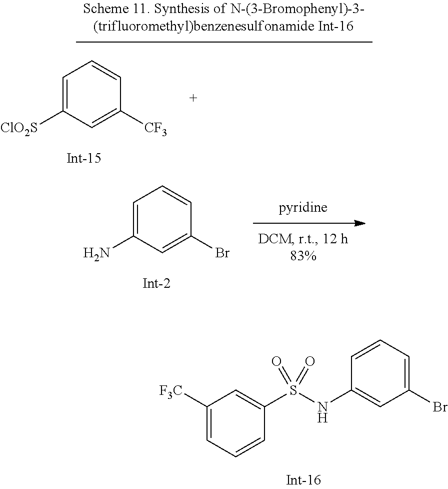 [SMILES: O=S(=O)(Cl)c1cccc(C(F)(F)F)c1 ; Nc1cccc(Br)c1 ; O=S(=O)(Nc1cccc(Br)c1)c1cccc(C(F)(F)F)c1]

### Experimental Procedure:

Pyridine (9.7 mL 122.6 mmol) was added to a stirred solution of 3-(trifluoromethyl) benzenesulfonyl chloride Int-15 (10.0 g, 40.08 mmol) in DCM (200 mL) at 0° C., then 3-bromoaniline Int-2 (4.45 mL, 40.08 mmol) was added dropwise at 0° C. The reaction mixture was stirred at room temperature for 16 hrs. Then the reaction mixture was diluted with 1 N HCl solution (200 mL) and extracted with Ethyl Acetate (2×300 mL). Combined organic layers were washed with water (2×180 mL), brine (180 mL), dried over anhydrous sodium sulfate and concentrated under reduced pressure to afford 13.0 g of N-(3-bromophenyl)-3-(trifluoromethyl)benzenesulfonamide Int-16 as a pale brown solid in 83% yield. ¹H-NMR (300 MHz, DMSO-d₆): δ 10.71 (s, 1H), 8.11-8.00 (m, 3H), 7.84 (t, J=7.8 Hz, 1H), 7.32-7.19 (m, 3H), 7.12 (td, J=7.8, 1.8 Hz, 1H); MS (ESI) m/z 381.96 [M+H]⁺.

### Ethyl (E)-3-(3-((3-(trifluoromethyl)phenyl)sulfonamido)phenyl)acrylate (Int-17)

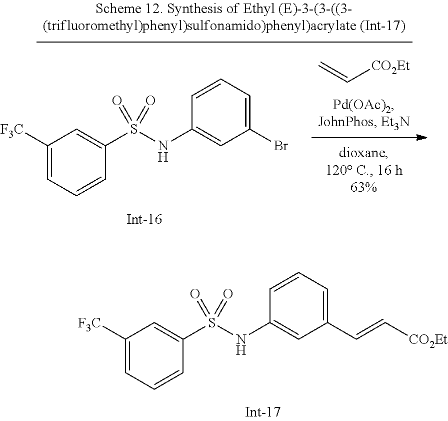

### Experimental Procedure:

Triethylamine (5.5 mL 39.47 mmol) and Ethyl Acrylate (1.6 mL 15.78 mmol) were added to a degassed solution of N-(3-bromophenyl)-3-(trifluoromethyl)benzene sulfonamide Int-16 (3.0 g, 7.89 mmol) in 1,4-Dioxane (45 mL) in a sealed tube at room temperature. Then JohnPhos (0.47 g, 1.58 mmol) and Pd(OAc)₂ (0.53 g, 0.80 mmol) were added to the reaction mixture and it was degassed for further 10 min. Then the reaction mixture was heated at 120° C. for 16 hrs. After cooling down to room temperature, the reaction mixture was diluted with Ethyl Acetate (100 mL), filtered through a pad of Celite and washed with Ethyl Acetate. The filtrate was concentrated under reduced pressure to give the crude product, which was purified by column chromatography on silica gel (100-200 mesh) using 20% Ethyl Acetate in Hexane as eluent to afford 2.0 g of ethyl (E)-3-(3-((3-(trifluoromethyl)phenyl)sulfonamido)phenyl) acrylate Int-17 as a pale brown liquid in 63% yield. ¹H-NMR (300 MHz, DMSO-d₆): δ 10.59 (s, 1H), 8.10-7.98 (m, 3H), 7.81 (t, J=8.1 Hz, 1H), 7.54 (d, J=16.2 Hz, 1H), 7.47 (d, J=7.8 Hz, 1H), 7.36-7.27 (m, 2H), 7.11 (d, J=8.1 Hz, 1H), 6.46 (d, J=15.9 Hz, 1H), 4.18 (q, J=6.9 Hz, 2H), 1.25 (t, J=7.2 Hz, 3H); MS (ESI) m/z 400.36 [M+H]⁺.

### (E)-3-(3-((3-(trifluoromethyl)phenyl)sulfonamido)phenyl)acrylic acid (Int-18)

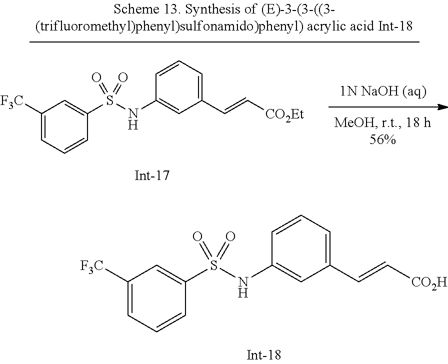 [SMILES: CCOC(=O)/C=C/c1cccc(NS(=O)(=O)c2cccc(C(F)(F)F)c2)c1 ; O=C(O)/C=C/c1cccc(NS(=O)(=O)c2cccc(C(F)(F)F)c2)c1]

### Experimental Procedure:

1 M NaOH solution (25 mL, 25.04 mmol) was added to a stirred solution of ethyl (E)-3-(3-((3-(trifluoromethyl)phenyl)sulfonamido)phenyl)acrylate Int-17 (2.5 g, 6.26 mmol) in methanol (50 mL) at 0° C. and the reaction mixture was stirred at room temperature for 12 hrs. Then methanol was removed under reduced pressure, the residue was diluted with water (10 mL) and the aqueous layer was adjusted to pH 3-4 with 1 N HCl solution. The resulting precipitate was filtered off, washed with water (20 mL) and dried in vacuo to afford 1.3 g of (E)-3-(3-((3-(trifluoromethyl)phenyl)sulfonamido) phenyl)acrylic acid Int-18 as a pale yellow solid in 56% yield. ¹H-NMR (400 MHz, DMSO-d₆): δ 12.42 (bs, 1H), 10.56 (bs, 1H), 8.08-7.98 (m, 3H), 7.81 (t, J=7.6 Hz, 1H), 7.46 (d, J=16.4 Hz, 1H), 7.41 (d, J=8.0 Hz, 1H), 7.34-7.26 (m, 2H), 7.11 (d, J=8.0 Hz, 1H), 6.36 (d, J=16.0 Hz, 1H); MS (ESI) m/z 370.11 [M−H].

### (E)-3-(3-((N-methyl-3-(trifluoromethyl)phenyl)sulfonamido)phenyl)acrylic acid (Int-19)

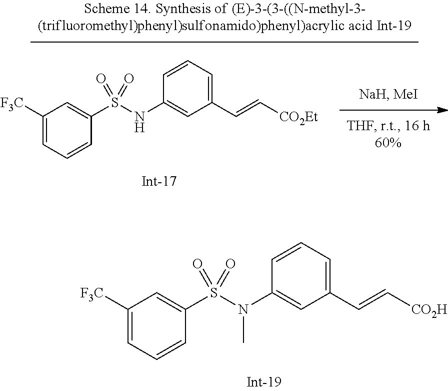 [SMILES: CCOC(=O)/C=C/c1cccc(NS(=O)(=O)c2cccc(C(F)(F)F)c2)c1 ; CN(c1cccc(/C=C/C(=O)O)c1)S(=O)(=O)c1cccc(C(F)(F)F)c1]

### Experimental Procedure:

Sodium hydride (0.75 g, 18.79 mmol) was added to a stirred solution of ethyl (E)-3-(3-((3-(trifluoromethyl)phenyl)sulfonamido)phenyl)acrylate Int-17 (2.5 g, 6.26 mmol) in THF (50 mL) at 0° C., and the reaction mixture was stirred at 0° C. for 10 min. Then iodomethane (1.95 mL, 31.32 mmol) was added to the reaction mixture, and the reaction mixture was stirred at room temperature for 48 hrs. Then aqueous saturated NH₄Cl solution (30 mL) was added, the excess amount of THF was distilled off under reduced pressure, and the pH was adjusted to 3-4 with 1 N HCl solution. The resulting precipitate was filtered off, washed with water (20 mL) and dried in vacuo to afford 1.45 g of (E)-3-(3-((N-methyl-3-(trifluoromethyl)phenyl)sulfonamido)phenyl)acrylic acid Int-19 as an off-white solid in 60% yield. ¹H-NMR (300 MHz, DMSO-d₆): δ 12.50 (bs, 1H), 8.13 (s, 1H), 7.88 (d, J=5.4 Hz, 2H), 7.56 (d, J=8.1 Hz, 1H), 7.57 (s, 1H), 7.50 (d, J=16.2 Hz, 1H), 7.46-7.36 (m, 2H), 7.21 (d, J=9.0 Hz, 1H), 6.47 (d, J=15.9 Hz, 1H), 3.18 (s, 3H); MS (ESI) m/z 386.07 [M+H]⁺.

### Synthesis of (E)-3-(3-(N-(3-(trifluoromethyl)phenyl)sulfamoyl)phenyl)acrylic acid (Int-23)

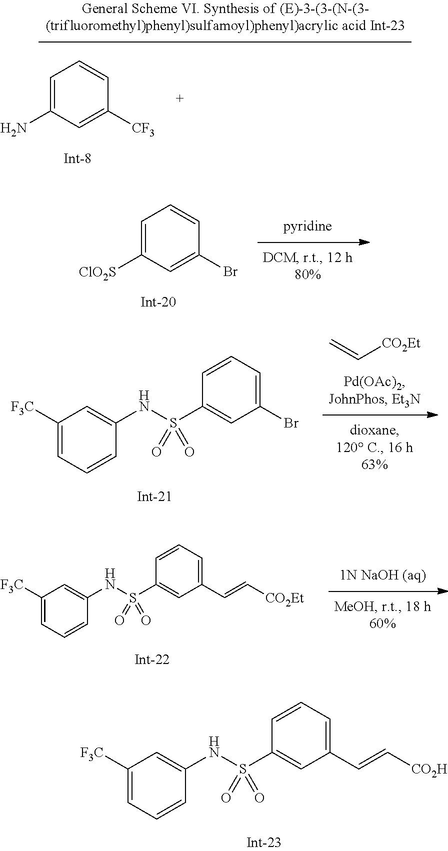

### 3-Bromo-N-(3-(trifluoromethyl)phenyl)benzenesulfonamide (Int-21)

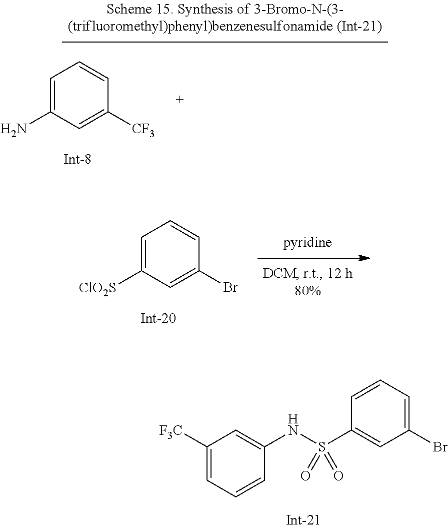 [SMILES: Nc1cccc(C(F)(F)F)c1 ; O=S(=O)(Cl)c1cccc(Br)c1 ; O=S(=O)(Nc1cccc(C(F)(F)F)c1)c1cccc(Br)c1]

### Experimental Procedure:

Pyridine (9.2 mL 117.4 mmol) was added to a stirred solution of 3-bromobenzene-sulfonyl chloride Int-20 (10.0 g, 39.13 mmol) in DCM (200 mL) at 0° C., followed by 3-(trifluoromethyl)aniline Int-8 (4.9 mL, 39.13 mmol) at 0° C. The reaction mixture was stirred at room temperature for 16 hrs, then it was diluted with 1 N HCl solution (200 mL) and extracted with Ethyl Acetate (2×300 mL). Combined organic layers were washed with water (2×120 mL), brine (120 mL), dried over anhydrous sodium sulfate and concentrated under reduced pressure to afford 12.0 g of 3-bromo-N-(3-(trifluoromethyl)phenyl)benzenesulfonamide Int-21 as a pale brown solid in 80% yield. ¹H-NMR (400 MHz, CDCl₃): δ 7.94 (t, J=1.7 Hz, 1H), 7.69 (app tdd, J=8.0, 1.8, 1.0 Hz, 2H), 7.42 (d, J=5.2 Hz, 2H), 7.35 (d, J=8.0 Hz, 1H), 7.32-7.28 (m, 2H), 6.76 (bs, 1H); MS (ESI) m/z 382.33 [M+H]⁺.

### Ethyl (E)-3-(3-(N-(3-(trifluoromethyl)phenyl)sulfamoyl)phenyl)acrylate (Int-22)

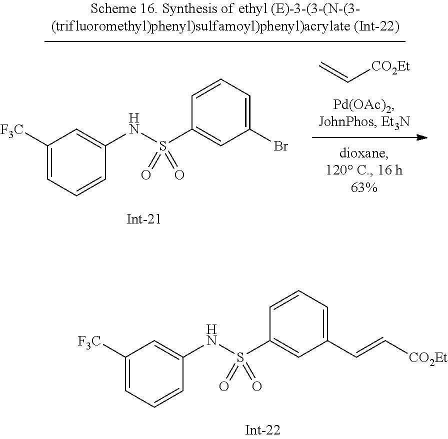

### Experimental Procedure:

Triethylamine (5.49 mL 39.47 mmol) and Ethyl Acrylate (1.59 mL 15.78 mmol) were added to a degassed solution of 3-bromo-N-(3-(trifluoromethyl)phenyl)benzene sulfonamide Int-21 (3.0 g, 7.89 mmol) in 1,4-Dioxane (45 mL) in a sealed tube at room temperature. Then JohnPhos (0.47 g, 1.58 mmol) and Pd(OAc)₂ (0.53 g 0.79 mmol) were added to the reaction mixture and it was degassed for additional 10 min. Then the reaction mixture was heated at 120° C. for 16 hrs. After cooling down to room temperature, the reaction mixture was diluted with Ethyl Acetate (100 mL), filtered through a pad of Celite and washed with Ethyl Acetate. The filtrate was concentrated under reduced pressure, and the crude residue was purified by column chromatography on silica gel (100-200 mesh) using 20% Ethyl Acetate in Hexane as eluent to afford 2.0 g of ethyl (E)-3-(3-(N-(3-(trifluoromethyl)phenyl)sulfamoyl)phenyl)acrylate Int-22 as a pale brown liquid in 63% yield. ¹H-NMR (300 MHz, DMSO-d₆): δ 10.78 (bs, 1H), 8.08 (s, 1H), 8.01 (d, J=7.8 Hz, 1H), 7.79 (d, J=7.8 Hz, 1H), 7.69 (d, J=15.9 Hz, 1H), 7.61 (t, J=7.8 Hz, 1H), 7.54-7.11 (m, 4H), 6.68 (d, J=16.2 Hz, 1H), 4.20 (q, J=7.5 Hz, 2H), 1.26 (t, J=7.2 Hz, 3H); MS (ESI) m/z 400.10 [M+H]⁺.

### (E)-3-(3-(N-(3-(trifluoromethyl)phenyl)sulfamoyl)phenyl)acrylic acid (Int-23)

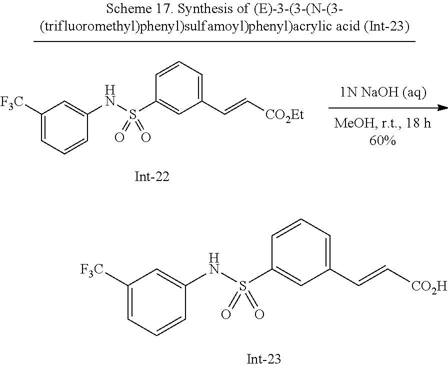 [SMILES: CCOC(=O)/C=C/c1cccc(S(=O)(=O)Nc2cccc(C(F)(F)F)c2)c1 ; O=C(O)/C=C/c1cccc(S(=O)(=O)Nc2cccc(C(F)(F)F)c2)c1]

### Experimental Procedure:

1 M NaOH solution (25 mL, 25.04 mmol) was added to a stirred solution of ethyl (E)-3-(3-(N-(3-(trifluoromethyl)phenyl)-sulfamoyl)phenyl)acrylate Int-22 (2.5 g, 6.26 mmol) in methanol (50 mL) at 0° C., and the reaction mixture was stirred at room temperature for 12 hrs. Then methanol was removed under reduced pressure, the residue was diluted with water (10 mL) and the aqueous layer was adjusted to pH 3-4 with 1 N HCl solution. The resulting precipitate was filtered, washed with water (20 mL) and dried in vacuo to afford 1.4 g of (E)-3-(3-(N-(3-(trifluoromethyl)phenyl)sulfamoyl)phenyl)acrylic acid Int-23 as a pale yellow solid in 60% yield. ¹H-NMR (300 MHz, DMSO-d₆): δ 11.42 (bs, 1H), 8.02 (s, 1H), 7.94 (d, J=7.8 Hz, 1H), 7.77 (d, J=8.1 Hz, 1H), 7.66-7.57 (m, 2H), 7.51-7.42 (m, 1H), 7.41-7.30 (m, 3H), 6.55 (d, J=15.9 Hz, 1H); MS (ESI) m/z 372.35 [M+H]⁺.

### (E)-3-(3-(N-methyl-N-(3-(trifluoromethyl)phenyl)sulfamoyl)phenyl)acrylic acid (Int-24)

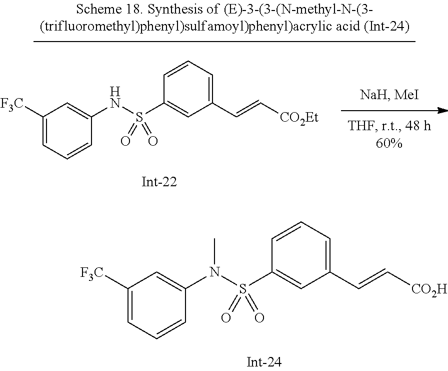 [SMILES: CCOC(=O)/C=C/c1cccc(S(=O)(=O)Nc2cccc(C(F)(F)F)c2)c1 ; CN(c1cccc(C(F)(F)F)c1)S(=O)(=O)c1cccc(/C=C/C(=O)O)c1]

### Experimental Procedure:

Sodium hydride (0.75 g, 18.79 mmol) was added to a stirred solution of ethyl (E)-3-(3-(N-(3-(trifluoromethyl)phenyl)sulfamoyl) phenyl)acrylate Int-22 (2.5 g, 6.26 mmol) in THF (50 mL) at 0° C. and the reaction mixture was stirred at 0° C. for 10 min. Then iodomethane (1.95 mL, 31.32 mmol) was added to the reaction mixture at 0° C., and the reaction mixture was left to stir at room temperature for 48 hrs. Then methanol was removed under reduced pressure, the residue was diluted with water (20 mL) and the aqueous reaction mixture was adjusted to pH 3-4 with 1 N HCl solution. The resulting precipitate was filtered, washed with water (20 mL) and dried in vacuo to afford 1.45 g of (E)-3-(3-(N-methyl-N-(3-(trifluoromethyl)phenyl)sulfamoyl)phenyl)acrylic acid Int-24 as an off-white solid in 60% yield. ¹H-NMR (300 MHz, DMSO-d₆): δ 12.58 (bs, 1H), 8.07 (d, J=7.5 Hz, 1H), 7.76 (s, 1H), 7.72-7.58 (m, 4H), 7.52-7.39 (m, 3H), 6.55 (d, J=16.2 Hz, 1H), 3.22 (s, 3H); MS (ESI) m/z 386.07 [M+H]⁺.

### Synthesis of (E)-3-(3-(N-methyl-N-phenylsulfamoyl)phenyl)acrylic acid (Int-28)

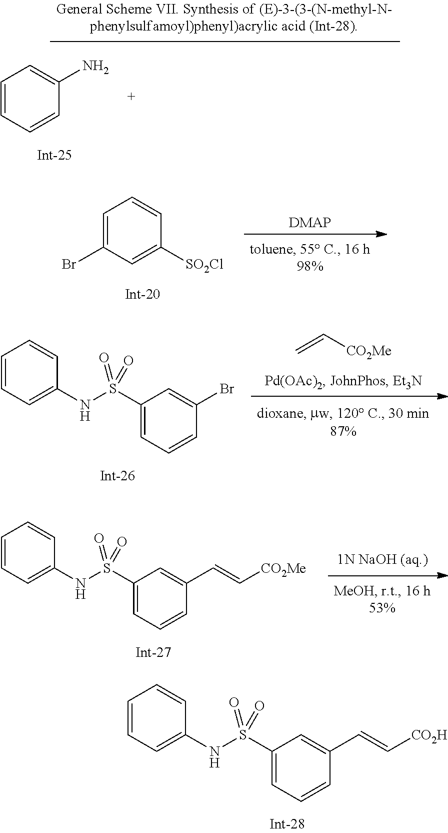

### 3-Bromo-N-phenylbenzenesulfonamide (Int-26)

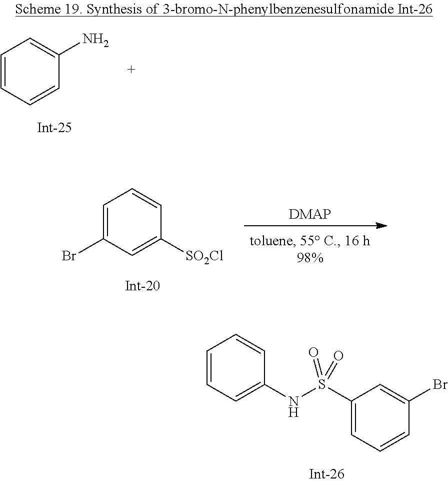

### Experimental Procedure:

3-Bromobenzenesulfonyl chloride Int-20 (751.2 mg, 2.94 mmol) was dissolved in toluene (4.5 mL), and aniline Int-25 (589 μL, 6.45 mmol) and DMAP (36 mg, 0.29 mmol) were added. The reaction mixture was heated at 55° C. in a sealed vial for 16 hrs. After cooling down to room temperature, the reaction mixture was diluted with toluene (5 mL) and washed with 2 N aqueous HCl solution (1×5 mL), saturated aqueous NaHCO₃ solution (1×5 mL), brine (1×5 mL), dried over sodium sulfate and concentrated in vacuo to give 3-bromo-N-phenylbenzenesulfonamide Int-26 as off-white solid in 98% yield (896 mg). ¹H-NMR (400 MHz, CDCl₃): δ 7.91 (t, J=1.8 Hz, 1H), 7.69-7.62 (m, J=1.9, 1.0 Hz, 2H), 7.32-7.27 (m, 3H), 7.19-7.15 (m, 1H), 7.08-7.05 (m, 2H), 6.52 (bs, 1H).

### Methyl (E)-3-(3-(N-phenylsulfamoyl)phenyl)acrylate (Int-27)

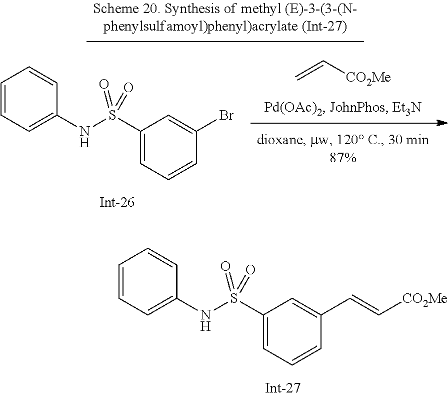

### Experimental Procedure:

3-Bromo-N-phenylbenzenesulfonamide Int-26 (311.2 mg, 1.0 mmol) was dissolved in 1,4-Dioxane (20 mL) in a 20 mL microwave vial, and triethylamine (277 μL, 2.0 mmol) was added, followed by JohnPhos (84.9 mg, 0.20 mmol) and palladium(II) acetate (22.5 mg, 0.10 mmol). The resulting solution was degassed with Nitrogen gas for 10 minutes before methylacrylate (227 p L, 2.5 mmol) was added. After addition, the vial was immediately sealed and heated at 120° C. in a microwave reactor for 30 minutes. After cooling down to room temperature, the reaction mixture was filtered through a pad of celite and washed with Ethyl Acetate (10 mL). The filtrate was concentrated under reduced pressure, and the residue was purified by column chromatography on silica gel using a mixture of Hexane and Ethyl Acetate as eluent (gradient: 100:0 to 40:60) to give 225 mg of methyl (E)-3-(3-(N-phenylsulfamoyl)phenyl)acrylate Int-27 as an orange solid in 71% yield. ¹H-NMR (400 MHz, CDCl₃): δ 7.87 (t, J=1.7 Hz, 1H), 7.74 (ddd, J=7.9, 1.8, 1.1 Hz, 1H), 7.65 (d, J=7.8 Hz, 1H), 7.62 (d, J=16.4 Hz, 1H), 7.46 (t, J=7.8 Hz, 1H), 7.29-7.24 (m, 1H), 7.17-7.13 (m, 1H), 7.08-7.05 (m, 2H), 6.57 (bs, 1H), 6.41 (d, J=16.0 Hz, 1H), 3.81 (s, 3H).

### (E)-3-(3-(N-phenylsulfamoyl)phenyl)acrylic acid (Int-28)

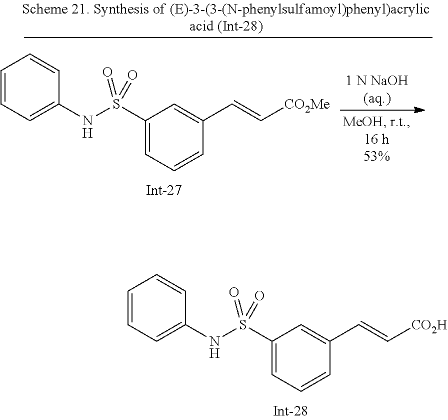 [SMILES: COC(=O)/C=C/c1cccc(S(=O)(=O)Nc2ccccc2)c1 ; O=C(O)/C=C/c1cccc(S(=O)(=O)Nc2ccccc2)c1]

### Experimental Procedure:

Methyl (E)-3-(3-(N-phenylsulfamoyl)phenyl)acrylate Int-27 (98 mg, 0.31 mmol) was dissolved in methanol (1.4 mL) and aqueous 1 N NaOH solution (1.0 mL) was added to this solution. The reaction mixture was stirred at room temperature for 16 hours. Then methanol was removed under reduced pressure, and the residue was diluted with water (5 mL). The aqueous phase was washed with Ethyl Acetate (1×5 mL) before it was acidified with aqueous 2 N HCl solution to pH of approx. 2-3. The resulting mixture was stirred for 30 minutes at room temperature. Formed precipitate was isolated by centrifugation, washed with water (1×5 mL) and dried in vacuo to give 50.3 mg of (E)-3-(3-(N-phenylsulfamoyl)phenyl)acrylic acid Int-28 as an off-white solid in 53% yield. ¹H-NMR (400 MHz, DMSO-d₆): δ 12.61 (bs, 1H), 10.30 (bs, 1H), 8.00 (s, 1H), 7.93 (d, J=7.8 Hz, 1H), 7.74 (ddd, J=7.9, 1.6, 1.1 Hz, 1H), 7.62-7.56 (m, 2H), 7.25-7.20 (m, 2H), 7.11-7.08 (m, 2H), 7.05-7.01 (m, 1H), 6.55 (d, J=16.0 Hz, 1H).

### (E)-3-(3-(N-methyl-N-phenylsulfamoyl)phenyl)acrylic acid (Int-32)

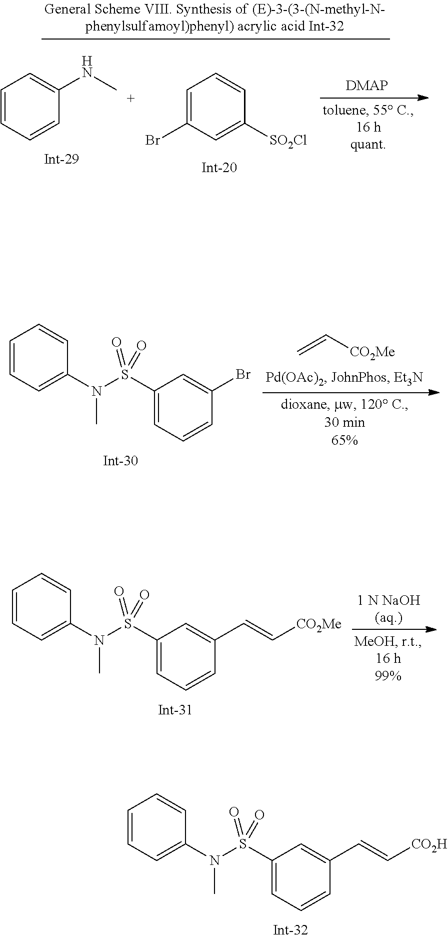

### 3-Bromo-N-methyl-N-phenylbenzenesulfonamide (Int-30)

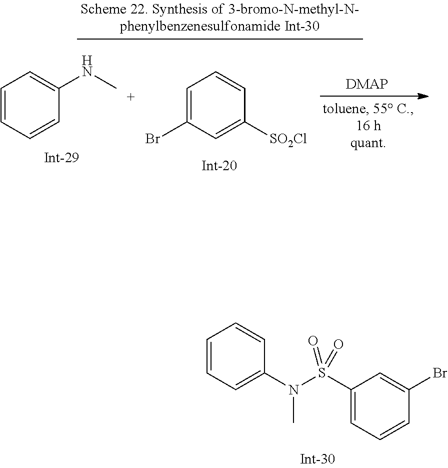

### Experimental Procedure:

3-Bromobenzenesulfonyl chloride Int-20 (1.0 g, 3.92 mmol) was dissolved in toluene (6.0 mL), then N-methyl aniline Int-29 (934 μL, 8.60 mmol) and DMAP (97.7 mg, 0.80 mmol) were added. The reaction mixture was heated at 55° C. in a sealed vial overnight. After cooling to room temperature, the reaction mixture was diluted with toluene (5 mL) and washed with 2 N aqueous HCl solution (1×5 mL), saturated aqueous NaHCO₃ solution (1×5 mL), brine (1×5 mL), dried over anhydrous sodium sulfate and concentrated in vacuo to give 3-bromo-N-methyl-N-phenylbenzenesulfonamide Int-30 as a light brown solid in quantitative yield (1.39 g, the product contains a small amount of toluene that does not interfere with the chemistry of the next step). ¹H-NMR (400 MHz, CDCl₃): δ 7.71-7.69 (m, 2H), 7.46-7.43 (m, 1H), 7.35-7.29 (m, 4H), 7.11-7.08 (m, 2H), 3.20 (s, 3H).

### Methyl (E)-3-(3-(N-methyl-N-phenylsulfamoyl)phenyl)acrylate (Int-31)

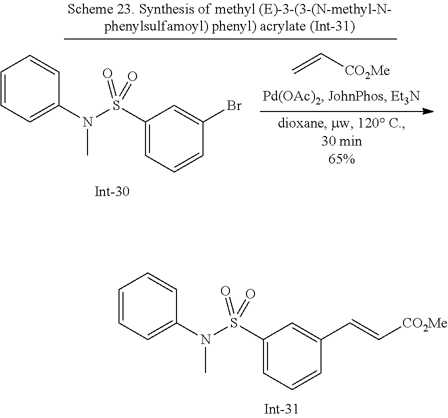

### Experimental Procedure:

3-Bromo-N-methyl-phenylbenzenesulfonamide Int-30 (1.39 g, 3.92 mmol) was dissolved in 1,4-Dioxane (20 mL) in a 20 mL microwave vial and triethylamine (1.1 mL, 7.84 mmol) was added, followed by JohnPhos (332.9 mg, 0.78 mmol) and palladium(II) acetate (87.6 mg, 0.39 mmol). The resulting solution was degassed with Nitrogen gas for 10 minutes before methyl acrylate (888 p L, 9.8 mmol) was added. After addition, the vial was immediately sealed and heated at 120° C. in a microwave reactor for 30 minutes. After cooling to room temperature, the reaction mixture was filtered through a pad of celite and washed with Ethyl Acetate (10 mL). The filtrate was concentrated under reduced pressure, and the residue was purified by column chromatography on silica gel using a mixture of Hexane and Ethyl Acetate as eluent (gradient: 100:0 to 20:80) to give 843.6 mg of methyl (E)-3-(3-(N-methyl-N-phenylsulfamoyl)phenyl)acrylate Int-31 as a light yellow solid in 65% yield. ¹H-NMR (400 MHz, CDCl₃): δ 7.70 (dt, J=7.5, 1.3 Hz, 1H), 7.65-7.61 (m, 2H), 7.55 (dt, J=7.9, 1.5 Hz, 1H), 7.48 (t, J=7.7 Hz, 1H), 7.34-7.28 (m, 3H), 7.13-7.08 (m, 2H), 6.38 (d, J=16.1 Hz, 1H), 3.82 (s, 3H), 3.19 (s, 3H).

### (E)-3-(3-(N-methyl-N-phenylsulfamoyl)phenyl)acrylic acid (Int-32)

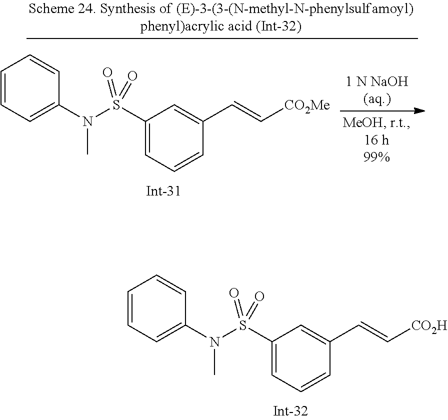 [SMILES: COC(=O)/C=C/c1cccc(S(=O)(=O)N(C)c2ccccc2)c1 ; CN(c1ccccc1)S(=O)(=O)c1cccc(/C=C/C(=O)O)c1]

### Experimental Procedure:

Methyl (E)-3-(3-(N-methyl-N-phenylsulfamoyl)phenyl)acrylate Int-31 (50 mg, 0.15 mmol) was dissolved in methanol (800 μL) before an aqueous 1 N NaOH solution (500 μL) was added. The reaction mixture was stirred at room temperature for 16 hours. Then methanol was removed under reduced pressure, and the residue was diluted with water (5 mL). The aqueous phase was washed with Diethyl Ether (1×5 mL), then it was acidified with aqueous 2 N HCl solution to pH of approx. 2-3. The resulting solution was stirred for 30 minutes at room temperature upon which time a white emulsion was formed. The aqueous layer was extracted with Ethyl Acetate (2×5 mL). Combined organic layers were washed with brine (1×5 mL), dried over anhydrous sodium sulfate and concentrated in vacuo to give 47.6 mg of (E)-3-(3-(N-methyl-N-phenylsulfamoyl)phenyl)acrylic acid Int-32 as an off-white solid in 99% yield. ¹H-NMR (400 MHz, DMSO-d₆): δ 12.53 (bs, 1H), 8.04 (d, J=7.8 Hz, 1H), 7.74 (t, J=1.6 Hz, 1H), 7.65-7.58 (m, 2H), 7.46 (ddd, J=7.8, 1.7, 1.1 Hz, 1H), 7.37-7.27 (m, 3H), 7.15-7.07 (m, 2H), 6.55 (d, J=16.1 Hz, 1H), 3.17 (s, 3H).

### Synthesis of (E)-3-(3-(N-(4-fluoro-3-(trifluoromethyl) phenyl) sulfamoyl)phenyl)acrylic acid

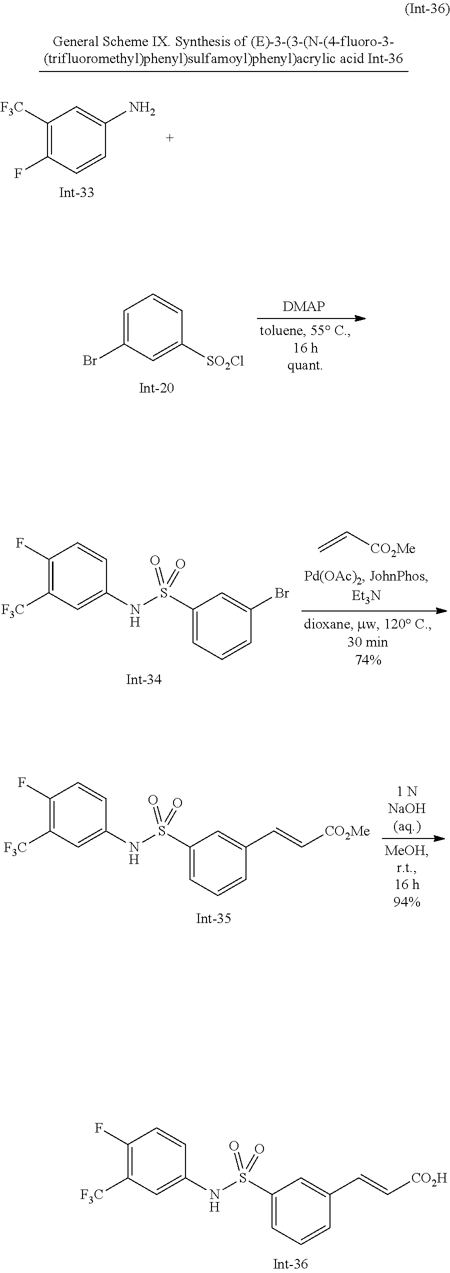

### 3-Bromo-N-(4-fluoro-3-(trifluoromethyl)phenyl)benzenesulfonamide Int-34

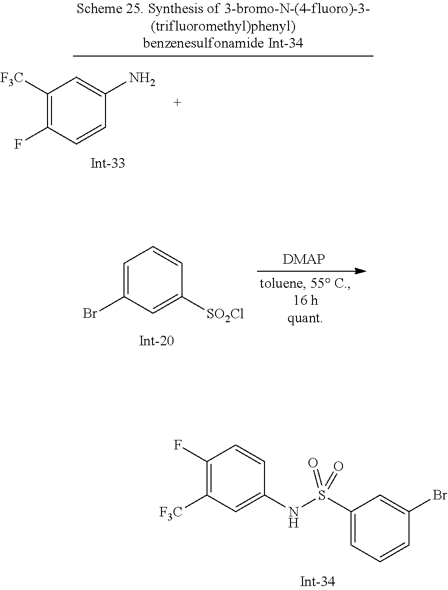 [SMILES: Nc1ccc(F)c(C(F)(F)F)c1 ; O=S(=O)(Cl)c1cccc(Br)c1 ; O=S(=O)(Nc1ccc(F)c(C(F)(F)F)c1)c1cccc(Br)c1]

### Experimental Procedure:

3-Bromobenzenesulfonyl chloride Int-20 (250 mg, 0.98 mmol) was dissolved in toluene (1.5 mL) before 5-amino-2-fluorobenzotrifluoride Int-33 (276 μL, 2.15 mmol) and DMAP (24.4 mg, 0.20 mmol) were added. The reaction mixture was heated at 55° C. in a sealed vial overnight. After cooling to room temperature, the reaction mixture was diluted with toluene (5 mL) and washed with 2 N aqueous HCl solution (1×5 mL), saturated aqueous NaHCO₃ solution (1×5 mL), brine (1×5 mL), dried over anhydrous sodium sulfate and concentrated in vacuo to give 3-bromo-N-(4-fluoro-3-(trifluoromethyl)phenyl)benzenesulfonamide Int-34 as a light pink solid in quantitative yield (407 mg, the product contains a small amount of toluene that does not interfere with the chemistry of the next step). ¹H-NMR (400 MHz, CDCl₃): δ 7.92 (t, J=1.7 Hz, 1H), 7.72 (ddd, J=8.0, 1.9, 1.0 Hz, 1H), 7.64 (ddd, J=7.9, 1.8, 1.0 Hz, 1H), 7.36 (t, J=8.0 Hz, 1H), 7.33-7.27 (m, 2H), 7.15 (q, J=8.8 Hz, 1H), 6.75 (s, 1H).

### Methyl (E)-3-(3-(N-(4-fluoro-3-(trifluoromethyl)phenyl)sulfamoyl) phenyl) acrylate Int-35

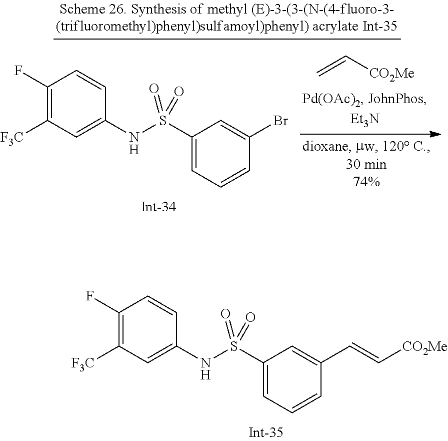

### Experimental Procedure:

3-Bromo-N-(4-fluoro-3-(trifluoromethyl)phenyl)benzenesulfonamide Int-34 (398.2 mg, 1.00 mmol) was dissolved in 1,4-Dioxane (10 mL) in a 20 mL microwave vial and Triethylamine (277 μL, 7.84 mmol) was added, followed by JohnPhos (84.9 mg, 0.20 mmol) and palladium(II) acetate (22.5 mg, 0.10 mmol). The resulting solution was degassed with Nitrogen gas for 10 minutes before methyl acrylate (227 p L, 2.5 mmol) was added. After addition, the vial was immediately sealed and heated at 120° C. in a microwave reactor for 30 minutes. After cooling to room temperature, the reaction mixture was filtered through a pad of Celite and washed with Ethyl Acetate (10 mL). The resulting filtrate was concentrated under reduced pressure. The residue was purified by column chromatography on silica gel using a mixture of Hexane and Ethyl Acetate as eluent (gradient: 100:0 to 30:70). Methyl (E)-3-(3-(N-(4-fluoro-3-(trifluoromethyl)phenyl)sulfamoyl)phenyl)acrylate Int-35 was obtained as an orange solid in 74% yield (298.9 mg). ¹H-NMR (400 MHz, CDCl₃): δ 7.75 (d, J=8.5 Hz, 2H), 7.66 (d, J=16.1 Hz, 1H), 7.60 (d, J=8.3 Hz, 2H), 7.30 (dd, J=5.8, 2.9 Hz, 2H), 7.12 (t, J=9.7 Hz, 1H), 6.51 (d, J=16.1 Hz, 1H), 3.83 (s, 3H).

### (E)-3-(3-(N-(4-fluoro-3-(trifluoromethyl)phenyl)sulfamoyl)phenyl)acrylic acid (Int-36)

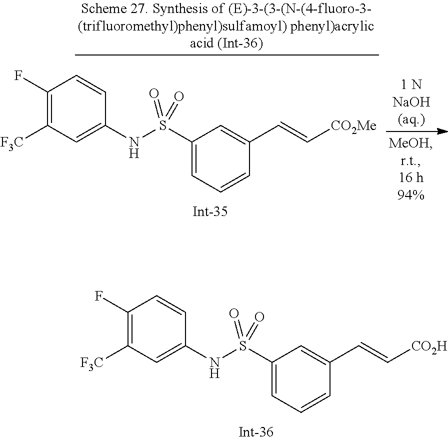 [SMILES: COC(=O)/C=C/c1cccc(S(=O)(=O)Nc2ccc(F)c(C(F)(F)F)c2)c1 ; O=C(O)/C=C/c1cccc(S(=O)(=O)Nc2ccc(F)c(C(F)(F)F)c2)c1]

### Experimental Procedure:

Aqueous 1 N NaOH solution (1.1 mL) was added to a solution of methyl (E)-3-(3-(N-(4-fluoro-3-(trifluoromethyl)phenyl)sulfamoyl)phenyl)acrylate Int-35 (150 mg, 0.73 mmol) in methanol (1.7 mL). The reaction mixture was stirred at room temperature for 16 hours. Then methanol was removed under reduced pressure and the residue was diluted with water (5 mL). The aqueous phase was washed with Diethyl Ether (1×5 mL) and acidified with aqueous 2N HCl solution to pH of approx. 2-3. The resulting solution was stirred for 30 minutes at room temperature, then extracted with Ethyl Acetate (2×5 mL). Combined organic layers were washed with brine (1×5 mL), dried over anhydrous sodium sulfate and concentrated in vacuo to give 136 mg of (E)-3-(3-(N-(4-fluoro-3-(trifluoromethyl)phenyl)sulfamoyl)phenyl)acrylic acid Int-36 as an off-white solid in 94% yield. ¹H-NMR (400 MHz, DMSO-d₆): δ 12.58 (bs, 1H), 10.66 (bs, 1H), 8.00 (t, J=1.6 Hz, 1H), 7.98 (d, J=7.8 Hz, 1H), 7.73 (ddd, J=7.9, 1.6, 1.1 Hz, 1H), 7.64 (d, J=5.4 Hz, 1H), 7.62-7.58 (m, 1H), 7.46-7.41 (m, 2H), 7.38 (d, J=7.1 Hz, 1H), 6.57 (d, J=16.1 Hz, 1H).

### Synthesis of (E)-3-(3N-(4-fluoro-3-(trifluoromethyl)phenyl)-N-methylsulfamoyl) phenyl)acrylic acid Int-38

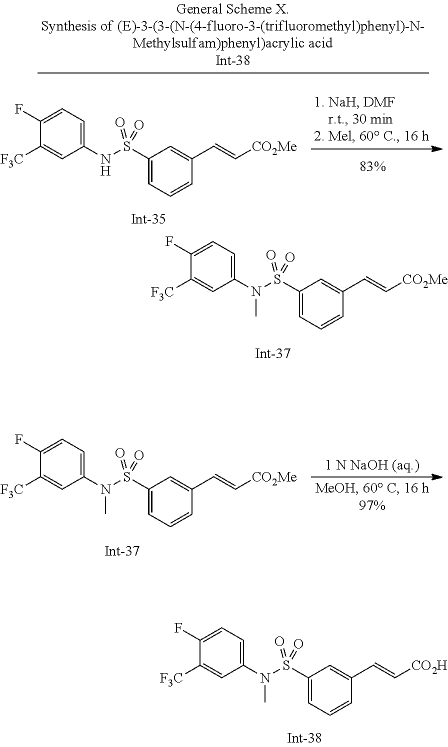

### Methyl (E)-3-(3-(N-(4-fluoro-3-(trifluoromethyl)phenyl)-N-methylsulfamoyl) phenyl)acrylate (Int-37)

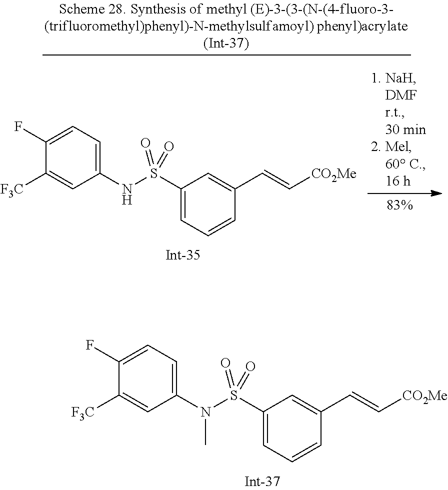 [SMILES: COC(=O)/C=C/c1cccc(S(=O)(=O)Nc2ccc(F)c(C(F)(F)F)c2)c1 ; COC(=O)/C=C/c1cccc(S(=O)(=O)N(C)c2ccc(F)c(C(F)(F)F)c2)c1]

### Experimental Procedure:

Methyl(E)-3-(3-(N-(4-fluoro-3-(trifluoromethyl)phenyl)sulfamoyl)phenyl)acrylate Int-35 (150.0 mg, 0.37 mmol) was dissolved in dry DMF (1 mL) and sodium hydride (19.3 mg, 0.48 mmol, 60% dispersion in mineral oil) was slowly added at room temperature. The reaction mixture was stirred at room temperature for 30 minutes, then iodomethane (58 μL, 0.93 mmol) was added dropwise. The reaction mixture was heated at 60° C. for 16 hours, then cooled down to room temperature, quenched with water (5 mL) and extracted with Ethyl Acetate (3×5 mL). Combined organic layers were washed with brine (1×5 mL), dried over anhydrous sodium sulfate and concentrated in vacuo. The residue was purified by column chromatography on silica gel using a mixture of Hexane and Ethyl Acetate as eluent (gradient: 100:0 to 20:80) to give 127.8 mg of methyl (E)-3-(3-(N-(4-fluoro-3-(trifluoromethyl)phenyl)-N-methylsulfamoyl)phenyl) acrylate Int-37 as a light yellow solid in 83% yield. ¹H-NMR (400 MHz, CDCl₃): δ 7.75 (dt, J=6.6, 1.9 Hz, 1H), 7.70 (bs, 1H), 7.66 (d, J=16.0 Hz, 1H), 7.55-7.50 (m, 2H), 7.42-7.32 (m, 2H), 7.26-7.24 (m, 1H), 7.18 (t, J=9.2 Hz, 1H), 6.44 (d, J=16.0 Hz, 1H), 3.83 (s, 3H), 3.18 (s, 3H).

### (E)-3-(3-(N-(4-fluoro-3-(trifluoromethyl)phenyl)-N-methylsulfamoyl) phenyl) acrylic acid (Int-38)

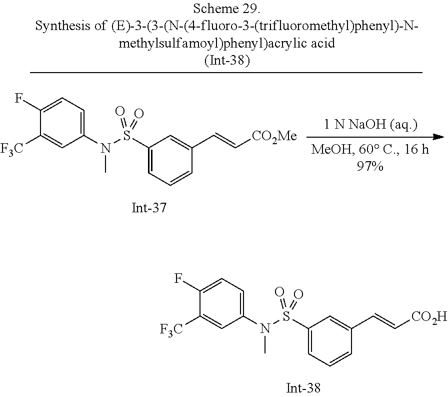

### Experimental Procedure:

Aqueous 1N NaOH solution (920 p L) was added to a solution of methyl (E)-3-(3-(N-(4-fluoro-3-(trifluoromethyl)phenyl)-N-methylsulfamoyl)phenyl)-acrylate Int-37 (127.8 mg, 0.31 mmol) in methanol (1.4 mL). The reaction mixture was stirred at room temperature for 16 hours. Then Methanol was removed under reduced pressure and the residue was diluted with water (5 mL). The aqueous phase was washed with Diethyl Ether (1×5 mL) before it was acidified with aqueous 2N HCl solution to pH of approx. 2-3. The resulting solution was stirred for 30 minutes at room temperature, and then extracted with Ethyl Acetate (2×5 mL). Combined organic layers were washed with brine (1×5 mL), dried over anhydrous sodium sulfate and concentrated in vacuo to give 121.8 mg of (E)-3-(3-(N-(4-fluoro-3-(trifluoromethyl)phenyl)-N-methylsulfamoyl) phenyl) acrylic acid Int-38 as an off-white solid in 97% yield. MS (ESI) m/z=402 [M−H⁺].

### Synthesis of (E)-3-(3-(N-(4-(trifluoromethyl)phenyl)sulfamoyl)phenyl)acrylic acid (Int-42)

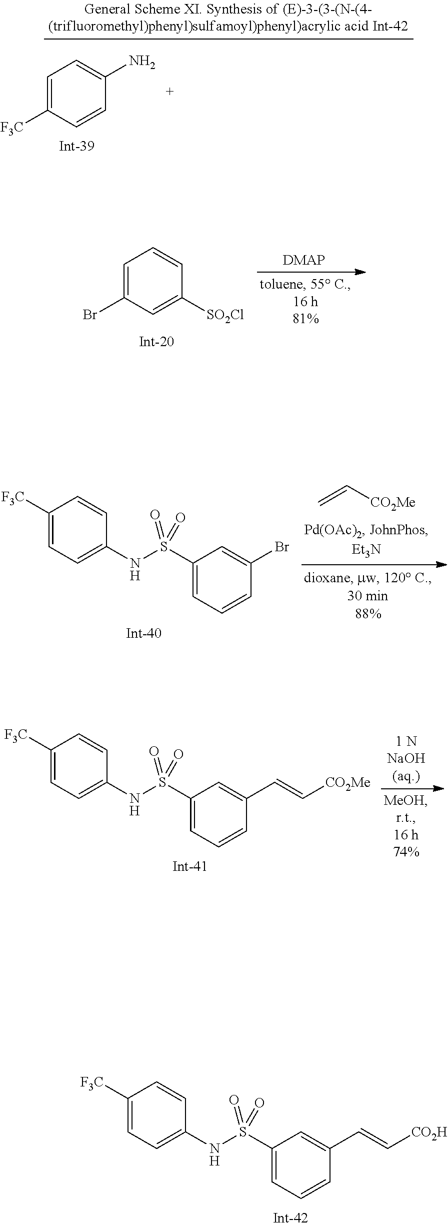

### 3-Bromo-N-(4-(trifluoromethyl)phenyl)benzenesulfonamide (Int-40)

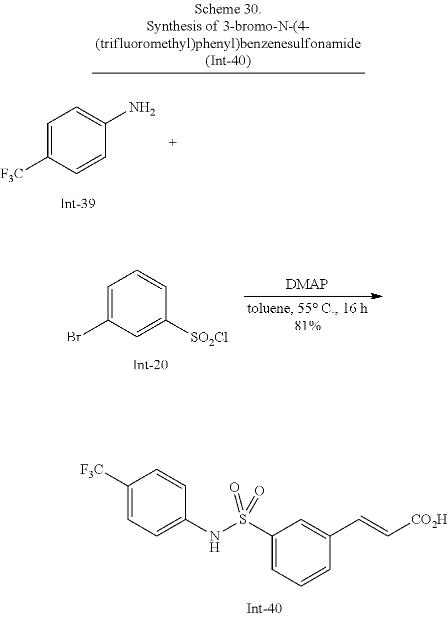

### Experimental Procedure:

4-Amino-benzotrifluoride Int-39 (270 μL, 2.15 mmol) and DMAP (12.2 mg, 0.10 mmol) were added to the solution of 3-bromobenzenesulfonyl chloride Int-20 (250 mg, 0.98 mmol) in toluene (1.5 mL). The reaction mixture was heated at 55° C. in a sealed vial overnight. After cooling to room temperature, the reaction mixture was diluted with toluene (5 mL) and washed with 2N aqueous HCl solution (1×5 mL), saturated aqueous NaHCO₃ solution (1×5 mL), brine (1×5 mL), dried over anhydrous sodium sulfate and concentrated in vacuo. The residue was purified via column chromatography on silica gel using Hexanes and Ethyl Acetate as eluent (gradient: 100:0 to 0:100) to give 301 mg of 3-bromo-N-(4-(trifluoromethyl)phenyl)benzenesulfonamide Int-40 as yellow oil in 81% yield. ¹H-NMR (400 MHz, CDCl₃): δ 8.00 (s, 1H), 7.74 (ddd, J=8.0, 1.6, 0.8 Hz, 1H), 7.71 (ddd, J=8.1, 1.6, 0.9 Hz, 1H), 7.54 (d, J=8.4 Hz, 2H), 7.36 (t, J=8.0 Hz, 1H), 7.20 (d, J=8.4 Hz, 2H).

### Methyl (E)-3-(3-(N-(4-(trifluoromethyl)phenyl)sulfamoyl)phenyl)acrylate (Int-41)

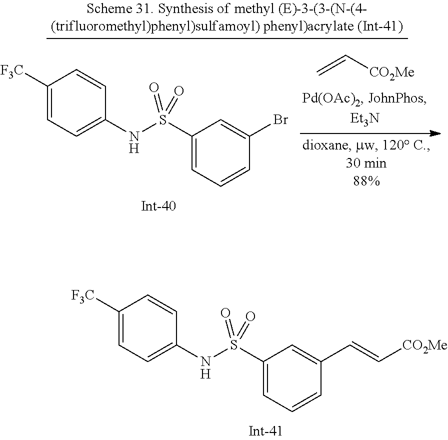

### Experimental Procedure:

3-Bromo-N-(4-(trifluoromethyl)phenyl)benzenesulfonamide Int-40 (75.8 mg, 0.20 mmol) was dissolved in 1,4-Dioxane (4 mL) in a 5 mL microwave vial and Triethylamine (55 μL, 0.40 mmol) was added, followed by JohnPhos (34.0 mg, 0.08 mmol) and palladium(II) acetate (9.0 mg, 0.04 mmol). The resulting solution was degassed with Nitrogen gas for 10 minutes, then methyl acrylate (45 p L, 0.5 mmol) was added. After addition, the vial was immediately sealed and heated at 120° C. in a microwave reactor for 30 minutes. After cooling down to room temperature, the reaction mixture was filtered through a pad of Celite and washed with Ethyl Acetate (10 mL). The filtrate was concentrated under reduced pressure and the residue was purified by column chromatography on silica gel using a mixture of Hexane and Ethyl Acetate as eluent (gradient: 100:0 to 40:60) to give 68 mg of methyl (E)-3-(3-(N-(4-(trifluoromethyl) phenyl)sulfamoyl)phenyl)acrylate Int-41 as yellow oil in 88% yield. ¹H-NMR (400 MHz, MeOD): δ 7.97 (d, J=1.7 Hz, 1H), 7.81 (d, J=7.9 Hz, 1H), 7.70 (d, J=7.8 Hz, 1H), 7.65 (d, J=16.1 Hz, 1H), 7.57-7.49 (m, 3H), 7.20 (d, J=8.4 Hz, 2H), 6.45 (d, J=16.1 Hz, 1H), 3.82 (s, 3H).

### (E)-3-(3-(N-(4-(trifluoromethyl)phenyl)sulfamoyl)phenyl)acrylic acid (Int-42)

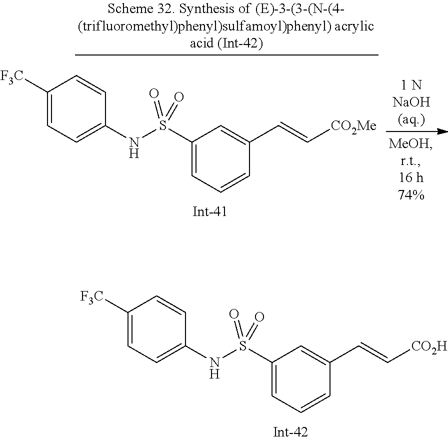 [SMILES: COC(=O)/C=C/c1cccc(S(=O)(=O)Nc2ccc(C(F)(F)F)cc2)c1 ; O=C(O)/C=C/c1cccc(S(=O)(=O)Nc2ccc(C(F)(F)F)cc2)c1]

### Experimental Procedure:

Aqueous 1N NaOH solution (600 μL) was added to the solution of methyl (E)-3-(3-(N-(4-(trifluoromethyl)phenyl)sulfamoyl)phenyl)acrylate Int-41 (68 mg, 0.19 mmol) in Methanol (850 μL). The reaction mixture was stirred at room temperature for 16 hours. Then Methanol was removed under reduced pressure and the residue was diluted with water (5 mL). The aqueous phase was washed with Diethyl Ether (1×5 mL) before it was acidified with aqueous 2 N HCl solution to pH of approx. 2-3. The resulting solution was stirred for 30 minutes at room temperature, and then extracted with Ethyl Acetate (2×5 mL). Combined organic layers were washed with brine (1×5 mL), dried over anhydrous sodium sulfate and concentrated in vacuo to give 52.3 mg of (E)-3-(3-(N-(4-(trifluoromethyl)phenyl)sulfamoyl)phenyl)acrylic acid Int-42 as an off-white solid in 74% yield. ¹H-NMR (400 MHz, DMSO-d₆): δ 12.39 (bs, 1H), 10.92 (bs, 1H), 8.09 (t, J=1.6 Hz, 1H), 7.98 (d, J=7.8 Hz, 1H), 7.81 (ddd, J=7.9, 1.7, 1.0 Hz, 1H), 7.64 (d, J=5.9 Hz, 1H), 7.62-7.60 (m, 3H), 7.30 (d, J=8.4 Hz, 2H), 6.59 (d, J=16.1 Hz, 1H).

### Synthesis of (E)-3-(3-(N-(3-(trifluoromethoxy)phenyl)sulfamoyl)phenyl)acrylic acid (Int-46)

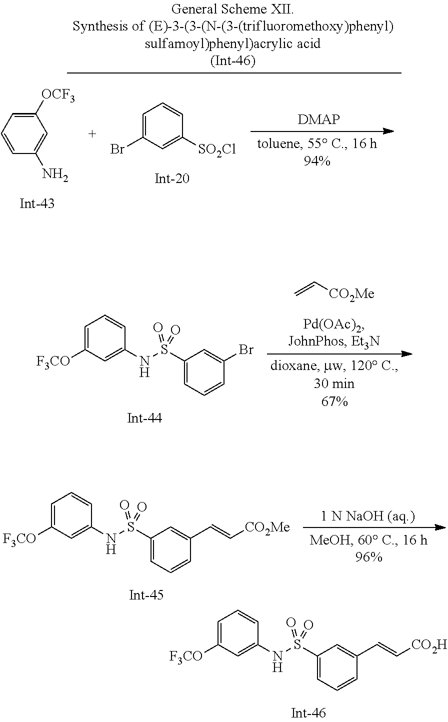

### 3-Bromo-N-(3-(trifluoromethoxy)phenyl)benzenesulfonamide (Int-44)

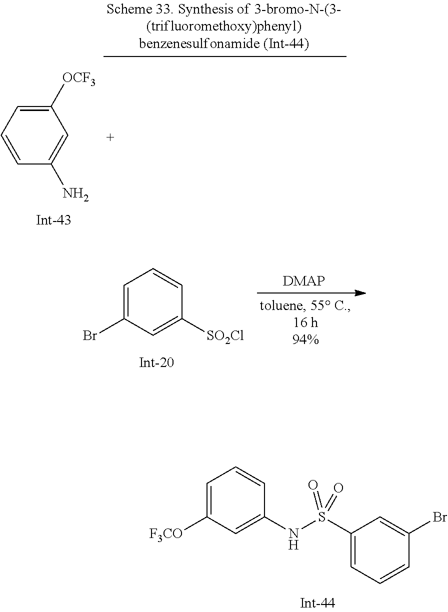 [SMILES: Nc1cccc(OC(F)(F)F)c1 ; O=S(=O)(Cl)c1cccc(Br)c1 ; O=S(=O)(Nc1cccc(OC(F)(F)F)c1)c1cccc(Br)c1]

### Experimental Procedure:

3-Bromobenzenesulfonyl chloride Int-20 (250 mg, 0.98 mmol) was dissolved in toluene (1.5 mL) and 3-(trifluoromethoxy)aniline Int-43 (287 μL, 2.15 mmol) was added, followed by DMAP (24.4 mg, 0.20 mmol). The reaction mixture was heated at 55° C. in a sealed vial overnight. After cooling to room temperature, the reaction mixture was diluted with toluene (5 mL), washed with 2N aqueous HCl solution (1×5 mL), saturated aqueous NaHCO₃ solution (1×5 mL), and brine (1×5 mL), dried over anhydrous sodium sulfate and concentrated in vacuo to give 365.6 mg of 3-bromo-N-(3-(trifluoromethoxy)phenyl)-benzenesulfonamide Int-44 as an off-white solid in 94% yield. ¹H-NMR (400 MHz, CDCl₃): δ 7.95 (t, J=1.8 Hz, 1H), 7.71-7.68 (m, 2H), 7.34 (t, J=8.0 Hz, 1H), 7.29 (td, J=7.9, 0.9 Hz, 1H), 7.02-6.98 (m, 3H).

### Methyl (E)-3-(3-(N-(3-(trifluoromethoxy)phenyl)sulfamoyl)phenyl)acrylate (Int-45)

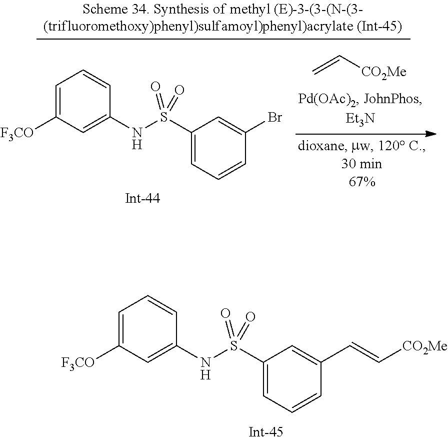

### Experimental Procedure:

3-Bromo-N-(3-(trifluoromethoxy)phenyl)benzenesulfonamide Int-44 (365.6 mg, 0.92 mmol) was dissolved in 1,4-Dioxane (10 mL) in a 20 mL microwave vial and triethylamine (255 μL, 1.84 mmol) was added, followed by JohnPhos (78.1 mg, 0.18 mmol) and palladium(II) acetate (20.7 mg, 0.09 mmol). The resulting solution was degassed with Nitrogen gas for 10 minutes, and then methyl acrylate (208 μL, 2.3 mmol) was added. After addition, the vial was immediately sealed and heated at 120° C. in a microwave reactor for 30 minutes. After cooling down to room temperature, the reaction mixture was filtered through a pad of Celite and washed with Ethyl Acetate (10 mL). The filtrate was concentrated under reduced pressure and the residue was purified by column chromatography on silica gel using a mixture of Hexane and Ethyl Acetate as eluent (gradient: 90:10 to 20:80) to give 264.2 mg of methyl (E)-3-(3-(N-(3-(trifluoromethoxy)phenyl)-sulfamoyl)phenyl)acrylate Int-45 as yellow oil in 67% yield. ¹H-NMR (400 MHz, CDCl₃): δ 7.92 (t, J=1.6 Hz, 1H), 7.77 (ddd, J=7.9, 1.8, 1.1 Hz, 1H), 7.68 (d, J=7.8 Hz, 1H), 7.63 (d, J=16.1 Hz, 1H), 7.49 (t, J=7.8 Hz, 1H), 7.28 (t, J=8.5 Hz, 1H), 7.02-6.97 (m, 3H), 6.44 (d, J=16.0 Hz, 1H), 3.82 (s, 3H).

### (E)-3-(3-(N-(3-(trifluoromethoxy)phenyl)sulfamoyl)phenyl)acrylic acid (Int-46)

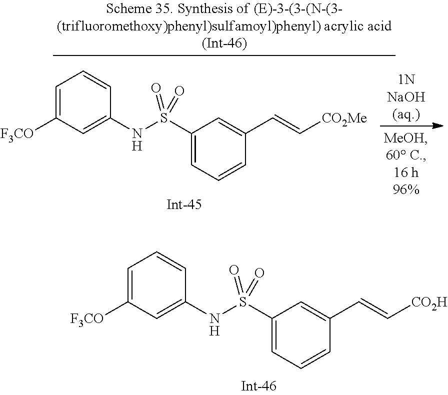 [SMILES: COC(=O)/C=C/c1cccc(S(=O)(=O)Nc2cccc(OC(F)(F)F)c2)c1 ; O=C(O)/C=C/c1cccc(S(=O)(=O)Nc2cccc(OC(F)(F)F)c2)c1]

### Experimental Procedure:

Aqueous 1N NaOH solution (790 μL) was added to the solution of methyl (E)-3-(3-(N-(3-(trifluoromethoxy)phenyl)sulfamoyl)phenyl)acrylate Int-45 (100 mg, 0.25 mmol) in Methanol (1.3 mL). The reaction mixture was heated at 60° C. for 16 hrs. Then Methanol was removed under reduced pressure and the residue was diluted with water (5 mL). The aqueous phase was washed with Diethyl Ether (1×5 mL) before it was acidified with aqueous 2 N HCl solution to pH of approx. 2-3. The resulting solution was stirred for 30 minutes at room temperature, and then extracted with Ethyl Acetate (2×5 mL). Combined organic layers were washed with brine (1×5 mL), dried over anhydrous sodium sulfate and concentrated in vacuo to give 92.9 mg of (E)-3-(3-(N-(3-(trifluoromethoxy)phenyl)sulfamoyl)phenyl)acrylic acid Int-46 as an orange solid in 96% yield. MS (ESI) m/z=386 [M−H⁺].

### Synthesis of (E)-3-(3-(N-(4-(trifluoromethoxy)phenyl)sulfamoyl)phenyl)acrylic acid (Int-50)

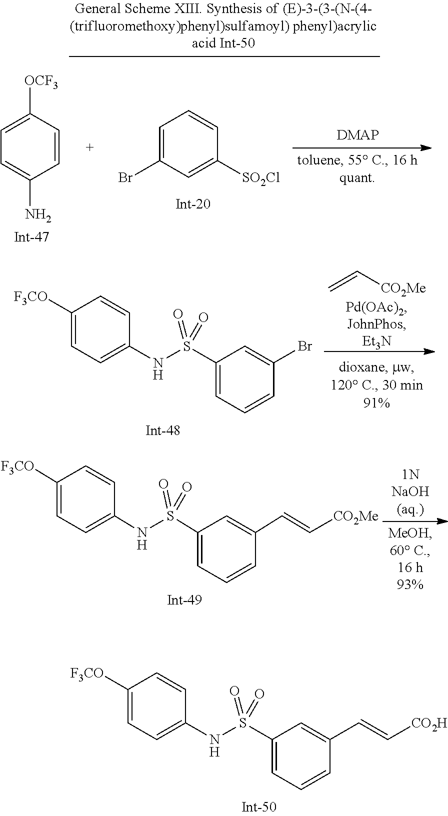

### 3-Bromo-N-(4-(trifluoromethoxy)phenyl)benzenesulfonamide (Int-48)

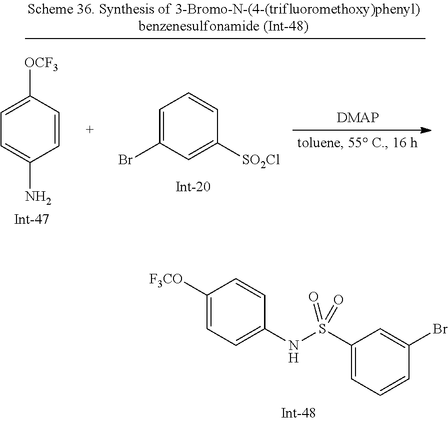 [SMILES: Nc1ccc(OC(F)(F)F)cc1 ; O=S(=O)(Cl)c1cccc(Br)c1 ; O=S(=O)(Nc1ccc(OC(F)(F)F)cc1)c1cccc(Br)c1]

### Experimental Procedure:

3-Bromobenzenesulfonyl chloride Int-20 (250 mg, 0.98 mmol) was dissolved in toluene (1.5 mL) and 4-(trifluoromethoxy)aniline Int-47 (291 μL, 2.15 mmol) was added, followed by DMAP (24.4 mg, 0.20 mmol). The reaction mixture was heated at 55° C. in a sealed vial overnight. After cooling to room temperature, the reaction mixture was diluted with toluene (5 mL), washed with 2N aqueous HCl solution (1×5 mL), saturated aqueous NaHCO₃ solution (1×5 mL), and brine (1×5 mL), dried over anhydrous sodium sulfate and concentrated in vacuo to give 401 mg of 3-bromo-N-(4-(trifluoromethoxy)phenyl)-benzenesulfonamide Int-48 as an off-white solid in quantitative yield (the product contained a small amount of toluene that did not interfere with the chemistry of the next step). ¹H-NMR (400 MHz, CDCl₃): δ 7.92 (t, J=1.7 Hz, 1H), 7.69 (ddd, J=8.0, 1.9, 1.0 Hz, 1H), 7.67 (ddd, J=8.0, 1.8, 1.0 Hz, 1H), 7.34 (t, J=8.0 Hz, 1H), 7.15-7.09 (m, 5H), 6.82 (bs, 1H).

### Methyl (E)-3-(3-(N-(4-(trifluoromethoxy)phenyl)sulfamoyl)phenyl)acrylate (Int-49)

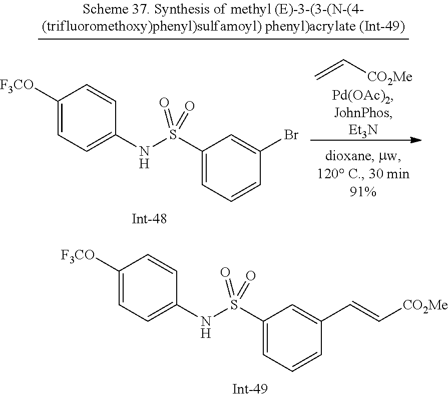

### Experimental Procedure:

3-Bromo-N-(4-(trifluoromethoxy)phenyl)benzenesulfonamide Int-48 (401.0 mg, 0.98 mmol) was dissolved in 1,4-Dioxane (10 mL) in a 20 mL microwave vial and Triethylamine (277 μL, 2.00 mmol) was added, followed by JohnPhos (84.9 mg, 0.20 mmol) and Palladium(II) Acetate (22.5 mg, 0.10 mmol). The resulting solution was degassed with Nitrogen gas for 10 minutes, and then methyl acrylate (227 μL, 2.5 mmol) was added. After addition, the vial was immediately sealed and heated at 120° C. in a microwave reactor for 30 minutes. After cooling to room temperature, the reaction mixture was filtered through a pad of Celite and washed with Ethyl Acetate (10 mL). The filtrate was concentrated under reduced pressure, and the residue was purified by column chromatography on silica gel using a mixture of Hexane and Ethyl Acetate as eluent (gradient: 100:0 to 30:70) to give 356.3 mg of methyl (E)-3-(3-(N-(4-(trifluoromethoxy)-phenyl)sulfamoyl)phenyl)acrylate Int-49 as orange oil in 91% yield. ¹H-NMR (400 MHz, CDCl₃): δ 7.90 (t, J=1.7 Hz, 1H), 7.74 (ddd, J=7.9, 1.8, 1.1 Hz, 1H), 7.68 (d, J=7.8 Hz, 1H), 7.63 (d, J=16.1 Hz, 1H), 7.49 (t, J=7.8 Hz, 1H), 7.12 (s, 4H), 6.86 (s, 1H), 6.44 (d, J=16.0 Hz, 1H), 3.82 (s, 3H).

### (E)-3-(3-(N-(4-(trifluoromethoxy)phenyl)sulfamoyl)phenyl)acrylic acid (Int-50)

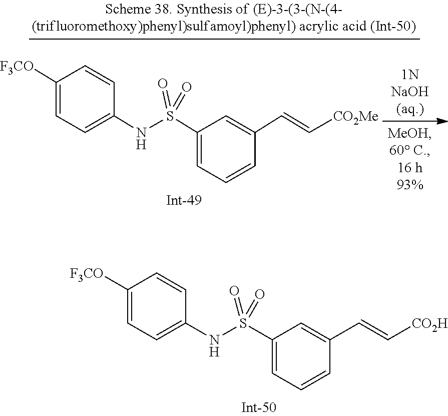 [SMILES: COC(=O)/C=C/c1cccc(S(=O)(=O)Nc2ccc(OC(F)(F)F)cc2)c1 ; O=C(O)/C=C/c1cccc(S(=O)(=O)Nc2ccc(OC(F)(F)F)cc2)c1]

### Experimental Procedure:

Aqueous 1N NaOH solution (790 μL) was added to the solution of methyl (E)-3-(3-(N-(4-(trifluoromethoxy)phenyl)sulfamoyl)phenyl)acrylate Int-49 (100 mg, 0.25 mmol) in Methanol (1.3 mL). The reaction mixture was heated at 60° C. for 16 hours. Then Methanol was removed under reduced pressure, and the residue was diluted with water (5 mL). The aqueous phase was washed with Diethyl Ether (1×5 mL) before it was acidified with aqueous 2N HCl solution to pH of approx. 2-3. The resulting solution was stirred for 30 minutes at room temperature upon which time an off-white precipitate was formed. The solid was isolated by centrifugation, washed with water (1×5 mL) and dried in vacuo to give 90.2 mg of (E)-3-(3-(N-(4-(trifluoromethoxy)phenyl)sulfamoyl)-phenyl)acrylic acid Int-50 as an off-white solid in 93% yield. MS (ESI) m/z=386 [M−H⁺].

### Synthesis of (E)-3-(3-(N-(2-chloro-5-(trifluoromethyl)phenyl)sulfamoyl)phenyl)acrylic acid (Int-54)

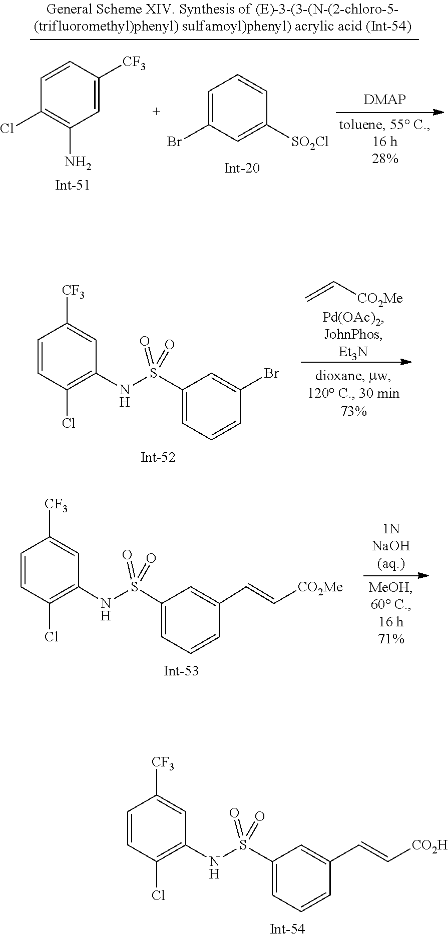

### 3-Bromo-N-(2-chloro-5-(trifluoromethyl)phenyl)benzenesulfonamide (Int-52)

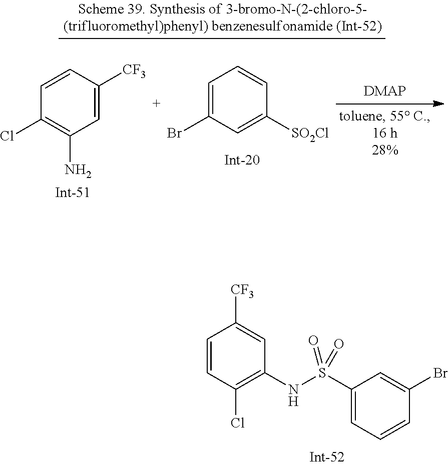 [SMILES: Nc1cc(C(F)(F)F)ccc1Cl ; O=S(=O)(Cl)c1cccc(Br)c1 ; O=S(=O)(Nc1cc(C(F)(F)F)ccc1Cl)c1cccc(Br)c1]

### Experimental Procedure:

3-Bromobenzenesulfonyl chloride Int-20 (250 mg, 0.98 mmol) was dissolved in toluene (1.5 mL) and 2-chloro-5-(trifluoromethyl)aniline Int-51 (295 μL, 2.15 mmol) was added, followed by DMAP (24.4 mg, 0.20 mmol). The reaction mixture was heated at 55° C. in a sealed vial overnight. After cooling down to room temperature, the reaction mixture was diluted with toluene (5 mL), washed with 2N aqueous HCl solution (1×5 mL), saturated aqueous NaHCO₃ solution (1×5 mL), and brine (1×5 mL), dried over anhydrous sodium sulfate and concentrated in vacuo. The residue was purified via column chromatography on silica gel using 0-80% gradient of Ethyl Acetate in Hexane as eluent to give 113.5 mg of 3-bromo-N-(2-chloro-5-(trifluoromethyl)phenyl)benzene-sulfonamide Int-52 as an off-white solid in 28% yield. ¹H-NMR (400 MHz, CDCl₃): δ 7.96-7.92 (m, 1H), 7.70 (dd, J=8.0, 1.7 Hz, 1H), 7.49-7.41 (m, 1H), 7.35-7.31 (m, 2H), 6.99 (d, J=2.0 Hz, 1H), 6.92 (ddd, J=8.3, 2.1, 0.6 Hz, 1H).

### Methyl (E)-3-(3-(N-(2-chloro-5-(trifluoromethyl)phenyl)sulfamoyl) phenyl) acrylate (Int-53)

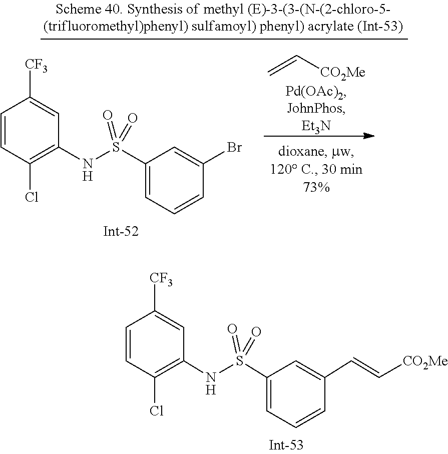

### Experimental Procedure:

3-Bromo-N-(2-chloro-5-(trifluoromethyl)phenyl)benzenesulfonamide Int-52 (113.5 mg, 0.27 mmol) was dissolved in 1,4-Dioxane (4 mL) in a 5 mL microwave vial and Triethylamine (76 p L, 0.55 mmol) was added, followed by JohnPhos (23.4 mg, 0.06 mmol) and palladium(II) acetate (6.2 mg, 0.03 mmol). The resulting solution was degassed with Nitrogen gas for 10 minutes before methyl acrylate (62 p L, 0.69 mmol) was added. After addition, the vial was immediately sealed and heated at 120° C. in a microwave reactor for 30 minutes. After cooling down to room temperature, the reaction mixture was filtered through a pad of Celite and washed with Ethyl Acetate (10 mL). The filtrate was concentrated under reduced pressure, and the residue was purified by column chromatography on silica gel using 0-80% gradient of Ethyl Acetate in Hexane as eluent to give 84.0 mg of methyl (E)-3-(3-(N-(2-chloro-5-(trifluoromethyl)phenyl)sulfamoyl)phenyl)acrylate Int-53 as a light yellow solid in 73% yield. ¹H-NMR (400 MHz, CDCl₃): δ 7.94-7.92 (m, J=3.8, 1.9 Hz, 1H), 7.77 (ddd, J=7.9, 1.8, 1.1 Hz, 1H), 7.71-7.68 (m, 1H), 7.62 (d, J=16.0 Hz, 1H), 7.49 (t, J=7.8 Hz, 1H), 7.39 (d, J=8.4 Hz, 1H), 7.31 (ddd, J=8.4, 2.1, 0.5 Hz, 1H), 7.14 (bs, 1H), 6.44 (d, J=16.0 Hz, 1H), 3.82 (s, 3H).

### (E)-3-(3-(N-(2-chloro-5-(trifluoromethyl)phenyl)sulfamoyl)phenyl)acrylic acid (Int-54)

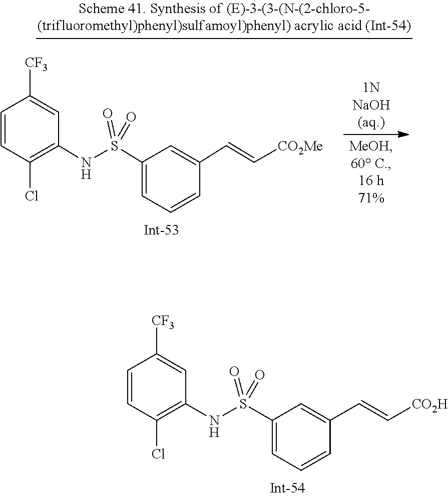 [SMILES: COC(=O)/C=C/c1cccc(S(=O)(=O)Nc2cc(C(F)(F)F)ccc2Cl)c1 ; O=C(O)/C=C/c1cccc(S(=O)(=O)Nc2cc(C(F)(F)F)ccc2Cl)c1]

### Experimental Procedure:

Aqueous 1N NaOH solution (790 μL) was added to the solution of methyl (E)-3-(3-(N-(2-chloro-5-(trifluoromethyl)phenyl)sulfamoyl)phenyl)acrylate Int-53 (100 mg, 0.24 mmol) in methanol (1.25 mL). The reaction mixture was heated at 60° C. for 16 hours. Then Methanol was removed under reduced pressure, and the residue was diluted with water (5 mL). The aqueous phase was washed with Diethyl Ether (1×5 mL) before it was acidified with aqueous 2N HCl solution to pH of approx. 2-3. The resulting solution was stirred for 30 minutes at room temperature, and then extracted with Ethyl Acetate (2×5 mL). Combined organic layers were washed with brine (1×5 mL), dried over anhydrous sodium sulfate and concentrated in vacuo to give 82.4 mg of (E)-3-(3-(N-(2-chloro-5-(trifluoromethyl)phenyl)sulfamoyl)phenyl)acrylic acid Int-54 as a yellow solid in 85% yield. MS (ESI) m/z=404 [M−H⁺].

### Synthesis of (E)-3-(3-(N-(4-chlorophenyl)-N-methylsulfamoyl)phenyl)acrylic acid (Int-58)

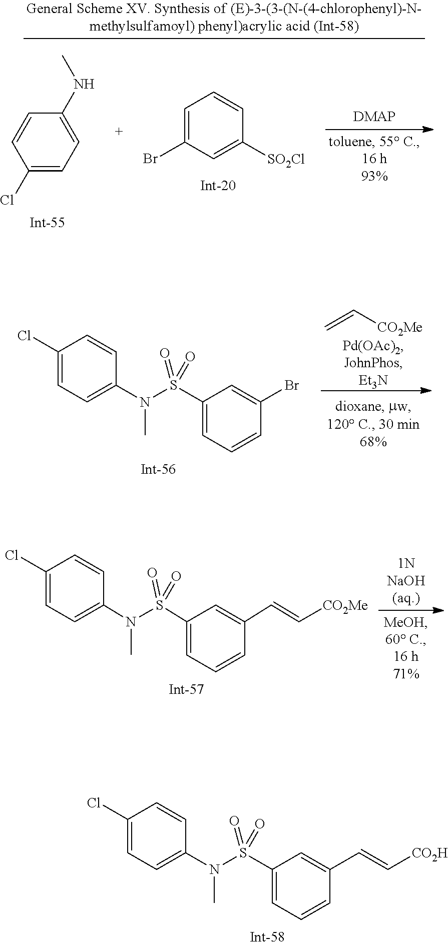

### 3-Bromo-N-(4-chlorophenyl)-N-methylbenzenesulfonamide (Int-56)

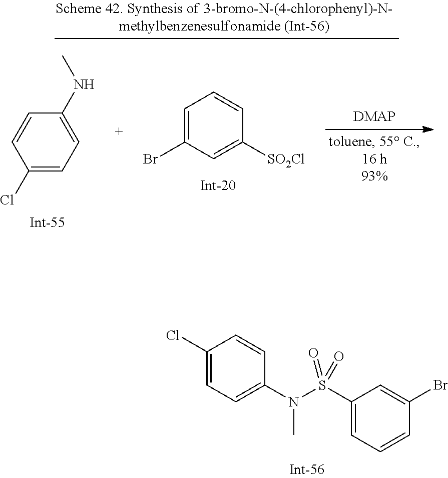

### Experimental Procedure:

3-Bromobenzenesulfonyl chloride Int-20 (500.8 mg, 1.96 mmol) was dissolved in toluene (3.0 mL), then 4-chloro-N-methylaniline Int-55 (608.9 mg, 4.30 mmol) was added, followed by DMAP (49.0 mg, 0.40 mmol). The reaction mixture was heated at 55° C. in a sealed vial overnight. After cooling down to room temperature, the reaction mixture was diluted with toluene (5 mL), washed with 2N aqueous HCl solution (1×5 mL), saturated aqueous NaHCO₃ solution (1×5 mL), brine (1×5 mL), dried over anhydrous sodium sulfate and concentrated in vacuo to give 650 mg of 3-bromo-N-(4-chlorophenyl)-N-methylbenzenesulfonamide Int-56 as dark brown oil in 93% yield. ¹H-NMR (400 MHz, CDCl₃): δ 7.73-7.70 (m, 2H), 7.42 (dt, J=8.0, 1.4 Hz, 1H), 7.35 (dd, J=7.6, 0.6 Hz, 1H), 7.32-7.27 (m, 2H), 7.05-7.02 (m, 2H), 3.17 (s, 3H).

### Methyl (E)-3-(3-(N-(4-chlorophenyl)-N-methylsulfamoyl)phenyl)acrylate (Int-57)

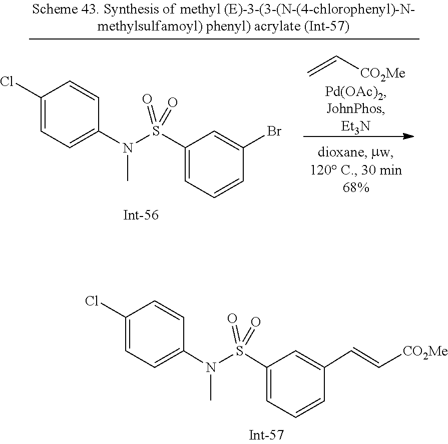

### Experimental Procedure:

3-Bromo-N-(4-chlorophenyl)-N-methylbenzenesulfonamide Int-56 (650.0 mg, 1.82 mmol) was dissolved in 1,4-Dioxane (18 mL) in a 20 mL microwave vial and Triethylamine (505 μL, 3.64 mmol) was added, followed by JohnPhos (152.9 mg, 0.36 mmol) and palladium(II) acetate (40.4 mg, 0.18 mmol). The resulting solution was degassed with Nitrogen gas for 10 minutes before methyl acrylate (392 μL, 4.55 mmol) was added. After the addition, the vial was immediately sealed and heated at 120° C. in a microwave reactor for 30 minutes. After cooling down to room temperature, the reaction mixture was filtered through a pad of Celite and washed with Ethyl Acetate (10 mL). The filtrate was concentrated under reduced pressure. The residue was purified by column chromatography on silica gel using 0-75% gradient of Ethyl Acetate in Hexane as eluent to give 451.6 mg of methyl (E)-3-(3-(N-(4-chlorophenyl)-N-methylsulfamoyl)phenyl)acrylate Int-57 as an off-white solid in 68% yield. ¹H-NMR (400 MHz, CDCl₃): δ 7.74-7.70 (m, 1H), 7.69 (s, 1H), 7.65 (d, J=16.0 Hz, 1H), 7.52-7.47 (m, 2H), 7.30-7.27 (m, 2H), 7.05-7.01 (m, 2H), 6.42 (d, J=16.0 Hz, 1H), 3.82 (s, 3H), 3.17 (s, 3H).

### (E)-3-(3-(N-(4-chlorophenyl)-N-methylsulfamoyl)phenyl)acrylic acid (Int-58)

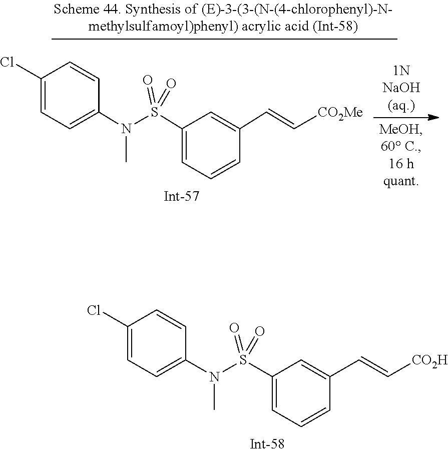 [SMILES: COC(=O)/C=C/c1cccc(S(=O)(=O)N(C)c2ccc(Cl)cc2)c1 ; CN(c1ccc(Cl)cc1)S(=O)(=O)c1cccc(/C=C/C(=O)O)c1]

### Experimental Procedure:

Aqueous 1N NaOH solution (790 μL) was added to the solution of methyl (E)-3-(3-(N-(4-chlorophenyl)-N-methylsulfamoyl)phenyl)acrylate Int-57 (100 mg, 0.27 mmol) in methanol (1.3 mL). The reaction mixture was heated at 60° C. for 16 hrs. Then, methanol was removed under reduced pressure. The residue was diluted with water (5 mL). The aqueous phase was washed with Diethyl Ether (1×5 mL) before it was acidified with aqueous 2 N HCl solution to a pH of approx. 2-3. The resulting solution was stirred for 30 minutes at room temperature, and then extracted with Ethyl Acetate (2×5 mL). Combined organic layers were washed with brine (1×5 mL), dried over anhydrous sodium sulfate and concentrated in vacuo to give 91 mg of (E)-3-(3-(N-(4-chlorophenyl)-N-methylsulfamoyl)phenyl)acrylic acid Int-58 as an off-white solid in quantitative yield. MS (ESI) m/z=350 [M−H⁺].

### Synthesis of (E)-3-(3-(N-(3,5-dimethylphenyl)sulfamoyl)phenyl)acrylic acid (Int-62)

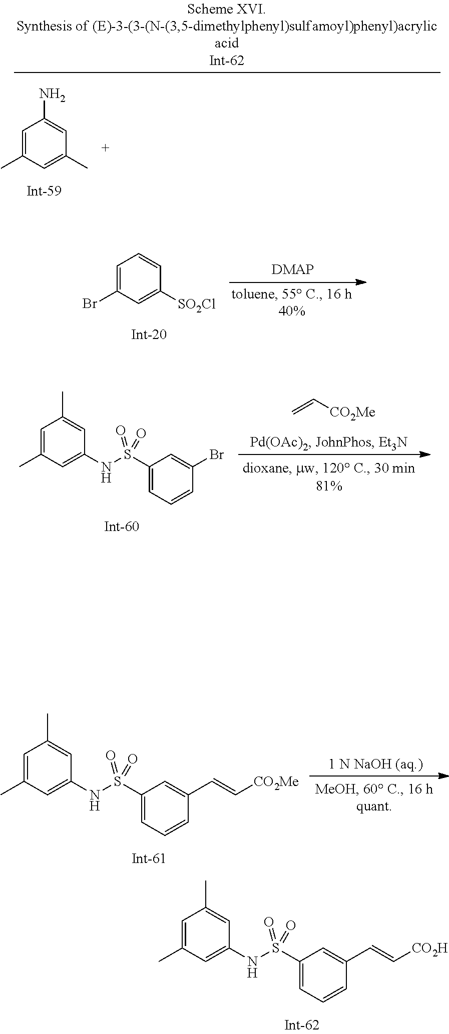

### 3-Bromo-N-(3,5-dimethylphenyl)benzenesulfonamide (Int-60)

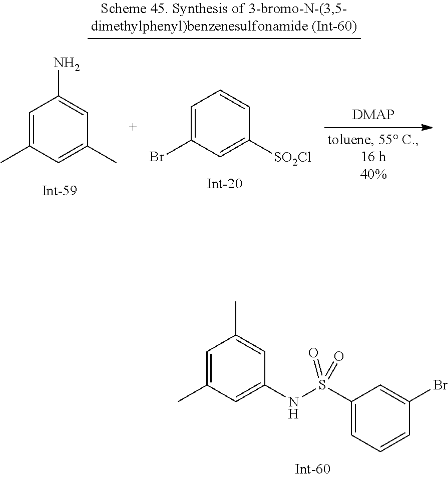

### Experimental Procedure:

3-Bromobenzenesulfonyl chloride Int-20 (500.8 mg, 1.96 mmol) was dissolved in toluene (3.0 mL), and 3,5-dimethylaniline Int-59 (537 μL, 4.30 mmol) was added, followed by DMAP (49.0 mg, 0.40 mmol). The reaction mixture was heated at 55° C. in a sealed vial overnight. After cooling down to room temperature, the reaction mixture was diluted with toluene (5 mL), washed with 2N aqueous HCl solution (1×5 mL), saturated aqueous NaHCO₃ solution (1×5 mL), and brine (1×5 mL), dried over anhydrous sodium sulfate and concentrated in vacuo to give 264.5 mg of 3-bromo-N-(3,5-dimethylphenyl)benzenesulfonamide Int-60 as an off-white solid in 40% yield. ¹H-NMR (400 MHz, CDCl₃): δ 7.93 (t, J=1.7 Hz, 1H), 7.72-7.63 (m, J=1.9, 1.0 Hz, 2H), 7.31 (t, J=8.0 Hz, 1H), 6.79 (s, 1H), 6.68 (s, 2H), 6.50 (s, 1H).

### Methyl (E)-3-(3-(N-(3,5-dimethylphenyl)sulfamoyl)phenyl)acrylate (Int-61)

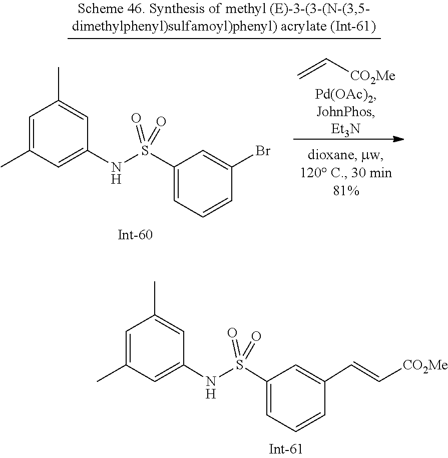

### Experimental Procedure:

3-Bromo-N-(3,5-dimethylphenyl)benzenesulfonamide Int-60 (264.5 mg, 0.78 mmol) was dissolved in 1,4-Dioxane (8 mL) in a 20 mL microwave vial and Triethylamine (216 μL, 1.56 mmol) was added, followed by JohnPhos (67.9 mg, 0.16 mmol) and Palladium(II) Acetate (18.0 mg, 0.08 mmol). The resulting solution was degassed with Nitrogen gas for 10 minutes before methyl acrylate (177 μL, 1.95 mmol) was added. After the addition, the vial was immediately sealed and heated at 120° C. in a microwave reactor for 30 minutes. After cooling down to room temperature, the reaction mixture was filtered through a pad of Celite and washed with Ethyl Acetate (10 mL). The filtrate was concentrated under reduced pressure, and the residue was purified by column chromatography on silica gel using 0-80% gradient of Ethyl Acetate in Hexane as eluent to give 218.1 mg of methyl (E)-3-(3-(N-(3,5-dimethylphenyl)sulfamoyl)phenyl)acrylate Int-61 as yellow oil in 81% yield. ¹H-NMR (400 MHz, CDCl₃): δ 7.89 (t, J=1.5 Hz, 1H), 7.75 (ddd, J=7.9, 1.7, 1.1 Hz, 1H), 7.65 (dt, J=8.0. 1.6 Hz, 1H), 7.63 (d, J=16.0 Hz, 1H), 7.47 (t, J=7.8 Hz, 1H), 6.78 (s, 1H), 6.68 (s, 2H), 6.42 (d, J=16.0 Hz, 2H), 3.82 (s, 3H), 2.23 (s, 6H).

### (E)-3-(3-(N-(3,5-dimethylphenyl)sulfamoyl)phenyl)acrylic acid (Int-62)

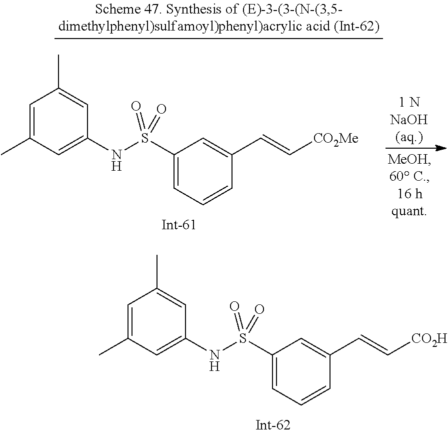 [SMILES: COC(=O)/C=C/c1cccc(S(=O)(=O)Nc2cc(C)cc(C)c2)c1 ; Cc1cc(C)cc(NS(=O)(=O)c2cccc(/C=C/C(=O)O)c2)c1]

### Experimental Procedure:

Aqueous 1N NaOH solution (920 μL) was added to the solution of methyl (E)-3-(3-(N-(3,5-dimethylphenyl)sulfamoyl)phenyl)acrylate Int-61 (100 mg, 0.29 mmol) in methanol (1.5 mL). The reaction mixture was heated at 60° C. for 16 hours. Then methanol was removed under reduced pressure, and the residue was diluted with water (5 mL). The aqueous phase was washed with Diethyl Ether (1×5 mL), and then acidified with aqueous 2 N HCl solution to pH of approx. 2-3. The resulting solution was stirred for 30 minutes at room temperature, and then extracted with Ethyl Acetate (2×5 mL). Combined organic layers were washed with brine (1×5 mL), dried over anhydrous sodium sulfate and concentrated in vacuo to give 96.2 mg of (E)-3-(3-(N-(3,5-dimethylphenyl)sulfamoyl)phenyl)acrylic acid Int-62 as an off-white solid in quantitative yield. MS (ESI) m/z=330 [M−H⁺].

### Synthesis of (E)-3-(3-(N-(4-chlorophenyl)sulfamoyl)phenyl)acrylic acid (Int-66)

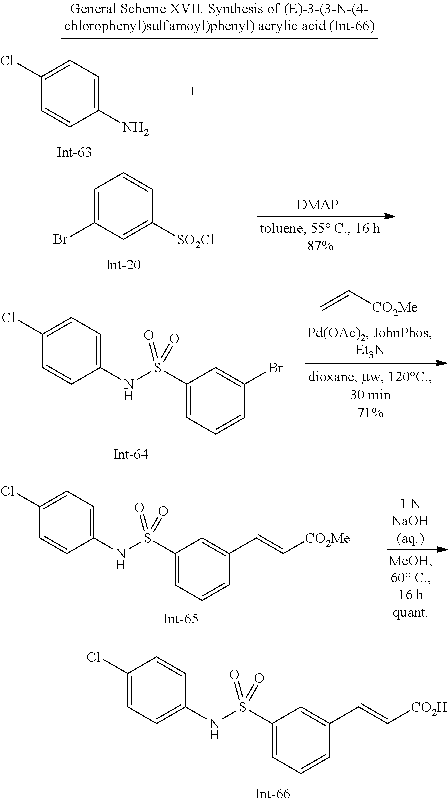

### 3-Bromo-N-(4-chlorophenyl)benzenesulfonamide (Int-64)

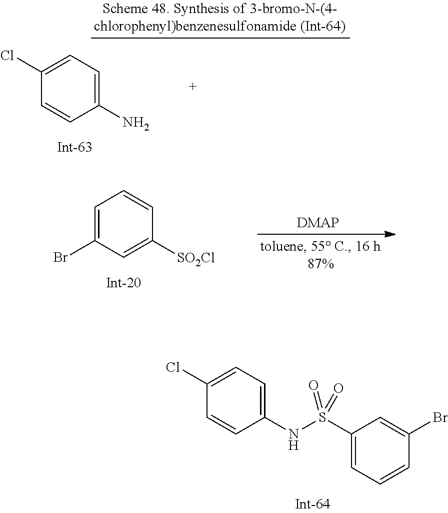 [SMILES: Nc1ccc(Cl)cc1 ; O=S(=O)(Cl)c1cccc(Br)c1 ; O=S(=O)(Nc1ccc(Cl)cc1)c1cccc(Br)c1]

### Experimental Procedure:

3-Bromobenzenesulfonyl chloride Int-20 (1.0 g, 3.92 mmol) was dissolved in toluene (6.0 mL) and 4-chloroaniline Int-63 (1.1 g, 8.6 mmol) was added, followed by DMAP (97.7 mg, 0.80 mmol). The reaction mixture was heated at 55° C. in a sealed vial overnight. After cooling down to room temperature, the reaction mixture was diluted with toluene (5 mL), washed with 2N aqueous HCl solution (1×5 mL), saturated aqueous NaHCO₃ solution (1×5 mL), and brine (1×5 mL), dried over anhydrous sodium sulfate and concentrated in vacuo to give 1.18 g of 3-bromo-N-(4-chlorophenyl) benzenesulfonamide Int-64 as an off-white solid in 87% yield. ¹H-NMR (400 MHz, CDCl₃): δ 7.87 (t, J=1.8 Hz, 1H), 7.61 (ddd, J=8.0, 1.9, 1.0 Hz, 1H), 7.59 (ddd, J=7.9, 1.7, 1.0 Hz, 1H), 7.26 (t, J=7.9 Hz, 1H), 7.22-7.14 (m, 2H), 6.98-6.94 (m, 2H).

### Methyl (E)-3-(3-(N-(4-chlorophenyl)sulfamoyl)phenyl)acrylate (Int-65)

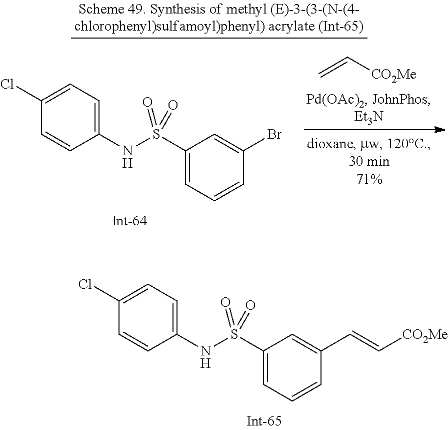

### Experimental Procedure:

3-Bromo-N-(4-chlorophenyl)benzenesulfonamide Int-64 (1.18 g, 3.40 mmol) was dissolved in 1,4-Dioxane (17.3 mL) in a 20 mL microwave vial and Triethylamine (471 μL, 3.40 mmol) was added, followed by JohnPhos (288.8 mg, 0.68 mmol) and Palladium(II) Acetate (76.3 mg, 0.34 mmol). The resulting solution was degassed with Nitrogen gas for 10 minutes before methyl acrylate (770 μL, 8.50 mmol) was added. After the addition, the vial was immediately sealed and heated at 120° C. in a microwave reactor for 30 minutes. After cooling down to room temperature, the reaction mixture was filtered through a pad of Celite and washed with Ethyl Acetate (10 mL). The filtrate was concentrated under reduced pressure and the residue was purified by column chromatography on silica gel using 0-75% gradient of Ethyl Acetate in Hexane as eluent to give 849 mg of methyl (E)-3-(3-(N-(4-chlorophenyl)sulfamoyl)phenyl)acrylate Int-65 as orange oil in 71% yield. ¹H-NMR (400 MHz, CDCl₃): δ 7.91 (t, J=1.7 Hz, 1H), 7.72 (ddd, J=7.9, 1.6, 1.2 Hz, 1H), 7.68 (d, J=7.8 Hz, 1H), 7.64 (d, J=16.0 Hz, 1H), 7.48 (t, J=7.8 Hz, 1H), 7.24-7.21 (m, 2H), 7.05-7.01 (m, 2H), 6.88 (bs, 1H), 6.45 (d, J=16.0 Hz, 1H), 3.82 (s, 3H).

### (E)-3-(3-(N-(4-chlorophenyl)sulfamoyl)phenyl)acrylic acid (Int-66)

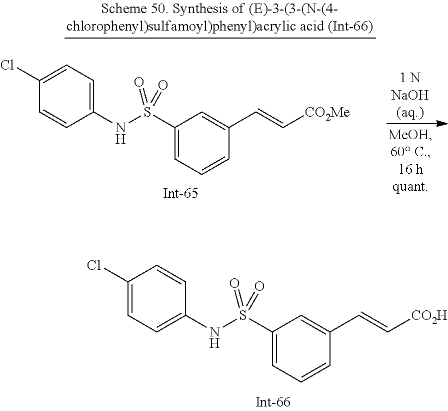 [SMILES: COC(=O)/C=C/c1cccc(S(=O)(=O)Nc2ccc(Cl)cc2)c1 ; O=C(O)/C=C/c1cccc(S(=O)(=O)Nc2ccc(Cl)cc2)c1]

### Experimental Procedure:

Aqueous 1N NaOH solution (900 μL) was added to the solution of methyl (E)-3-(3-(N-(4-chlorophenyl)sulfamoyl)phenyl)acrylate Int-65 (100 mg, 0.28 mmol) in methanol (1.5 mL). The reaction mixture was heated at 60° C. for 16 hours. Then methanol was removed under reduced pressure, and the residue was diluted with water (5 mL). The aqueous phase was washed with Diethyl Ether (1×5 mL) before it was acidified with aqueous 2N HCl solution to pH of approx. 2-3. The resulting solution was stirred for 30 minutes at room temperature, and then extracted with Ethyl Acetate (3×5 mL). Combined organic layers were washed with brine (1×5 mL), dried over anhydrous sodium sulfate and concentrated in vacuo to give 97 mg of (E)-3-(3-(N-(4-chlorophenyl) sulfamoyl)phenyl)acrylic acid Int-66 as a light yellow solid in quantitative yield. MS (ESI) m/z=336 [M−H⁺].

### Synthesis of (E)-3-(3-(N-(4-chloro-3-(trifluoromethyl)phenyl)sulfamoyl)phenyl)acrylic acid (Int-70)

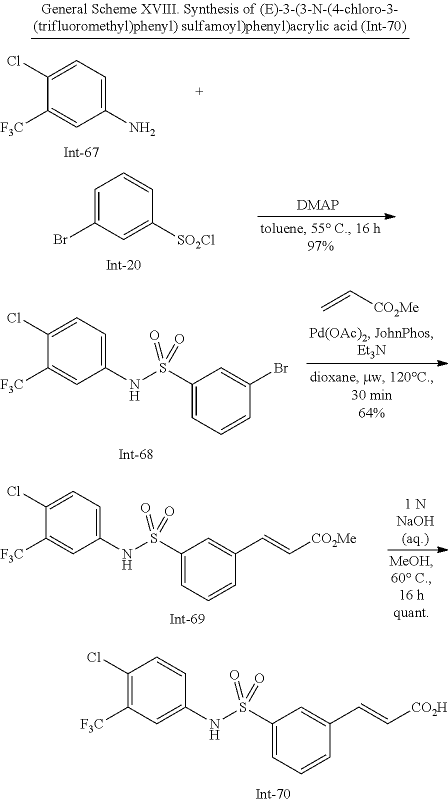

### 3-Bromo-N-(4-chloro-3-(trifluoromethyl)phenyl)benzenesulfonamide (Int-68)

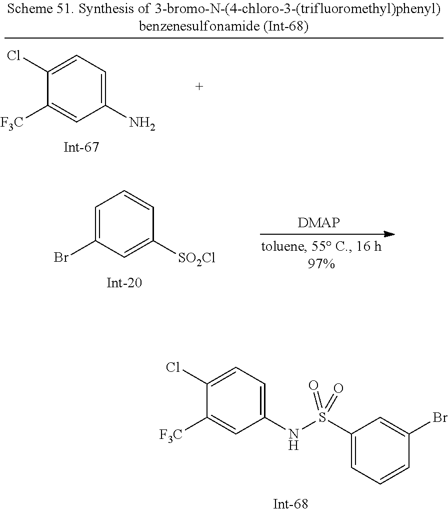 [SMILES: Nc1ccc(Cl)c(C(F)(F)F)c1 ; O=S(=O)(Cl)c1cccc(Br)c1 ; O=S(=O)(Nc1ccc(Cl)c(C(F)(F)F)c1)c1cccc(Br)c1]

### Experimental Procedure:

3-Bromobenzenesulfonyl chloride Int-20 (500.8 mg, 1.96 mmol) was dissolved in toluene (3.0 mL), and 4-chloro-3-(trifluoromethyl)aniline Int-67 (841 mg, 4.30 mmol) and DMAP (49.0 mg, 0.40 mmol) were added to this solution. The reaction mixture was heated at 55° C. in a sealed vial overnight. After cooling to room temperature, the reaction mixture was diluted with toluene (5 mL), and washed with 2N aqueous HCl solution (1×5 mL), saturated aqueous NaHCO₃ solution (1×5 mL), brine (1×5 mL), dried over anhydrous sodium sulfate and concentrated in vacuo to give 788 mg of 3-bromo-N-(4-chloro-3-(trifluoromethyl)phenyl)benzenesulfonamide Int-68 as an off-white solid in 97% yield. ¹H-NMR (400 MHz, CDCl₃): δ 7.96 (t, J=1.8 Hz, 1H), 7.73 (ddd, J=8.0, 1.9, 1.0 Hz, 1H), 7.68 (ddd, J=7.9, 1.8, 1.0 Hz, 1H), 7.42 (d, J=8.6 Hz, 1H), 7.39-7.35 (m, 2H), 6.74 (ddd, J=8.6, 2.8, 0.6 Hz, 1H).

### Methyl (E)-3-(3-(N-(4-chloro-3-(trifluoromethyl)phenyl)sulfamoyl)phenyl) acrylate (Int-69)

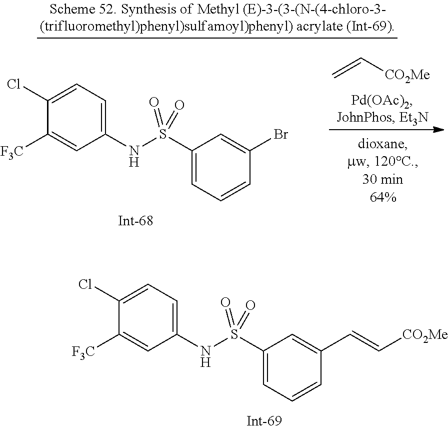

### Experimental Procedure:

3-Bromo-N-(4-chloro-3-(trifluoromethyl)phenyl)benzenesulfonamide Int-68 (788.0 mg, 1.95 mmol) was dissolved in 1,4-Dioxane (10 mL) in a 20 mL microwave vial, and Triethylamine (541 p L, 3.90 mmol) was added, followed by JohnPhos (169.9 mg, 0.40 mmol) and Palladium(II) Acetate (44.9 mg, 0.20 mmol). The resulting solution was degassed with Nitrogen gas for 10 minutes, and then Methyl Acrylate (442 μL, 4.88 mmol) was added. After the addition the vial was immediately sealed and heated at 120° C. in a microwave reactor for 30 minutes. After cooling down to room temperature, the reaction mixture was filtered through a pad of Celite and washed with Ethyl Acetate (10 mL). The filtrate was concentrated under reduced pressure, and the residue was purified by column chromatography on silica gel using a 0-80% gradient of Ethyl Acetate in Hexane as eluent to give 522.5 mg of methyl (E)-3-(3-(N-(4-chloro-3-(trifluoromethyl)phenyl)sulfamoyl)phenyl)acrylate Int-69 as orange oil in 64% yield.

### (E)-3-(3-(N-(4-chloro-3-(trifluoromethyl)phenyl)sulfamoyl)phenyl)acrylic acid (Int-70)

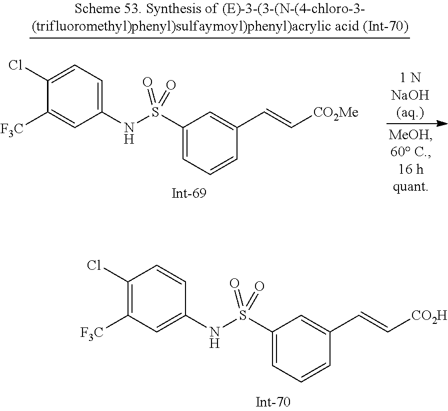 [SMILES: COC(=O)/C=C/c1cccc(S(=O)(=O)Nc2ccc(Cl)c(C(F)(F)F)c2)c1 ; O=C(O)/C=C/c1cccc(S(=O)(=O)Nc2ccc(Cl)c(C(F)(F)F)c2)c1]

### Experimental Procedure:

Aqueous 1N NaOH solution (760 p L) was added to a solution of methyl (E)-3-(3-(N-(4-chloro-3-(trifluoromethyl)phenyl)sulfamoyl)phenyl)acrylate Int-69 (100 mg, 0.24 mmol) in Methanol (1.25 mL). The reaction mixture was heated at 60° C. for 16 hours. Then Methanol was removed under reduced pressure, and the residue was diluted with water (5 mL). The aqueous phase was washed with Diethyl Ether (1×5 mL), and then was acidified with aqueous 2N HCl solution to pH of approx. 2-3. The resulting solution was stirred for 30 minutes at room temperature, and then extracted with Ethyl Acetate (3×5 mL). Combined organic layers were washed with brine (1×5 mL), dried over anhydrous sodium sulfate and concentrated in vacuo to give 97 mg of (E)-3-(3-(N-(4-chloro-3-(trifluoromethyl)phenyl)sulfamoyl)phenyl)acrylic acid Int-70 as orange solid in quantitative yield. MS (ESI) m/z=404 [M−H⁺].

### Synthesis of (E)-3-(3-((4-fluoro-3-(trifluoromethyl)phenyl)carbamoyl)phenyl)acrylic acid (Int-3)

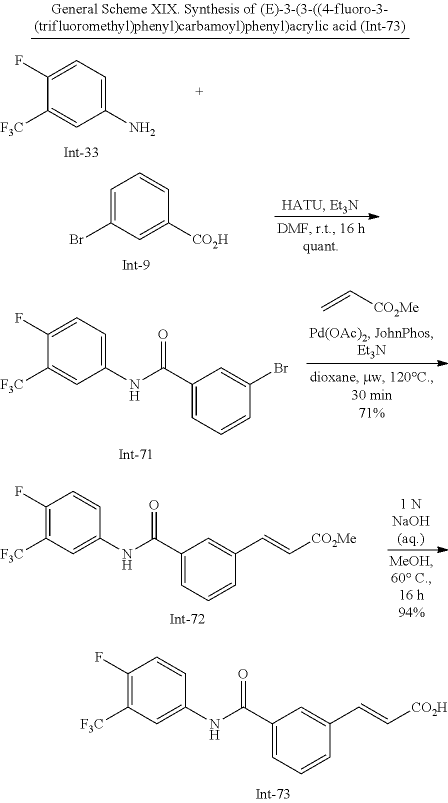

### 3-Bromo-N-(4-fluoro-3-(trifluoromethyl)phenyl)benzamide (Int-71)

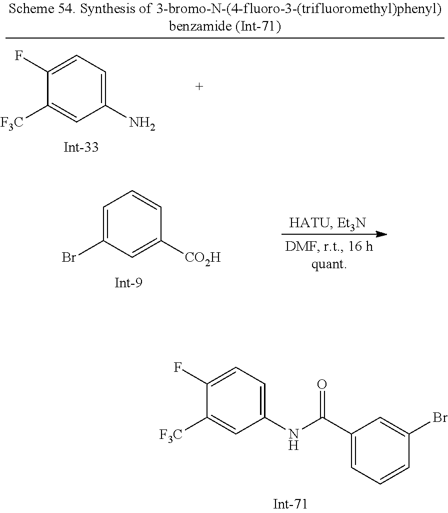 [SMILES: Nc1ccc(F)c(C(F)(F)F)c1 ; O=C(O)c1cccc(Br)c1 ; O=C(Nc1ccc(F)c(C(F)(F)F)c1)c1cccc(Br)c1]

### Experimental Procedure:

Triethylamine (416 p L, 3.00 mmol) was added to a solution of 3-bromo-benzoic acid Int-9 (402.0 mg, 2.00 mmol) and 4-fluoro-3-(trifluoromethyl)aniline Int-33 (0.13 mmol) in anhydrous N,N-Dimethyl Formamide (5 mL). The reaction flask was placed in an ice-water bath, and HATU (912.6 mg, 2.40 mmol) was added at 0° C. The reaction mixture was left to stir at room temperature for 16 hours. Then the reaction mixture was diluted with water and was extracted with Ethyl Acetate (3×10 mL). Combined organic layers were dried over anhydrous sodium sulfate, filtered and concentrated in vacuo. The crude product was purified via column chromatography on silica gel using 0-100% gradient of Ethyl Acetate in Hexane as eluent to give 723.8 mg of 3-bromo-N-(4-fluoro-3-(trifluoromethyl)phenyl)benzamide Int-71 as a light pink solid in quantitative yield. ¹H-NMR (400 MHz, CDCl₃): δ 8.00 (t, J=1.8 Hz, 1H), 7.91 (s, 1H), 7.87 (dd, J=7.0, 4.5 Hz, 2H), 7.79 (ddd, J=7.8, 1.7, 1.0 Hz, 1H), 7.71 (ddd, J=8.0, 1.9, 1.0 Hz, 1H), 7.39 (t, J=7.9 Hz, 1H), 7.22 (t, J=9.8 Hz, 1H).

### Methyl (E)-3-(3-((4-fluoro-3-(trifluoromethyl)phenyl)carbamoyl)phenyl)acrylate (Int-72)

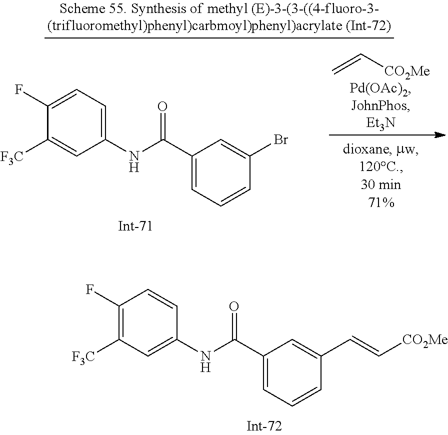

### Experimental Procedure:

3-Bromo-N-(4-fluoro-3-(trifluoromethyl)phenyl)benzamide Int-71 (723.8 mg, 2.00 mmol) was dissolved in 1,4-Dioxane (10 mL) in a 20 mL microwave vial, and Triethylamine (554 μL, 4.00 mmol) was added, followed by JohnPhos (169.9 mg, 0.40 mmol) and Palladium(II) Acetate (44.9 mg, 0.20 mmol). The resulting solution was degassed with Nitrogen gas for 10 minute, and then Methyl Acrylate (453 μL, 5.00 mmol) was added. After the addition, the vial was immediately sealed and heated at 120° C. in a microwave reactor for 30 minutes. After cooling down to room temperature, the reaction mixture was filtered through a pad of Celite and washed with Ethyl Acetate (10 mL). The filtrate was concentrated under reduced pressure, and the residue was purified by column chromatography on silica gel using a 0-80% gradient of Ethyl Acetate in Hexane as eluent to give 521 mg of methyl (E)-3-(3-((4-fluoro-3-(trifluoromethyl) phenyl)carbamoyl)phenyl)acrylate Int-72 as a light yellow solid in 71% yield. ¹H-NMR (400 MHz, CDCl₃): δ 8.01 (t, J=1.7 Hz, 1H), 7.96 (bs, 1H), 7.92-7.87 (m, J=3.3 Hz, 2H), 7.86 (dt, J=7.7, 1.4 Hz, 1H), 7.74-7.70 (m, 2H), 7.53 (t, J=7.8 Hz, 1H), 7.23-7.20 (m, 1H), 6.53 (d, J=16.0 Hz, 1H), 3.82 (s, 3H).

### (E)-3-(3-((4-fluoro-3-(trifluoromethyl)phenyl)carbamoyl)phenyl)acrylic acid (Int-73)

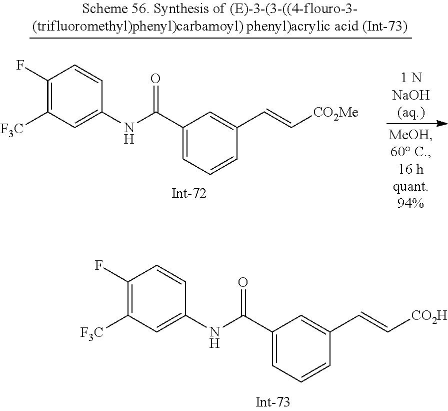 [SMILES: COC(=O)/C=C/c1cccc(C(=O)Nc2ccc(F)c(C(F)(F)F)c2)c1 ; O=C(O)/C=C/c1cccc(C(=O)Nc2ccc(F)c(C(F)(F)F)c2)c1]

### Experimental Procedure:

Aqueous 1N NaOH solution (790 μL) was added to a solution of methyl (E)-3-(3-((4-fluoro-3-(trifluoromethyl)phenyl)carbamoyl)phenyl)acrylate Int-72 (100 mg, 0.27 mmol) in Methanol (1.3 mL). The reaction mixture was heated at 60° C. for 16 hours. Then Methanol was removed under reduced pressure, and the residue was diluted with water (5 mL). The aqueous phase was washed with Diethyl Ether (1×5 mL), and then acidified with aqueous 2N HCl solution to pH of approx. 2-3. The resulting solution was stirred for 30 minutes at room temperature, and then extracted with Ethyl Acetate (3×5 mL). Combined organic layers were washed with brine (1×5 mL), dried over anhydrous sodium sulfate and concentrated in vacuo to give 89.4 mg of (E)-3-(3-((4-fluoro-3-(trifluoromethyl)phenyl)carbamoyl)phenyl)acrylic acid Int-73 as an off-white solid in 94% yield. MS (ESI) m/z=368 [M−H⁺].

### Synthesis of (E)-3-(3-((4-fluoro-3-(trifluoromethyl)phenyl) (methyl)carbamoyl)phenyl)acrylic acid (Int-75)

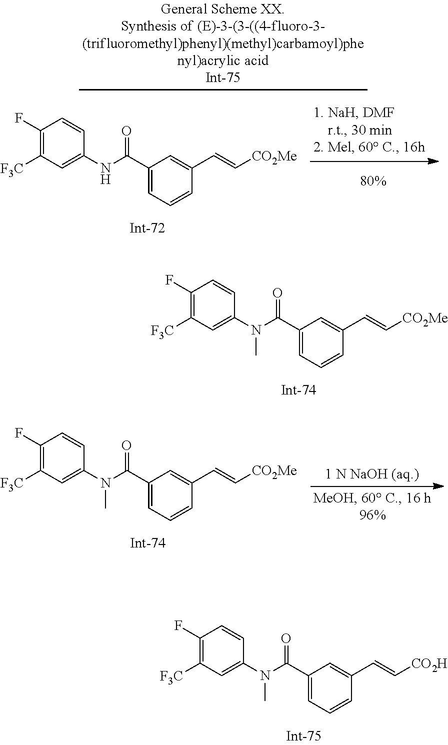

### Methyl (E)-3-(3-((4-fluoro-3-(trifluoromethyl)phenyl) (methyl)carbamoyl) phenyl)acrylate (Int-74)

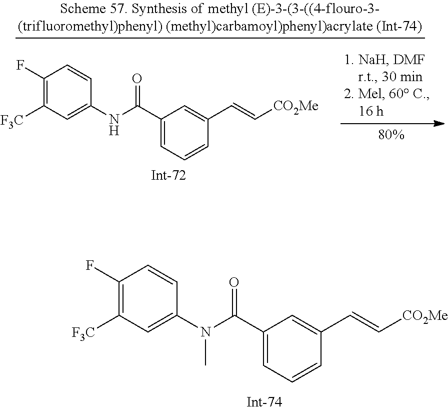 [SMILES: COC(=O)/C=C/c1cccc(C(=O)Nc2ccc(F)c(C(F)(F)F)c2)c1 ; COC(=O)/C=C/c1cccc(C(=O)N(C)c2ccc(F)c(C(F)(F)F)c2)c1]

### Experimental Procedure:

Methyl (E)-3-(3-((4-fluoro-3-(trifluoromethyl)phenyl)carbamoyl)phenyl)acrylate Int-72 (250.0 mg, 0.68 mmol) was dissolved in anhydrous DMF (1.4 mL), and Sodium Hydride (36.0 mg, 0.90 mmol, 60% dispersion in mineral oil) was slowly added at room temperature. The reaction mixture was stirred at room temperature for 30 minutes, and then Methyl Iodide (106 μL, 1.70 mmol) was added dropwise. The reaction mixture was stirred at 60° C. for 16 hours, then it was cooled down to room temperature, quenched with water (5 mL) and extracted with Ethyl Acetate (3×5 mL). Combined organic layers were washed with brine (1×5 mL), dried over anhydrous sodium sulfate and concentrated in vacuo. The residue was purified by column chromatography on silica gel using a 0-80% gradient of Ethyl Acetate in Hexane as eluent (gradient: 100:0 to 20:80) to give 208 mg of methyl (E)-3-(3-((4-fluoro-3-(trifluoromethyl)phenyl) (methyl) carbamoyl)phenyl)acrylate Int-74 as light yellow oil in 80% yield. ¹H-NMR (400 MHz, CDCl₃): δ 7.57 (d, J=16.0 Hz, 1H), 7.51 (s, 1H), 7.46 (dt, J=7.3, 1.4 Hz, 1H), 7.35 (dd, J=5.8, 2.4 Hz, 1H), 7.27-7.21 (m, 3H), 7.08 (t, J=9.2 Hz, 1H), 6.35 (d, J=16.0 Hz, 1H), 3.80 (s, 3H), 3.50 (s, 3H).

### (E)-3-(3-((4-fluoro-3-(trifluoromethyl)phenyl)(methyl)carbamoyl)phenyl)acrylic acid (Int-75)

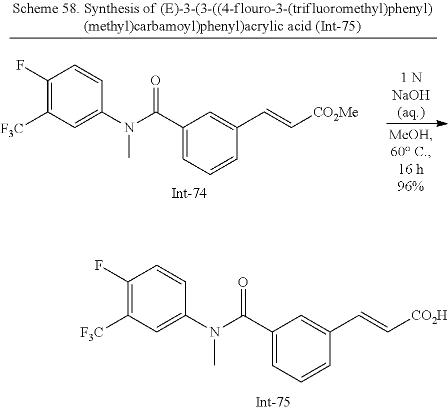 [SMILES: COC(=O)/C=C/c1cccc(C(=O)N(C)c2ccc(F)c(C(F)(F)F)c2)c1 ; CN(C(=O)c1cccc(/C=C/C(=O)O)c1)c1ccc(F)c(C(F)(F)F)c1]

### Experimental Procedure:

Aqueous 1N NaOH solution (1.8 mL) was added to a solution of methyl (E)-3-(3-((4-fluoro-3-(trifluoromethyl)phenyl)(methyl)carbamoyl)phenyl) acrylate Int-74 (208 mg, 0.55 mmol) in Methanol (3.1 mL). The reaction mixture was heated at 60° C. for 16 hours. Then Methanol was removed under reduced pressure, and the residue was diluted with water (10 mL). The aqueous phase was washed with Diethyl Ether (1×10 mL), and then acidified with aqueous 2N HCl solution to pH of approx. 2-3. The resulting solution was stirred for 30 minutes at room temperature, and then extracted with Ethyl Acetate (3×10 mL). Combined organic layers were washed with brine (1×10 mL), dried over anhydrous sodium sulfate and concentrated in vacuo to give 193.8 mg of (E)-3-(3-((4-fluoro-3-(trifluoromethyl)phenyl)(methyl)carbamoyl)phenyl)acrylic acid Int-75 as an off-white solid in 96% yield. ¹H-NMR (400 MHz, DMSO-d₆): δ 12.92 (bs, 1H), 8.14 (s, 1H), 7.94 (t, J=8.9 Hz, 1H), 7.64 (d, J=16.0 Hz, 1H), 7.52 (t, J=7.7 Hz, 1H), 7.20 (t, J=9.7 Hz, 1H), 6.76 (ddt, J=15.1, 7.3, 3.6 Hz, 2H), 6.59 (d, J=16.0 Hz, 1H), 6.04 (bs, 1H), 2.68 (s, 3H).

### Synthesis of (E)-3-(3-((4-(trifluoromethyl)phenyl)sulfonamido)phenyl)acrylic acid (Int-79)

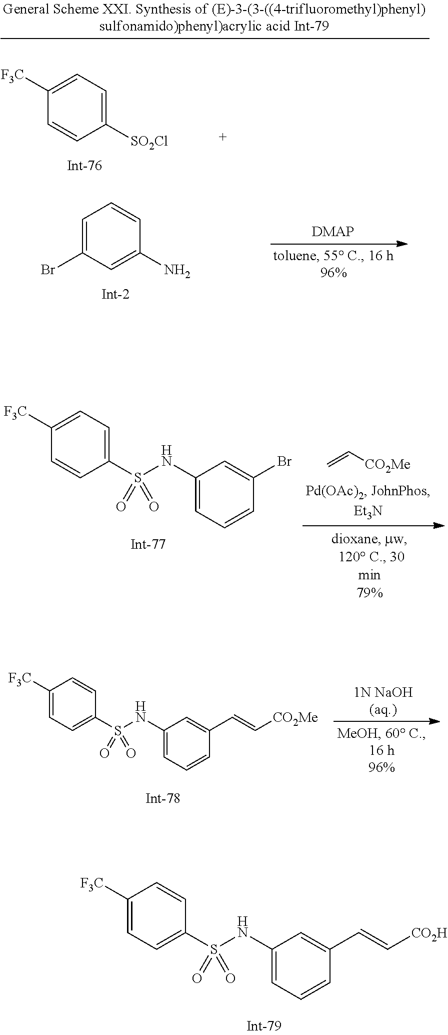

### N-(3-bromophenyl)-4-(trifluoromethyl)benzenesulfonamide (Int-77)

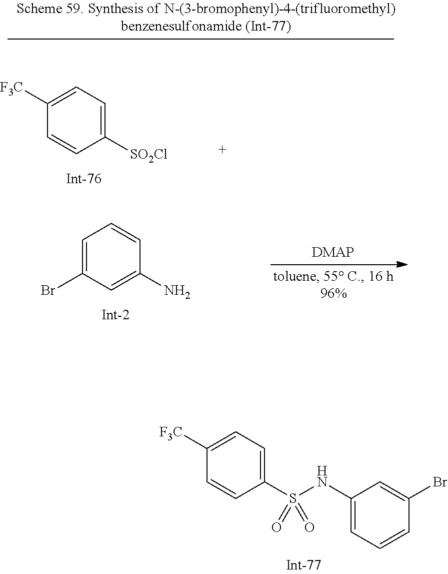 [SMILES: O=S(=O)(Cl)c1ccc(C(F)(F)F)cc1 ; Nc1cccc(Br)c1 ; O=S(=O)(Nc1cccc(Br)c1)c1ccc(C(F)(F)F)cc1]

### Experimental Procedure:

4-(Trifluoromethyl)benzenesulfonyl chloride Int-76 (479.5 mg, 1.96 mmol) was dissolved in toluene (3.0 mL), then 3-bromoaniline Int-2 (468 μL, 4.30 mmol) and DMAP (49.0 mg, 0.40 mmol) were added. The reaction mixture was heated at 55° C. in a sealed vial overnight. After cooling down to room temperature, the reaction mixture was diluted with toluene (5 mL), washed with 2N aqueous HCl solution (1×5 mL), saturated aqueous NaHCO₃ solution (1×5 mL), and brine (1×5 mL), dried over anhydrous sodium sulfate and concentrated in vacuo to give 718.5 mg of N-(3-bromophenyl)-4-(trifluoromethyl)benzenesulfonamide Int-77 as a light brown solid in 96% yield. ¹H-NMR (400 MHz, CDCl₃): δ 7.92 (d, J=8.2 Hz, 2H), 7.75 (d, J=8.2 Hz, 2H), 7.29 (dd, J=7.3, 1.2 Hz, 2H), 7.14 (t, J=8.2 Hz, 1H), 7.03 (d, J=8.7 Hz, 1H), 6.79 (s, 1H).

### Methyl (E)-3-(3-((4-(trifluoromethyl)phenyl)sulfonamido)phenyl)acrylate (Int-78)

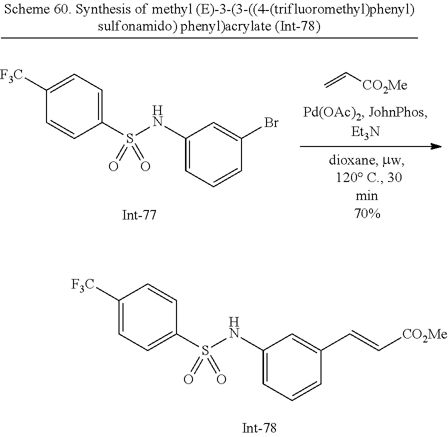

### Experimental Procedure:

N-(3-Bromophenyl)-4-(trifluoromethyl)benzenesulfonamide Int-77 (718.5 mg, 1.89 mmol) was dissolved in 1,4-Dioxane (10 mL) in a 20 mL microwave vial and Triethylamine (524 μL, 3.78 mmol) was added, followed by JohnPhos (160.5 mg, 0.38 mmol) and Palladium(II) Acetate (42.7 mg, 0.19 mmol). The resulting solution was degassed with Nitrogen gas for 10 minutes before Methyl Acrylate (428 μL, 4.73 mmol) was added. After the addition, the vial was immediately sealed and heated at 120° C. in a microwave reactor for 30 minutes. After cooling down to room temperature, the reaction mixture was filtered through a pad of Celite and washed with Ethyl Acetate (10 mL). The filtrate was concentrated under reduced pressure, and the residue was purified by column chromatography on silica gel using 0-80% gradient of Ethyl Acetate in Hexane as eluent to give 576.8 mg of methyl (E)-3-(3-((4-(trifluoromethyl)-phenyl) sulfonamido)phenyl)acrylate Int-78 as a yellow solid in 79% yield. ¹H-NMR (400 MHz, CDCl₃): δ 7.89 (d, J=8.2 Hz, 2H), 7.72 (d, J=8.3 Hz, 2H), 7.58 (d, J=16.0 Hz, 1H), 7.38-7.24 (m, 3H), 7.09 (d, J=7.4 Hz, 1H), 6.80 (bs, 1H), 6.38 (d, J=16.0 Hz, 1H), 3.81 (s, 3H).

### (E)-3-(3-((4-(trifluoromethyl)phenyl)sulfonamido)phenyl)acrylic acid (Int-79)

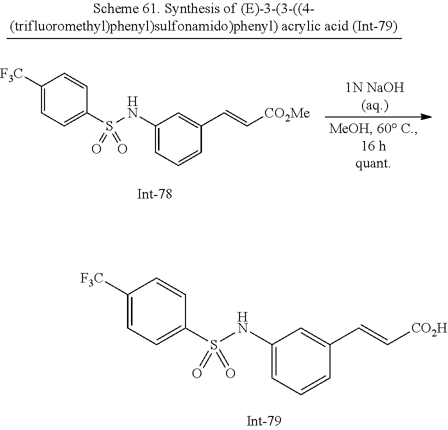 [SMILES: COC(=O)/C=C/c1cccc(NS(=O)(=O)c2ccc(C(F)(F)F)cc2)c1 ; O=C(O)/C=C/c1cccc(NS(=O)(=O)c2ccc(C(F)(F)F)cc2)c1]

### Experimental Procedure:

Aqueous 1N NaOH solution (925 p L) was added to a solution of methyl (E)-3-(3-((4-(trifluoromethyl)phenyl)sulfonamido)phenyl)acrylate Int-78 (100 mg, 0.26 mmol) in Methanol (1.5 mL). The reaction mixture was heated at 60° C. for 16 hours. Then Methanol was removed under reduced pressure, and the residue was diluted with water (5 mL). The aqueous phase was washed with Diethyl Ether (1×5 mL), and then acidified with aqueous 2N HCl solution to a pH of approx. 2-3. The resulting solution was stirred for 30 minutes at room temperature, and then extracted with Ethyl Acetate (3×5 mL). Combined organic layers were washed with brine (1×5 mL), dried over anhydrous sodium sulfate and concentrated in vacuo to give 97 mg of (E)-3-(3-((4-(trifluoromethyl) phenyl)sulfonamido)phenyl)acrylic acid Int-79 as a yellow solid in quantitative yield. MS (ESI) m/z=370 [M−H⁺].

### Synthesis of 2-thiazol-2-yl)aniline (Int-83)

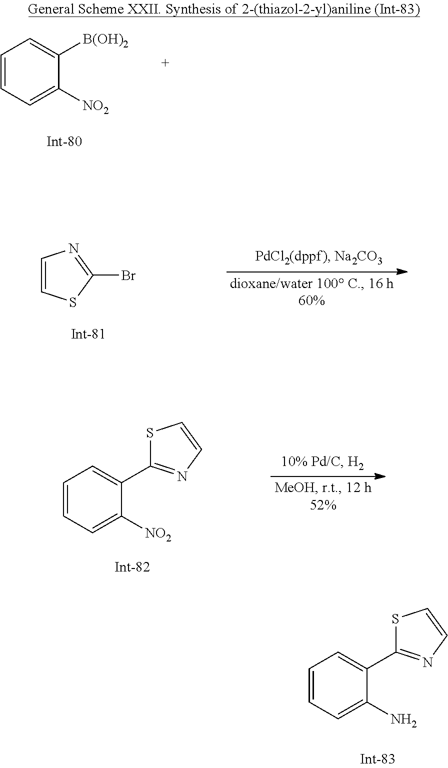 [SMILES: O=[N+]([O-])c1ccccc1B(O)O ; Brc1nccs1 ; O=[N+]([O-])c1ccccc1-c1nccs1 ; Nc1ccccc1-c1nccs1]

### 2-(2-Nitrophenyl)thiazole (Int-82)

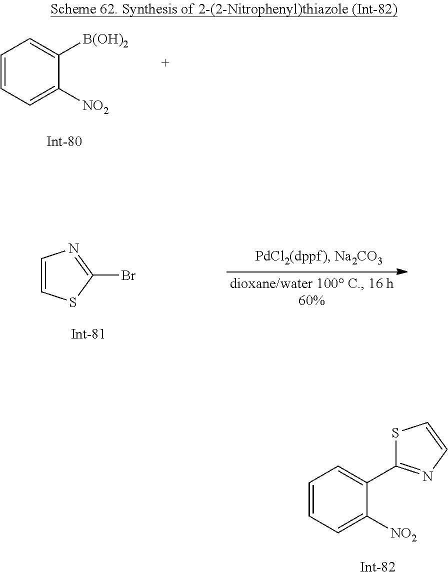 [SMILES: O=[N+]([O-])c1ccccc1B(O)O ; Brc1nccs1 ; O=[N+]([O-])c1ccccc1-c1nccs1]

### Experimental Procedure:

Solid Na₂CO₃ (7.7 g, 73.17 mmol) was added to a solution of (2-nitrophenyl)boronic acid Int-80 (4.0 g, 24.39 mmol) and 2-bromothiazole Int-81 (4.1 g, 24.39 mmol) in 1,4-Dioxane/Water (4:1, 80 mL) at room temperature. The reaction mixture was degassed with Argon for 20 min, before the PdCl₂(dppf)*DCM complex (2.0 g, 2.43 mmol) was added. The solution was degassed for additional 5 min, and then heated at 100° C. for 16 hours. After cooling down to room temperature, the reaction mixture was diluted with water (80 mL) and extracted with Ethyl Acetate (3×80 mL). Combined organic layers were washed with water (2×48 mL), brine (48 mL), dried over anhydrous sodium sulfate and concentrated under reduced pressure. The residue was purified by column chromatography on silica gel (100-200 mesh) using 10% Ethyl Acetate in Hexane as eluent to afford 3.0 g of 2-(2-nitrophenyl)thiazole int-82 as pale yellow solid in 60% yield. ¹H-NMR (400 MHz, CDCl₃): δ 7.91 (d, J=3.6 Hz, 1H), 7.82 (d, J=8.4 Hz, 1H), 7.75 (d, J=7.6 Hz, 1H), 7.65 (t, J=7.6 Hz, 1H), 7.58 (t, J=7.6 Hz, 1H), 7.48 (d, J=3.6 Hz, 1H); MS (ESI) m/z 206.94 [M+H]⁺.

### 2-(thiazol-2-yl)aniline (Int-83)

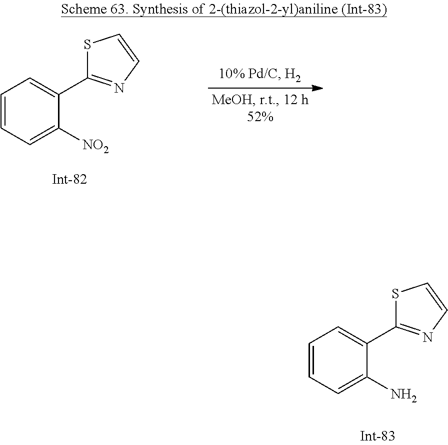 [SMILES: O=[N+]([O-])c1ccccc1-c1nccs1 ; Nc1ccccc1-c1nccs1]

### Experimental Procedure:

10% Pd/C (20% by weight) was added to a stirred solution of 2-(2-nitrophenyl)thiazole Int-82 (3.0 g, 14.56 mmol) in Methanol (30 mL). The flask was flushed with Hydrogen gas, sealed and connected to a hydrogen balloon before the reaction mixture was stirred at room temperature for 12 hrs. Then the reaction mixture was filtered through a pad of Celite, and the filtrate was concentrated under reduced pressure. The residue was purified by column chromatography on silica gel (100-200 mesh) using 40% Ethyl Acetate in Hexane as eluent to afford 1.3 g of 2-(thiazol-2-yl)aniline Int-83 as an off-white solid in 52% yield. ¹H-NMR (400 MHz, DMSO-d₆): δ 7.88 (d, J=3.4 Hz, 1H), 7.66 (d, J=3.4 Hz, 1H), 7.57 (dd, J=7.9, 1.3 Hz, 1H), 7.13 (ddd, J=8.3, 7.0, 1.4 Hz, 1H), 6.99 (bs, 2H), 6.82 (dd, J=8.3, 0.8 Hz, 1H), 6.60 (ddd, J=8.0, 7.0, 1.1 Hz, 1H); MS (ESI) m/z 176.95[M+H]⁺.

### Synthesis of 2-(1,3,4-thiadiazol-2-yl)aniline (Int-89)

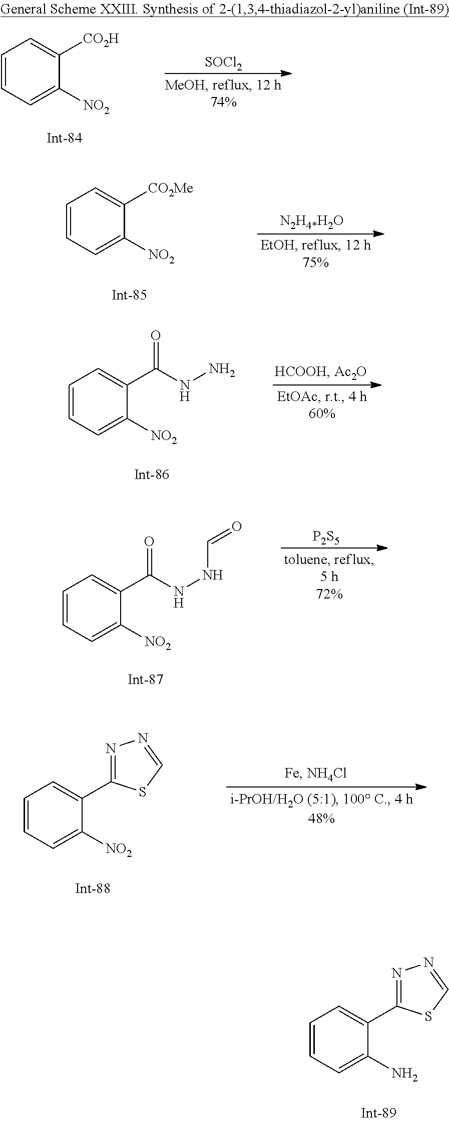

### Methyl 2-nitrobenzoate (Int-85)

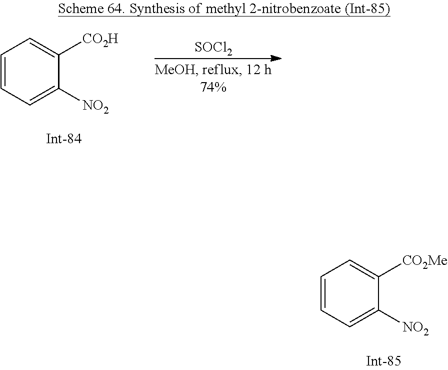 [SMILES: O=C(O)c1ccccc1[N+](=O)[O-] ; "SOCl2" ; COC(=O)c1ccccc1[N+](=O)[O-]]

### Experimental Procedure:

SOCl₂ (20 mL) was added to a solution of 2-nitrobenzoic acid Int-84 (10.0 g, 59.88 mmol) in Methanol (100 mL) at 0° C. The reaction mixture was heated at 80° C. for 16 hrs. After cooling down to room temperature, the reaction mixture was diluted with aqueous saturated NaHCO₃ solution (200 mL) and extracted with Ethyl Acetate (2×200 mL). Combined organic layers were washed with water (2×80 mL), brine (80 mL), dried over anhydrous sodium sulfate and concentrated under reduced pressure to afford 8.0 g of methyl 2-nitrobenzoate Int-85 as a pale yellow liquid in 74% yield. ¹H-NMR (300 MHz, CDCl₃): δ 7.92 (dd, J=7.8, 1.5 Hz, 1H), 7.75 (dd, J=7.8, 2.4 Hz, 1H), 7.69-7.59 (m, 2H), 3.93 (s, 3H); MS (ESI) m/z 182.03 [M+H]⁺.

### 2-Nitrobenzohydrazide (Int-86)

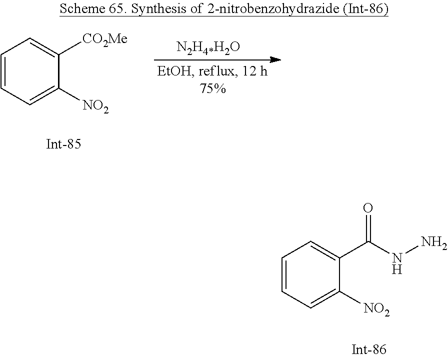 [SMILES: COC(=O)c1ccccc1[N+](=O)[O-] ; NNC(=O)c1ccccc1[N+](=O)[O-]]

### Experimental Procedure:

Hydrazine hydrate (11.0 mL, 220.9 mmol) was added to a solution of methyl 2-nitrobenzoate Int-85 (8.0 g, 44.19 mmol) in Ethanol (80 mL) at room temperature. The reaction mixture was heated at 100° C. for 16 hrs. After cooling down to room temperature, Ethanol was removed under reduced pressure. The residue was diluted with water (200 mL) and extracted with Ethyl Acetate (3×100 mL). Combined organic layers were washed with water (2×60 mL), brine (60 mL), dried over anhydrous sodium sulfate and concentrated under reduced pressure to afford 6.0 g of 2-nitrobenzohydrazide Int-86 as a pale yellow solid in 75% yield. ¹H-NMR (300 MHz, DMSO-d₆): δ 9.79 (bs, 1H), 8.03 (dd, J=8.1, 0.6 Hz, 1H), 7.78 (td, J=7.5, 1.2 Hz, 1H), 7.69 (td, J=7.2, 1.2 Hz, 1H), 7.57 (dd, J=7.2, 1.5 Hz, 1H), 4.51 (bs, 2H); MS (ESI) m/z 181.99 [M+H]⁺.

### N′-formyl-2-nitrobenzohydrazide (Int-87)

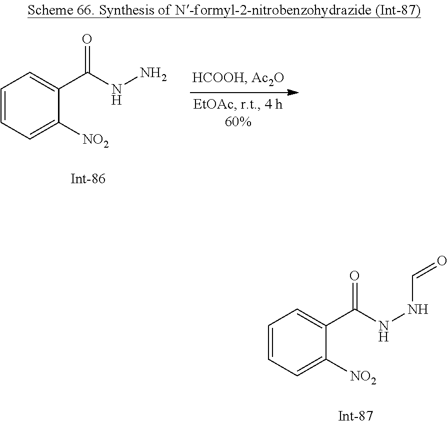 [SMILES: NNC(=O)c1ccccc1[N+](=O)[O-] ; O=CNNC(=O)c1ccccc1[N+](=O)[O-]]

### Experimental Procedure:

A mixture of formic acid (6.2 mL, 165.7 mmol) and acetic anhydride (15.5 mL, 165.7 mmol) was stirred at 0° C. for 1 hr, and then a solution of 2-nitrobenzohydrazide Int-86 (6.0 g, 33.14 mmol) in Ethyl Acetate (60 mL) was added dropwise over a period of 15 min at 0° C. The reaction mixture was stirred at room temperature for 2 hrs. Formed white precipitate was filtered off, washed with Ethyl Acetate (20 mL) and dried in vacuo to afford 4.2 g of N′-formyl-2-nitrobenzohydrazide Int-87 as a white solid in 60% yield. ¹H-NMR (300 MHz, DMSO-d₆): δ 10.72 (bs, 1H), 10.35 (bs, 1H), 9.81 (d, J=10.5 Hz, 1H), 8.20-8.05 (m, 2H), 7.92-7.64 (m, 2H); MS (ESI) m/z 210.04 [M+H]⁺.

### 2-(2-nitrophenyl)-1,3,4-thiadiazole (Int-88)

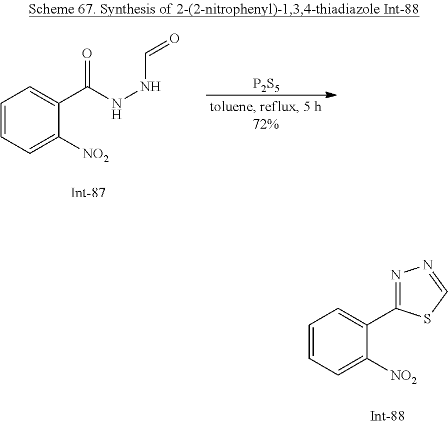

P₂S₅ (9.45 g, 43.19 mmol) was added to a solution of N′-formyl-2-nitrobenzohydrazide Int-87 (4.2 g, 20.09 mmol) in Toluene (50 mL) at room temperature. Then the reaction mixture was heated at 120° C. for 16 hrs. After cooling down to room temperature, the reaction mixture was diluted with water (100 mL) and extracted with Ethyl Acetate (3×100 mL). Combined organic layers were washed with 2N NaOH solution (2×60 mL), brine (90 mL), dried over anhydrous sodium sulfate and concentrated under reduced pressure. The residue was purified by column chromatography on silica gel (100-200 mesh) using 20% Ethyl Acetate in Hexane as eluent to afford 3.0 g of 2-(2-nitrophenyl)-1,3,4-thiadiazole Int-88 as an off-white solid in 72% yield. ¹H-NMR (300 MHz, CDCl₃): δ 9.29 (s, 1H), 8.08 (dd, J=6.9, 1.8 Hz, 1H), 7.79-7.68 (m, 3H); MS (ESI) m/z 208.23 [M+H]⁺.

### 2-(1,3,4-thiadiazol-2-yl)aniline (Int-89)

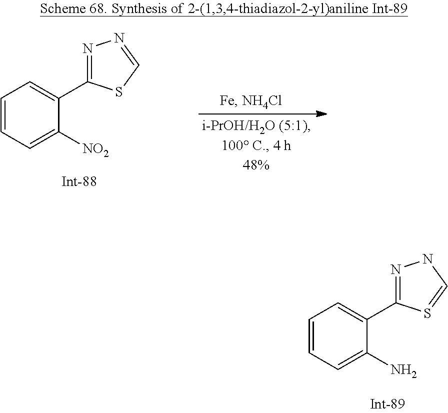

### Experimental Procedure:

Iron (4.0 g, 72.46 mmol) and NH₄Cl (0.92 g, 17.39 mmol) were added to a stirred solution of 2-(2-nitrophenyl)-1,3,4-thiadiazole Int-88 (3.0 g, 14.49 mmol) in Iso-propanol/Water (5:1, 60 mL), and the reaction mixture was heated at 100° C. for 4 hrs. After cooling down to room temperature, the reaction mixture was filtered through a Buchner funnel with a Celite pad and washed with Methanol (60 mL). The filtrate was concentrated under reduced pressure, diluted with water (100 mL) and extracted with Ethyl Acetate (2×100 mL). Combined organic layers were washed with water (2×100 mL), brine (150 mL), dried over anhydrous sodium sulfate and concentrated under reduced pressure. The residue was purified by column chromatography on silica gel (100-200 mesh) using 10% Ethyl Acetate in Hexane as eluent to afford 1.2 g of 2-(1,3,4-thiadiazol-2-yl)aniline Int-89 as a pale yellow solid in 48% yield. ¹H-NMR (400 MHz, DMSO-d₆): δ 9.53 (s, 1H), 7.54 (dd, J=7.9, 1.3 Hz, 1H), 7.21 (ddd, J=8.3, 7.1, 1.3 Hz, 1H), 7.02 (bs, 2H), 6.90 (dd, J=8.3, 0.9 Hz, 1H), 6.66-6.62 (m, 1H); MS (ESI) m/z 177.83 [M+H]⁺.

### Synthesis of 2-(1,2,4-oxadiazol-3-yl)aniline (Int-93)

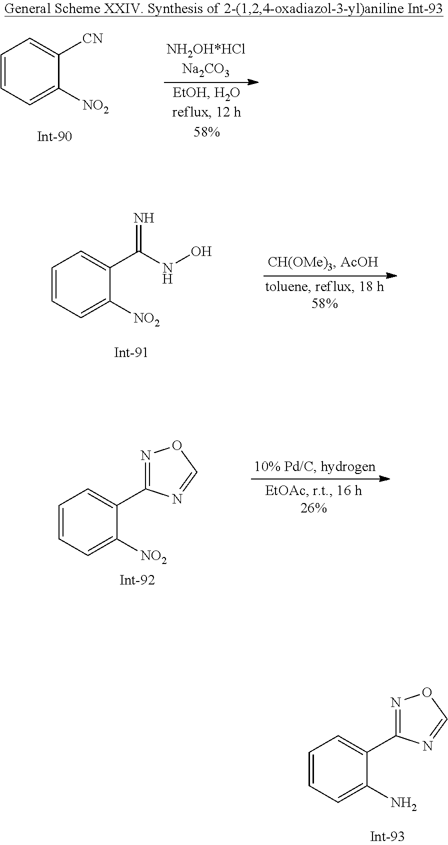 [SMILES: N#Cc1ccccc1[N+](=O)[O-] ; N=C(NO)c1ccccc1[N+](=O)[O-] ; O=[N+]([O-])c1ccccc1-c1ncon1 ; Nc1ccccc1-c1ncon1]

### N-hydroxy-2-nitrobenzimidamide (Int-91)

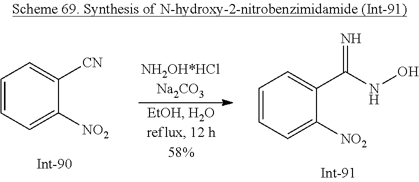 [SMILES: N#Cc1ccccc1[N+](=O)[O-] ; N=C(NO)c1ccccc1[N+](=O)[O-]]

### Experimental Procedure:

NH₂OH.HCl (14.1 g 202.5 mmol) and Na₂CO₃ (21.45 g 202.5 mmol) were added to a solution of 2-nitrobenzonitrile Int-90 (10.0 g, 67.51 mmol) in a mixture of EtOH/H₂O (3:1, 200 mL) at room temperature, and the reaction mixture was heated at 100° C. for 12 hrs. After cooling down to room temperature, the reaction mixture was concentrated under reduced pressure, and the residue was diluted with water (300 mL) and extracted with Ethyl Acetate (3×200 mL). Combined organic layers were washed with water (2×120 mL), brine (120 mL), dried over anhydrous sodium sulfate and concentrated under reduced pressure. The residue was purified by column chromatography on silica gel (100-200 mesh) using 20% Ethyl Acetate in Hexane as eluent to afford 7.0 g of N-hydroxy-2-nitrobenzimidamide Int-91 as a pale yellow solid in 58% yield. ¹H-NMR (300 MHz, DMSO-d₆): δ 9.72 (s, 1H), 7.84 (dd, J=7.8, 0.9 Hz, 1H), 7.75-7.56 (m, 3H), 6.01 (bs, 2H); MS (ESI) m/z 181.98 [M+H]⁺.

### 3-(2-nitrophenyl)-1,2,4-oxadiazole (Int-92)

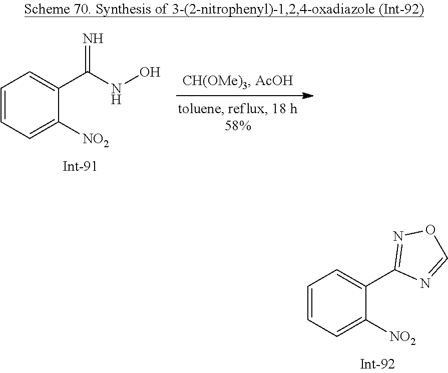 [SMILES: N=C(NO)c1ccccc1[N+](=O)[O-] ; O=[N+]([O-])c1ccccc1-c1ncon1]

### Experimental Procedure:

Acetic acid (28 mL) was added to a solution of N-hydroxy-2-nitrobenzimidamide Int-91 (7.0 g, 38.67 mmol) in Toluene (70 mL) at room temperature. Then Triethylorthoformate (22 mL, 194.6 mmol) was added, and the reaction mixture was heated at 110° C. for 18 hrs. Then the reaction mixture was diluted with water (100 mL) and extracted with Ethyl Acetate (3×150 mL). Combined organic layers were washed with water (2×90 mL), and brine (90 mL), dried over anhydrous sodium sulfate and concentrated under reduced pressure. The residue was purified by column chromatography on silica gel (100-200 mesh) using 10% Ethyl Acetate in Hexane as eluent to afford 1.2 g of 3-(2-nitrophenyl)-1,2,4-oxadiazole Int-92 as a pale yellow solid in 58% yield. ¹H-NMR (300 MHz, CDCl₃): δ 8.82 (s, 1H), 7.99 (dd, J=7.2 Hz, 1H), 7.88 (dd, J=7.5, 2.1 Hz, 1H), 7.79-7.65 (m, 2H); MS (ESI) m/z 192.26 [M+H]⁺.

### 2-(1,2,4-oxadiazol-3-yl)aniline (Int-93)

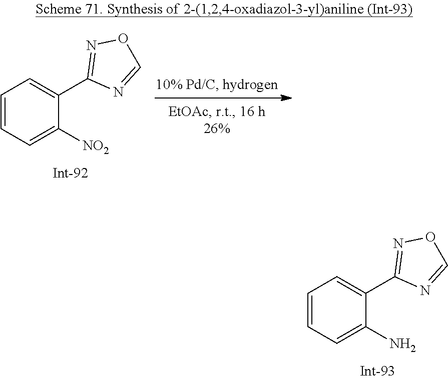 [SMILES: O=[N+]([O-])c1ccccc1-c1ncon1 ; Nc1ccccc1-c1ncon1]

### Experimental Procedure:

10% Pd/C (20% by weight) was added to a stirred solution of 3-(2-nitrophenyl)-1,2,4-oxadiazole Int-92 (2.5 g, 13.08 mmol) in Ethyl Acetate (100 mL). The flask was flushed with Hydrogen gas, sealed and connected to a hydrogen balloon before the reaction mixture was stirred at room temperature for 16 hrs. Then the reaction mixture was filtered through a pad of Celite, and the filtrate was concentrated under reduced pressure. The residue was purified by column chromatography on silica gel (100-200 mesh) using 10% Ethyl Acetate in Hexane as eluent to afford 560 mg of 2-(1,2,4-oxadiazol-3-yl)aniline Int-93 as off-white solid in 26% yield. ¹H-NMR (400 MHz, DMSO-d₆): δ 9.65 (s, 1H), 7.91 (dd, J=8.0, 1.5 Hz, 1H), 7.25 (ddd, J=8.4, 7.0, 1.5 Hz, 1H), 6.88 (dd, J=8.3, 0.8 Hz, 1H), 6.68 (ddd, J=8.0, 7.0, 1.1 Hz, 1H), 6.31 (s, 2H); MS (ESI) m/z 161.95 [M+H]⁺.

### Synthesis of 2-(1-((2-(trimethylsilyl)ethoxy)methyl)-1H-imidazol-2-yl)aniline (Int-97)

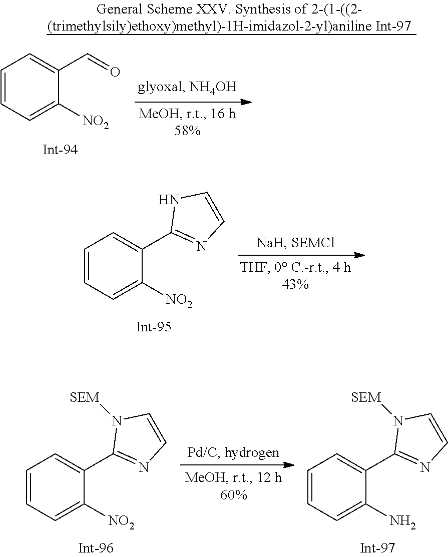 [SMILES: O=Cc1ccccc1[N+](=O)[O-] ; O=[N+]([O-])c1ccccc1-c1ncc[nH]1 ; C[Si](C)(C)CCOCn1ccnc1-c1ccccc1[N+](=O)[O-] ; C[Si](C)(C)CCOCn1ccnc1-c1ccccc1N]

### 2-(2-nitrophenyl)-1H-imidazole (Int-95)

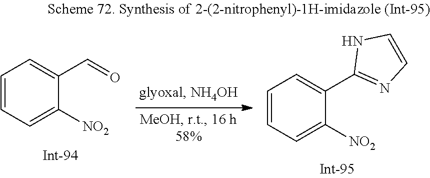 [SMILES: O=Cc1ccccc1[N+](=O)[O-] ; O=[N+]([O-])c1ccccc1-c1ncc[nH]1]

### Experimental Procedure:

Glyoxal (15.0 mL 264.6 mmol) was added to a stirred solution of 2-nitrobenzaldehyde Int-94 (20.0 g, 132.3 mmol) in Methanol (40 mL) at room temperature, followed by the addition of aqueous ammonia solution (30%, 162 mL) at 0° C. The reaction mixture was stirred at room temperature for 16 hrs, and then concentrated under reduced pressure. The residue was diluted with water (500 mL) and extracted with Ethyl Acetate (2×300 mL). Combined organic layers were washed with water (2×200 mL), brine (200 mL), dried over anhydrous sodium sulfate and concentrated under reduced pressure. The residue was purified by column chromatography on silica gel (100-200 mesh) using 20% Methanol in DCM as eluent to afford 10.5 g of 2-(2-nitrophenyl)-1H-imidazole Int-95 as pale yellow liquid in 58% yield. ¹H-NMR (300 MHz, DMSO-d₆): δ 7.87-7.71 (m, 3H), 7.59 (app. t, J=7.2 Hz, 1H), 7.32 (s, 1H), 7.03 (s, 1H), 4.10 (q, J=5.1 Hz, 1H); MS (ESI) m/z 190.20 [M+H]⁺.

### 2-(2-nitrophenyl)-1-((2-(trimethylsilyl)ethoxy)methyl)-1H-imidazole (Int-96)

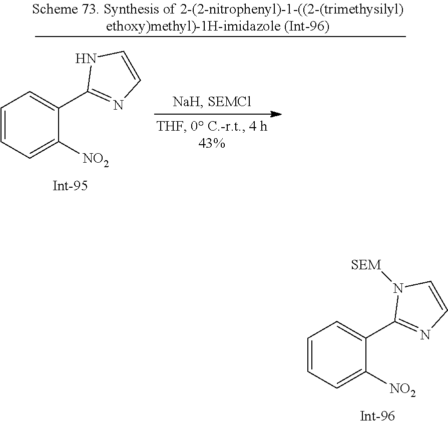 [SMILES: O=[N+]([O-])c1ccccc1-c1ncc[nH]1 ; C[Si](C)(C)CCOCn1ccnc1-c1ccccc1[N+](=O)[O-]]

### Experimental Procedure:

Sodium hydride (60% dispersion in mineral oil, 0.45 g, 11.42 mmol) was added to a stirred solution of 2-(2-nitrophenyl)-1H-imidazole Int-95 (1.8 g, 9.52 mmol) in THF (18 mL) at 0° C., and the reaction mixture was stirred for 15 min, and then SEM-Cl (2.0 ml, 19.04 mmol) was added. The reaction mixture was stirred at room temperature for 4 hrs, then diluted with water (30 mL) and extracted with Ethyl Acetate (2×50 mL). Combined organic layers were washed with water (2×30 mL), brine (30 mL), dried over anhydrous sodium sulfate and concentrated under reduced pressure. The residue was purified by column chromatography on silica gel (100-200 mesh) using 10% Ethyl Acetate in Hexane as eluent to afford 1.3 g of 2-(2-nitrophenyl)-1-((2-(trimethylsilyl)-ethoxy)methyl)-1H-imidazole Int-96 as pale yellow liquid in 43% yield. ¹H-NMR (300 MHz, CDCl₃): δ 8.12 (d, J=8.1 Hz, 1H), 7.80-7.62 (m, 3H), 7.20 (d, J=1.5 Hz, 1H), 5.14 (s, 2H), 3.51-3.44 (m, 2H), 0.91-0.85 (m, 2H), 0.00 (s, 9H); MS (ESI) m/z 320.38 [M+H]⁺.

### 2-(1-((2-(Trimethylsilyl)ethoxy)methyl)-1H-imidazol-2-yl)aniline (Int-97)

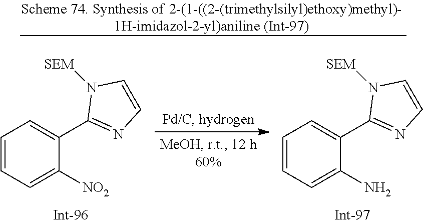 [SMILES: C[Si](C)(C)CCOCn1ccnc1-c1ccccc1[N+](=O)[O-] ; C[Si](C)(C)CCOCn1ccnc1-c1ccccc1N]

### Experimental Procedure:

10% Pd/C (20% by weight) was added to a stirred solution of 2-(2-nitrophenyl)-1-((2-(trimethylsilyl)ethoxy)methyl)-1H-imidazole Int-96 (2.7 g, 8.46 mmol) in Methanol (54 mL). The flask was flushed with Hydrogen gas, sealed and connected to a hydrogen balloon before the reaction mixture was stirred at room temperature for 12 hrs. Then the reaction mixture was filtered through a pad of Celite, and the filtrate was concentrated under reduced pressure. The residue was purified by column chromatography on silica gel (100-200 mesh) using 10% Ethyl Acetate in Hexane as eluent to afford 1.45 g of 2-(1-((2-(trimethylsilyl)ethoxy)methyl)-1H-imidazol-2-yl) aniline Int-97 as a brown solid in 60% yield. ¹H-NMR (400 MHz, DMSO-d₆): δ 7.41-7.39 (m, 2H), 7.10 (td, J=7.7, 1.4 Hz, 1H), 7.06 (d, J=1.3 Hz, 1H), 6.79 (dd, J=8.1, 0.9 Hz, 1H), 6.59 (td, J=7.5, 0.9 Hz, 1H), 5.89 (s, 2H), 5.25 (s, 2H), 3.52-3.48 (m, 2H), 0.85-0.81 (m, 2H), −0.05 (s, 9H); MS (ESI) m/z 290 [M+H]⁺.

### Synthesis of 2-(pyridin-2-yl)aniline (Int-100)

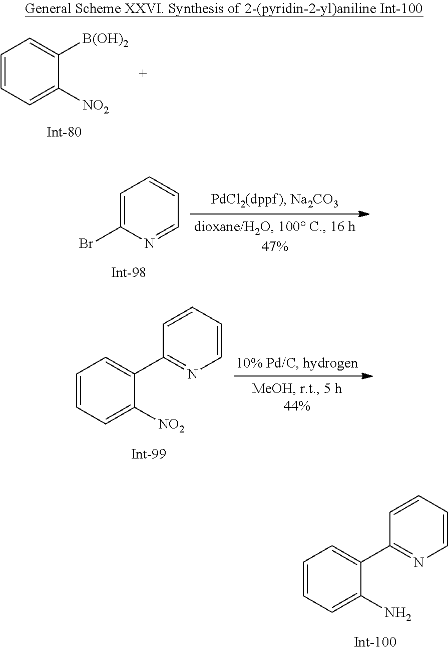 [SMILES: O=[N+]([O-])c1ccccc1B(O)O ; Brc1ccccn1 ; O=[N+]([O-])c1ccccc1-c1ccccn1 ; Nc1ccccc1-c1ccccn1]

### 2-(2-nitrophenyl)pyridine (Int-99)

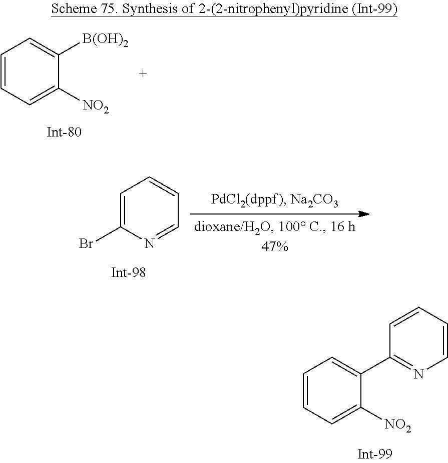 [SMILES: O=[N+]([O-])c1ccccc1B(O)O ; Brc1ccccn1 ; O=[N+]([O-])c1ccccc1-c1ccccn1]

### Experimental Procedure:

Na₂CO₃ (10.2 g, 73.17 mmol) was added to a solution of (2-nitrophenyl)boronic acid Int-80 (5.2 g, 32.05 mmol) and 2-bromopyridine Int-98 (5.0 g, 32.05 mmol) in a mixture 1,4-Dioxane/Water (4:1, 100 mL) at room temperature. The reaction mixture was degassed with Argon gas for 20 min, then the PdCl₂(dppf)-DCM complex (2.6 g, 3.21 mmol) was added, and the reaction mixture was degassed for additional 5 min. The reaction mixture was heated at 100° C. for 16 hrs. After cooling down to room temperature, the reaction mixture was diluted with water (150 mL) and extracted with Ethyl Acetate (3×150 mL). Combined organic layers were washed with water (2×40 mL), brine (40 mL), dried over anhydrous sodium sulfate and concentrated under reduced pressure. The residue was purified by column chromatography on silica gel (100-200 mesh) using 15% Ethyl Acetate in Hexane as eluent to afford 3.0 g of 2-(2-nitrophenyl)pyridine Int-99 as a pale yellow solid in 47% yield. ¹H-NMR (400 MHz, CDCl₃): δ 8.65 (d, J=4.8 Hz, 1H), 7.90 (d, J=1.2 Hz, 1H), 7.79 (td, J=7.6, 1.6 Hz, 1H), 7.69-7.58 (m, 2H), 7.56 (td, J=8.4, 2.0 Hz, 1H), 7.46 (d, J=8.0 Hz, 1H), 7.30 (ddd, J=7.2, 4.8, 0.8 Hz, 1H); MS (ESI) m/z 200.97 [M+H]⁺.

### 2-(Pyridin-2-yl)aniline (Int-100)

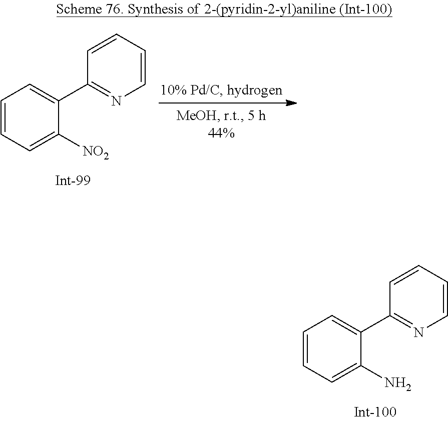 [SMILES: O=[N+]([O-])c1ccccc1-c1ccccn1 ; Nc1ccccc1-c1ccccn1]

### Experimental Procedure:

10% Pd/C (20% by weight) was added to a stirred solution of 2-(2-nitrophenyl)pyridine Int-99 (4.0 g, 20.0 mmol) in Methanol (40 mL). The flask was flushed with Hydrogen gas, sealed, connected to a hydrogen balloon, and the reaction mixture was stirred at room temperature for 5 hrs. Then the reaction mixture was filtered through a pad of Celite and the filtrate was concentrated under reduced pressure. The residue was purified by column chromatography on silica gel (100-200 mesh) using 40% Ethyl Acetate in Hexane as eluent to afford 1.5 g of 2-(thiazol-2-yl)aniline Int-100 as an off-white solid in 44% yield. ¹H-NMR (400 MHz, DMSO-d₆): δ 8.60 (ddd, J=4.9, 1.9, 0.9 Hz, 1H), 7.86 (ddd, J=8.0, 7.6, 2.0 Hz, 1H), 7.79-7.76 (dt, J=8.0, 0.8 Hz, 1H), 7.53 (dd, J=7.9, 1.5 Hz, 1H), 7.28 (ddd, J=7.4, 4.9, 1.1 Hz, 1H), 7.09 (ddd, J=8.2, 7.0, 1.4 Hz, 1H), 6.76 (dd, J=8.1, 1.2 Hz, 1H), 6.62 (ddd, J=7.9, 7.1, 1.2 Hz, 1H), 6.57 (bs, 2H); MS (ESI) m/z 171.02[M+H]⁺[M+H]⁺.

### (E)-5-(3-(3-(3-(trifluoromethyl)benzamido)phenyl)acrylamido)nicotinic acid (Int-102)

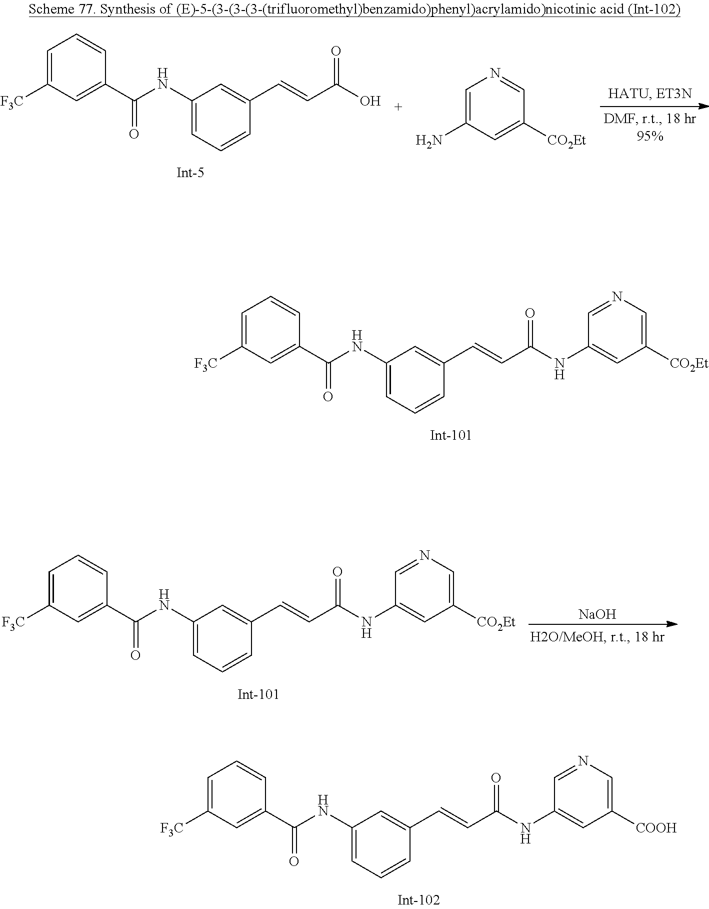 [SMILES: O=C(O)/C=C/c1cccc(NC(=O)c2cccc(C(F)(F)F)c2)c1 ; CCOC(=O)c1cncc(N)c1 ; CCOC(=O)c1cncc(NC(=O)/C=C/c2cccc(NC(=O)c3cccc(C(F)(F)F)c3)c2)c1 ; CCOC(=O)c1cncc(NC(=O)/C=C/c2cccc(NC(=O)c3cccc(C(F)(F)F)c3)c2)c1 ; "NaOH" ; O=C(/C=C/c1cccc(NC(=O)c2cccc(C(F)(F)F)c2)c1)Nc1cncc(C(=O)O)c1]

### Experimental Procedure:

Ethyl (E)-5-(3-(3-(3-(trifluoromethyl)benzamido)phenyl)acrylamido)nicotinate Int-101 was synthesized from acid Int-5 and amine and the product was obtained as off-white solid in 95% yield. Int-101 (71.4 mg, 0.15 mmol) was dissolved in methanol (1.1 mL) and 1 N NaOH solution in water (620 μL) was added. The reaction mixture was stirred at room temperature for 18 hours. Methanol was removed in vacuo and the aqueous solution was acidified with 2 N HCl solution in water to a pH of about 4-5. The formed off-white precipitate was isolated by centrifugation, washed with water (2×) and diethyl ether (1×), and dried in vacuo. Acid Int-102 was obtained as an off-white solid in 57% yield (39.6 mg). The product was used in the next step without any further purification.

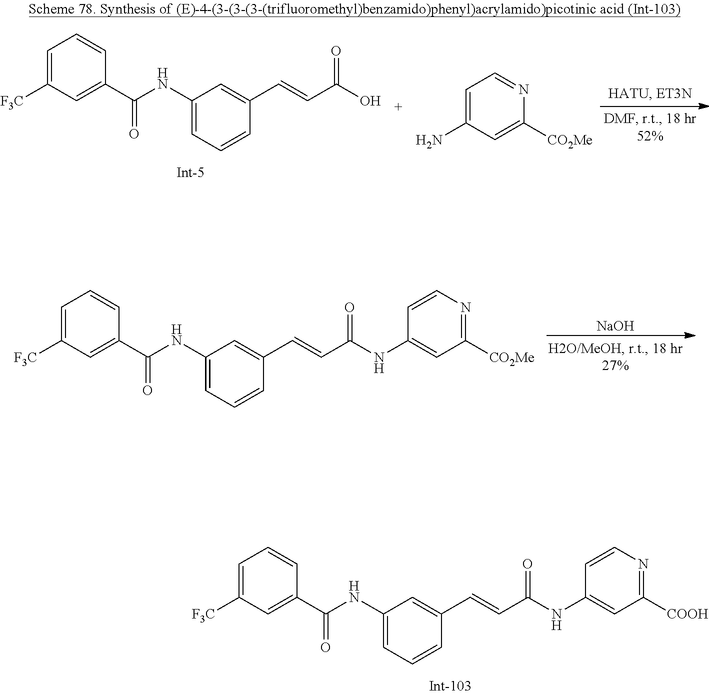 [SMILES: O=C(O)/C=C/c1cccc(NC(=O)c2cccc(C(F)(F)F)c2)c1 ; COC(=O)c1cc(N)ccn1 ; COC(=O)c1cc(NC(=O)/C=C/c2cccc(NC(=O)c3cccc(C(F)(F)F)c3)c2)ccn1 ; "NaOH" ; O=C(/C=C/c1cccc(NC(=O)c2cccc(C(F)(F)F)c2)c1)Nc1ccnc(C(=O)O)c1]

### Experimental Procedure:

Int-5 (67.1 mg, 0.20 mmol) was dissolved in DMF (600 μL) and triethylamine (42 μL, 0.30 mmol) was added, followed by methyl 4-aminopyridine-2-carboxylate (39.6 mg, 0.26 mmol). Then, HATU (98.9 mg, 0.26 mmol) was added, and the reaction mixture was stirred at RT for 18 hours. The solution was diluted with Ethyl Acetate (10 mL) and water (10 mL) and the phases were separated. The aqueous layer was extracted with ethyl acetate (3×10 mL). Combined organic layers were washed with water (1×20 mL) and brine (1×0 mL), dried over sodium sulfate and concentrated in vacuo. The crude product was purified on reversed phase silica gel using a gradient of water and acetonitrile with 0.1% trifluoroacetic acid from 90:10 to 0:100. Fractions containing the desired product were combined and treated with saturated aqueous sodium bicarbonate solution and extracted with DCM (3×). The combined organic phases were washed with brine (1×), dried over sodium sulfate and concentrated in vacuo to give the product as an off-white solid in 52% (48.8 mg). Ester (48.8 mg, 0.11 mmol) was dissolved in methanol (1.5 mL) and a 1 N NaOH solution in water (800 μL) was added. The reaction mixture was stirred at room temperature for 24 hours. Methanol was removed in vacuo and the residue was diluted with water (2 mL) before it was acidified to a pH of about 3 with a 2 N HCl solution in water. The precipitate was isolated by centrifugation, washed with water and dried in vacuo to give 24.6 mg of (E)-4-(3-(3-(3-(trifluoromethyl)benzamido) phenyl)acrylamido)picolinic acid Int-103 as a light-yellow solid in 27% yield.

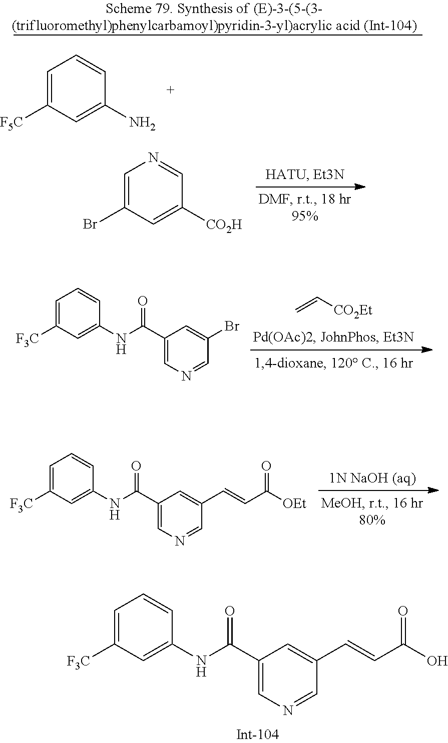

### Experimental Procedure:

DIPEA (21.7 mL, 124.30 mmol) was added to a stirred solution of 5-bromonicotinic acid (5.00 g, 24.47 mmol) in DMF (50 mL), followed by HATU (14.20 g, 37.36 mmol) at 0° C., and the reaction mixture was stirred for 15 minutes. Then, 3-(trifluoromethyl)aniline (3.75 mL, 29.80 mmol) was added dropwise at 0° C. and the reaction mixture was stirred at room temperature for 16 hrs. Then the reaction mixture was diluted with DI water (100 mL) and extracted into Ethyl Acetate (2×100 mL). Combined organic layers were washed with water (3×100 mL), brine (50 mL), dried over anhydrous sodium sulfate and concentrated under reduced pressure. The crude product was purified by column chromatography (100-200 silica) using 20% ethyl acetate in hexane as eluent to give 5-bromo-N-(3-(trifluoromethyl)phenyl)nicotinamide as a yellow solid in 70% yield (6.00 g). MS (ESI) m/z=345.07 [M+H]⁺.

Triethylamine (5.0 mL, 36.00 mmol) and ethyl acrylate (1.54 mL 13.00 mmol) were added to a solution of 5-bromo-N-(3-(trifluoromethyl)phenyl)nicotinamide (2.50 g, 7.26 mmol) in 1,4-dioxane (35 mL) at room temperature, and the solution was degassed by bubbling with nitrogen gas for 15 minutes. Then JohnPhos (0.40 g, 1.30 mmol) was added, followed by Pd(OAc)₂ (0.46 g, 0.71 mmol) and degassing was continued for another 10 minutes. Then the reaction mixture was heated at 120° C. for 16 hrs. The reaction mixture was diluted with Ethyl Acetate (50 mL) and filtered through a pad of celite that was washed with ethyl acetate. The filtrate was concentrated under reduced pressure, and the crude product was purified by column chromatography (100-200 silica) using 20% ethyl acetate in hexane as eluent to afford (E)-ethyl 3-(5-(3-(trifluoromethyl)phenylcarbamoyl) pyridin-3-yl)acrylate as a pale brown liquid in 79% yield (2.10 g). MS (ESI) m/z=363.0 [M+H]⁺.

To a stirred solution of (E)-ethyl 3-(5-(3-(trifluoromethyl)phenylcarbamoyl)pyridin-3-yl)acrylate (2.00 g, 5.50 mmol) in methanol (40 mL), 1 N NaOH solution in water (40.0 mL, 27.50 mmol) was added at 0° C., and the reaction mixture was stirred at room temperature for 12 hrs. Then, methanol was removed under reduced pressure and the residue was diluted with water (10 m.L). The pH was adjusted to about 3-4 with a 1N HCl solution in water. The resulting precipitate was filtered off, washed with water (25 mL) and dried in vacuo to afford (E)-3-(5-(3-(trifluoromethyl)phenyl carbamoyl)pyridin-3-yl)acrylic acid (Int-104) as an off-white solid in 80% yield (1.48 g). ¹H-NMR (DMSO-d₆, 400 MHz) δ 12.67 (bs, 1H), 10.74 (s, 1H), 9.08 (d, J=1.6 Hz 1H), 9.05 (d, J=1.6 Hz, 1H), 8.66-8.65 (m, 1H), 8.22 (s, 1H), 8.06-8.04 (m, 1H), 7.74-7.70 (d, J=16 Hz, 1H), 7.66-7.62 (m, 1H), 7.51-7.49 (m, 1H), 6.83-6.79 (d, J=16, Hz 1H) ppm; MS (ESI) m/z=337.22 [M+H]⁺.

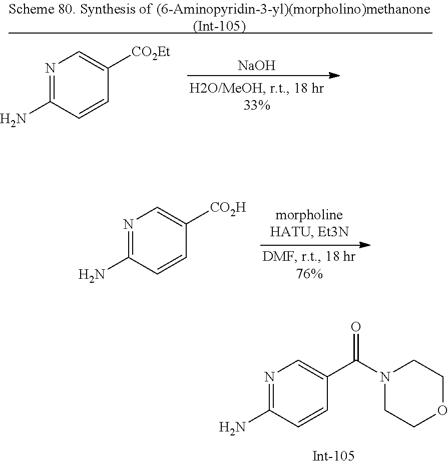 [SMILES: CCOC(=O)c1ccc(N)nc1 ; "NaOH" ; Nc1ccc(C(=O)O)cn1 ; Nc1ccc(C(=O)N2CCOCC2)cn1]

### Experimental Procedure:

Ethyl 6-aminonicotinate (831.0 mg, 5.00 mmol) was dissolved in methanol (36 mL) and a 1 N solution of NaOH in water (20.0 mL) was added. The reaction mixture was stirred at room temperature for 18 hours. Then, methanol was removed in vacuo and the residue was diluted with water (2 mL). The solution was acidified with 2 N HCL solution in water to a pH of about 3. The precipitate was isolated by centrifugation, washed with water and dried in vacuo. The product was obtained as an off-white solid in 33% yield (229.0 mg) and used in the next step without further purification. 6-Aminonicotinic acid (100.0 mg, 0.72 mmol) was dissolved in DMF (1.5 mL) and triethylamine (201 p L, 1.45 mmol) was added, followed by morpholine (188 μL, 2.18 mmol) and HATU (441.0 mg, 1.16 mmol). The reaction mixture was stirred at room temperature overnight. Ethyl acetate (50 mL) and water (50 mL) were added and the layers were separated. The aqueous layer was extracted with Ethyl Acetate (3×50 mL) and the combined organic layers were washed with water (2×50 mL), brine (1×50 mL), dried over anhydrous sodium sulfate and concentrated in vacuo. The crude product was purified on silica gel with a gradient of DCM and MeOH with 0.1% triethylamine from 100:0 to 20:80. Int-105 was obtained as yellow needles in 66% yield (98.1 mg). ¹H-NMR (400 MHz, DMSO-d₆): δ 8.84 (bs, 1H), 8.02 (d, J=2.3 Hz, 1H), 7.49-7.45 (m, 1H), 6.50 (bs, 2H), 6.48-6.44 (m, 1H), 3.64-3.55 (m, 4H), 3.53-3.44 (m, 4H) ppm; MS m/z=208.2 [M+H]⁺.

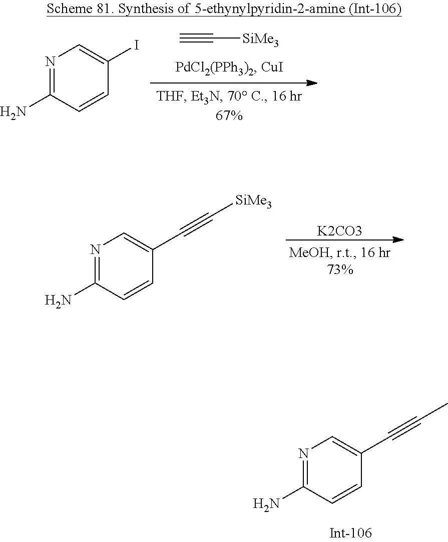

### Experimental Procedure:

TMS-acetylene (6.7 g, 67.0 mmol) dissolved in THF (50 mL) was added to a stirred solution of 5-iodo-2-aminopyridine (10.0 g, 45.0 mmol), followed by triethylamine (30.0 mL, 220.0 mmol). The solution was degassed by purging with nitrogen gas for 5 minutes before PdCl₂(PPh₃)₂ (316.0 mg, 4.50 mmol) and copper(I) iodide (860.0 mg, 4.50 mmol) were added. The reaction mixture was refluxed at 70° C. for 16 hrs. After cooling down to room temperature, the reaction mixture was diluted with water (100 mL) and extracted with Ethyl Acetate (2×100 mL). The combined organic layers were washed with water (3×50 mL), brine (1×50 mL), dried over anhydrous sodium sulfate and concentrated under reduced pressure. The crude product was purified by column chromatography (100-200 silica-gel) using 5% Ethyl Acetate in hexane as eluent to afford 5-((trimethylsilyl)ethynyl)pyridin-2-amine as a brown gummy-like solid in 67% yield (5.8 g).

Anhydrous potassium carbonate (8.4 g, 60.0 mmol) was added to a stirred solution of 5-((trimethylsilyl)ethynyl)pyridin-2-amine (5.8 g, 30.0 mmol) in methanol (50 mL), and the reaction mixture was stirred at room temperature for 16 hrs. Then, the reaction mixture was concentrated in vacuo to dryness. The residue was diluted with water (100 mL) and extracted with Ethyl Acetate (2×100 mL). The combined organic layers were washed with water (2×50 mL), brine (1×50 mL), dried over anhydrous sodium sulfate and concentrated under reduced pressure. The crude product was purified by column chromatography (100-200 silica-gel) using 5% ethyl acetate in hexane as eluent to afford 5-ethynylpyridin-2-amine Int-106 as a brown solid in 73% yield (2.6 g).

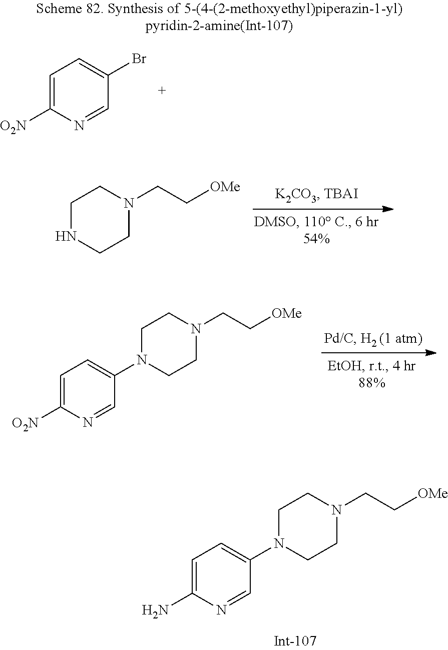 [SMILES: O=[N+]([O-])c1ccc(Br)cn1 ; COCCN1CCNCC1 ; COCCN1CCN(c2ccc([N+](=O)[O-])nc2)CC1 ; COCCN1CCN(c2ccc(N)nc2)CC1]

### Experimental Procedure:

Potassium carbonate (1.36 g, 9.85 mmol), piperazine (0.82 mL, 7.40 mmol) and tetrabutylammonium iodide (20.0 mg, 0.049 mmol) were added to a stirred solution of 5-bromo-2-nitro-pyridine (1.00 g, 4.92 mmol) in dimethylsulfoxide (10 mL). The reaction mixture was heated at 120° C. for 16 hours. After cooling down to room temperature, the mixture was acidified with a 1 N HCL solution in water and partitioned between dichloromethane (50 mL) and water (50 mL). The aqueous layer was made basic with saturated aqueous sodium carbonate solution and extracted with dichloromethane. The organic layer was dried over anhydrous sodium sulfate and concentrated in vacuo. The crude product was purified by column chromatography (100-200 silica) using 20% ethyl acetate in hexane as eluent to afford 1-(2-methoxyethyl)-4-(6-nitropyridin-3-yl)piperazine in 54% yield (0.70 g). MS (ESI) m/z=267.1 [M+H]⁺.

10% Pd/C (20% by wt) was added to a stirred solution of 1-(2-methoxyethyl)-4-(6-nitropyridin-3-yl)piperazine (0.70 g, 2.63 mmol) in Ethanol (50 mL), and the flask was equipped with a hydrogen balloon. The reaction mixture was stirred at room temperature under hydrogen atmosphere for 4 hrs, before it was filtered through a pad of celite and the filtrate was concentrated under reduced pressure to afford 5-(4-(2-methoxyethyl)piperazin-1-yl)pyridin-2-amine Int-107 in 88% yield (0.55 g). The product was without further purification in the next step. MS (ESI) m/z=237.1 [M+H]⁺.

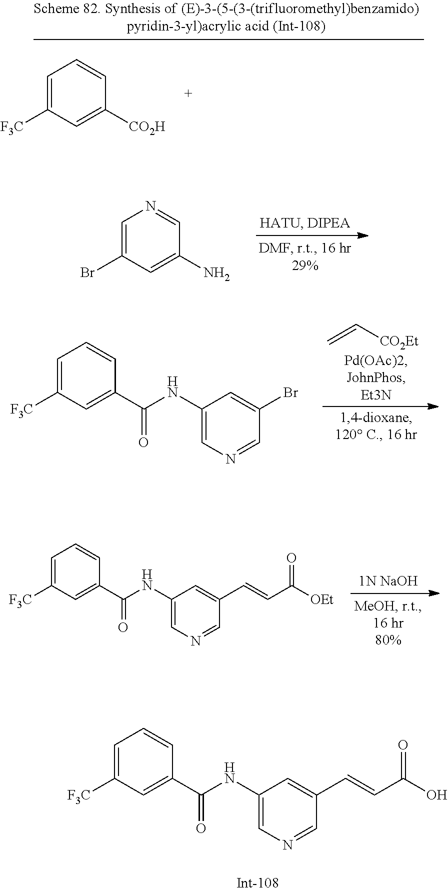

### Experimental Procedure:

DIPEA (45.8 mL 263.1 mmol) was added to a stirred solution of 3-(trifluoromethyl)benzoic acid (10 g, 52.63 mmol) in DMF (100 mL), followed by HATU (29.9 g, 78.9 mmol) at 0° C., and the reaction mixture was stirred for 15 min. Then 5-bromopyridin-3-amine (10.9 g, 63.51 mmol) was added to the reaction mixture dropwise at 0° C. The reaction mixture was stirred at RT for 16 hrs. After completion of the reaction, the reaction mixture was diluted with water (100 mL) and extracted with Ethyl Acetate (2×100 mL). The combined organic layers were washed with water (3×100 mL), brine (50 mL), dried over anhydrous sodium sulphate and concentrated under reduced pressure. The resultant crude compound was purified by column chromatography (100-200 silica) using 20% Ethyl Acetate in Hexane as eluent to afford 5.2 g of N-(5-bromopyridin-3-yl)-3-(trifluoromethyl)benzamide as a yellow solid in 29% yield. MS (ESI) m/z 345.29 [M+H]⁺

To a degassed solution of N-(5-bromopyridin-3-yl)-3-(trifluoromethyl)benzamide (5 g, 14.49 mmol) in Dioxane (50 mL) in a sealed tube was added TEA (10.2 mL 72.5 mmol) and Ethyl Acrylate (3 mL, 26 mmol) at RT for 15 min. Then JohnPhos (0.89 g 2.8 mmol) was added, followed by Pd(OAc)₂ (0.96 g 1.45 mmol) and degassing was continued for another 10 min. The reaction mixture was heated at 120° C. for 16 h. After completion of the reaction, the reaction mixture was diluted with Ethyl Acetate (100 mL) and filtered through a pad of Celite, and Celite was washed with Ethyl Acetate. The filtrate was concentrated under reduced pressure The resultant crude compound was purified by column chromatography (100-200 silica) using 20% Ethyl Acetate in Hexane as eluent to afford 3.8 g of (E)-ethyl 3-(5-(3-(trifluoromethyl)benzamido)pyridin-3-yl)acrylate Int-108 as a pale brown liquid in 72% yield.

1M NaOH solution (40 mL, 41.6 mmol) was added to a stirred solution of (E)-Ethyl 3-(5-(3-(trifluoromethyl)phenylcarbamoyl)pyridin-3-yl)acrylate (3.8, 10.4 mmol) in Methanol (75 mL) at 0° C. and the reaction mixture was stirred at RT for 12 h. After completion of the reaction, the methanolic solution was concentrated under reduced pressure and the residue was diluted with water (10 mL) and pH was adjusted with 1N HCl to approx. 3-4. The resultant precipitate was filtered, washed with water (50 mL) and dried under vacuum to afford 1 g of (E)-3-(5-(3-(trifluoromethyl)benzamido)pyridin-3-yl)acrylic acid as an off-white solid in 29% yield. MS (ESI) m/z 335.0 [M−H]. ¹H NMR (DMSO-d₆, 400 MHz): δ 12.76 (br s, 1H), 8.93 (s, 1H), 8.65 (m, 1H), 8.44 (m, 1H), 8.34-8.29 (m, 2H), 8.03-8.01 (d, J=1.8 Hz, 1H), 7.65-7.61 (d, J=16 Hz, 1H), 6.62-6.58 (d, J=16 Hz, 1H).

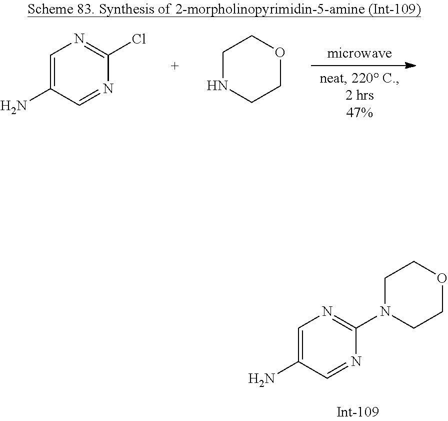 [SMILES: Nc1cnc(Cl)nc1 ; C1COCCN1 ; Nc1cnc(N2CCOCC2)nc1]

### Experimental Procedure:

A microwave vial was charged with 2-chloro-5-aminopyrimidine (129.5 mg, 1.0 mmol) and morpholine (1 mL). The vial was sealed and heated in a microwave reactor to 220° C. for 2 hours. Then the reaction mixture was diluted with water (10 mL) and extracted with Ethyl Acetate (3×10 mL). Combined organic phases were washed with brine (1×25 mL), dried over anhydrous sodium sulfate and concentrated in vacuo. The crude product was purified on reverse phase silica gel using a gradient of water and acetonitrile with 0.1% trifluoroacetic acid (90:10 to 0:100). The fractions containing the desired product were combined and treated with aqueous saturated sodium bicarbonate solution. The mixture was extracted with dichloromethane (3×) and the combined organic layers were washed with brine (1×), dried over anhydrous sodium sulfate, filtered and concentrated in vacuo to give 85.0 mg of Int-109 as an orange solid in 47% yield.

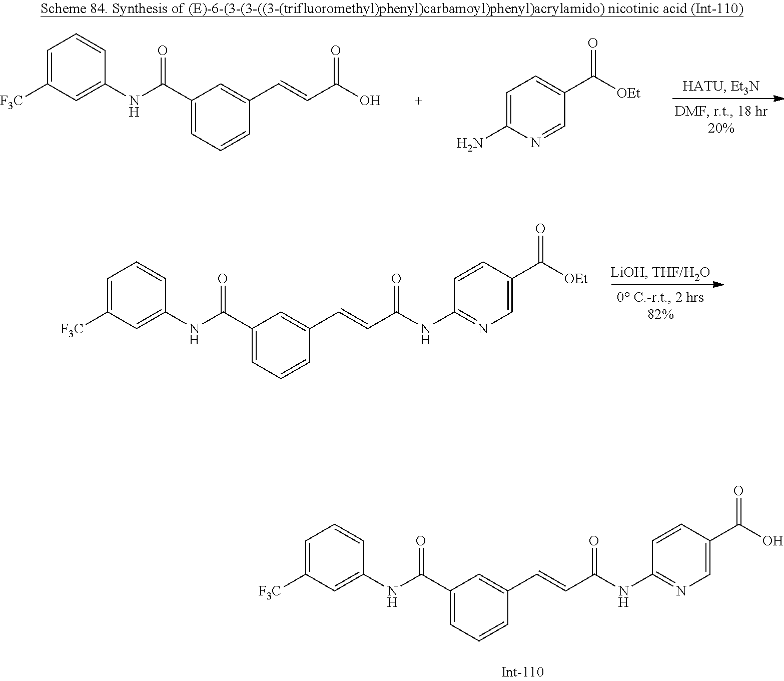 [SMILES: O=C(O)/C=C/c1cccc(C(=O)Nc2cccc(C(F)(F)F)c2)c1 ; CCOC(=O)c1ccc(N)nc1 ; CCOC(=O)c1ccc(NC(=O)/C=C/c2cccc(C(=O)Nc3cccc(C(F)(F)F)c3)c2)nc1 ; O=C(/C=C/c1cccc(C(=O)Nc2cccc(C(F)(F)F)c2)c1)Nc1ccc(C(=O)O)cn1]

### Experimental Procedure:

Ethyl (E)-6-(3-(3-((3-(trifluoromethyl)phenyl)carbamoyl)phenyl)acrylamido)nicotinate was synthesized according to the General Procedure I from Int-12 and ethyl 6-aminonicotinate, and the product was obtained as an off-white solid in 20% yield. Ethyl (E)-6-(3-(3-((3-(trifluoromethyl)phenyl)carbamoyl)phenyl)acrylamido)nicotinate (48.9 mg, 0.10 mmol) was dissolved in dry THF (1 mL). The reaction mixture was cooled to 0° C. and a solution of LiOH (42.0 mg, 1.00 mmol) in water (1 mL) was slowly added. The reaction mixture was allowed to reach room temperature and it was stirred at RT for 2 hours. The reaction mixture was acidified with 5 M HCl solution in 1,4-Dioxane to a pH of 2-3. THF and dioxane were removed in vacuo and the off-white precipitate was isolated by centrifugation and recrystallized from a mixture of hot ethanol and water. The product was obtained as an off-white solid in 82% yield.

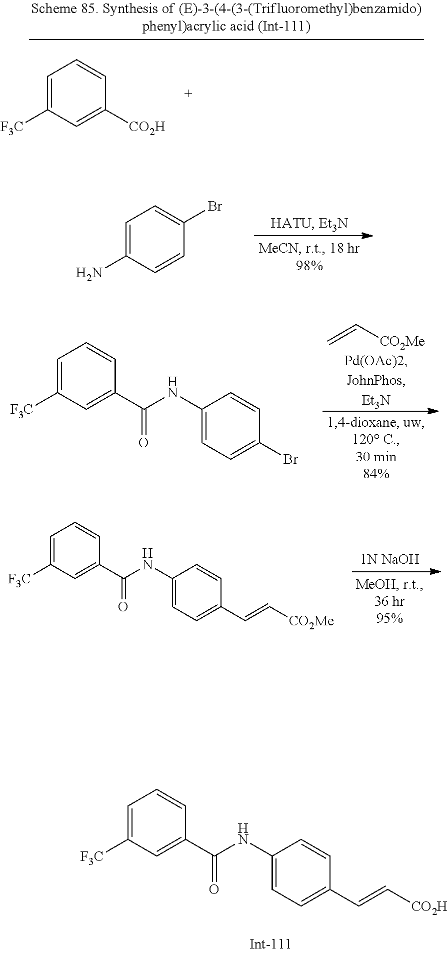

### Experimental Procedure:

3-(Trifluoromethyl)benzoic acid (1.90 g, 10.00 mmol) and 4-bromoaniline (1.89 g, 11.00 mmol) were suspended in dry acetonitrile (100 mL) and triethylamine (2.1 mL, 15.00 mmol) was added. Then, HATU (4.56 g, 12.00 mmol) was added, and the reaction mixture was stirred at RT for 18 hours. The reaction mixture was diluted with water (350 mL) and stored at room temperature for 30 minutes. The precipitate was filtered off, washed with water and dried in vacuo to give N-(4-bromophenyl)-3-(trifluoromethyl)benzamide as an off-white solid in 98% yield. ¹H-NMR (400 MHz, DMSO-d₆): δ 10.57 (s, 1H), 8.28 (s, 1H), 8.26 (d, J=7.9 Hz, 1H), 7.98 (d, J=7.8 Hz, 1H), 7.81-7.74 (m, 3H), 7.58-7.55 (m, 2H).

N-(4-bromophenyl)-3-(trifluoromethyl)benzamide (1.38 g, 4.00 mmol), JohnPhos (339.7 mg, 0.80 mmol) and Pd(OAc)₂ (89.8 mg, 0.40 mmol) were placed in a 20 mL microwave vial and 1,4-Dioxane was added, followed by triethylamine (1.11 mL, 8.00 mmol). The reaction mixture was degassed by bubbling with nitrogen gas for 10 minutes before Methyl Acrylate (906 μL, 10.00 mmol) was added and the vial was immediately sealed. The reaction mixture was heated at 120° C. for 30 minutes in a microwave reactor. After cooling down to room temperature, the reaction mixture was filtered through a Celite plug (DCM) and the filtrate was concentrated in vacuo to give an orange solid. The crude product was purified on silica gel with Hexanes/Ethyl Acetate (gradient from 100:0 to 0:100) and the product was obtained as a light yellow solid in 84% yield (1.17 g). ¹H-NMR (400 MHz, DMSO-d₆): δ 10.65 (s, 1H), 8.29-8.26 (m, 2H), 7.98 (d, J=7.8 Hz, 1H), 7.89-7.84 (m, 2H), 7.80 (t, J=7.8 Hz, 1H), 7.76-7.71 (m, 2H), 7.64 (d, J=16.0 Hz, 1H), 6.58 (d, J=16.0 Hz, 1H), 3.72 (s, 3H).

Methyl (E)-3-(4-(3-(trifluoromethyl)benzamido)phenyl)acrylate (1.17 g, 3.35 mmol) was dissolved in methanol (25 mL) and a 1 N aqueous NaOH solution (13.5 mL) was added. The reaction mixture was stirred at RT for 36 hours, then methanol was removed in vacuo. The solution was diluted with water (10 mL) and the pH was adjusted to about 3 with 2N aqueous HCl. The solution was extracted with Ethyl Acetate (3×15 mL) and combined organic layers were washed with brine (1×25 mL), dried over anhydrous sodium sulfate and concentrated in vacuo to give (E)-3-(4-(3-(trifluoromethyl)benzamido)phenyl)acrylic acid as a yellow solid in 95% yield (1.07 g). ¹H-NMR (400 MHz, DMSO-d₆): δ 12.31 (bs, 1H), 10.63 (s, 1H), 8.32-8.24 (m, 2H), 7.99 (d, J=7.9 Hz, 1H), 7.88-7.84 (m, 2H), 7.80 (t, J=7.8 Hz, 1H), 7.74-7.68 (m, 2H), 7.56 (d, J=16.0 Hz, 1H), 6.47 (d, J=16.0 Hz, 1H).

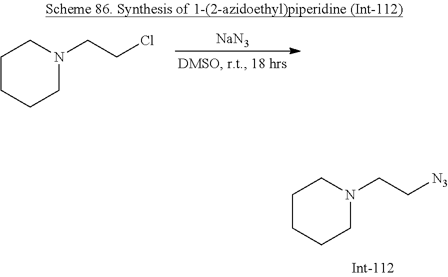 [SMILES: ClCCN1CCCCC1 ; [N-]=[N+]=NCCN1CCCCC1 ; "NaN3"]

### Experimental Procedure:

Sodium azide (1.1 equiv.) was suspended in DMSO (0.5 M) and stirred for 2 hours at room temperature until all solids dissolved. Then 1-(2-chloroethyl)piperidine (1.0 equiv.) was added and the reaction mixture was stirred at room temperature for 18 hours. Then the reaction mixture was quenched with DI water and stirred until the solution cooled down to room temperature. The mixture was extracted with Diethyl Ether (3×) and combined organic phases were washed with water (2×), brine (1×), dried over anhydrous sodium sulfate and concentrated in vacuo to give the desired product as a light-yellow oil in 74% yield.

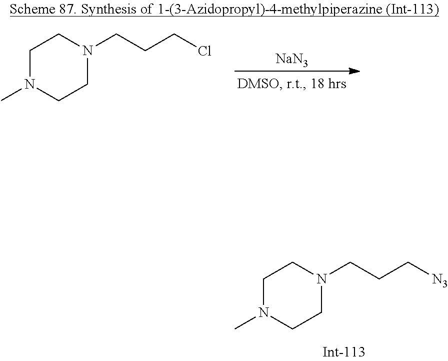

### Experimental Procedure:

Sodium azide (1.1 equiv.) was suspended in DMSO (0.5 M) and stirred for 2 hours at RT until all solids dissolved. Then 1-(3-chloropropyl)-4-methylpiperazine (1.0 equiv.) was added and the reaction mixture was stirred at room temperature for 18 hours. Then the reaction mixture was quenched with DI water and extracted with Diethyl Ether (3×). Combined organic phases were washed with water (2×), brine (1×), dried over anhydrous sodium sulfate and concentrated in vacuo to give the desired product as colorless oil in 65% yield. ¹H-NMR (400 MHz, DMSO-d₆): δ 3.36-3.31 (m, 2H), 2.38-2.24 (m, 10H), 2.14 (s, 3H), 1.67 (quintet, J=6.9 Hz, 2H).

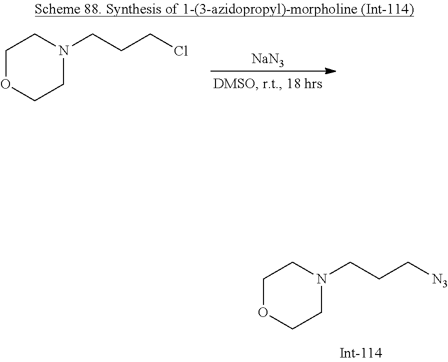 [SMILES: ClCCCN1CCOCC1 ; [N-]=[N+]=NCCCN1CCOCC1 ; "NaN3"]

### Experimental Procedure:

Sodium azide (1.1 equiv.) was suspended in DMSO (0.5 M) and stirred for 2 hours at RT until all solids dissolved. Then 1-(3-chloropropyl)morpholine (1.0 equiv.) was added and the reaction mixture was stirred at RT for 18 hours. Then the reaction mixture was quenched with DI water and extracted with Diethyl Ether (3×). Combined organic phases were washed with water (2×), brine (1×), dried over anhydrous sodium sulfate and concentrated in vacuo to give the desired product as colorless oil in 36% yield. ¹H-NMR (400 MHz, DMSO-d₆): δ 3.56 (t, J=4.7 Hz, 4H), 3.35 (t, J=6.8 Hz, 2H), 2.33-2.30 (m, 6H), 1.68 (quintet, J=6.9 Hz, 2H).

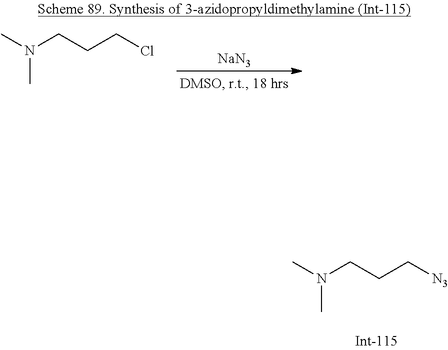

### Experimental Procedure:

Sodium azide (1.1 equiv.) was suspended in DMSO (0.5 M) and stirred for 2 hours at RT until all solids dissolved. Then (3-chloropropyl)dimethylamine (1.0 equiv.) was added and the reaction mixture was stirred at RT for 18 hours. Then the reaction mixture was quenched with DI water and extracted with Diethyl Ether (3×). Combined organic phases were washed with water (2×), brine (1×), dried over anhydrous sodium sulfate and concentrated in vacuo to give the desired product as pale yellow oil in 55% yield. ¹H-NMR (400 MHz, DMSO-d₆): δ 3.37-3.33 (m, 2H), 2.24 (t, J=7.0 Hz, 2H), 2.11 (s, 6H), 1.65 (quintet, J=6.9 Hz, 2H).

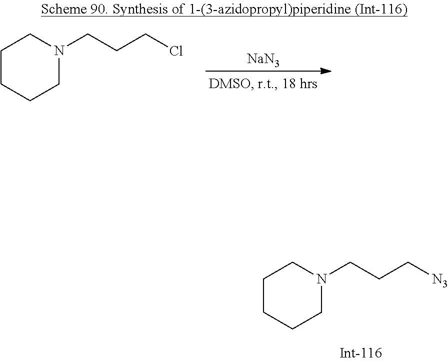 [SMILES: ClCCCN1CCCCC1 ; [N-]=[N+]=NCCCN1CCCCC1 ; "NaN3"]

Sodium azide (1.1 equiv.) was suspended in DMSO (0.5 M) and stirred for 2 hours at RT until all solids dissolved. Then 1-(3-chloropropyl)piperidine (1.0 equiv.) was added and the reaction mixture was stirred at RT for 18 hours. Then the reaction mixture was quenched with DI water and extracted with Diethyl Ether (3×). Combined organic phases were washed with water (2×), brine (1×), dried over anhydrous sodium sulfate and concentrated in vacuo to give the desired product as pale-yellow oil in 48% yield. ¹H-NMR (400 MHz, DMSO-d₆): δ 3.33 (t, J=6.7 Hz, 2H), 2.27 (m, 6H), 1.66 (quintet, J=6.9 Hz, 2H), 1.47 (quintet, J=5.5 Hz, 4H), 1.39-1.31 (m, 2H).

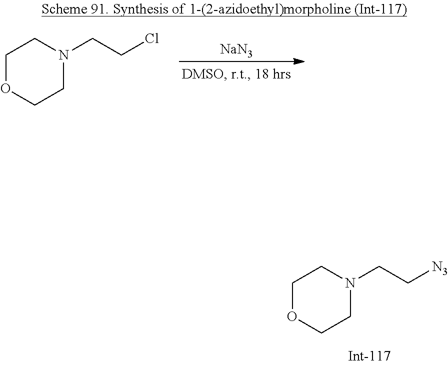 [SMILES: ClCCN1CCOCC1 ; [N-]=[N+]=NCCN1CCOCC1 ; "NaN3"]

Sodium azide (1.1 equiv.) was suspended in DMSO (0.5 M) and stirred for 2 hours at RT until all solids dissolved. Then 1-(2-chloroethyl)morpholine (1.0 equiv.) was added and the reaction mixture was stirred at RT for 18 hours. Then the reaction mixture was quenched with DI water and extracted with Diethyl Ether (3×). Combined organic phases were washed with water (2×), brine (1×), dried over anhydrous sodium sulfate and concentrated in vacuo to give the desired product as colorless oil in 61% yield.

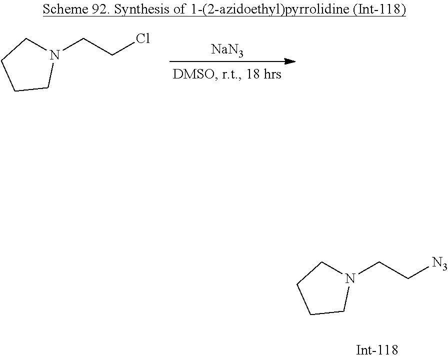 [SMILES: ClCCN1CCCC1 ; [N-]=[N+]=NCCN1CCCC1 ; "NaN3"]

Sodium azide (1.1 equiv.) was suspended in DMSO (0.5 M) and stirred for 2 hours at RT until all solids dissolved. Then 1-(2-chloroethyl)pyrrolidine (1.0 equiv.) was added and the reaction mixture was stirred at RT for 18 hours. Then the reaction mixture was quenched with DI water and extracted with Diethyl Ether (3×). Combined organic phases were washed with water (2×), brine (1×), dried over anhydrous sodium sulfate and concentrated in vacuo to give the desired product as orange oil in 70% yield.

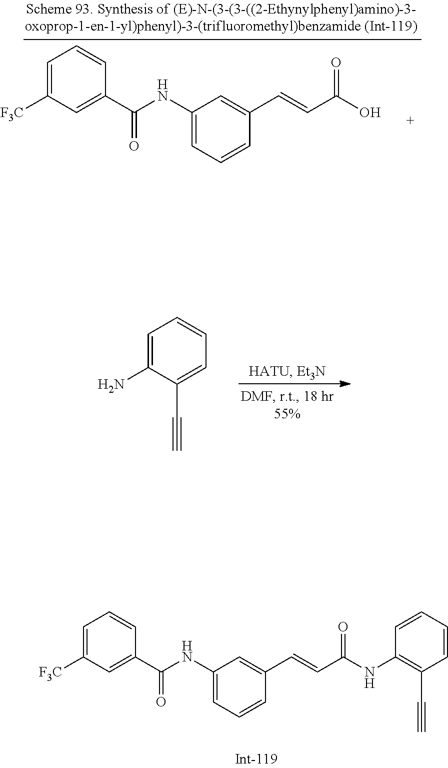

### Experimental Procedure:

(E)-3-(3-(3-(trifluoromethyl)benzamido)phenyl)acrylic acid Int-5 was reacted with 2-ethynylaniline using experimental conditions outlined in the General Procedure I to give Int-119 as a yellow solid in 55% yield. ¹H-NMR (400 MHz, DMSO-d₆): δ 10.61 (s, 1H), 9.68 (s, 1H), 8.34 (s, 1H), 8.30 (d, J=7.8 Hz, 1H), 8.18 (s, 1H), 8.00 (d, J=7.8 Hz, 1H), 7.94 (d, J=8.0 Hz, 1H), 7.82 (t, J=7.8 Hz, 1H), 7.75 (dt, J=8.0, 1.5 Hz, 1H), 7.59 (d, J=15.7 Hz, 1H), 7.52 (dd, J=7.7, 1.5 Hz, 1H), 7.47 (t, J=7.8 Hz, 1H), 7.44-7.39 (m, 2H), 7.17 (td, J=7.6, 1.2 Hz, 1H), 7.13 (d, J=15.6 Hz, 1H), 4.55 (s, 1H).

### (E)-N-(2-aminophenyl)-3-(3-(N-(4-fluoro-3-(trifluoromethyl)phenyl) sulfamoyl) phenyl)acrylamide (Compound 1)

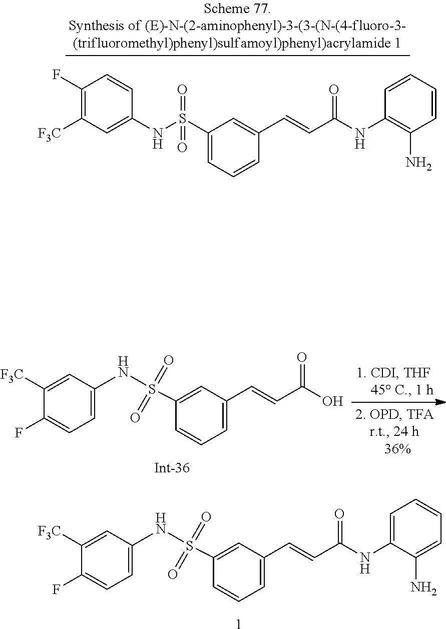

### Experimental Procedure:

N,N′-Carbonyldiimidazole (20.8 mg, 0.13 mmol) was suspended in anhydrous THF (1.1 mL) and (E)-3-(3-(N-(4-fluoro-3-(trifluoromethyl)phenyl)sulfamoyl)phenyl)acrylic acid Int-36 (50.0 mg, 0.13 mmol) was added. The reaction mixture was heated at 45° C. for 1 hr, and then cooled down to room temperature. In a second flask, o-Phenylenediamine (OPD) (17.3 mg, 0.16 mmol) was dissolved in anhydrous THF (720 p L) and trifluoroacetic acid (10 p L, 0.13 mmol) was added. Both solutions were combined at room temperature, and the resulting reaction mixture was stirred at room temperature for 24 hrs. Then it was diluted with DCM (5 mL) and treated with saturated aqueous sodium bicarbonate solution (5 mL). The aqueous layer was extracted with DCM (1×5 mL) and the combined organic layers were dried over anhydrous sodium sulfate and concentrated in vacuo. The residue was purified via column chromatography on silica gel using a 0-60% gradient of Methanol in DCM and methanol as eluent. The product was dissolved in DCM, filtered through a PTFE-syringe filter and the filtrate was evaporated to dryness to give 22 mg of (E)-N-(2-aminophenyl)-3-(3-(N-(4-fluoro-3-(trifluoromethyl)phenyl)sulfamoyl)phenyl)acrylamide 1 as a light orange solid in 36% yield. ¹H-NMR (400 MHz, DMSO-d₆): δ 9.43 (s, 1H), 8.00 (s, 1H), 7.87 (d, J=7.5 Hz, 1H), 7.72 (d, J=8.0 Hz, 1H), 7.65 (d, J=7.8 Hz, 1H), 7.58 (d, J=15.8 Hz, 1H), 7.48-7.34 (m, 3H), 6.98 (d, J=15.8 Hz, 1H), 6.94-6.90 (m, 1H), 6.75 (dd, J=8.0, 1.3 Hz, 1H), 6.60-6.56 (m, 1H), 6.49 (dd, J=5.8, 3.4 Hz, 1H), 6.36 (dd, J=5.7, 3.5 Hz, 1H), 4.97 (bs, 2H); MS m/z=480 [M+H]⁺.

### General Procedure I.

The corresponding acid (0.10 mmol) and the corresponding amine (0.13 mmol) were dissolved in anhydrous N,N-Dimethyl Formamide (300 p L) and Triethylamine (0.15 mmol, 21 μL) was added. The reaction mixture was cooled down to 0° C., and HATU (1-[Bis(dimethylamino)methylene]-1H-1,2,3-triazolo[4,5-b]pyridinium3-oxid hexafluorophosphate) (0.13 mmol, 49.4 mg) was added. The reaction mixture was stirred at room temperature for 72 hours. Then the reaction mixture was diluted with water and extracted with Ethyl Acetate (3×10 mL). Combined organic layers were dried over anhydrous sodium sulfate, filtered and concentrated in vacuo. The crude product was purified on reversed-phase silica gel with Water/Acetonitrile+0.1% Trifluoroacetic acid. Fractions containing the desired product were combined and treated with saturated sodium bicarbonate solution. The resulting mixture was extracted with Ethyl Acetate (3×50 mL). Combined organic layers were washed with brine (1×50 mL), dried over anhydrous sodium sulfate, filtered and concentrated in vacuo to give the desired product.

### (E)-N-(2-(1H-benzo[d]imidazol-2-yl)phenyl)-3-(3-(N-(4-fluoro-3-(trifluoromethyl) phenyl)sulfamoyl)phenyl)acrylamide (Compound 2)

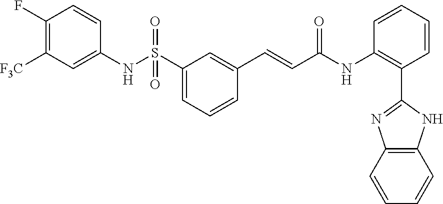 [SMILES: O=C(/C=C/c1cccc(S(=O)(=O)Nc2ccc(F)c(C(F)(F)F)c2)c1)Nc1ccccc1-c1nc2ccccc2[nH]1]

(E)-3-(3-(N-(4-fluoro-3-(trifluoromethyl)phenyl)sulfamoyl)phenyl)acrylic acid (Int-36) was reacted with 2-(2-aminophenyl)-1H-benzimidazole using the experimental conditions outlined in the General Procedure I to give Compound 2 as a yellow solid in 13% yield. ¹H-NMR (400 MHz, MeOD): δ 8.74 (d, J=8.4 Hz, 1H), 8.06 (s, 1H), 7.98 (dd, J=7.9, 1.3 Hz, 1H), 7.93 (d, J=7.8 Hz, 1H), 7.76-7.72 (m, 2H), 7.69-7.58 (m, 3H), 7.49-7.38 (m, 3H), 7.32-7.19 (m, 4H), 6.88 (d, J=15.8 Hz, 1H); MS m/z=581 [M+H]⁺.

### (E)-N-(2-(1H-benzo[d]imidazol-2-yl)phenyl)-3-(3-(N-(4-(trifluoromethyl)phenyl) sulfamoyl)phenyl)acrylamide (Compound 3)

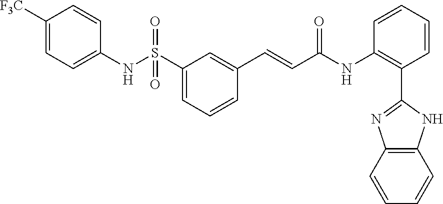 [SMILES: O=C(/C=C/c1cccc(S(=O)(=O)Nc2ccc(C(F)(F)F)cc2)c1)Nc1ccccc1-c1nc2ccccc2[nH]1]

(E)-3-(3-(N-(4-(trifluoromethyl)phenyl)sulfamoyl)phenyl)acrylic acid (Int-42) was reacted with 2-(2-Aminophenyl)-1H-benzimidazole using the experimental conditions outlined in the General Procedure I to give Compound 3 as an off-white solid in 13% yield. ¹H-NMR (400 MHz, DMSO-d₆): δ 13.50 (s, 1H), 13.23 (s, 1H), 10.96 (s, 1H), 8.82 (d, J=8.4 Hz, 1H), 8.26-8.08 (m, 3H), 7.87 (t, J=7.8 Hz, 2H), 7.79-7.75 (m, 1H), 7.69 (t, J=7.8 Hz, 1H), 7.61 (d, J=8.0 Hz, 4H), 7.54 (t, J=8.0 Hz, 1H), 7.41-7.24 (m, 4H), 7.01 (d, J=15.8 Hz, 1H); MS m/z=563 [M+H]⁺.

### (E)-N-(2-(1H-benzo[d]imidazol-2-yl)phenyl)-3-(3-(N-(4-(trifluoromethoxy) phenyl)sulfamoyl)phenyl)acrylamide (Compound 4)

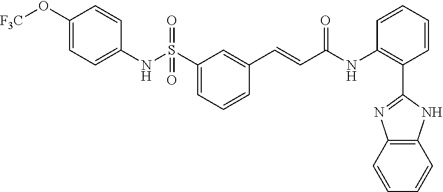 [SMILES: O=C(/C=C/c1cccc(S(=O)(=O)Nc2ccc(OC(F)(F)F)cc2)c1)Nc1ccccc1-c1nc2ccccc2[nH]1]

(E)-3-(3-(N-(4-(trifluoromethoxy)phenyl)sulfamoyl)phenyl)acrylic acid (Int-50) was reacted with 2-(2-Aminophenyl)-1H-benzimidazole using the experimental conditions outlined in the General Procedure I to give Compound 4 as a yellow solid in 19% yield. ¹H-NMR (400 MHz, DMSO-d₆): δ 13.49 (s, 1H), 13.23 (s, 1H), 10.61 (s, 1H), 8.82 (dd, J=8.4, 1.0 Hz, 1H), 8.17 (dd, J=8.0, 1.4 Hz, 1H), 8.12-8.11 (m, 2H), 7.87 (d, J=6.8 Hz, 1H), 7.80 (d, J=7.8 Hz, 1H), 7.75 (d, J=15.8 Hz, 1H), 7.67 (app t, J=8.1 Hz, 1H), 7.62 (d, J=6.9 Hz, 1H), 7.56-7.52 (m, 1H), 7.36-7.28 (m, 3H), 7.28-7.21 (m, 4H), 6.99 (d, J=15.8 Hz, 1H); MS m/z=579 [M+H]⁺.

### (E)-N-(2-(1H-benzo[d]imidazol-2-yl)phenyl)-3-(3-(N-(4-chlorophenyl)-N-methyl sulfamoyl)phenyl)acrylamide (Compound 5)

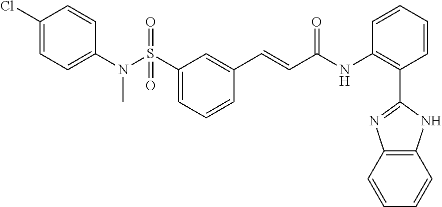 [SMILES: CN(c1ccc(Cl)cc1)S(=O)(=O)c1cccc(/C=C/C(=O)Nc2ccccc2-c2nc3ccccc3[nH]2)c1]

(E)-3-(3-(N-(4-chlorophenyl)-N-methylsulfamoyl)phenyl) acrylic acid (Int-58) was reacted with 2-(2-Aminophenyl)-1H-benzimidazole using the experimental conditions outlined in the General Procedure I to give Compound 5 as a yellow solid in 21% yield. ¹H-NMR (400 MHz, DMSO-d₆): δ 13.51 (s, 1H), 13.23 (bs, 1H), 8.83 (d, J=8.4 Hz, 1H), 8.22 (d, J=7.9 Hz, 1H), 8.17 (d, J=7.1 Hz, 1H), 7.92 (s, 1H), 7.83 (bs, 1H), 7.79 (d, J=15.8 Hz, 1H), 7.69 (app t, J=7.8 Hz, 1H), 7.62 (d, J=7.2 Hz, 1H), 7.54 (app t, J=7.8 Hz, 1H), 7.49 (d, J=8.4 Hz, 1H), 7.44-7.39 (m, 2H), 7.37-7.29 (m, 3H), 7.24-7.17 (m, 2H), 7.02 (d, J=15.8 Hz, 1H), 3.21 (s, 3H); MS m/z=544 [M+H]⁺.

### (E)-N-(2-(1H-benzo[d]imidazol-2-yl)phenyl)-3-(3-(N-methyl-N-phenylsulfamoyl) phenyl)acrylamide (Compound 6)

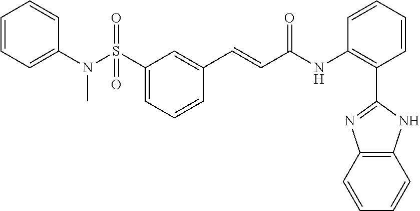 [SMILES: CN(c1ccccc1)S(=O)(=O)c1cccc(/C=C/C(=O)Nc2ccccc2-c2nc3ccccc3[nH]2)c1]

(E)-3-(3-(N-methyl-N-phenylsulfamoyl)phenyl)acrylic acid (Int-32) was reacted with 2-(2-Aminophenyl)-1H-benzimidazole using the experimental conditions outlined in the General Procedure I to give Compound 6 as a light yellow solid in 11% yield. ¹H-NMR (400 MHz, DMSO-d₆): δ 13.50 (s, 1H), 13.24 (bs, 1H), 8.82 (d, J=8.3 Hz, 1H), 8.20 (d, J=7.9 Hz, 1H), 8.17 (d, J=7.9 Hz, 1H), 7.86 (s, 1H), 7.76 (d, J=15.7 Hz, 1H), 7.78-7.65 (m, 1H), 7.67 (t, J=7.8 Hz, 1H), 7.56-7.49 (m, 3H), 7.38-7.26 (m, 6H), 7.17-7.15 (m, 2H), 6.98 (d, J=15.7 Hz, 1H), 3.22 (s, 3H); MS m/z=509 [M+H]⁺.

### (E)-N-(2-(1H-benzo[d]imidazol-2-yl)phenyl)-3-(3-(N-(4-chlorophenyl)sulfamoyl) phenyl)acrylamide (Compound 7)

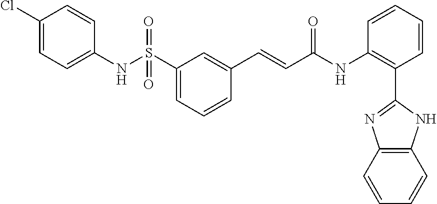 [SMILES: O=C(/C=C/c1cccc(S(=O)(=O)Nc2ccc(Cl)cc2)c1)Nc1ccccc1-c1nc2ccccc2[nH]1]

(E)-3-(3-(N-(4-chlorophenyl)sulfamoyl)phenyl)acrylic acid (Int-66) was reacted with 2-(2-aminophenyl)-1H-benzimidazole using the experimental conditions outlined in the General Procedure I to give Compound 7 as a light yellow solid in 13% yield. ¹H-NMR (400 MHz, DMSO-d₆): δ 13.50 (s, 1H), 13.26 (s, 1H), 10.53 (bs, 1H), 8.82 (dd, J=8.4, 0.9 Hz, 1H), 8.17 (dd, J=7.9, 1.3 Hz, 1H), 8.12-8.10 (m, 2H), 7.87 (d, J=7.2 Hz, 1H), 7.79-7.73 (m, 2H), 7.67 (app t, J=8.1 Hz, 1H), 7.62-7.59 (m, 1H), 7.56-7.52 (m, 1H), 7.36-7.27 (m, 5H), 7.18-7.14 (m, 2H), 6.99 (d, J=15.8 Hz, 1H); MS m/z=529 [M+H]⁺.

### (E)-N-(2-(1H-benzo[d]imidazol-2-yl)phenyl)-3-(3-(N-(3-(trifluoromethoxy) phenyl)sulfamoyl)phenyl)acrylamide (Compound 8)

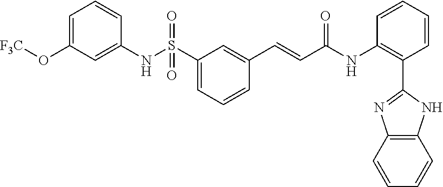 [SMILES: O=C(/C=C/c1cccc(S(=O)(=O)Nc2cccc(OC(F)(F)F)c2)c1)Nc1ccccc1-c1nc2ccccc2[nH]1]

(E)-3-(3-(N-(3-(trifluoromethoxy)phenyl)sulfamoyl)phenyl) acrylic acid (Int-46) was reacted with 2-(2-aminophenyl)-1H-benzimidazole using the experimental conditions outlined in the General Procedure I to give Compound 8 as a light yellow solid in 11% yield. ¹H-NMR (400 MHz, DMSO-d₆): δ 13.52 (s, 1H), 13.24 (s, 1H), 10.77 (bs, 1H), 8.83 (d, J=8.4 Hz, 1H), 8.17 (d, J=8.0 Hz, 1H), 8.14-8.09 (m, 2H), 7.87 (d, J=7.4 Hz, 1H), 7.81 (d, J=7.9 Hz, 1H), 7.75 (d, J=15.7 Hz, 1H), 7.68 (app t, J=7.8 Hz, 1H), 7.61 (d, J=7.0 Hz, 1H), 7.54 (app t, J=7.3 Hz, 1H), 7.41-7.29 (m, 4H), 7.17 (d, J=9.4 Hz, 1H), 7.11 (s, 1H), 7.03 (s, 1H), 6.99 (d, J=15.7 Hz, 1H); MS m/z=579 [M+H]⁺.

### (E)-N-(2-(1H-benzo[d]imidazol-2-yl)phenyl)-3-(3-(N-(4-chloro-3-(trifluoromethyl) phenyl)sulfamoyl)phenyl)acrylamide (Compound 9)

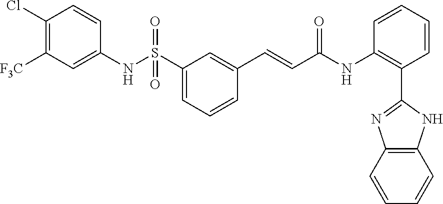 [SMILES: O=C(/C=C/c1cccc(S(=O)(=O)Nc2ccc(Cl)c(C(F)(F)F)c2)c1)Nc1ccccc1-c1nc2ccccc2[nH]1]

(E)-3-(3-(N-(4-chloro-3-(trifluoromethyl)phenyl)sulfamoyl)phenyl)acrylic acid (Int-70) was reacted with 2-(2-aminophenyl)-1H-benzimidazole using the experimental conditions outlined in the General Procedure I to give Compound 9 as a light yellow solid in 8% yield. ¹H-NMR (400 MHz, DMSO-d₆): δ 13.51 (s, 1H), 13.24 (s, 1H), 10.96 (bs, 1H), 8.83 (d, J=8.3 Hz, 1H), 8.23-8.14 (m, 3H), 7.87 (d, J=7.3 Hz, 1H), 7.81 (d, J=7.9 Hz, 1H), 7.77 (d, J=15.8 Hz, 1H), 7.69 (app t, J=7.8 Hz, 1H), 7.62 (s, 1H), 7.60 (s, 1H), 7.58-7.52 (m, 2H), 7.46 (dd, J=8.7, 2.5 Hz, 1H), 7.39-7.28 (m, 3H), 7.02 (d, J=15.8 Hz, 1H); MS m/z=598 [M+H]⁺.

### (E)-N-(2-(1H-benzo[d]imidazol-2-yl)phenyl) sulfamoyl)phenyl)acrylamide (Compound 10)

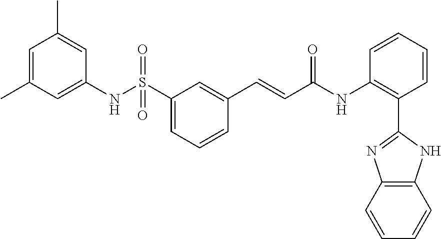 [SMILES: Cc1cc(C)cc(NS(=O)(=O)c2cccc(/C=C/C(=O)Nc3ccccc3-c3nc4ccccc4[nH]3)c2)c1]

(E)-3-(3-(N-(3,5-dimethylphenyl)sulfamoyl)phenyl)acrylic acid (Int-62) was reacted with 2-(2-aminophenyl)-1H-benzimidazole using the experimental conditions outlined in the General Procedure I to give Compound 10 as an orange solid in 4% yield. ¹H-NMR (400 MHz, DMSO-d₆): δ 13.52 (s, 1H), 13.24 (s, 1H), 10.26 (s, 1H), 8.83 (d, J=8.4 Hz, 1H), 8.22-8.20 (m, 2H), 8.07 (d, J=7.7 Hz, 1H), 7.90 (d, J=7.1 Hz, 1H), 7.79 (d, J=8.2 Hz, 1H), 7.76 (d, J=15.8 Hz, 1H), 7.67 (d, J=7.8 Hz, 1H), 7.64-7.60 (m, 1H), 7.54 (app t, J=7.8 Hz, 1H), 7.35-7.28 (m, 3H), 6.99 (d, J=15.8 Hz, 1H), 6.79 (s, 2H), 6.65 (s, 1H), 2.11 (s, 6H); MS m/z=523 [M+H]⁺.

### (E)-N-(2-(1H-benzo[d]imidazol-2-yl)phenyl)-3-(3-(N-phenylsulfamoyl)phenyl) acrylamide (Compound 11)

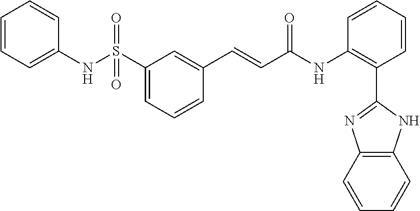 [SMILES: O=C(/C=C/c1cccc(S(=O)(=O)Nc2ccccc2)c1)Nc1ccccc1-c1nc2ccccc2[nH]1]

(E)-3-(3-(N-phenylsulfamoyl)phenyl)acrylic acid (Int-28) was reacted with 2-(2-aminophenyl)-1H-benzimidazole using the experimental conditions outlined in the General Procedure I to give Compound 11 as an orange solid in 10% yield. ¹H-NMR (400 MHz, DMSO-d₆): δ 13.33 (bs, 3H), 8.04 (d, J=7.7 Hz, 2H), 7.79-7.64 (m, 9H), 7.48 (app t, J=7.8 Hz, 2H), 7.37-7.27 (m, 6H); MS m/z=495 [M+H]⁺.

### (E)-N-(2-(1H-benzo[d]imidazol-2-yl)phenyl)-3-(3-(N-(3-(trifluoromethyl)phenyl) sulfamoyl)phenyl)acrylamide (Compound 12)

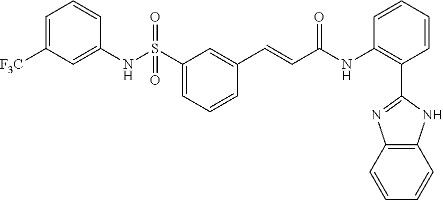 [SMILES: O=C(/C=C/c1cccc(S(=O)(=O)Nc2cccc(C(F)(F)F)c2)c1)Nc1ccccc1-c1nc2ccccc2[nH]1]

(E)-3-(3-(N-(3-(trifluoromethyl)phenyl)sulfamoyl)phenyl)acrylic acid (Int-23) was reacted with 2-(2-aminophenyl)-1H-benzimidazole using the experimental conditions outlined in the General Procedure I to give Compound 12 as an orange solid in 26% yield. ¹H-NMR (400 MHz, DMSO-d₆): δ 13.51 (s, 1H), 13.37 (s, 1H), 13.24 (bs, 3H), 10.83 (bs, 1H), 8.86-8.78 (m, 2H), 8.21-8.14 (m, 3H), 8.12 (d, J=7.6 Hz, 1H), 8.06 (s, 1H), 8.00 (d, J=7.9 Hz, 1H), 7.97 (d, J=8.0 Hz, 1H), 7.88-7.84 (m, 2H), 7.81 (d, J=8.1 Hz, 1H), 7.75 (d, J=15.8 Hz, 1H), 7.71-7.59 (m, 5H), 7.56-7.23 (m, 13H), 7.19-7.14 (m, 2H), 7.00 (d, J=15.8 Hz, 1H), 6.81 (d, J=15.8 Hz, 1H) (The ¹H-NMR contains rotamers); MS m/z=563 [M+H]⁺.

### (E)-N-(2-(1H-benzo[d]imidazol-2-yl)phenyl)-3-(3-((3-(trifluoromethyl)phenyl) sulfonamido)phenyl)acrylamide (Compound 13)

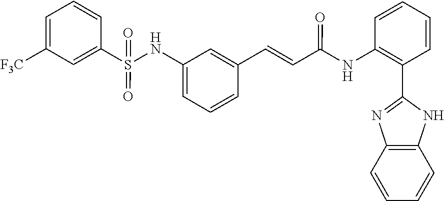 [SMILES: O=C(/C=C/c1cccc(NS(=O)(=O)c2cccc(C(F)(F)F)c2)c1)Nc1ccccc1-c1nc2ccccc2[nH]1]

(E)-3-(3-((3-(trifluoromethyl)phenyl)sulfonamido)phenyl)acrylic acid Int-18 was reacted with 2-(2-aminophenyl)-1H-benzimidazole using the experimental conditions outlined in the General Procedure I to give Compound 13 as a white solid in 14% yield. ¹H-NMR (400 MHz, DMSO-d₆): δ 13.54 (s, 1H), 13.24 (s, 1H), 10.67 (bs, 1H), 8.82 (d, J=8.4 Hz, 1H), 8.17 (d, J=7.9 Hz, 1H), 8.13-8.07 (m, 2H), 8.02 (d, J=7.9 Hz, 1H), 7.87 (d, J=6.9 Hz, 1H), 7.81 (app t, J=7.8 Hz, 1H), 7.66-7.51 (m, 4H), 7.46 (s, 1H), 7.42-7.28 (m, 4H), 7.15 (d, J=9.1 Hz, 1H), 6.79 (d, J=15.8 Hz, 1H); MS m/z=563 [M+H]⁺.

### (E)-3-(3-((2-(1H-benzo[d]imidazol-2-yl)phenyl)amino)-3-oxoprop-1-en-1-yl)-N-(4-fluoro-3-(trifluoromethyl)phenyl)benzamide (Compound 14)

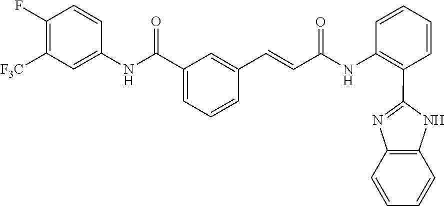 [SMILES: O=C(/C=C/c1cccc(C(=O)Nc2ccc(F)c(C(F)(F)F)c2)c1)Nc1ccccc1-c1nc2ccccc2[nH]1]

(E)-3-(3-((4-fluoro-3-(trifluoromethyl)phenyl)carbamoyl)phenyl)acrylic acid (Int-73) was reacted with 2-(2-aminophenyl)-1H-benzimidazole using the experimental conditions outlined in the General Procedure I to give Compound 14 as a yellow solid in 9% yield. ¹H-NMR (400 MHz, DMSO-d₆): δ 13.58 (s, 1H), 13.25 (bs, 1H), 10.75 (s, 1H), 8.87 (d, J=8.4 Hz, 1H), 8.42 (s, 1H), 8.31 (dd, J=6.5, 2.5 Hz, 1H), 8.23-8.14 (m, 2H), 8.05 (d, J=7.8 Hz, 1H), 8.01 (d, J=7.9 Hz, 1H), 7.97-7.93 (m, 1H), 7.84 (d, J=15.7 Hz, 1H), 7.67 (app t, J=7.7 Hz, 1H), 7.62-7.56 (m, 2H), 7.53 (d, J=7.5 Hz, 1H), 7.33-7.25 (m, 3H), 7.11 (d, J=15.7 Hz, 1H); MS m/z=545 [M+H]⁺.

### (E)-N-(2-(1H-benzo[d]imidazol-2-yl)phenyl) sulfonamido)phenyl)acrylamide (Compound 15)

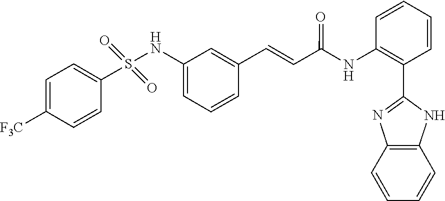 [SMILES: O=C(/C=C/c1cccc(NS(=O)(=O)c2ccc(C(F)(F)F)cc2)c1)Nc1ccccc1-c1nc2ccccc2[nH]1]

(E)-3-(3-((4-(trifluoromethyl)phenyl)sulfonamido) phenyl)acrylic acid Int-79 was reacted with 2-(2-aminophenyl)-1H-benzimidazole using the experimental conditions outlined in the General Procedure I to give Compound 15 as an orange solid in 9% yield. ¹H-NMR (400 MHz, DMSO-d₆): δ 13.52 (s, 1H), 13.24 (s, 1H), 10.74 (bs, 1H), 8.82 (d, J=8.4 Hz, 1H), 8.17 (d, J=8.0 Hz, 1H), 8.02 (d, J=8.3 Hz, 2H), 7.95 (d, J=8.6 Hz, 2H), 7.87 (d, J=6.9 Hz, 1H), 7.67-7.60 (m, 2H), 7.58-7.51 (m, 2H), 7.47 (s, 1H), 7.40-7.26 (m, 4H), 7.15 (d, J=8.1 Hz, 1H), 6.79 (d, J=15.8 Hz, 1H); MS m/z=563 [M+H]⁺.

### (E)-N-(2-(1,3,4-oxadiazol-2-yl)phenyl)-3-(3-(N-(4-fluoro-3-(trifluoromethyl) phenyl)-N-methylsulfamoyl)phenyl)acrylamide (Compound 16)

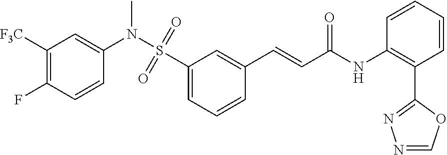 [SMILES: CN(c1ccc(F)c(C(F)(F)F)c1)S(=O)(=O)c1cccc(/C=C/C(=O)Nc2ccccc2-c2nnco2)c1]

(E)-3-(3-(N-(4-fluoro-3-(trifluoromethyl)phenyl)-N-methylsulfamoyl)phenyl)acrylic acid Int-38 was reacted with Int-93 using the experimental conditions outlined in the General Procedure I to give Compound 16 as a white solid in 10% yield. ¹H-NMR (400 MHz, DMSO-d₆): δ 11.51 (bs, 1H), 8.34 (s, 1H), 8.21 (dd, J=8.0, 1.5 Hz, 1H), 8.10-8.04 (m, 1H), 7.94-7.87 (m, 2H), 7.81-7.76 (m, 2H), 7.69 (app t, J=7.8 Hz, 1H), 7.65-7.61 (m, 1H), 7.58-7.46 (m, 4H), 6.96 (d, J=16.0 Hz, 1H), 3.22 (s, 3H); MS m/z=547 [M+H]⁺.

### (E)-N-(2-(1H-benzo[d]imidazol-2-yl)phenyl)-3-(3-(N-(2-chloro-5-(trifluoromethyl)phenyl)sulfamoyl)phenyl)acrylamide (Compound 17)

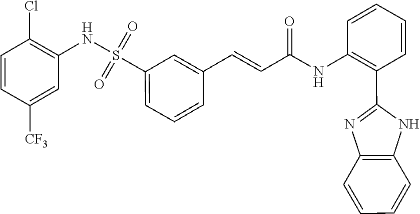 [SMILES: O=C(/C=C/c1cccc(S(=O)(=O)Nc2cc(C(F)(F)F)ccc2Cl)c1)Nc1ccccc1-c1nc2ccccc2[nH]1]

(E)-3-(3-(N-(2-chloro-5-(trifluoromethyl)phenyl)sulfamoyl)phenyl)acrylic acid Int-54 was reacted with 2-(2-aminophenyl)-1H-benzimidazole using the experimental conditions outlined in the General Procedure I to give Compound 17 as an off-white solid in 10% yield. ¹H-NMR (400 MHz, DMSO-d₆): δ 13.51 (s, 1H), 13.23 (bs, 1H), 10.57 (bs, 1H), 8.83 (d, J=8.4 Hz, 1H), 8.17 (d, J=7.9 Hz, 1H), 8.14-8.09 (m, 2H), 7.84 (bs, 1H), 7.78-7.74 (m, 2H), 7.71-7.65 (m, 2H), 7.61 (bs, 1H), 7.55-7.51 (m, 3H), 7.33-7.29 (m, 3H), 6.98 (d, J=15.8 Hz, 1H); MS m/z=597 [M+H]⁺.

### (E)-3-(3-(N-(4-fluoro-3-(trifluoromethyl)phenyl)-N-methylsulfamoyl)phenyl)-N-(2-(thiazol-2-yl)phenyl)acrylamide (Compound 18)

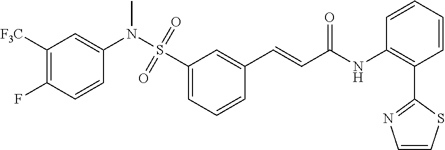 [SMILES: CN(c1ccc(F)c(C(F)(F)F)c1)S(=O)(=O)c1cccc(/C=C/C(=O)Nc2ccccc2-c2nccs2)c1]

(E)-3-(3-(N-(4-fluoro-3-(trifluoromethyl)phenyl)-N-methylsulfamoyl)phenyl)acrylic acid Int-38 was reacted with Int-83 using the experimental conditions outlined in the General Procedure I to give Compound 18 as a white solid in 35% yield. ¹H-NMR (400 MHz, DMSO-d₆): δ 12.04 (s, 1H), 8.53 (d, J=7.6 Hz, 1H), 8.15 (d, J=7.8 Hz, 1H), 8.06 (d, J=3.3 Hz, 1H), 7.97 (dd, J=7.9, 1.4 Hz, 1H), 7.91 (d, J=3.3 Hz, 1H), 7.88 (bs, J=1.6 Hz, 1H), 7.73 (d, J=15.7 Hz, 1H), 7.67 (app t, J=7.8 Hz, 1H), 7.59-7.45 (m, 5H), 7.30-7.26 (m, 1H), 6.99 (d, J=15.7 Hz, 1H), 3.21 (s, 3H); MS m/z=562 [M+H]⁺.

### (E)-3-(3-(N-(4-fluoro-3-(trifluoromethyl)phenyl)-N-methylsulfamoyl)phenyl)-N-(2-(pyridin-2-yl)phenyl)acrylamide (Compound 19)

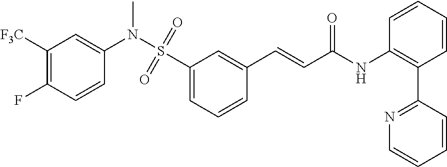 [SMILES: CN(c1ccc(F)c(C(F)(F)F)c1)S(=O)(=O)c1cccc(/C=C/C(=O)Nc2ccccc2-c2ccccn2)c1]

(E)-3-(3-(N-(4-fluoro-3-(trifluoromethyl)phenyl)-N-methylsulfamoyl)phenyl)acrylic acid Int-38 was reacted with Int-100 using the experimental conditions outlined in the General Procedure I to give Compound 19 as a mixture of E/Z-isomers (9:1) in 24% yield as an off-white solid. Major isomer ¹H-NMR (400 MHz, DMSO-d₆): δ 12.11 (s, 1H), 8.78 (d, J=4.0 Hz, 1H), 8.40 (d, J=8.0 Hz, 1H), 8.11 (d, J=7.6 Hz, 1H), 8.00 (app t, J=7.8 Hz, 1H), 7.92 (d, J=8.1 Hz, 1H), 7.87-7.80 (m, 2H), 7.67-7.63 (m, 2H), 7.59-7.45 (m, 6H), 7.27 (app t, J=7.0 Hz, 1H), 6.99 (d, J=15.7 Hz, 1H), 3.20 (s, 3H); MS m/z=556 [M+H]⁺.

### (E)-N-(4-fluoro-3-(trifluoromethyl)phenyl)-N-methyl-3-(3-oxo-3-((2-(pyridin-2-yl)phenyl)amino)prop-1-en-1-yl)benzamide (Compound 20)

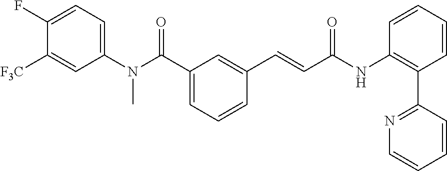 [SMILES: CN(C(=O)c1cccc(/C=C/C(=O)Nc2ccccc2-c2ccccn2)c1)c1ccc(F)c(C(F)(F)F)c1]

(E)-3-(3-((4-fluoro-3-(trifluoromethyl)phenyl)(methyl)carbamoyl)phenyl)acrylic acid Int-75 was reacted with Int-100 using the experimental conditions outlined in the General Procedure I to give Compound 20 as an off-white solid in 19% yield. ¹H-NMR (400 MHz, MeOD): δ 8.59 (d, J=4.1 Hz, 1H), 8.50 (d, J=8.2 Hz, 1H), 8.17-8.15 (m, 1H), 8.00-7.90 (m, 4H), 7.85-7.80 (m, 1H), 7.77-7.72 (m, 2H), 7.68-7.61 (m, 2H), 7.59-7.49 (m, 1H), 7.48-7.41 (m, J=1.6 Hz, 1H), 7.41-7.31 (m, 1H), 7.29-7.25 (m, 1H), 6.66 (d, J=16.0 Hz, 1H), 3.43 (s, 3H); MS m/z=520 [M+H]⁺.

### (E)-N-(4-fluoro-3-(trifluoromethyl)phenyl)-N-methyl-3-(3-oxo-3-((2-(thiazol-2-yl)phenyl)amino)prop-1-en-1-yl)benzamide (Compound 21)

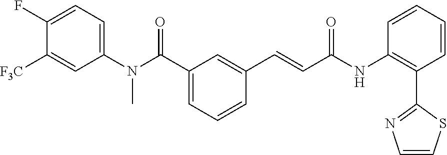 [SMILES: CN(C(=O)c1cccc(/C=C/C(=O)Nc2ccccc2-c2nccs2)c1)c1ccc(F)c(C(F)(F)F)c1]

(E)-3-(3-((4-fluoro-3-(trifluoromethyl)phenyl)methyl)carbamoyl)phenyl)acrylic acid Int-75 was reacted with Int-83 using the experimental conditions outlined in the General Procedure I to give Compound 21 as an off-white solid in 11% yield. ¹H-NMR (400 MHz, DMSO-d₆): δ 12.75 (s, 1H), 8.64 (d, J=8.3 Hz, 1H), 8.09 (bs, 2H), 8.01 (d, J=7.1 Hz, 2H), 7.97 (d, J=3.0 Hz, 1H), 7.90 (d, J=3.4 Hz, 1H), 7.86 (d, J=6.3 Hz, 1H), 7.82 (bs, 1H), 7.78-7.74 (m, 1H), 7.66 (d, J=15.5 Hz, 1H), 7.63-7.50 (m, 3H), 7.29 (app t, J=7.6 Hz, 1H), 3.37 (s, 3H); MS m/z=526 [M+H]⁺.

### (E)-3-(3-((2-hydroxyphenyl)amino)-3-oxoprop-1-en-1-yl)-N-(3-(trifluoromethyl) phenyl)benzamide (Compound 22)

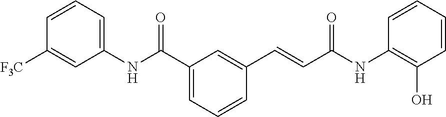 [SMILES: O=C(/C=C/c1cccc(C(=O)Nc2cccc(C(F)(F)F)c2)c1)Nc1ccccc1O]

(E)-3-(3-((3-(trifluoromethyl)phenyl)carbamoyl)phenyl)acrylic acid Int-12 was reacted with 2-aminophenol using the experimental conditions outlined in the General Procedure I to give Compound 22 as a light pink solid in 22% yield. ¹H-NMR (400 MHz, DMSO-d₆): δ 10.66 (s, 1H), 9.99 (bs, 1H), 9.52 (s, 1H), 8.26 (s, 1H), 8.24 (s, 1H), 8.09 (d, J=8.5 Hz, 1H), 8.01-7.96 (m, 2H), 7.86 (d, J=8.0 Hz, 1H), 7.66 (d, J=7.2 Hz, 1H), 7.63 (s, 1H), 7.62 (d, J=7.5 Hz, 1H), 7.48 (d, J=7.8 Hz, 1H), 7.35 (d, J=15.7 Hz, 1H), 6.98-6.93 (m, 1H), 6.89 (dd, J=8.0, 1.5 Hz, 1H), 6.82-6.78 (m, 1H); MS m/z=427 [M+H]⁺.

### (E)-3-(3-((1,3,4-thiadiazol-2-yl)amino)-3-oxoprop-1-en-1-yl)-N-(3-(trifluoromethyl)phenyl)benzamide (Compound 23)

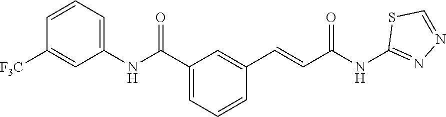 [SMILES: O=C(/C=C/c1cccc(C(=O)Nc2cccc(C(F)(F)F)c2)c1)Nc1nncs1]

(E)-3-(3-((3-(trifluoromethyl)phenyl)carbamoyl)phenyl)acrylic acid Int-12 was reacted with 2-Amino-1,3,4-thiadiazole using the experimental conditions outlined in the General Procedure I to give Compound 23 as a white solid in 16% yield. ¹H-NMR (400 MHz, DMSO-d₆): δ 12.88 (bs, 1H), 10.77 (s, 1H), 9.11 (s, 1H), 8.29 (s, 1H), 8.26 (s, 1H), 8.11 (d, J=8.2 Hz, 1H), 8.04 (d, J=7.9 Hz, 1H), 7.88 (d, J=7.6 Hz, 1H), 7.81 (d, J=16.0 Hz, 1H), 7.63 (app q, J=7.7 Hz, 2H), 7.48 (d, J=7.8 Hz, 1H), 7.08 (d, J=15.9 Hz, 1H); MS m/z=419 [M+H]⁺.

### (E)-3-(3-oxo-3-((6-(trifluoromethoxy)benzo[d]thiazol-2-yl)amino)prop-1-en-1-yl)-N-(3-(trifluoromethyl)phenyl)benzamide (Compound 24)

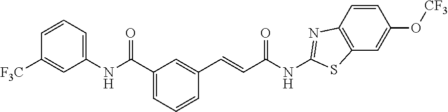 [SMILES: O=C(/C=C/c1cccc(C(=O)Nc2cccc(C(F)(F)F)c2)c1)Nc1nc2ccc(OC(F)(F)F)cc2s1]

(E)-3-(3-((3-(trifluoromethyl)phenyl)carbamoyl)phenyl)acrylic acid Int-12 was reacted with 2-amino-6-(trifluoromethoxy)benzothiazole using the experimental conditions outlined in the General Procedure I to give Compound 24 as an off-white solid in 21% yield. ¹H-NMR (400 MHz, DMSO-d₆): δ 12.78 (bs, 1H), 10.69 (s, 1H), 8.25 (s, 2H), 8.16 (d, J=1.9 Hz, 1H), 8.08 (d, J=10.0 Hz, 1H), 8.04 (d, J=7.8 Hz, 1H), 7.95-7.88 (m, 2H), 7.86 (d, J=8.8 Hz, 1H), 7.70-7.61 (m, 2H), 7.49 (d, J=7.7 Hz, 1H), 7.44 (dd, J=8.8, 1.6 Hz, 1H), 7.09 (d, J=15.9 Hz, 1H); MS m/z=552 [M+H]⁺.

### (E)-N-(3-(3-((2-(1,3,4-thiadiazol-2-yl)phenyl)amino)-3-oxoprop-1-en-1-yl)phenyl)-N-methyl-3-(trifluoromethyl)benzamide (Compound 25)

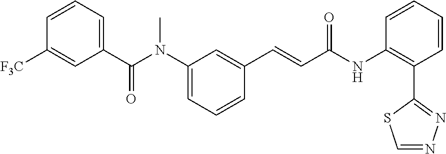 [SMILES: CN(C(=O)c1cccc(C(F)(F)F)c1)c1cccc(/C=C/C(=O)Nc2ccccc2-c2nncs2)c1]

(E)-3-(3-(N-methyl-3-(trifluoromethyl)benzamido)phenyl)acrylic acid Int-7 was reacted with Int-89 using the experimental conditions outlined in the General Procedure I to give Compound 25 as an off-white solid in 6% yield. ¹H-NMR (400 MHz, D₂O): δ 9.53 (s, 1H), 8.58 (d, J=8.4 Hz, 1H), 7.91 (dd, J=7.9, 1.2 Hz, 1H), 7.72-7.44 (m, 8H), 7.39-7.28 (m, 2H), 7.22 (d, J=7.7 Hz, 1H), 6.75 (d, J=15.7 Hz, 1H), 3.54 (s, 3H); MS m/z=509 [M+H]⁺.

### (E)-3-(3-oxo-3-(quinolin-8-ylamino)prop-1-en-1-yl)-N-(3-(trifluoromethyl) phenyl)benzamide (Compound 26)

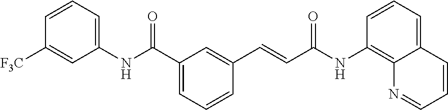 [SMILES: O=C(/C=C/c1cccc(C(=O)Nc2cccc(C(F)(F)F)c2)c1)Nc1cccc2cccnc12]

(E)-3-(3-((3-(trifluoromethyl)phenyl)carbamoyl)phenyl)acrylic acid Int-12 was reacted with 8-aminoquinoline using the experimental conditions outlined in the General Procedure I to give Compound 26 as a brown solid in 45% yield. ¹H-NMR (400 MHz, MeOD): δ 8.88 (dd, J=4.2, 1.6 Hz, 1H), 8.75 (dd, J=7.6, 1.2 Hz, 1H), 8.28 (dd, J=8.3, 1.6 Hz, 1H), 8.23 (s, 1H), 8.19 (s, 1H), 8.03-7.94 (m, 2H), 7.85 (d, J=7.8 Hz, 1H), 7.77 (d, J=15.7 Hz, 1H), 7.61 (dd, J=8.3, 1.3 Hz, 1H), 7.61-7.52 (m, 4H), 7.44 (d, J=7.7 Hz, 1H), 7.21 (d, J=15.6 Hz, 1H); MS m/z=462 [M+H]⁺.

### (E)-N-methyl-N-(3-(3-oxo-3-((2-(thiazol-2-yl)phenyl)amino)prop-1-en-1-yl)phenyl)-3-(trifluoromethyl)benzamide (Compound 27)

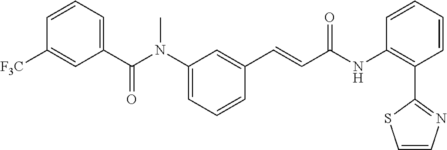 [SMILES: CN(C(=O)c1cccc(C(F)(F)F)c1)c1cccc(/C=C/C(=O)Nc2ccccc2-c2nccs2)c1]

(E)-3-(3-(N-methyl-3-(trifluoromethyl)benzamido)phenyl)acrylic acid Int-7 was reacted with Int-83 using the experimental conditions outlined in the General Procedure I to give Compound 27 as an off-white solid in 65% yield. ¹H-NMR (400 MHz, MeOD): δ 8.66 (d, J=8.3 Hz, 1H), 8.01 (d, J=3.4 Hz, 1H), 7.87 (d, J=7.9 Hz, 1H), 7.68-7.55 (m, 6H), 7.49 (d, J=7.7 Hz, 1H), 7.42 (app t, J=7.9 Hz, 2H), 7.33 (app t, J=7.8 Hz, 1H), 7.20-7.16 (m, 2H), 6.70 (d, J=15.7 Hz, 1H), 3.53 (s, 3H); MS m/z=508 [M+H]⁺.

### (E)-3-(3-((5-chloropyridin-2-yl)amino)-3-oxoprop-1-en-1-yl)-N-(3-(trifluoromethyl)phenyl)benzamide (Compound 28)

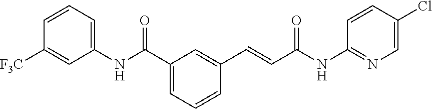 [SMILES: O=C(/C=C/c1cccc(C(=O)Nc2cccc(C(F)(F)F)c2)c1)Nc1ccc(Cl)cn1]

(E)-3-(3-((3-(trifluoromethyl)phenyl)carbamoyl)phenyl)acrylic acid Int-12 was reacted with 2-amino-5-chloropyridine using the experimental conditions outlined in the General Procedure I to give Compound 28 as an off-white solid in 18% yield. ¹H-NMR (400 MHz, DMSO-d₆): δ 10.94 (s, 1H), 10.67 (s, 1H), 8.41 (d, J=2.7 Hz, 1H), 8.29 (d, J=9.0 Hz, 1H), 8.25 (s, 1H), 8.21 (s, 1H), 8.08 (d, J=8.0 Hz, 1H), 8.00 (d, J=7.6 Hz, 1H), 7.95 (dd, J=8.9, 2.6 Hz, 1H), 7.85 (d, J=7.6 Hz, 1H), 7.75 (d, J=15.7 Hz, 1H), 7.64 (app q, J=7.8 Hz, 2H), 7.48 (d, J=6.9 Hz, 1H), 7.16 (d, J=15.8 Hz, 1H); MS m/z=446 [M+H]⁺.

### (E)-5-Chloro-2-(3-(3-((3-(trifluoromethyl)phenyl)carbamoyl)phenyl) acrylamido) pyridin-1-ium methanesulfonate

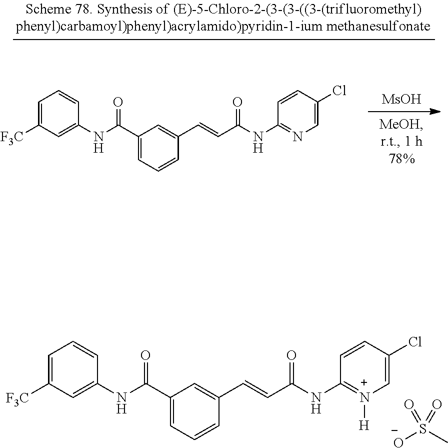

### Experimental Procedure:

(E)-3-(3-((5-chloropyridin-2-yl)amino)-3-oxoprop-1-en-1-yl)-N-(3-(trifluoromethyl)-phenyl)benzamide 28 (20.0 mg, 0.045 mmol) was dissolved in a 0.09 M solution of methanesulfonic acid in methanol (500 μL) and the reaction mixture was stirred at room temperature for 1 hr. Then Diethyl Ether (1 mL) was added, and the resulting white precipitate was isolated by centrifugation, washed with Diethyl Ether (1×2 mL) and dried in vacuo to give 19.0 mg of (E)-5-Chloro-2-(3-(3-((3-(trifluoromethyl)phenyl) carbamoyl)-phenyl)acrylamido)pyridin-1-ium methanesulfonate as a white solid in 78% yield. ¹H-NMR (400 MHz, DMSO-d₆): δ 10.95 (s, 1H), 10.67 (s, 1H), 8.41 (d, J=2.1 Hz, 1H), 8.29 (d, J=9.0 Hz, 1H), 8.25 (s, 1H), 8.21 (s, 1H), 8.08 (d, J=6.9 Hz, 1H), 8.00 (d, J=7.5 Hz, 1H), 7.95 (dd, J=9.0, 2.5 Hz, 1H), 7.86 (d, J=8.7 Hz, 1H), 7.75 (d, J=15.8 Hz, 1H), 7.64 (app q, J=7.7 Hz, 2H), 7.48 (d, J=7.7 Hz, 1H), 7.16 (d, J=15.8 Hz, 1H), 3.17 (s, 1H), 2.30 (s, 3H).

### (E)-5-chloro-2-(3-(3-((3-(trifluoromethyl)phenyl)carbamoyl) phenyl) acrylamido)pyridin-1-ium 2,2,2-trifluoroacetate

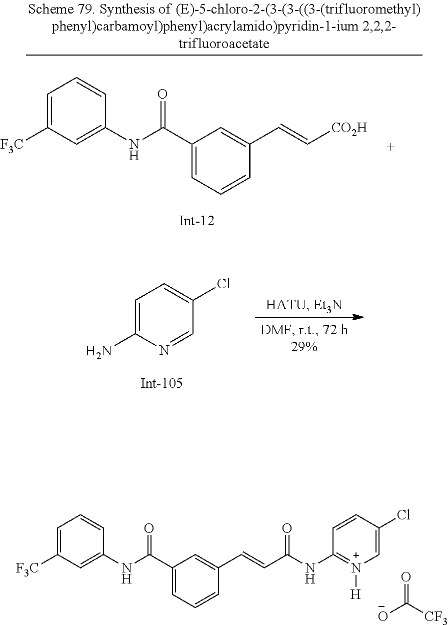 [SMILES: O=C(O)/C=C/c1cccc(C(=O)Nc2cccc(C(F)(F)F)c2)c1 ; Nc1ccc(Cl)cn1 ; O=C(/C=C/c1cccc(C(=O)Nc2cccc(C(F)(F)F)c2)c1)Nc1ccc(Cl)c[nH+]1 ; O=C([O-])C(F)(F)F]

### Experimental Procedure:

(E)-3-(3-((3-(trifluoromethyl)phenyl)carbamoyl)phenyl)acrylic acid Int-12 (33.5 mg, 0.10 mmol) and 5-chloropyridin-2-amine Int-105 (16.7 mg, 0.13 mmol) were dissolved in anhydrous N,N-dimethyl formamide (300 μL) and Triethylamine (21 μL, 0.15 mmol) was added. The reaction mixture was cooled down to 0° C. on an ice-water bath, and HATU (1-[Bis(dimethylamino)methylene]-1H-1,2,3-triazolo[4,5-b]pyridinium 3-oxid hexafluorophosphate) (49.4 mg, 0.13 mmol) was added. The reaction mixture was left to stir at room temperature for 72 hours. Then it was diluted with water and extracted with Ethyl Acetate (3×10 mL). Combined organic layers were dried over anhydrous sodium sulfate, filtered and concentrated in vacuo. The crude product was purified on reversed-phase silica gel with water/acetonitrile+0.1% trifluoroacetic acid. A significant amount the desired product in form of a white precipitate was observed in some of the fractions. The solid was isolated by centrifugation, washed with water and dried in vacuo to afford 13.0 mg of (E)-5-chloro-2-(3-(3-((3-(trifluoromethyl)phenyl) carbamoyl)phenyl)acrylamido) pyridin-1-ium 2,2,2-trifluoroacetate as a white solid in 29% yield. ¹H-NMR (400 MHz, DMSO-d₆): δ 10.95 (s, 1H), 10.67 (s, 1H), 8.41 (d, J=2.2 Hz, 1H), 8.29 (d, J=8.9 Hz, 1H), 8.25 (s, 1H), 8.21 (s, 1H), 8.08 (d, J=9.3 Hz, 1H), 8.00 (d, J=7.5 Hz, 1H), 7.95 (dd, J=8.9, 2.7 Hz, 1H), 7.86 (d, J=7.5 Hz, 1H), 7.75 (d, J=15.8 Hz, 1H), 7.64 (app q, J=7.7 Hz, 2H), 7.48 (d, J=7.8 Hz, 1H), 7.16 (d, J=15.7 Hz, 1H); MS m/z=446 [M+H]⁺.

### (E)-3-(3-oxo-3-(pyrimidin-4-ylamino)prop-1-en-1-yl)-N-(3-(trifluoromethyl) phenyl)benzamide (Compound 29)

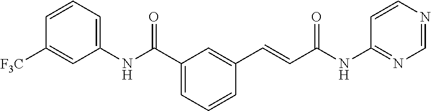 [SMILES: O=C(/C=C/c1cccc(C(=O)Nc2cccc(C(F)(F)F)c2)c1)Nc1ccncn1]

(E)-3-(3-((3-(trifluoromethyl)phenyl)carbamoyl)phenyl)acrylic acid Int-12 was reacted with 4-aminopyrimidine using the experimental conditions outlined in the General Procedure I to give Compound 29 as a white solid in 40% yield. ¹H-NMR (400 MHz, DMSO-d₆): δ 11.16 (s, 1H), 10.68 (s, 1H), 8.91 (s, 1H), 8.70 (d, J=5.7 Hz, 1H), 8.28-8.17 (m, 3H), 8.08 (d, J=8.0 Hz, 1H), 8.01 (d, J=7.7 Hz, 1H), 7.87 (d, J=7.9 Hz, 1H), 7.79 (d, J=15.7 Hz, 1H), 7.68-7.58 (m, 2H), 7.48 (d, J=7.6 Hz, 1H), 7.17 (d, J=15.8 Hz, 1H); MS m/z=413 [M+H]⁺.

### (E)-3-(3-((5-methylpyridin-2-yl)amino)-3-oxoprop-1-en-1-yl)-N-(3-(trifluoromethyl) phenyl)benzamide (Compound 30)

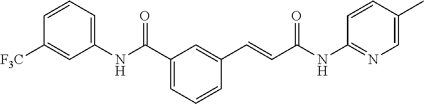 [SMILES: Cc1ccc(NC(=O)/C=C/c2cccc(C(=O)Nc3cccc(C(F)(F)F)c3)c2)nc1]

(E)-3-(3-((3-(trifluoromethyl)phenyl)carbamoyl)phenyl)acrylic acid Int-12 was reacted with 4-amino-5-methylpyridine using the experimental conditions outlined in the General Procedure I to give Compound 30 as a yellow solid in 43% yield. ¹H-NMR (400 MHz, DMSO-d₆): δ 10.67 (s, 2H), 8.25 (s, 1H), 8.19 (s, 2H), 8.15 (d, J=8.5 Hz, 1H), 8.08 (d, J=8.6 Hz, 1H), 7.99 (d, J=7.9 Hz, 1H), 7.84 (d, J=7.7 Hz, 1H), 7.71 (d, J=15.8 Hz, 1H), 7.66-7.61 (m, 3H), 7.48 (d, J=7.8 Hz, 1H), 7.16 (d, J=15.7 Hz, 1H), 2.27 (s, 3H); MS m/z=426 [M+H]⁺.

### (E)-N-(2-(thiazol-2-yl)phenyl)-3-(3-(N-(3-(trifluoromethyl)phenyl)sulfamoyl) phenyl)acrylamide (Compound 31)

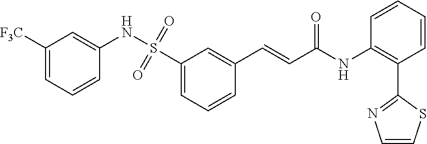 [SMILES: O=C(/C=C/c1cccc(S(=O)(=O)Nc2cccc(C(F)(F)F)c2)c1)Nc1ccccc1-c1nccs1]

(E)-3-(3-(N-(3-(trifluoromethyl)phenyl)sulfamoyl)phenyl)acrylic acid (Int-23) was reacted with Int-83 using the experimental conditions outlined in the General Procedure I to give Compound 31 as a yellow solid in 24% yield. ¹H-NMR (400 MHz, DMSO-d₆): δ 12.06 (bs, 1H), 10.82 (bs, 1H), 8.52 (d, J=8.2 Hz, 1H), 8.10 (s, 1H), 8.07 (d, J=3.4 Hz, 1H), 8.03 (d, J=7.7 Hz, 1H), 7.97 (d, J=7.8 Hz, 1H), 7.91 (d, J=3.3 Hz, 1H), 7.79 (d, J=8.7 Hz, 1H), 7.73-7.63 (m, 2H), 7.56-7.47 (m, 2H), 7.46-7.38 (m, 3H), 7.28 (app t, J=7.6 Hz, 1H), 6.96 (d, J=15.8 Hz, 1H); MS m/z=530 [M+H]⁺.

### (E)-N-(2-(pyridin-2-yl)phenyl)-3-(3-(N-(3-(trifluoromethyl)phenyl)sulfamoyl) phenyl)acrylamide (Compound 32)

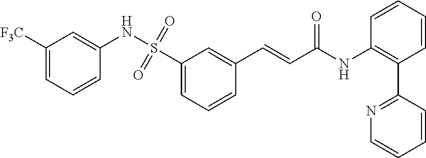 [SMILES: O=C(/C=C/c1cccc(S(=O)(=O)Nc2cccc(C(F)(F)F)c2)c1)Nc1ccccc1-c1ccccn1]

(E)-3-(3-(N-(3-(trifluoromethyl)phenyl)sulfamoyl)phenyl)acrylic acid (Int-23) was reacted with Int-100 using the experimental conditions outlined in the General Procedure I to give Compound 32 as an off-white solid in 33% yield. ¹H-NMR (400 MHz, DMSO-d₆): δ 12.12 (bs, 1H), 10.79 (bs, 1H), 8.80 (d, J=4.9 Hz, 1H), 8.39 (d, J=9.1 Hz, 1H), 8.07 (s, 1H), 8.04-7.97 (m, 2H), 7.92 (d, J=9.1 Hz, 1H), 7.83 (d, J=7.8 Hz, 1H), 7.77 (d, J=7.9 Hz, 1H), 7.68-7.56 (m, 2H), 7.56-7.37 (m, 6H), 7.27 (app t, J=7.7 Hz, 1H), 6.95 (d, J=15.7 Hz, 1H); MS m/z=524 [M+H]⁺.

### (E)-N-(2-(1,3,4-thiadiazol-2-yl)phenyl)-3-(3-(N-(3-(trifluoromethyl)phenyl) sulfamoyl)phenyl)acrylamide (Compound 33)

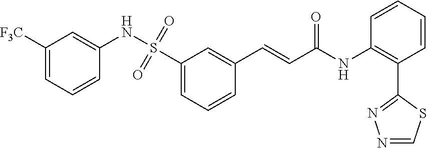 [SMILES: O=C(/C=C/c1cccc(S(=O)(=O)Nc2cccc(C(F)(F)F)c2)c1)Nc1ccccc1-c1nncs1]

(E)-3-(3-(N-(3-(trifluoromethyl)phenyl)sulfamoyl)phenyl)acrylic acid (Int-23) was reacted with Int-89 using the experimental conditions outlined in the General Procedure I to give Compound 33 as an off-white solid in 28% yield. ¹H-NMR (400 MHz, DMSO-d₆): δ 11.02 (s, 1H), 10.84 (bs, 1H), 9.70 (s, 1H), 8.15 (d, J=7.5 Hz, 1H), 8.07 (s, 1H), 8.00 (dd, J=7.9, 1.1 Hz, 1H), 7.94 (d, J=7.6 Hz, 1H), 7.77 (d, J=8.0 Hz, 1H), 7.72-7.59 (m, 3H), 7.51 (app t, J=7.8 Hz, 1H), 7.47-7.36 (m, 4H), 6.95 (d, J=15.7 Hz, 1H); MS m/z=531 [M+H]⁺.

### (E)-3-(3-(N-methyl-N-(3-(trifluoromethyl)phenyl)sulfamoyl)phenyl)-N-(2-(pyridin-2-yl)phenyl)acrylamide (Compound 34)

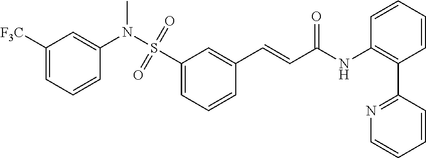 [SMILES: CN(c1cccc(C(F)(F)F)c1)S(=O)(=O)c1cccc(/C=C/C(=O)Nc2ccccc2-c2ccccn2)c1]

(E)-3-(3-(N-methyl-N-(3-(trifluoromethyl)phenyl)sulfamoyl)phenyl)acrylic acid Int-24 was reacted with Int-100 using the experimental conditions outlined in the General Procedure I to give Compound 34 as an off-white solid in 98% yield. ¹H-NMR (400 MHz, DMSO-d₆): δ 12.10 (bs, 1H), 8.78 (d, J=5.0 Hz, 1H), 8.39 (d, J=8.3 Hz, 1H), 8.10 (d, J=7.8 Hz, 1H), 8.00 (app t, J=7.8 Hz, 1H), 7.92 (d, J=8.1 Hz, 1H), 7.87-7.81 (m, 2H), 7.72-7.60 (m, 4H), 7.54-7.44 (m, 5H), 7.27 (app t, J=7.6 Hz, 1H), 6.96 (d, J=15.7 Hz, 1H), 3.23 (s, 3H); MS m/z=538 [M+H]⁺.

### (E)-3-(3-(N-methyl-N-(3-(trifluoromethyl)phenyl)sulfamoyl)phenyl)-N-(2-(thiazol-2-yl)phenyl)acrylamide (Compound 35)

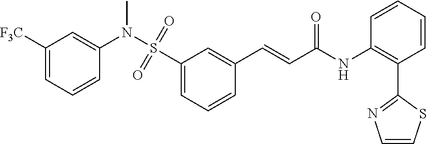 [SMILES: CN(c1cccc(C(F)(F)F)c1)S(=O)(=O)c1cccc(/C=C/C(=O)Nc2ccccc2-c2nccs2)c1]

(E)-3-(3-(N-methyl-N-(3-(trifluoromethyl)phenyl)sulfamoyl)phenyl)acrylic acid Int-24 was reacted with Int-83 using the experimental conditions outlined in the General Procedure I to give Compound 35 as an off-white solid in 77% yield. ¹H-NMR (400 MHz, DMSO-d₆): δ 12.03 (s, 1H), 8.52 (d, J=8.4 Hz, 1H), 8.14 (d, J=7.2 Hz, 1H), 8.06 (d, J=3.4 Hz, 1H), 7.97 (d, J=7.9 Hz, 1H), 7.92 (d, J=3.3 Hz, 1H), 7.84 (bs, 1H), 7.76-7.61 (m, 4H), 7.57-7.45 (m, 4H), 7.28 (app t, J=7.6 Hz, 1H), 6.96 (d, J=15.7 Hz, 1H), 3.24 (s, 3H); MS m/z=544 [M+H]⁺.

### (E)-N-(2-(1,3,4-thiadiazol-2-yl)phenyl)-3-(3-(N-methyl-N-(3-(trifluoromethyl) phenyl)sulfamoyl)phenyl)acrylamide (Compound 36)

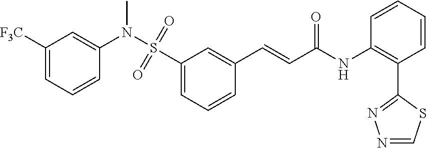 [SMILES: CN(c1cccc(C(F)(F)F)c1)S(=O)(=O)c1cccc(/C=C/C(=O)Nc2ccccc2-c2nncs2)c1]

(E)-3-(3-(N-methyl-N-(3-(trifluoromethyl)phenyl)sulfamoyl)phenyl)acrylic acid Int-24 was reacted with Int-89 using the experimental conditions outlined in the General Procedure I to give Compound 36 as an off-white solid in 24% yield. ¹H-NMR (400 MHz, DMSO-d₆): δ 10.97 (s, 1H), 9.72 (s, 1H), 8.15 (d, J=8.2 Hz, 1H), 8.05 (d, J=8.0 Hz, 1H), 7.99 (dd, J=7.8, 1.1 Hz, 1H), 7.79 (bs, 1H), 7.75-7.59 (m, 5H), 7.53-7.46 (m, 2H), 7.43 (bs, 1H), 7.38 (app t, J=7.6 Hz, 1H), 6.92 (d, J=15.8 Hz, 1H), 3.23 (s, 3H); MS m/z=545 [M+H]⁺.

### (E)-N-(2-(5-(pyridin-4-yl)-1,3,4-oxadiazol-2-yl)phenyl)-3-(3-(N-(3-(trifluoromethyl)phenyl)sulfamoyl)phenyl)acrylamide (Compound 37)

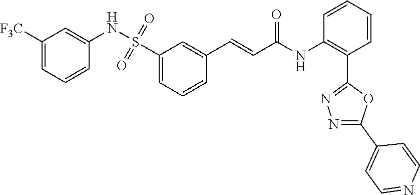 [SMILES: O=C(/C=C/c1cccc(S(=O)(=O)Nc2cccc(C(F)(F)F)c2)c1)Nc1ccccc1-c1nnc(-c2ccncc2)o1]

(E)-3-(3-(N-(3-(trifluoromethyl)phenyl)sulfamoyl)phenyl)acrylic acid Int-23 was reacted with 2-(5-pyridin-4-yl-[1,3,4]oxadiazol-2-yl)phenylamine using the experimental conditions outlined in the General Procedure I to give Compound 37 as a light yellow solid in 16% yield. ¹H-NMR (400 MHz, DMSO-d₆): δ 10.82 (bs, 1H), 10.79 (s, 1H), 8.91-8.80 (m, 2H), 8.40 (d, J=8.0 Hz, 1H), 8.20 (dd, J=7.8, 1.3 Hz, 1H), 8.10 (s, 1H), 8.08-8.03 (m, 2H), 7.98 (d, J=7.9 Hz, 1H), 7.78 (d, J=8.0 Hz, 1H), 7.74-7.61 (m, 3H), 7.54-7.49 (m, 1H), 7.46-7.40 (m, 3H), 7.38 (s, 1H), 7.00 (d, J=15.8 Hz, 1H); MS m/z=592 [M+H]⁺.

### (E)-3-(3-(N-methyl-N-(3-(trifluoromethyl)phenyl)sulfamoyl)phenyl)-N-(2-(5-(pyridin-4-yl)-1,3,4-oxadiazol-2-yl)phenyl)acrylamide (Compound 38)

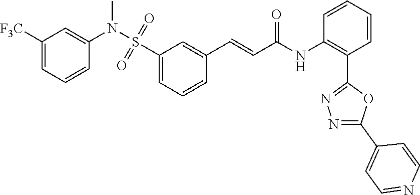 [SMILES: CN(c1cccc(C(F)(F)F)c1)S(=O)(=O)c1cccc(/C=C/C(=O)Nc2ccccc2-c2nnc(-c3ccncc3)o2)c1]

(E)-3-(3-(N-methyl-N-(3-(trifluoromethyl)phenyl)sulfamoyl)phenyl)acrylic acid Int-24 was reacted with 2-(5-pyridin-4-yl-[1,3,4]oxadiazol-2-yl)phenylamine using the experimental conditions outlined in the General Procedure I to give Compound 38 as an off-white solid in 10% yield. ¹H-NMR (400 MHz, DMSO-d₆): δ 10.75 (s, 1H), 8.85 (app d, J=6.1 Hz, 2H), 8.39 (d, J=8.4 Hz, 1H), 8.19 (d, J=8.6 Hz, 1H), 8.14-8.06 (m, 3H), 7.81 (bs, 1H), 7.75-7.58 (m, 5H), 7.54-7.40 (m, 4H), 6.97 (d, J=15.7 Hz, 1H), 3.23 (s, 3H); MS m/z=606 [M+H]⁺.

### (E)-3-(3-(N-(3-(trifluoromethyl)phenyl)sulfamoyl)phenyl)-N-(2-(1-((2-(trimethylsilyl)ethoxy)methyl)-1H-imidazol-2-yl)phenyl)acrylamide (Compound 39)

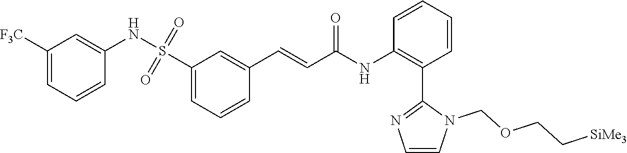 [SMILES: C[Si](C)(C)CCOCn1ccnc1-c1ccccc1NC(=O)/C=C/c1cccc(S(=O)(=O)Nc2cccc(C(F)(F)F)c2)c1]

(E)-3-(3-(N-(3-(trifluoromethyl)phenyl)sulfamoyl)phenyl)acrylic acid Int-23 was reacted with Int-97 using the experimental conditions outlined in the General Procedure I to give Compound 39 as a light yellow solid in 16% yield. ¹H-NMR (400 MHz, DMSO-d₆): δ 11.10 (s, 1H), 10.81 (bs, 1H), 8.40 (d, J=8.3 Hz, 1H), 8.03 (s, 1H), 7.93 (d, J=7.3 Hz, 1H), 7.75 (app t, J=8.2 Hz, 2H), 7.65-7.54 (m, 3H), 7.48 (app t, J=8.0 Hz, 2H), 7.42-7.32 (m, 3H), 7.26-7.22 (m, 2H), 6.84 (d, J=15.7 Hz, 1H), 5.31 (s, 2H), 3.51 (t, J=8.1 Hz, 2H), 0.81 (t, J=8.1 Hz, 2H), −0.09 (s, 9H); MS m/z=643 [M+H]⁺.

### (E)-N-(2-(1,2,4-oxadiazol-3-yl)phenyl)-3-(3-(N-methyl-N-(3-(trifluoromethyl) phenyl)sulfamoyl)phenyl)acrylamide (Compound 40)

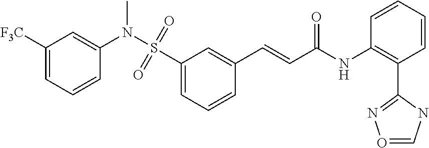

(E)-3-(3-(N-methyl-N-(3-(trifluoromethyl)phenyl)sulfamoyl)phenyl)acrylic acid Int-24 was reacted with Int-93 using the experimental conditions outlined in the General Procedure I to give Compound 40 as an off-white solid in 12% yield. ¹H-NMR (400 MHz, MeOD): δ 9.43 (s, 1H), 8.44 (d, J=7.7 Hz, 1H), 8.23 (dd, J=7.9, 1.4 Hz, 1H), 7.97 (d, J=8.9 Hz, 1H), 7.80-7.51 (m, 8H), 7.45-7.39 (m, 2H), 7.34 (td, J=7.6, 1.1 Hz, 1H), 6.82 (d, J=15.6 Hz, 1H), 3.25 (s, 3H); MS m/z=529 [M+H]⁺.

### (E)-N-(2-(1,2,4-oxadiazol-3-yl)phenyl)-3-(3-(N-methyl-N-(3-(trifluoromethyl) phenyl)sulfamoyl)phenyl)acrylamide (Compound 41)

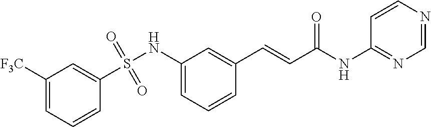 [SMILES: O=C(/C=C/c1cccc(NS(=O)(=O)c2cccc(C(F)(F)F)c2)c1)Nc1ccncn1]

(E)-3-(3-((3-(trifluoromethyl)phenyl)sulfonamido)phenyl) acrylic acid Int-18 was reacted with 4-aminopyrimidine using the experimental conditions outlined in the General Procedure I to give Compound 41 as an off-white solid in 12% yield. ¹H-NMR (400 MHz, MeOD): δ 9.43 (s, 1H), 8.44 (d, J=7.7 Hz, 1H), 8.23 (dd, J=7.9, 1.4 Hz, 1H), 7.97 (d, J=8.9 Hz, 1H), 7.80-7.51 (m, 8H), 7.45-7.39 (m, 2H), 7.34 (td, J=7.6, 1.1 Hz, 1H), 6.82 (d, J=15.6 Hz, 1H), 3.25 (s, 3H); MS m/z=529 [M+H]⁺.

### (E)-N-(pyrimidin-4-yl)-3-(3-((3-(trifluoromethyl)phenyl)sulfonamido)phenyl) acrylamide (Compound 42)

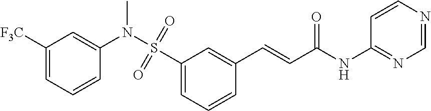 [SMILES: CN(c1cccc(C(F)(F)F)c1)S(=O)(=O)c1cccc(/C=C/C(=O)Nc2ccncn2)c1]

(E)-3-(3-(N-methyl-N-(3-(trifluoromethyl)phenyl)sulfamoyl)phenyl)acrylic acid Int-24 was reacted with 4-aminopyrimidine using the experimental conditions outlined in the General Procedure I to give Compound 42 as an off-white solid in 24% yield. ¹H-NMR (400 MHz, DMSO-d₆): δ 11.17 (s, 1H), 10.65 (bs, 1H), 8.91 (d, J=1.1 Hz, 1H), 8.68 (d, J=5.8 Hz, 1H), 8.19 (dd, J=5.8, 1.2 Hz, 1H), 8.09-8.06 (m, 2H), 8.02 (d, J=7.9 Hz, 1H), 7.82 (app t, J=7.7 Hz, 1H), 7.57 (d, J=15.8 Hz, 1H), 7.42 (s, 1H), 7.34-7.28 (m, 2H), 7.08 (d, J=6.7 Hz, 1H), 6.99 (d, J=15.8 Hz, 1H); MS m/z=449 [M+H]⁺.

### (E)-3-(3-((2-(1H-imidazol-2-yl)phenyl)amino)-3-oxoprop-1-en-1-yl)-N-(3-(trifluoromethyl)phenyl)benzamide (Compound 43)

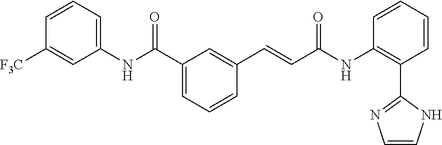 [SMILES: O=C(/C=C/c1cccc(C(=O)Nc2cccc(C(F)(F)F)c2)c1)Nc1ccccc1-c1ncc[nH]1]

(E)-3-(3-((3-(trifluoromethyl)phenyl)carbamoyl)phenyl)acrylic acid Int-12 (33.5 mg, 0.10 mmol) and Int-97 (37.6 mg, 0.13 mmol) were dissolved in anhydrous N,N-dimethyl formamide (300 μL) and triethylamine (0.15 mmol, 21 μL) was added. After cooling down to 0° C., HATU (1-[Bis(dimethylamino)methylene]-1H-1,2,3-triazolo[4,5-b]pyridinium 3-oxid hexafluoro phosphate) (0.13 mmol, 49.4 mg) was added, and the reaction mixture was stirred at room temperature for 72 hours. After dilution with water, the mixture was extracted with dichloromethane (3×10 mL). Combined organic layers were dried over anhydrous sodium sulfate, filtered and concentrated in vacuo. The resulting orange oil was treated with DCM (1.5 mL) and trifluoroacetic acid (1.5 mL) for 90 minutes at room temperature. The crude product was purified on reversed-phase silica gel with water/acetonitrile+0.1% trifluoroacetic acid. Fractions containing the desired product were combined and treated with saturated sodium bicarbonate solution. The resulting mixture was extracted with Ethyl Acetate (3×50 mL). Combined organic layers were washed with brine (1×50 mL), dried over anhydrous sodium sulfate, filtered and concentrated in vacuo to give Compound 43 as an off-white solid in 70% yield. ¹H-NMR (400 MHz, DMSO-d₆): δ 13.38 (s, 1H), 12.89 (bs, 1H), 10.67 (s, 1H), 8.77 (d, J=8.3 Hz, 1H), 8.33 (s, 1H), 8.28 (s, 1H), 8.09 (d, J=8.7 Hz, 1H), 8.04-7.94 (m, 3H), 7.77 (d, J=15.7 Hz, 1H), 7.64 (app t, J=8.1 Hz, 2H), 7.49 (d, J=7.5 Hz, 1H), 7.44-7.35 (m, 2H), 7.29 (s, 1H), 7.22-7.18 (m, 1H), 6.96 (d, J=15.7 Hz, 1H); MS m/z=477 [M+H]⁺.

### (E)-N-(quinolin-8-yl)-3-(3-(N-(3-(trifluoromethyl)phenyl)sulfamoyl)phenyl) acrylamide (Compound 44)

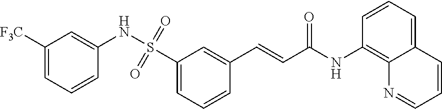 [SMILES: O=C(/C=C/c1cccc(S(=O)(=O)Nc2cccc(C(F)(F)F)c2)c1)Nc1cccc2cccnc12]

(E)-3-(3-(N-(3-(trifluoromethyl)phenyl)sulfamoyl)phenyl)acrylic acid Int-23 was reacted with 8-aminoquinoline using the experimental conditions outlined in the General Procedure I to give Compound 44 as a brown solid in 17% yield. ¹H-NMR (400 MHz, DMSO-d₆): δ 10.52 (s, 1H), 10.12 (bs, 1H), 8.99 (dd, J=4.3, 1.6 Hz, 1H), 8.86 (d, J=4.4 Hz, 1H), 8.80 (d, J=7.8 Hz, 1H), 8.45 (dd, J=8.2, 1.4 Hz, 1H), 8.38-8.32 (m, 2H), 7.90 (d, J=7.9 Hz, 1H), 7.86 (d, J=7.8 Hz, 1H), 7.75-7.71 (m, 2H), 7.68 (dd, J=8.3, 4.2 Hz, 1H), 7.64 (d, J=8.1 Hz, 1H), 7.61-7.51 (m, 3H), 7.60 (s, 1H); MS m/z=498[M+H]⁺.

### (E)-N-(2-(1H-imidazol-2-yl)phenyl)-3-(3-(N-(3-(trifluoromethyl)phenyl) sulfamoyl)phenyl)acrylamide (Compound 45)

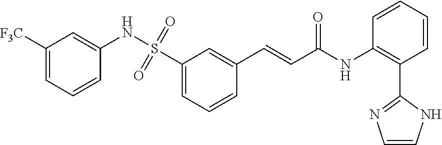 [SMILES: O=C(/C=C/c1cccc(S(=O)(=O)Nc2cccc(C(F)(F)F)c2)c1)Nc1ccccc1-c1ncc[nH]1]

(E)-3-(3-(N-(3-(trifluoromethyl)phenyl)sulfamoyl)phenyl)acrylic acid Int-23 (37.1 mg, 0.10 mmol) and Int-97 (37.6 mg, 0.13 mmol) were dissolved in anhydrous N,N-dimethyl formamide (300 μL) and triethylamine (0.15 mmol, 21 μL) was added. After cooling down to 0° C., HATU (1-[Bis(dimethylamino)methylene]-1H-1,2,3-triazolo[4,5-b]pyridinium 3-oxid hexafluorophosphate) (0.13 mmol, 49.4 mg) was added, and the reaction mixture was stirred at room temperature for 72 hours. After dilution with water, the mixture was extracted with Ethyl Acetate (3×10 mL). Combined organic layers were washed with brine (1×10 mL), dried over anhydrous sodium sulfate, filtered and concentrated in vacuo. The residue was treated with DCM (1.0 mL) and trifluoroacetic acid (0.8 mL) for 60 minutes at room temperature. The crude product was purified on reversed-phase silica gel with water/acetonitrile+0.1% trifluoroacetic acid. Fractions containing the desired product were combined and treated with saturated sodium bicarbonate solution. The resulting mixture was extracted with Ethyl Acetate (3×50 mL). Combined organic layers were washed with brine (1×50 mL), dried over anhydrous sodium sulfate, filtered and concentrated in vacuo to give Compound 45 as a yellow solid in 42% yield. ¹H-NMR (400 MHz, DMSO-d₆): δ 13.33 (s, 1H), 12.89 (bs, 1H), 10.83 (bs, 1H), 8.74 (d, J=8.4 Hz, 1H), 8.08 (s, 1H), 8.02 (d, J=7.5 Hz, 1H), 7.97 (dd, J=8.0, 1.3 Hz, 1H), 7.78 (d, J=7.8 Hz, 1H), 7.68 (d, J=15.7 Hz, 1H), 7.64 (app t, J=7.8 Hz, 1H), 7.52-7.46 (m, 1H), 7.45-7.35 (m, 5H), 7.25 (s, 1H), 7.22-7.18 (m, 1H), 6.84 (d, J=15.8 Hz, 1H); MS m/z=513 [M+H]⁺.

### (E)-N-(quinolin-8-yl)-3-(3-((3-(trifluoromethyl)phenyl)sulfonamido)phenyl) acrylamide (Compound 46)

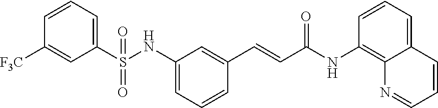 [SMILES: O=C(/C=C/c1cccc(NS(=O)(=O)c2cccc(C(F)(F)F)c2)c1)Nc1cccc2cccnc12]

(E)-3-(3-((3-(trifluoromethyl)phenyl)sulfonamido)phenyl)acrylic acid Int-18 was reacted with 8-aminoquinoline using the experimental conditions outlined in the General Procedure I to give Compound 46 as a brown solid in 6% yield. ¹H-NMR (400 MHz, CDCl₃): δ 10.03 (s, 1H), 8.88 (dd, J=7.2, 1.7 Hz, 1H), 8.83 (dd, J=4.2, 1.7 Hz, 1H), 8.17 (dd, J=8.3, 1.6 Hz, 1H), 8.08 (s, 1H), 7.95 (d, J=7.8 Hz, 1H), 7.80 (d, J=7.8 Hz, 1H), 7.70 (d, J=15.6 Hz, 1H), 7.61-7.52 (m, 2H), 7.48 (dd, J=8.3, 4.2 Hz, 1H), 7.40 (d, J=7.7 Hz, 1H), 7.35 (bs, 1H), 7.31 (app t, J=7.8 Hz, 1H), 7.26 (s, 1H), 7.21 (bs, 1H), 7.14 (d, J=8.0 Hz, 1H), 6.75 (d, J=15.5 Hz, 1H); MS m/z=498 [M+H]⁺.

### (E)-3-(3-(N-methyl-N-(3-(trifluoromethyl)phenyl)sulfamoyl)phenyl)-N-(quinolin-8-yl)acrylamide (Compound 47

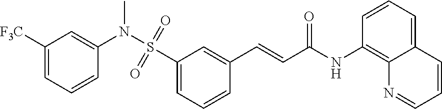 [SMILES: CN(c1cccc(C(F)(F)F)c1)S(=O)(=O)c1cccc(/C=C/C(=O)Nc2cccc3cccnc23)c1]

(E)-3-(3-(N-methyl-N-(3-(trifluoromethyl)phenyl)sulfamoyl)phenyl)acrylic acid Int-24 was reacted with 8-aminoquinoline using the experimental conditions outlined in the General Procedure I to give Compound 47 as a brown solid in 22% yield. ¹H-NMR (400 MHz, DMSO-d₆): δ 10.53 (s, 1H), 8.98 (dd, J=4.2, 1.7 Hz, 1H), 8.82 (dd, J=7.7, 1.3 Hz, 1H), 8.44 (dd, J=8.3, 1.6 Hz, 1H), 8.05 (d, J=7.8 Hz, 1H), 7.99 (s, 1H), 7.73-7.60 (m, 8H), 7.49-7.42 (m, 3H), 3.26 (s, 3H); MS m/z=512 [M+H]⁺.

### (E)-N-(2-(1,3,4-oxadiazol-2-yl)phenyl)-3-(3-(N-methyl-N-(3-(trifluoromethyl) phenyl)sulfamoyl)phenyl)acrylamide (Compound 48)

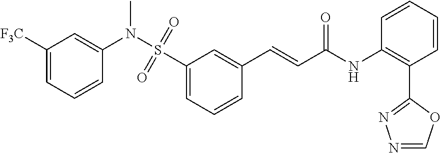 [SMILES: CN(c1cccc(C(F)(F)F)c1)S(=O)(=O)c1cccc(/C=C/C(=O)Nc2ccccc2-c2nnco2)c1]

(E)-3-(3-(N-methyl-N-(3-(trifluoromethyl)phenyl)sulfamoyl)phenyl)acrylic acid Int-24 was reacted with 2-(1,3,4-oxadiazol-2-yl)aniline using the experimental conditions outlined in the General Procedure I to give Compound 48 as an off-white solid in 52% yield. ¹H-NMR (400 MHz, DMSO-d₆): δ 11.52 (bs, 1H), 8.34 (s, 1H), 8.21 (dd, J=8.0, 1.0 Hz, 1H), 8.06 (d, J=7.8 Hz, 1H), 7.92 (ddd, J=8.3, 7.1, 1.4 Hz, 1H), 7.85 (s, 1H), 7.78 (s, 1H), 7.75 (d, J=7.0 Hz, 1H), 7.71-7.67 (m, 2H), 7.66-7.59 (m, 2H), 7.53-7.45 (m, 3H), 6.93 (d, J=16.0 Hz, 1H), 3.25 (s, 3H); MS m/z=529 [M+H]⁺.

### (E)-N-(2-(1H-imidazol-2-yl)phenyl)-3-(3-(N-methyl-N-(3-(trifluoromethyl) phenyl)sulfamoyl)phenyl)acrylamide (Compound 49)

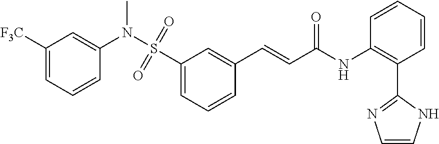 [SMILES: CN(c1cccc(C(F)(F)F)c1)S(=O)(=O)c1cccc(/C=C/C(=O)Nc2ccccc2-c2ncc[nH]2)c1]

Int-24 (38.5 mg, 0.10 mmol) and Int-97 (37.6 mg, 0.13 mmol) were dissolved in anhydrous N,N-dimethyl formamide (300 p L) and triethylamine (0.15 mmol, 21 p L) was added. After cooling down to 0° C. HATU (1-[Bis(dimethylamino)methylene]-1H-1,2,3-triazolo[4,5-b]pyridinium 3-oxid hexafluorophosphate) (0.13 mmol, 49.4 mg) was added, and the reaction mixture was stirred at room temperature for 72 hours. After dilution with water, the mixture was extracted with dichloromethane (3×10 mL). The combined organic layers were dried over sodium sulfate, filtered and concentrated in vacuo. The residue was treated with DCM (1.0 mL) and trifluoroacetic acid (1.0 mL) for 60 minutes at room temperature. The crude product was purified on reversed-phase silica gel with water/acetonitrile+0.1% trifluoroacetic acid. Fractions containing the desired product were combined and treated with saturated sodium bicarbonate solution. The resulting mixture was extracted with Ethyl Acetate (3×50 mL). Combined organic layers were washed with brine (1×50 mL), dried over anhydrous sodium sulfate, filtered and concentrated in vacuo to give Compound 49 as a yellow solid in 67% yield. ¹H-NMR (400 MHz, DMSO-d₆): δ 13.29 (s, 1H), 12.88 (bs, 1H), 8.74 (d, J=8.3 Hz, 1H), 8.14 (d, J=7.9 Hz, 1H), 7.96 (dd, J=7.9, 1.2 Hz, 1H), 7.78 (s, 1H), 7.73-7.61 (m, 4H), 7.50 (d, J=8.4 Hz, 2H), 7.45 (s, 1H), 7.40-7.36 (m, 2H), 7.23-7.18 (m, 2H), 6.83 (d, J=15.7 Hz, 1H), 3.24 (s, 3H); MS m/z=527 [M+H]⁺.

### (E)-N-methyl-3-(3-oxo-3-(pyrimidin-4-ylamino)prop-1-en-1-yl)-N-(3-(trifluoromethyl)phenyl)benzamide (Compound 50)

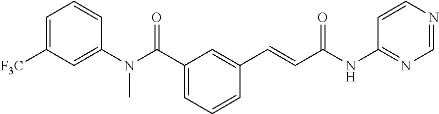 [SMILES: CN(C(=O)c1cccc(/C=C/C(=O)Nc2ccncn2)c1)c1cccc(C(F)(F)F)c1]

(E)-3-(3-(methyl(3-(trifluoromethyl)phenyl)carbamoyl)phenyl)acrylic acid Int-14 was reacted with 4-aminopyrimidine using the experimental conditions outlined in the General Procedure I to give Compound 50 as an off-white solid in 47% yield. ¹H-NMR (400 MHz, DMSO-d₆): δ 11.09 (s, 1H), 8.90 (s, 1H), 8.68 (d, J=5.8 Hz, 1H), 8.19 (dd, J=5.8, 1.3 Hz, 1H), 7.64-7.51 (m, 7H), 7.32 (app t, J=7.7 Hz, 1H), 7.26 (d, J=7.3 Hz, 1H), 6.99 (d, J=15.7 Hz, 1H), 3.43 (s, 3H); MS m/z=427 [M+H]⁺.

### (E)-3-(3-oxo-3-(pyrimidin-5-ylamino)prop-1-en-1-yl)-N-(3-(trifluoromethyl) phenyl)benzamide (Compound 51)

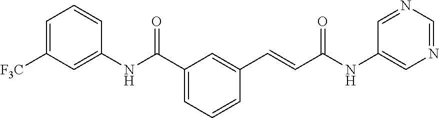 [SMILES: O=C(/C=C/c1cccc(C(=O)Nc2cccc(C(F)(F)F)c2)c1)Nc1cncnc1]

(E)-3-(3-((3-(trifluoromethyl)phenyl)acrylic acid Int-12 was reacted with 5-aminopyrimidine using the experimental conditions outlined in the General Procedure I to give Compound 51 as a white solid in 31% yield. ¹H-NMR (400 MHz, DMSO-d₆): δ 10.73 (s, 1H), 10.68 (s, 1H), 9.11 (s, 2H), 8.92 (s, 1H), 8.26 (s, 2H), 8.09 (d, J=8.1 Hz, 1H), 8.02 (d, J=7.5 Hz, 1H), 7.90 (d, J=7.8 Hz, 1H), 7.77 (d, J=15.6 Hz, 1H), 7.71-7.60 (m, 2H), 7.48 (d, J=7.9 Hz, 1H), 6.98 (d, J=15.7 Hz, 1H); MS m/z=413 [M+H]⁺.

### (E)-N-methyl-3-(3-((5-methylpyridin-2-yl)amino)-3-oxoprop-1-en-1-yl)-N-(3-(trifluoromethyl)phenyl)benzamide (Compound 52)

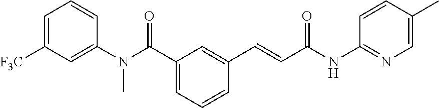 [SMILES: Cc1ccc(NC(=O)/C=C/c2cccc(C(=O)N(C)c3cccc(C(F)(F)F)c3)c2)nc1]

(E)-3-(3-(methyl(3-(trifluoromethyl)phenyl)carbamoyl)phenyl)acrylic acid Int-14 was reacted with 2-amino-5-methylpyridine using the experimental conditions outlined in the General Procedure I to give Compound 52 as a yellow solid in 62% yield. ¹H-NMR (400 MHz, DMSO-d₆): δ 10.59 (s, 1H), 8.18 (s, 1H), 8.12 (d, J=8.4 Hz, 1H), 7.64-7.61 (m, 2H), 7.57-7.53 (m, 5H), 7.49 (d, J=15.9 Hz, 1H), 7.31 (app t, J=7.6 Hz, 1H), 7.24 (d, J=7.6 Hz, 1H), 6.97 (d, J=15.7 Hz, 1H), 3.43 (s, 3H), 2.26 (s, 3H); MS m/z=440 [M+H]⁺.

### (E)-N-(3-(3-oxo-3-(quinolin-8-ylamino)prop-1-en-1-yl)phenyl)-3-(trifluoromethyl)benzamide (Compound 53)

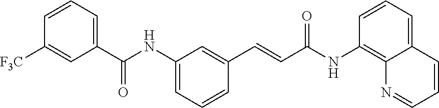 [SMILES: O=C(/C=C/c1cccc(NC(=O)c2cccc(C(F)(F)F)c2)c1)Nc1cccc2cccnc12]

(E)-3-(3-(3-(trifluoromethyl)benzamido)phenyl)acrylic acid Int-5 was reacted with 8-aminoquinoline using the experimental conditions outlined in the General Procedure I to give Compound 53 as a brown solid in 20% yield. ¹H-NMR (400 MHz, DMSO-d₆): δ 10.60 (s, 1H), 10.53 (s, 1H), 8.98 (dd, J=4.2, 1.7 Hz, 1H), 8.80 (dd, J=7.6, 1.3 Hz, 1H), 8.44 (dd, J=8.4, 1.6 Hz, 1H), 8.35 (s, 1H), 8.31 (d, J=8.4 Hz, 1H), 8.19 (s, 1H), 8.00 (d, J=7.7 Hz, 1H), 7.82 (app t, J=7.9 Hz, 1H), 7.76 (d, J=7.9 Hz, 1H), 7.71 (dd, J=8.2, 1.2 Hz, 1H), 7.69-7.64 (m, 2H), 7.63-7.60 (m, 1H), 7.54-7.45 (m, 3H); MS m/z=462 [M+H]⁺.

### (E)-N-(3-(3-oxo-3-(pyrimidin-4-ylamino)prop-1-en-1-yl)phenyl)-3-(trifluoromethyl)benzamide (Compound 54)

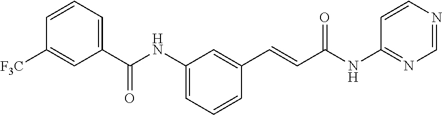 [SMILES: O=C(/C=C/c1cccc(NC(=O)c2cccc(C(F)(F)F)c2)c1)Nc1ccncn1]

(E)-3-(3-(3-(trifluoromethyl)benzamido)phenyl)acrylic acid Int-5 was reacted with 4-aminopyrimidine using the experimental conditions outlined in the General Procedure I to give Compound 54 as a white solid in 44% yield. ¹H-NMR (400 MHz, DMSO-d₆): δ 11.19 (s, 1H), 10.62 (s, 1H), 8.91 (s, 1H), 8.69 (d, J=5.8 Hz, 1H), 8.33 (s, 1H), 8.29 (d, J=7.8 Hz, 1H), 8.22 (dd, J=5.8, 1.2 Hz, 1H), 8.18 (s, 1H), 8.00 (d, J=7.7 Hz, 1H), 7.82 (d, J=7.7 Hz, 1H), 7.80-7.77 (m, 1H), 7.69 (d, J=15.7 Hz, 1H), 7.48 (app t, J=7.8 Hz, 1H), 7.41 (d, J=7.4 Hz, 1H), 7.08 (d, J=15.7 Hz, 1H); MS m/z=413 [M+H]⁺.

### (E)-N-(3-(3-oxo-3-((4-(trifluoromethyl)pyridin-2-yl)amino)prop-1-en-1-yl)phenyl)-3-(trifluoromethyl)benzamide (Compound 55)

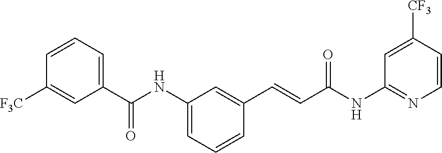 [SMILES: O=C(/C=C/c1cccc(NC(=O)c2cccc(C(F)(F)F)c2)c1)Nc1cc(C(F)(F)F)ccn1]

(E)-3-(3-(3-(trifluoromethyl)benzamido)phenyl)acrylic acid Int-5 was reacted with 2-amino-4-trifluoromethylpyridine using the experimental conditions outlined in the General Procedure I to give Compound 55 as an off-white solid in 21% yield. ¹H-NMR (400 MHz, DMSO-d₆): δ 11.23 (s, 1H), 10.68 (s, 1H), 8.64 (d, J=5.1 Hz, 1H), 8.59 (s, 1H), 8.25 (s, 1H), 8.22 (s, 1H), 8.08 (d, J=8.0 Hz, 1H), 8.01 (d, J=7.7 Hz, 1H), 7.87 (d, J=7.8 Hz, 1H), 7.78 (d, J=15.6 Hz, 1H), 7.71-7.60 (m, 2H), 7.51-7.47 (m, 2H), 7.19 (d, J=15.8 Hz, 1H); MS m/z=480 [M+H]⁺.

### (E)-N-(3-(3-((5-(4-methylpiperazin-1-yl)pyridin-2-yl)amino)-3-oxoprop-1-en-1-yl)phenyl)-3-(trifluoromethyl)benzamide (Compound 56)

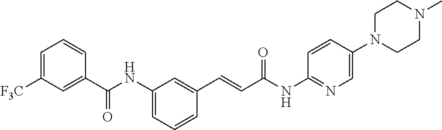 [SMILES: CN1CCN(c2ccc(NC(=O)/C=C/c3cccc(NC(=O)c4cccc(C(F)(F)F)c4)c3)nc2)CC1]

(E)-3-(3-(3-(trifluoromethyl)benzamido)phenyl)acrylic acid Int-5 was reacted with 5-(4-methylpiperazin-1-yl)pyridin-2-amine using the experimental conditions outlined in the General Procedure I to give Compound 56 as a yellow solid in 49% yield. ¹H-NMR (400 MHz, DMSO-d₆): δ 10.60 (s, 1H), 10.56 (s, 1H), 8.32 (s, 1H), 8.29 (d, J=7.7 Hz, 1H), 8.11 (d, J=8.3 Hz, 1H), 8.10 (s, 1H), 8.04 (d, J=2.9 Hz, 1H), 7.99 (d, J=7.7 Hz, 1H), 7.82 (d, J=7.8 Hz, 1H), 7.79-7.75 (m, 1H), 7.57 (d, J=15.6 Hz, 1H), 7.50-7.40 (m, 2H), 7.37 (d, J=7.7 Hz, 1H), 7.03 (d, J=15.6 Hz, 1H), 3.15 (app t, J=4.8 Hz, 4H), 2.46 (app t, J=4.8 Hz, 4H), 2.22 (s, 3H); MS m/z=510 [M+H]⁺.

### (E)-N-(3-(3-((5-morpholinopyridin-2-yl)amino)-3-oxoprop-1-en-1-yl)phenyl)-3-(trifluoromethyl)benzamide (Compound 57)

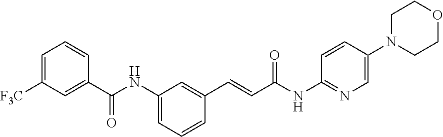 [SMILES: O=C(/C=C/c1cccc(NC(=O)c2cccc(C(F)(F)F)c2)c1)Nc1ccc(N2CCOCC2)cn1]

(E)-3-(3-(3-(trifluoromethyl)benzamido)phenyl)acrylic acid Int-5 was reacted with 5-morpholinopyridin-2-amine using the experimental conditions outlined in the General Procedure I to give Compound 57 as a yellow solid in 53% yield. ¹H-NMR (400 MHz, DMSO-d₆): δ 10.60 (s, 1H), 10.58 (s, 1H), 8.32 (s, 1H), 8.29 (d, J=7.9 Hz, 1H), 8.14-8.10 (m, 2H), 8.05 (d, J=3.0 Hz, 1H), 7.99 (d, J=7.9 Hz, 1H), 7.82 (d, J=7.8 Hz, 1H), 7.79-7.75 (m, 1H), 7.57 (d, J=15.7 Hz, 1H), 7.51-7.42 (m, 2H), 7.37 (d, J=7.7 Hz, 1H), 7.03 (d, J=15.7 Hz, 1H), 3.75 (app t, J=4.7 Hz, 4H), 3.12 (app t, J=4.7 Hz, 4H); MS m/z=497 [M+H]⁺.

### (E)-N-(3-(3-oxo-3-(thiazol-2-ylamino)prop-1-en-1-yl)phenyl)-3-(trifluoromethyl) benzamide (Compound 58)

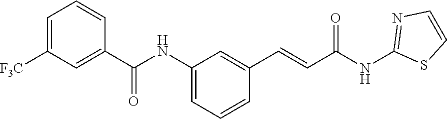 [SMILES: O=C(/C=C/c1cccc(NC(=O)c2cccc(C(F)(F)F)c2)c1)Nc1nccs1]

(E)-3-(3-(3-(trifluoromethyl)benzamido)phenyl)acrylic acid Int-5 was reacted with 2-aminothiazole using the experimental conditions outlined in the General Procedure I to give Compound 58 as an off-white solid in 31% yield. ¹H-NMR (400 MHz, DMSO-d₆): δ 12.40 (bs, 1H), 10.63 (s, 1H), 8.33 (s, 1H), 8.29 (d, J=8.3 Hz, 1H), 8.15 (s, 1H), 8.00 (d, J=7.6 Hz, 1H), 7.86-7.77 (m, 2H), 7.71 (d, J=15.7 Hz, 1H), 7.52 (d, J=3.5 Hz, 1H), 7.48 (app t, J=7.9 Hz, 1H), 7.41 (d, J=7.8 Hz, 1H), 7.26 (d, J=3.6 Hz, 1H), 6.96 (d, J=15.8 Hz, 1H); MS m/z=418 [M+H]⁺.

### (E)-N-(3-(3-((4-morpholinopyridin-2-yl)amino)-3-oxoprop-1-en-1-yl)phenyl)-3-(trifluoromethyl)benzamide (Compound 59)

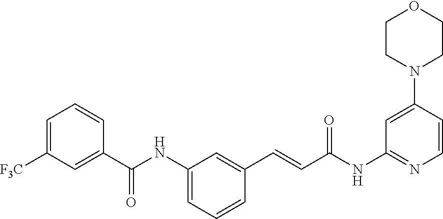

(E)-3-(3-(3-(trifluoromethyl)benzamido)phenyl)acrylic acid Int-5 was reacted with 4-morpholinopyridin-2-amine using the experimental conditions outlined in the General Procedure I to give Compound 59 as an off-white solid in 17% yield. ¹H-NMR (400 MHz, DMSO-d₆): δ 10.60 (s, 1H), 10.50 (s, 1H), 8.32 (s, 1H), 8.29 (d, J=8.2 Hz, 1H), 8.08 (s, 1H), 8.01-7.98 (m, 2H), 7.87-7.76 (m, 3H), 7.57 (d, J=15.7 Hz, 1H), 7.46 (app t, J=8.0 Hz, 1H), 7.37 (d, J=7.6 Hz, 1H), 7.07 (d, J=15.7 Hz, 1H), 6.64 (dd, J=5.9, 2.3 Hz, 1H), 3.74 (app t, J=4.8 Hz, 4H), 3.27 (app t, J=4.8 Hz, 4H); MS m/z=497 [M+H]⁺.

### (E)-N-(3-(3-((2-morpholinopyridin-3-yl)amino)-3-oxoprop-1-en-1-yl)phenyl)-3-(trifluoromethyl)benzamide (Compound 60)

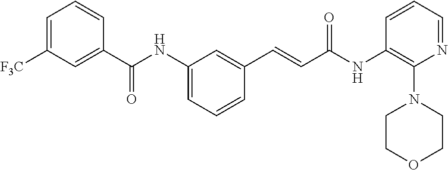

(E)-3-(3-(3-(trifluoromethyl)benzamido)phenyl)acrylic acid Int-5 was reacted with 2-morpholinopyridin-3-amine using the experimental conditions outlined in the General Procedure I to give Compound 60 as a brown solid in 85% yield. ¹H-NMR (400 MHz, DMSO-d₆): δ 10.62 (s, 1H), 9.45 (s, 1H), 8.33 (s, 1H), 8.29 (d, J=7.7 Hz, 1H), 8.19 (d, J=7.5 Hz, 1H), 8.14 (s, 1H), 8.08 (dd, J=4.7, 1.7 Hz, 1H), 8.00 (d, J=7.8 Hz, 1H), 7.82 (app t, J=7.8 Hz, 1H), 7.76 (d, J=7.9 Hz, 1H), 7.59 (d, J=15.7 Hz, 1H), 7.47 (app t, J=7.8 Hz, 1H), 7.42 (d, J=7.6 Hz, 1H), 7.16 (d, J=15.8 Hz, 1H), 7.07 (dd, J=7.9, 4.8 Hz, 1H), 3.80 (app t, J=4.4 Hz, 4H), 3.09 (app t, J=4.5 Hz, 4H); MS m/z=497 [M+H]⁺.

### (E)-N-(3-(3-((2-morpholinopyridin-4-yl)amino)-3-oxoprop-1-en-1-yl)phenyl)-3-(trifluoromethyl)benzamide (Compound 61)

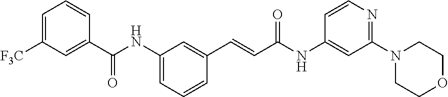 [SMILES: O=C(/C=C/c1cccc(NC(=O)c2cccc(C(F)(F)F)c2)c1)Nc1ccnc(N2CCOCC2)c1]

(E)-3-(3-(3-(trifluoromethyl)benzamido)phenyl)acrylic acid Int-5 was reacted with 2-morpholinopyridin-4-amine using the experimental conditions outlined in the General Procedure I to give Compound 61 as a yellow solid in 55% yield. ¹H-NMR (400 MHz, DMSO-d₆): δ 10.62 (s, 1H), 10.46 (s, 1H), 8.33 (s, 1H), 8.29 (d, J=8.2 Hz, 1H), 8.24 (s, 1H), 8.04 (d, J=5.6 Hz, 1H), 8.00 (d, J=8.0 Hz, 1H), 7.82 (t, J=7.8 Hz, 1H), 7.73 (d, J=7.5 Hz, 1H), 7.60 (d, J=15.5 Hz, 1H), 7.47 (t, J=7.8 Hz, 1H), 7.40 (d, J=7.7 Hz, 1H), 7.22 (s, 1H), 6.95 (d, J=5.6 Hz, 1H), 6.85 (d, J=15.6 Hz, 1H), 3.75-3.62 (m, 4H), 3.43-3.38 (m, 4H).

### (E)-N-(3-(3-((2-(4-methylpiperazin-1-yl)pyridin-3-yl)amino)-3-oxoprop-1-en-1-yl)phenyl)-3-(trifluoromethyl)benzamide (Compound 62)

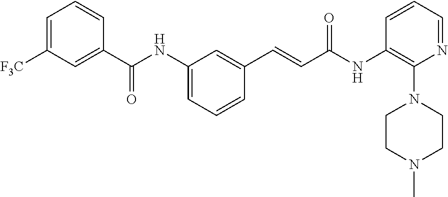

(E)-3-(3-(3-(trifluoromethyl)benzamido)phenyl)acrylic acid Int-5 was reacted with 2-(4-methylpiperazin-1-yl)pyridin-3-amine using the experimental conditions outlined in the General Procedure I to give Compound 62 as a brown solid in 35% yield. ¹H-NMR (400 MHz, MeOD): δ 8.28 (s, 1H), 8.25-8.18 (m, 2H), 8.10 (s, 1H), 8.08 (dd, J=4.9, 1.7 Hz, 1H), 7.90 (d, J=7.8 Hz, 1H), 7.76-7.69 (m, 3H), 7.45-7.41 (m, 2H), 7.07 (dd, J=7.9, 4.9 Hz, 1H), 6.97 (d, J=15.7 Hz, 1H), 3.21 (app t, J=4.6 Hz, 4H), 2.79-2.63 (m, 4H), 2.39 (s, 3H); MS m/z=510 [M+H]⁺.

### (E)-N-(3-(3-oxo-3-(pyridin-4-ylamino)prop-1-en-1-yl)phenyl)-3-(trifluoromethyl) benzamide (Compound 63)

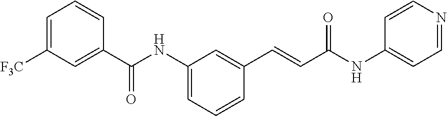 [SMILES: O=C(/C=C/c1cccc(NC(=O)c2cccc(C(F)(F)F)c2)c1)Nc1ccncc1]

(E)-3-(3-(3-(trifluoromethyl)benzamido)phenyl)acrylic acid Int-5 was reacted with 4-aminopyridine using the experimental conditions outlined in the General Procedure I to give Compound 63 as a white solid in 61% yield. ¹H-NMR (400 MHz, DMSO-d₆): δ 10.67 (s, 1H), 10.62 (s, 1H), 8.50-8.43 (m, 2H), 8.33 (s, 1H), 8.29 (d, J=7.8 Hz, 1H), 8.26 (s, 1H), 8.00 (d, J=7.9 Hz, 1H), 7.82 (app t, J=7.8 Hz, 1H), 7.73 (d, J=8.7 Hz, 1H), 7.68-7.63 (m, 3H), 7.47 (app t, J=7.8 Hz, 1H), 7.42 (d, J=7.7 Hz, 1H), 6.87 (d, J=15.7 Hz, 1H); MS m/z=412 [M+H]⁺.

### (E)-N-(3-(3-((5-methylpyridin-3-yl)amino)-3-oxoprop-1-en-1-yl)phenyl)-3-(trifluoromethyl)benzamide (Compound 64)

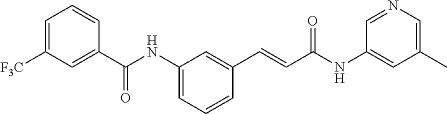 [SMILES: Cc1cncc(NC(=O)/C=C/c2cccc(NC(=O)c3cccc(C(F)(F)F)c3)c2)c1]

(E)-3-(3-(3-(trifluoromethyl)benzamido)phenyl)acrylic acid Int-5 was reacted with 3-amino-5-methylpyridine using the experimental conditions outlined in the General Procedure I to give Compound 64 as an off-white solid in 63% yield. ¹H-NMR (400 MHz, DMSO-d₆): δ 10.62 (s, 1H), 10.47 (s, 1H), 8.64 (d, J=2.2 Hz, 1H), 8.33 (s, 1H), 8.30 (d, J=8.0 Hz, 1H), 8.24 (s, 1H), 8.14 (s, 1H), 8.05-7.99 (m, 2H), 7.82 (app t, J=7.8 Hz, 1H), 7.74 (d, J=8.0 Hz, 1H), 7.61 (d, J=15.7 Hz, 1H), 7.47 (app t, J=7.8 Hz, 1H), 7.41 (d, J=7.7 Hz, 1H), 6.87 (d, J=15.7 Hz, 1H), 2.31 (s, 3H); MS m/z=426 [M+H]⁺.

### (E)-3-(3-((5-ethynylpyridin-2-yl)amino)-3-oxoprop-1-en-1-yl)-N-(3-(trifluoromethyl) phenyl)benzamide (Compound 65)

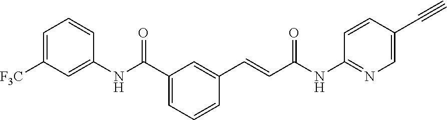 [SMILES: C#Cc1ccc(NC(=O)/C=C/c2cccc(C(=O)Nc3cccc(C(F)(F)F)c3)c2)nc1]

(E)-3-(3-((3-(trifluoromethyl)phenyl)carbamoyl)phenyl)acrylic acid Int-12 was reacted with 5-ethynylpyridin-2-amine using the experimental conditions outlined in the General Procedure I to give Compound 65 as a light brown solid in 24% yield. ¹H-NMR (400 MHz, DMSO-d₆): δ 10.98 (s, 1H), 10.68 (s, 1H), 8.48 (d, J=2.2 Hz, 1H), 8.27 (d, J=8.7 Hz, 1H), 8.25 (s, 1H), 8.21 (s, 1H), 8.08 (d, J=7.7 Hz, 1H), 8.00 (d, J=7.9 Hz, 1H), 7.94 (dd, J=8.7, 2.3 Hz, 1H), 7.86 (d, J=7.7 Hz, 1H), 7.75 (d, J=15.6 Hz, 1H), 7.68-7.61 (m, 2H), 7.48 (d, J=8.0 Hz, 1H), 7.18 (d, J=15.7 Hz, 1H), 4.36 (s, 1H); MS m/z=436 [M+H]⁺.

### (E)-2-(3-(3-((3-(trifluoromethyl)phenyl)carbamoyl)phenyl)acrylamido)benzoic acid (Compound 66)

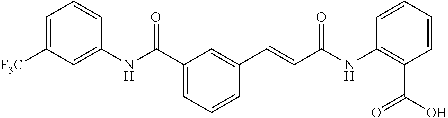 [SMILES: O=C(/C=C/c1cccc(C(=O)Nc2cccc(C(F)(F)F)c2)c1)Nc1ccccc1C(=O)O]

(E)-3-(3-((3-(trifluoromethyl)phenyl)carbamoyl)phenyl)acrylic acid Int-12 was reacted with anthranilic acid using the experimental conditions outlined in the General Procedure I to give Compound 66 as an off-white solid in 40% yield. ¹H-NMR (400 MHz, DMSO-d₆): δ 14.87 (bs, 1H), 10.69 (s, 1H), 8.59 (dd, J=8.2, 1.0 Hz, 1H), 8.31 (s, 1H), 8.27 (s, 1H), 8.09 (d, J=8.4 Hz, 1H), 7.99 (dd, J=7.8, 1.8 Hz, 1H), 7.98-7.96 (d, J=8.1 Hz, 1H), 7.92 (d, J=7.7 Hz, 1H), 7.67-7.58 (m, 3H), 7.48 (d, J=7.8 Hz, 1H), 7.33-7.28 (m, 1H), 6.97 (app td, J=7.5, 1.0 Hz, 1H), 6.86 (d, J=15.7 Hz, 1H); MS m/z=455 [M+H]⁺.

### (E)-N-(3-(3-(methyl(pyridin-2-yl)amino)-3-oxoprop-1-en-1-yl)phenyl)-3-(trifluoromethyl)benzamide (Compound 67)

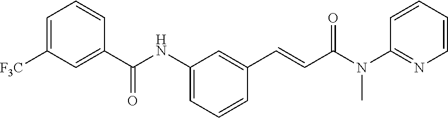 [SMILES: CN(C(=O)/C=C/c1cccc(NC(=O)c2cccc(C(F)(F)F)c2)c1)c1ccccn1]

(E)-3-(3-(3-(trifluoromethyl)benzamido)phenyl)acrylic acid Int-5 was reacted with 2-(methylamino)pyridine using the experimental conditions outlined in the General Procedure I to give Compound 67 as a yellow solid in 47% yield. ¹H-NMR (400 MHz, DMSO-d₆): δ 10.51 (s, 1H), 8.52 (ddd, J=4.8, 1.9, 0.8 Hz, 1H), 8.27 (s, 1H), 8.24 (d, J=7.9 Hz, 1H), 7.98 (d, J=7.9 Hz, 1H), 7.93 (ddd, J=8.0, 7.5, 2.0 Hz, 1H), 7.85-7.76 (m, 3H), 7.55 (d, J=15.5 Hz, 1H), 7.46 (d, J=8.1 Hz, 1H), 7.41-7.34 (m, 2H), 7.30 (d, J=7.9 Hz, 1H), 6.64 (d, J=15.5 Hz, 1H), 3.40 (s, 3H); MS m/z=426 [M+H]⁺.

### (E)-N-(3-(3-oxo-3-((6-(trifluoromethyl)pyridin-3-yl)amino)prop-1-en-1-yl)phenyl)-3-(trifluoromethyl)benzamide (Compound 68)

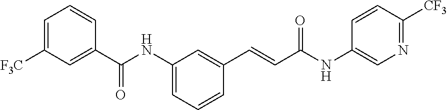 [SMILES: O=C(/C=C/c1cccc(NC(=O)c2cccc(C(F)(F)F)c2)c1)Nc1ccc(C(F)(F)F)nc1]

(E)-3-(3-(3-(trifluoromethyl)benzamido)phenyl)acrylic acid Int-5 was reacted with 3-amino-6-(trifluoromethyl)pyridine using the experimental conditions outlined in the General Procedure I to give Compound 68 as a light brown solid in 48% yield. ¹H-NMR (400 MHz, DMSO-d₆): δ 10.91 (s, 1H), 10.62 (s, 1H), 8.97 (d, J=2.3 Hz, 1H), 8.44 (dd, J=8.5, 2.1 Hz, 1H), 8.33 (s, 1H), 8.30-8.27 (m, 2H), 8.00 (d, J=7.8 Hz, 1H), 7.91 (d, J=8.7 Hz, 1H), 7.82 (t, J=8.0 Hz, 1H), 7.74 (d, J=8.0 Hz, 1H), 7.68 (d, J=15.7 Hz, 1H), 7.48 (t, J=7.8 Hz, 1H), 7.43 (d, J=7.7 Hz, 1H), 6.89 (d, J=15.7 Hz, 1H); MS m/z=480 [M+H]⁺.

### (E)-3-(3-((6-morpholinopyridin-3-yl)amino)-3-oxoprop-1-en-1-yl)-N-(3-(trifluoromethyl)phenyl)benzamide (Compound 69)

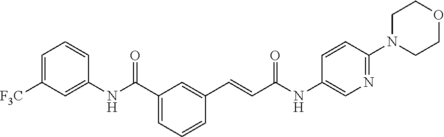 [SMILES: O=C(/C=C/c1cccc(C(=O)Nc2cccc(C(F)(F)F)c2)c1)Nc1ccc(N2CCOCC2)nc1]

(E)-3-(3-((3-(trifluoromethyl)phenyl)carbamoyl)phenyl)acrylic acid (Int-12) was reacted with 6-morpholinopyridin-3-amine using the experimental conditions outlined in the General Procedure I to give Compound 69 as a yellow solid in 56% yield. ¹H-NMR (500 MHz, DMSO-d₆): δ 10.68 (s, 1H), 10.23 (s, 1H), 8.45 (d, J=2.6 Hz, 1H), 8.26 (s, 1H), 8.22 (s, 1H), 8.09 (d, J=7.9 Hz, 1H), 7.99 (d, J=7.8 Hz, 1H), 7.94 (dd, J=9.1, 2.7 Hz, 1H), 7.85 (d, J=7.8 Hz, 1H), 7.67-7.61 (m, 3H), 7.48 (d, J=7.9 Hz, 1H), 6.94 (d, J=15.7 Hz, 1H), 6.86 (d, J=9.1 Hz, 1H), 3.70 (t, J=4.8 Hz, 4H), 3.39 (t, J=4.9 Hz, 4H).

### (E)-N-methyl-3-(3-oxo-3-((2-(pyridin-2-yl)phenyl)amino)prop-1-en-1-yl)-N-(3-(trifluoromethyl)phenyl)benzamide (Compound 70)

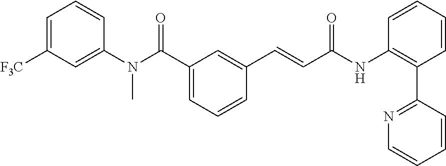 [SMILES: CN(C(=O)c1cccc(/C=C/C(=O)Nc2ccccc2-c2ccccn2)c1)c1cccc(C(F)(F)F)c1]

(E)-3-(3-(methyl(3-(trifluoromethyl)phenyl)carbamoyl)phenyl)acrylic acid Int-14 was reacted with Int-100 using the experimental conditions outlined in the General Procedure I to give Compound 70 as an off-white solid in 61% yield. ¹H-NMR (500 MHz, DMSO-d₆): δ 12.27 (s, 1H), 8.85 (d, J=4.0 Hz, 1H), 8.44 (d, J=8.1 Hz, 1H), 8.01 (td, J=7.8, 1.8 Hz, 1H), 7.94 (d, J=8.1 Hz, 1H), 7.85 (dd, J=7.9, 1.5 Hz, 1H), 7.71-7.62 (m, 3H), 7.56-7.44 (m, 6H), 7.33-7.24 (m, 3H), 6.79 (d, J=15.7 Hz, 1H), 3.43 (s, 3H).

### (E)-3-(3-((2-morpholinopyrimidin-5-yl)amino)-3-oxoprop-1-en-1-yl)-N-(3-(trifluoromethyl)phenyl)benzamide (Compound 71)

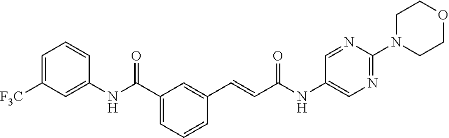 [SMILES: O=C(/C=C/c1cccc(C(=O)Nc2cccc(C(F)(F)F)c2)c1)Nc1cnc(N2CCOCC2)nc1]

(E)-3-(3-((3-(trifluoromethyl)phenyl)carbamoyl)phenyl)acrylic acid (Int-12) was reacted with 2-(morpholin-4-yl)pyrimidin-5-amine using the experimental conditions outlined in the General Procedure I to give Compound 71 as a yellow solid in 71% yield. ¹H-NMR (500 MHz, DMSO-d₆): δ 10.66 (s, 1H), 10.26 (s, 1H), 8.69 (s, 2H), 8.25 (s, 1H), 8.22 (s, 1H), 8.08 (d, J=8.2 Hz, 1H), 7.99 (d, J=7.8 Hz, 1H), 7.86 (d, J=7.8 Hz, 1H), 7.67 (d, J=15.7 Hz, 1H), 7.63 (td, J=7.8, 5.0 Hz, 2H), 7.48 (d, J=7.8 Hz, 1H), 6.91 (d, J=15.8 Hz, 1H), 3.66 (bs, 8H).

### (E)-N-(3-(3-((6-morpholinopyridin-3-yl)amino)-3-oxoprop-1-en-1-yl)phenyl)-3-(trifluoromethyl)benzamide (Compound 72)

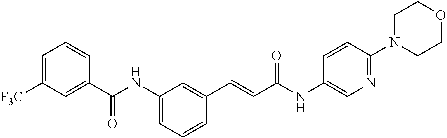 [SMILES: O=C(/C=C/c1cccc(NC(=O)c2cccc(C(F)(F)F)c2)c1)Nc1ccc(N2CCOCC2)nc1]

(E)-3-(3-(3-(trifluoromethyl)benzamido)phenyl)acrylic acid Int-5 was reacted with 6-morpholinopyridin-3-amine using the experimental conditions outlined in the General Procedure I to give Compound 72 as a yellow solid in 29% yield. ¹H-NMR (400 MHz, DMSO-d₆): δ 10.61 (s, 1H), 10.21 (s, 1H), 8.45 (d, J=2.5 Hz, 1H), 8.34 (s, 1H), 8.30 (d, J=7.9 Hz, 1H), 8.21 (s, 1H), 8.00 (d, J=7.4 Hz, 1H), 7.94 (dd, J=9.1, 2.6 Hz, 1H), 7.82 (t, J=7.7 Hz, 1H), 7.73 (d, J=7.5 Hz, 1H), 7.56 (d, J=15.5 Hz, 1H), 7.47 (t, J=7.9 Hz, 1H), 7.39 (d, J=7.2 Hz, 1H), 6.87 (d, J=9.1 Hz, 1H), 6.83 (d, J=15.8 Hz, 1H), 3.71 (t, J=4.8 Hz, 4H), 3.39 (t, J=4.8 Hz, 4H).

### (E)-N-(3-(3-((5-(Morpholine-4-carbonyl)pyridin-3-yl)amino)-3-oxoprop-1-en-1-yl)phenyl)-3-(trifluoromethyl)benzamide (Compound 73)

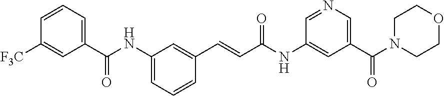 [SMILES: O=C(/C=C/c1cccc(NC(=O)c2cccc(C(F)(F)F)c2)c1)Nc1cncc(C(=O)N2CCOCC2)c1]

Compound 73 was synthesized from Int-102 and morpholine using the experimental conditions outlined in the General Procedure I to give Compound 73 as a white solid in 55% yield. ¹H-NMR (400 MHz, CD3CN): δ 9.05 (s, 1H), 8.90 (s, 1H), 8.80 (d, J=1.9 Hz, 1H), 8.31 (s, 1H), 8.26 (s, 1H), 8.22-8.18 (m, 2H), 8.15 (t, J=1.7 Hz, 1H), 7.90 (d, J=7.8 Hz, 1H), 7.73 (d, J=7.8 Hz, 1H), 7.70-7.67 (m, 2H), 7.43 (t, J=7.8 Hz, 1H), 7.38 (d, J=7.7 Hz, 1H), 6.75 (d, J=15.6 Hz, 1H), 3.68 (bs, 4H), 3.60 (bs, 2H), 3.41 (bs, 2H). MS m/z=525.1 [M+H]⁺.

### (E)-N-(2-Morpholinoethyl)-5-(3-(3-(3-(trifluoromethyl)benzamido)phenyl)acrylamido) nicotinamide (Compound 74)

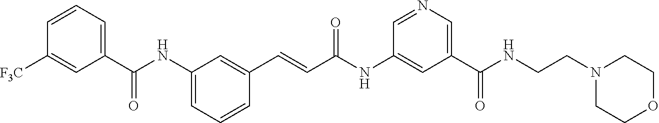 [SMILES: O=C(/C=C/c1cccc(NC(=O)c2cccc(C(F)(F)F)c2)c1)Nc1cncc(C(=O)NCCN2CCOCC2)c1]

Compound 74 was synthesized from Int-102 and 4-(2-aminoethyl)morpholine using the experimental conditions outlined in the General Procedure I to give Compound 74 as an off-white solid in 50% yield. ¹H-NMR (400 MHz, DMSO-d₆): δ 10.66 (s, 1H), 10.61 (s, 1H), 8.95 (d, J=2.4 Hz, 1H), 8.70 (d, J=2.0 Hz, 1H), 8.66-8.60 (m, 1H), 8.53 (t, J=2.2 Hz, 1H), 8.33 (s, 1H), 8.30 (d, J=8.6 Hz, 1H), 8.25 (s, 1H), 8.00 (d, J=8.4 Hz, 1H), 7.82 (t, J=7.5 Hz, 1H), 7.74 (d, J=8.7 Hz, 1H), 7.64 (d, J=15.6 Hz, 1H), 7.48 (t, J=7.7 Hz, 1H), 7.42 (d, J=8.2 Hz, 1H), 6.87 (d, J=15.6 Hz, 1H), 3.58 (t, J=4.6 Hz, 4H), 3.43-3.36 (m, 4H), 2.45-2.40 (m, 4H). MS m/z=568.3 [M+H]⁺.

### (E)-3-(3-((5-Morpholinopyridin-2-yl)amino)-3-oxoprop-1-en-1-yl)-N-(3-(trifluoromethyl) phenyl)benzamide (Compound 75)

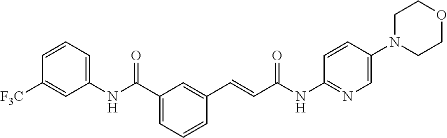 [SMILES: O=C(/C=C/c1cccc(C(=O)Nc2cccc(C(F)(F)F)c2)c1)Nc1ccc(N2CCOCC2)cn1]

Compound 75 was synthesized from (E)-3-(3-((3-(trifluoromethyl)phenyl)carbamoyl)phenyl)acrylic acid (Int-12) and 2-amino-5-morpholinopyridine using the experimental conditions outlined in the General Procedure I, and the product was obtained as a brown solid in 68% yield. ¹H-NMR (400 MHz, DMSO-d₆): δ 10.67 (s, 1H), 10.55 (s, 1H), 8.25 (s, 1H), 8.19 (s, 1H), 8.13 (d, J=9.1 Hz, 1H), 8.08 (d, J=8.9 Hz, 1H), 8.06 (d, J=3.0 Hz, 1H), 7.98 (d, J=7.9 Hz, 1H), 7.83 (d, J=7.8 Hz, 1H), 7.68 (d, J=15.7 Hz, 1H), 7.65-7.60 (m, J=2.9 Hz, 2H), 7.51-7.42 (m, 2H), 7.14 (d, J=15.8 Hz, 1H), 3.75 (t, J=4.8 Hz, 4H), 3.13 (t, J=4.8 Hz, 4H) ppm; MS m/z=497.0 [M+H]⁺.

### (E)-3-(3-((2-(1H-Imidazol-2-yl)phenyl)amino)-3-oxoprop-1-en-1-yl)-N-methyl-N-(3-(trifluoromethyl)phenyl)benzamide (Compound 76)

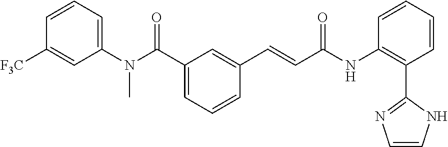 [SMILES: CN(C(=O)c1cccc(/C=C/C(=O)Nc2ccccc2-c2ncc[nH]2)c1)c1cccc(C(F)(F)F)c1]

(E)-3-(3-(methyl(3-(trifluoromethyl)phenyl)carbamoyl)phenyl)acrylic acid Int-14 was reacted with 1-((2-(trimethylsilyl)ethoxy)methyl)-1H-imidazol-2-yl)phenylamine using the experimental conditions outlined in the General Procedure I to give the SEM-protected product as an off-white solid in 56% yield. The resulting solid (87.3 mg, 0.14 mmol) was dissolved in DCM (1.1 mL) and TFA (900 μL) was slowly added. The reaction mixture was stirred at room temperature for 3 hours. Then, the volatiles were removed in vacuo and the crude product was purified on reverse phased silica gel using a gradient (90:10 to 0:100) of water and acetonitrile with 0.1% trifluoroacetic acid. Fractions containing the desired product were combined, treated with saturated aqueous sodium bicarbonate solution and the mixture was extracted with dichloromethane (3×). Combined organic phases were washed with brine (1×), dried over anhydrous sodium sulfate and concentrated in vacuo to give compound 76 as an off-white solid in 71% yield (49.2 mg). ¹H-NMR (400 MHz, DMSO-d6): δ 13.30 (s, 1H), 12.87 (s, 1H), 8.73 (dd, J=8.4, 1.0 Hz, 1H), 7.96 (dd, J=7.9, 1.3 Hz, 1H), 7.67 (bs, 3H), 7.61-7.49 (m, 4H), 7.41-7.28 (m, 5H), 7.19 (td, J=7.6, 0.9 Hz, 1H), 6.70 (d, J=15.7 Hz, 1H), 3.44 (s, 3H) ppm; MS m/z=621.1 [M+H]⁺.

### (E)-N-(3-(3-((2-(4-Methylpiperazine-1-carbonyl)pyridin-4-yl)amino)-3-oxoprop-1-en-1-yl) phenyl)-3-(trifluoromethyl)benzamide (Compound 77)

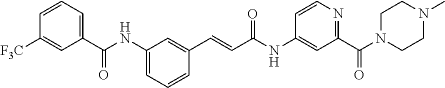 [SMILES: CN1CCN(C(=O)c2cc(NC(=O)/C=C/c3cccc(NC(=O)c4cccc(C(F)(F)F)c4)c3)ccn2)CC1]

(E)-4-(3-(3-(3-(trifluoromethyl)benzamido)phenyl)acrylamido)picolinic acid (Int-103) (22.6 mg, 0.05 mmol) was dissolved in dry DMF (150 μL) and triethylamine (12 μL, 0.09 mmol) was added, followed by N-methylpiperazine (11 μL, 0.10 mmol). The reaction mixture was stirred at room temperature for 18 hours. The solution was diluted with water (10 mL) and extracted with Ethyl Acetate (10 mL) and the phases were separated. The aqueous layer was extracted with Ethyl Acetate (3×10 mL) and combined organic phases were washed with water (1×20 mL), brine (1×20 mL), dried over anhydrous sodium sulfate, filtered and concentrated in vacuo. The crude product was purified on reversed phase silica using a gradient of water and acetonitrile with 0.1% trifluoroacetic acid from 90:10 to 0:100. Fractions containing the desired product were combined, treated with saturated aqueous sodium bicarbonate solution and extracted with DCM (3×). Combined organic layers were washed with brine (1×), dried over anhydrous sodium sulfate, filtered and concentrated in vacuo to give Compound 77 as an off-white solid in 65% yield (17.4 mg). ¹H-NMR (400 MHz, DMSO-d₆): δ 10.81 (s, 1H), 10.61 (s, 1H), 8.46 (d, J=5.5 Hz, 1H), 8.33 (s, 1H), 8.31-8.27 (m, 2H), 8.00 (d, J=7.8 Hz, 1H), 7.86 (d, J=2.0 Hz, 1H), 7.82 (t, J=7.8 Hz, 1H), 7.74 (d, J=8.3 Hz, 1H), 7.69-7.64 (m, 2H), 7.48 (t, J=7.8 Hz, 1H), 7.43 (d, J=7.7 Hz, 1H), 6.85 (d, J=15.7 Hz, 1H), 3.64 (t, J=4.7 Hz, 2H), 3.40 (t, J=5.1 Hz, 2H), 2.38 (t, J=4.8 Hz, 2H), 2.28 (t, J=4.5 Hz, 2H), 2.20 (s, 3H) ppm; MS m/z=538.0 [M+H]⁺.

### (E)-N-(5-Morpholinopyridin-2-yl)-3-(3-(N-phenylsulfamoyl)phenyl)acrylamide (Compound 78)

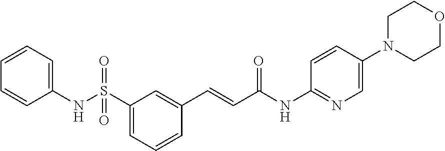 [SMILES: O=C(/C=C/c1cccc(S(=O)(=O)Nc2ccccc2)c1)Nc1ccc(N2CCOCC2)cn1]

Int-28 was reacted with 2-amino-5-morpholinopyridine using the experimental conditions outlined in the General Procedure I to give Compound 78 as a green solid in 47% yield (33.0 mg). ¹H-NMR (400 MHz, DMSO-d₆): δ 10.55 (s, 1H), 10.35 (s, 1H), 8.11 (d, J=9.0 Hz, 1H), 8.05 (d, J=3.0 Hz, 1H), 7.98 (s, 1H), 7.80 (d, J=7.8 Hz, 1H), 7.73 (d, J=7.9 Hz, 1H), 7.66-7.56 (m, 2H), 7.44 (dd, J=9.1, 3.0 Hz, 1H), 7.26-7.21 (m, 2H), 7.12-6.99 (m, 4H), 3.75 (t, J=4.8 Hz, 4H), 3.13 (t, J=4.8 Hz, 4H) ppm; MS m/z=464.9 [M+H]⁺.

### (E)-N-Methyl-N-(3-(3-((5-morpholinopyridin-2-yl)amino)-3-oxoprop-1-en-1-yl)phenyl)-3-(trifluoromethyl)benzamide (Compound 79)

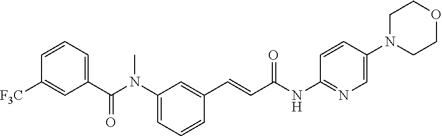 [SMILES: CN(C(=O)c1cccc(C(F)(F)F)c1)c1cccc(/C=C/C(=O)Nc2ccc(N3CCOCC3)cn2)c1]

Int-7 was reacted with 2-amino-5-morpholinopyridine using the experimental conditions outlined in the General Procedure I to give Compound 79 as a brown solid in 61% yield. ¹H-NMR (400 MHz, DMSO-d₆): δ 10.45 (s, 1H), 8.09 (d, J=9.1 Hz, 1H), 8.04 (d, J=2.9 Hz, 1H), 7.64 (d, J=7.8 Hz, 1H), 7.59 (bs, 2H), 7.52-7.40 (m, 5H), 7.33 (t, J=7.8 Hz, 1H), 7.23 (d, J=7.8 Hz, 1H), 6.94 (d, J=15.8 Hz, 1H), 3.75 (t, J=4.7 Hz, 4H), 3.42 (s, 3H), 3.12 (t, J=4.8 Hz, 4H) ppm; MS m/z=511.1 [M+H]⁺.

### (E)-5-(3-((5-Morpholinopyridin-2-yl)amino)-3-oxoprop-1-en-1-yl)-N-(3-(trifluoromethyl) phenyl)nicotinamide (Compound 80)

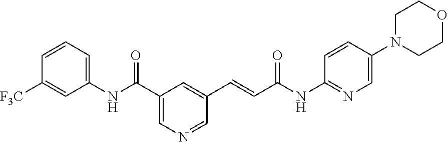 [SMILES: O=C(/C=C/c1cncc(C(=O)Nc2cccc(C(F)(F)F)c2)c1)Nc1ccc(N2CCOCC2)cn1]

(E)-3-(5-(3-(trifluoromethyl)phenylcarbamoyl)pyridin-3-yl)acrylic acid (Int-104) was reacted with 2-amino-5-morpholinopyridine using the experimental conditions outlined in the General Procedure I to give Compound 80 as an off-white solid in 6% yield (4.2 mg). ¹H-NMR (400 MHz, DMSO-d₆): δ 10.87 (s, 1H), 10.59 (s, 1H), 9.11 (d, J=2.0 Hz, 1H), 8.99 (d, J=2.1 Hz, 1H), 8.50 (t, J=2.1 Hz, 1H), 8.24 (bs, 1H), 8.13 (d, J=9.3 Hz, 1H), 8.09-8.05 (m, 2H), 7.72 (d, J=15.9 Hz, 1H), 7.65 (t, J=8.0 Hz, 1H), 7.51 (d, J=7.9 Hz, 1H), 7.46 (dd, J=9.2, 3.2 Hz, 1H), 7.24 (d, J=15.8 Hz, 1H), 3.75 (t, J=4.8 Hz, 4H), 3.30 (s, 3H), 3.13 (t, J=4.8 Hz, 4H) ppm; MS m/z=497.9 [M+H]⁺.

### (E)-3-(3-((5-(Morpholine-4-carbonyl)pyridin-2-yl)amino)-3-oxoprop-1-en-1-yl)-N-(3-(trifluoromethyl)phenyl)benzamide (Compound 81)

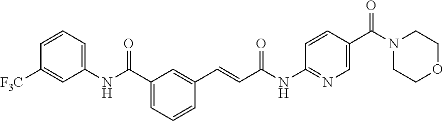 [SMILES: O=C(/C=C/c1cccc(C(=O)Nc2cccc(C(F)(F)F)c2)c1)Nc1ccc(C(=O)N2CCOCC2)cn1]

(E)-3-(3-((3-(trifluoromethyl)phenyl)carbamoyl)phenyl)acrylic acid Int-12 was reacted with (6-Aminopyridin-3-yl)(morpholino)methanone (Int-105) using the experimental conditions outlined in the General Procedure I to give Compound 81 as an off-white solid in 13% yield (6.7 mg). The NMR spectrum shows rotamers: ¹H-NMR (400 MHz, MeOD): δ 8.44 (d, J=1.8 Hz, 0.6H), 8.37 (d, J=8.7 Hz, 0.6H), 8.23-8.16 (m, 2.4H), 8.00-7.94 (m, 2.4H), 7.90-7.81 (m, 2.2H), 7.74 (d, J=16.1 Hz, 0.4H), 7.62-7.53 (m, 2.4H), 7.46-7.43 (m, 1H), 7.02 (d, J=15.7 Hz, 0.6H), 6.64 (d, J=16.0 Hz, 0.4H), 3.64 (bs, 6H), 3.28 (bs, 2H) ppm; MS m/z=525.0 [M+H]⁺.

### (E)-N-(3-(3-((6-Morpholinopyridazin-3-yl)amino)-3-oxoprop-1-en-1-yl)phenyl)-3-(trifluoromethyl)benzamide (Compound 82)

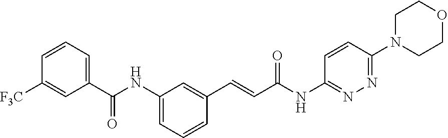 [SMILES: O=C(/C=C/c1cccc(NC(=O)c2cccc(C(F)(F)F)c2)c1)Nc1ccc(N2CCOCC2)nn1]

(E)-3-(3-(3-(trifluoromethyl)benzamido)phenyl)acrylic acid Int-5 was reacted with 3-amino-6-morpholinopyridazine using the experimental conditions outlined in the General Procedure I to give Compound 82 as an orange solid in 17% yield (45.3 mg). ¹H-NMR (400 MHz, DMSO-d₆): δ 11.04 (s, 1H), 10.63 (s, 1H), 8.33 (s, 1H), 8.29 (d, J=7.9 Hz, 1H), 8.25 (d, J=9.8 Hz, 1H), 8.13 (s, 1H), 8.00 (d, J=7.9 Hz, 1H), 7.82 (t, J=7.8 Hz, 1H), 7.78-7.74 (m, 1H), 7.62 (d, J=15.7 Hz, 1H), 7.47 (t, J=7.9 Hz, 1H), 7.42-7.36 (m, 2H), 7.06 (d, J=15.7 Hz, 1H), 3.74 (t, J=4.8 Hz, 4H), 3.49 (t, J=4.8 Hz, 4H) ppm; MS m/z=498.7 [M+H]⁺.

### (E)-N-Methyl-3-(3-oxo-3-((2-(thiazol-2-yl)phenyl)amino)prop-1-en-1-yl)-N-(3-(trifluoromethyl)phenyl)benzamide (Compound 83)

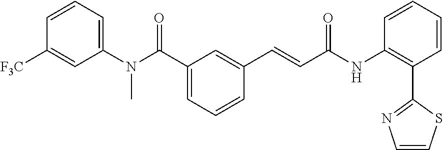 [SMILES: CN(C(=O)c1cccc(/C=C/C(=O)Nc2ccccc2-c2nccs2)c1)c1cccc(C(F)(F)F)c1]

(E)-3-(3-(methyl(3-(trifluoromethyl)phenyl)carbamoyl)phenyl)acrylic acid Int-14 was reacted with 2-(thiazol-2-yl)aniline Int-83 using the experimental conditions outlined in the General Procedure I to give Compound 83 as a light brown solid in 51% yield. ¹H-NMR (400 MHz, DMSO-d₆): δ 12.17 (s, 1H), 8.58 (d, J=8.2 Hz, 1H), 8.13 (d, J=3.4 Hz, 1H), 7.97 (dd, J=7.9, 1.3 Hz, 1H), 7.92 (d, J=3.3 Hz, 1H), 7.72-7.68 (m, 3H), 7.59-7.49 (m, 5H), 7.38-7.31 (m, 2H), 7.27 (td, J=7.6, 0.9 Hz, 1H), 6.81 (d, J=15.7 Hz, 1H), 3.45 (s, 3H) ppm; MS m/z=508.7 [M+H]⁺.

### (E)-N-Methyl-3-(3-oxo-3-((2-(5-(pyridin-4-yl)-1,3,4-oxadiazol-2-yl)phenyl)amino)prop-1-en-1-yl)-N-(3-(trifluoromethyl)phenyl)benzamide (Compound 84)

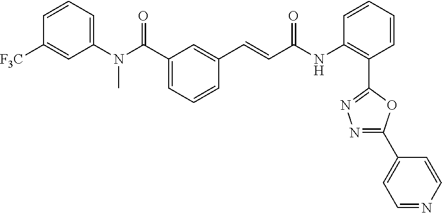 [SMILES: CN(C(=O)c1cccc(/C=C/C(=O)Nc2ccccc2-c2nnc(-c3ccncc3)o2)c1)c1cccc(C(F)(F)F)c1]

(E)-3-(3-(methyl(3-(trifluoromethyl)phenyl)carbamoyl)phenyl)acrylic acid Int-14 was reacted with 2-(5-pyridin-4-yl-1,3,4-oxadiazol-2-yl)aniline using the experimental conditions outlined in the General Procedure I to give Compound 84 as a brown solid in 62% yield. ¹H-NMR (400 MHz, MeOD): δ 8.88-8.82 (m, 2H), 8.67 (d, J=8.4 Hz, 1H), 8.22 (dd, J=7.9, 1.1 Hz, 1H), 8.19-8.16 (m, 2H), 7.71-7.44 (m, 9H), 7.45-7.29 (m, 3H), 6.70 (d, J=15.6 Hz, 1H), 3.55 (s, 3H) ppm; MS m/z=570.1 [M+H]⁺.

### (E)-N-Methyl-3-(3-((6-morpholinopyridazin-3-yl)amino)-3-oxoprop-1-en-1-yl)-N-(3-(trifluoromethyl)phenyl)benzamide (Compound 85)

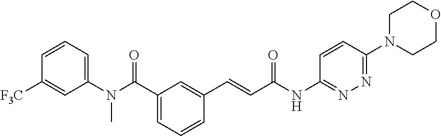 [SMILES: CN(C(=O)c1cccc(/C=C/C(=O)Nc2ccc(N3CCOCC3)nn2)c1)c1cccc(C(F)(F)F)c1]

(E)-3-(3-(methyl(3-(trifluoromethyl)phenyl)carbamoyl)phenyl)acrylic acid Int-14 was reacted with 3-amino-6-morpholinopyridazine using the experimental conditions outlined in the General Procedure I to give Compound 85 as an orange solid in 50% yield. ¹H-NMR (400 MHz, DMSO-d₆): δ 10.94 (s, 1H), 8.22 (d, J=9.8 Hz, 1H), 7.62 (s, 1H), 7.58 (s, 1H), 7.58-7.48 (m, 5H), 7.39 (d, J=9.9 Hz, 1H), 7.31 (t, J=7.6 Hz, 1H), 7.25 (d, J=7.7 Hz, 1H), 6.97 (d, J=15.7 Hz, 1H), 3.74 (t, J=4.8 Hz, 4H), 3.49 (t, J=4.9 Hz, 4H), 3.43 (s, 3H) ppm; MS m/z=512.7 [M+H]⁺.

### (E)-3-(3-((2-(1,2,4-Oxadiazol-3-yl)phenyl)amino)-3-oxoprop-1-en-1-yl)-N-methyl-N-(3-(trifluoromethyl)phenyl)benzamide (Compound 86)

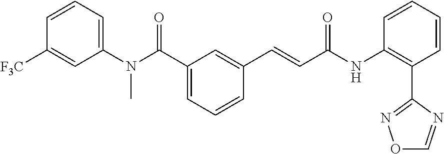 [SMILES: CN(C(=O)c1cccc(/C=C/C(=O)Nc2ccccc2-c2ncon2)c1)c1cccc(C(F)(F)F)c1]

(E)-3-(3-(methyl(3-(trifluoromethyl)phenyl)carbamoyl)phenyl)acrylic acid Int-14 was reacted with 2-(1,2,4-oxadiazol-3-yl)aniline Int-93 using experimental conditions outlined in the General Procedure I to give Compound 86 as a white solid in 16% yield. ¹H-NMR (400 MHz, MeOD): δ 8.31 (d, J=8.5 Hz, 1H), 7.76 (d, J=15.7 Hz, 1H), 7.71 (ddd, J=7.8, 1.5, 0.4 Hz, 1H), 7.68-7.64 (m, 2H), 7.62-7.58 (m, 1H), 7.56-7.45 (m, 5H), 7.34-7.30 (m, 2H), 7.26 (td, J=7.7, 1.0 Hz, 1H), 6.68 (d, J=15.7 Hz, 1H), 3.53 (s, 3H) ppm; MS m/z=493.1 [M+H]⁺.

### (E)-3-(3-((2-(1,3,4-Thiadiazol-2-yl)phenyl)amino)-3-oxoprop-1-en-1-yl)-N-methyl-N-(3-(trifluoromethyl)phenyl)benzamide (Compound 87)

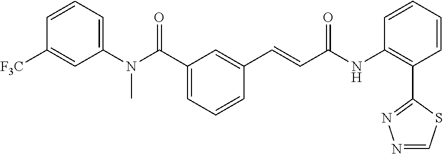 [SMILES: CN(C(=O)c1cccc(/C=C/C(=O)Nc2ccccc2-c2nncs2)c1)c1cccc(C(F)(F)F)c1]

(E)-3-(3-(methyl(3-(trifluoromethyl)phenyl)carbamoyl)phenyl)acrylic acid Int-14 was reacted with 2-(1,3,4-thiadiazol-2-yl)aniline Int-89 using experimental conditions outlined in the General Procedure I to give Compound 87 as an orange solid in 35% yield. ¹H-NMR (400 MHz, CDCl₃): δ 11.95 (s, 1H), 9.15 (s, 1H), 8.94 (dd, J=8.5, 0.7 Hz, 1H), 7.71 (dd, J=7.9, 1.1 Hz, 1H), 7.65 (d, J=15.6 Hz, 1H), 7.58-7.51 (m, 3H), 7.48-7.43 (m, 2H), 7.38-7.25 (m, 4H), 7.19 (td, J=7.6, 0.8 Hz, 1H), 6.57 (d, J=15.6 Hz, 1H), 3.55 (s, 3H) ppm; MS m/z=509.1 [M+H]⁺.

### (E)-N-(3-(3-((6-Morpholinopyrimidin-4-yl)amino)-3-oxoprop-1-en-1-yl)phenyl)-3-(trifluoromethyl)benzamide (Compound 88)

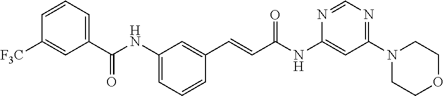 [SMILES: O=C(/C=C/c1cccc(NC(=O)c2cccc(C(F)(F)F)c2)c1)Nc1cc(N2CCOCC2)ncn1]

(E)-3-(3-(3-(trifluoromethyl)benzamido)phenyl)acrylic acid Int-5 was reacted with 4-amino-6-morpholinopyrimidine using experimental conditions outlined in the General Procedure I to give Compound 88 as a white solid in 5% yield. ¹H-NMR (400 MHz, MeOD): δ 8.34-8.29 (m, 2H), 8.23 (d, J=7.7 Hz, 1H), 8.08 (s, 1H), 7.92 (d, J=7.3 Hz, 1H), 7.81-7.71 (m, 3H), 7.67-7.59 (m, 1H), 7.52-7.43 (m, 2H), 6.90 (d, J=15.7 Hz, 1H), 3.78 (t, J=4.8 Hz, 4H), 3.65 (t, J=4.8 Hz, 4H) ppm; MS m/z=497.9 [M+H]⁺.

### (E)-3-(3-((6-Methoxypyridazin-3-yl)amino)-3-oxoprop-1-en-1-yl)-N-methyl-N-(3-(trifluoromethyl)phenyl)benzamide (Compound 89)

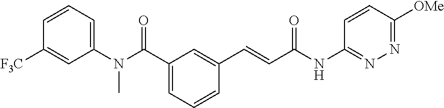 [SMILES: COc1ccc(NC(=O)/C=C/c2cccc(C(=O)N(C)c3cccc(C(F)(F)F)c3)c2)nn1]

(E)-3-(3-(methyl(3-(trifluoromethyl)phenyl)carbamoyl)phenyl)acrylic acid Int-14 was reacted with 3-amino-6-methoxypyridazine using experimental conditions outlined in the General Procedure I to give Compound 89 as an orange solid in 40% yield. ¹H-NMR (400 MHz, DMSO-d₆): δ 11.13 (s, 1H), 8.38 (d, J=9.6 Hz, 1H), 7.62 (s, 1H), 7.59 (s, 1H), 7.56-7.49 (m, 5H), 7.33 (d, J=7.6 Hz, 1H), 7.29 (d, J=9.5 Hz, 1H), 7.26 (d, J=7.8 Hz, 1H), 6.99 (d, J=15.8 Hz, 1H), 4.00 (s, 3H), 3.43 (s, 3H) ppm; MS m/z=457.0 [M+H]⁺.

### (E)-N-(3-(3-((6-Methoxypyridazin-3-yl)amino)-3-oxoprop-1-en-1-yl)phenyl)-3-(trifluoromethyl)benzamide (Compound 90)

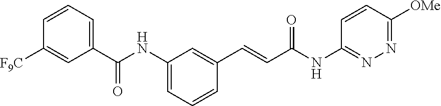

(E)-3-(3-(3-(trifluoromethyl)benzamido)phenyl)acrylic acid Int-5 was reacted with 3-amino-6-methoxypyridazine using experimental conditions outlined in the General Procedure I to give Compound 90 as an off-white solid in 7% yield. ¹H-NMR (400 MHz, DMSO-d₆): δ 11.23 (s, 1H), 10.63 (s, 1H), 8.41 (d, J=9.6 Hz, 1H), 8.33 (s, 1H), 8.29 (d, J=7.5 Hz, 1H), 8.15 (s, 1H), 8.00 (d, J=7.8 Hz, 1H), 7.82 (t, J=7.8 Hz, 1H), 7.77 (d, J=8.9 Hz, 1H), 7.65 (d, J=15.7 Hz, 1H), 7.47 (t, J=7.8 Hz, 1H), 7.40 (d, J=7.7 Hz, 1H), 7.29 (d, J=9.5 Hz, 1H), 7.08 (d, J=15.7 Hz, 1H), 4.00 (s, 3H) ppm; MS m/z=443.0 [M+H]⁺.

### (E)-N-Methyl-3-(3-((6-morpholinopyrimidin-4-yl)amino)-3-oxoprop-1-en-1-yl)-N-(3-(trifluoromethyl)phenyl)benzamide (Compound 91)

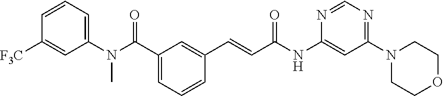 [SMILES: CN(C(=O)c1cccc(/C=C/C(=O)Nc2cc(N3CCOCC3)ncn2)c1)c1cccc(C(F)(F)F)c1]

(E)-3-(3-(methyl(3-(trifluoromethyl)phenyl)carbamoyl)phenyl)acrylic acid Int-14 was reacted with 4-amino-6-morpholinopyrimidine using experimental conditions outlined in the General Procedure I to give Compound 91 as a yellow solid in 40% yield. ¹H-NMR (400 MHz, DMSO-d₆): δ 10.66 (s, 1H), 8.33 (d, J=0.9 Hz, 1H), 7.62 (s, 1H), 7.57 (s, 1H), 7.56-7.52 (m, 5H), 7.49 (d, J=15.7 Hz, 1H), 7.31 (t, J=7.6 Hz, 1H), 7.25 (d, J=7.5 Hz, 1H), 6.97 (d, J=15.7 Hz, 1H), 3.68 (t, J=4.8 Hz, 4H), 3.55 (t, J=4.7 Hz, 4H), 3.43 (s, 3H) ppm; MS m/z=512.2 [M+H]⁺.

### (E)-N-Methyl-N-(3-(3-oxo-3-(pyrimidin-4-ylamino)prop-1-en-1-yl)phenyl)-3-(trifluoromethyl)benzamide (Compound 92)

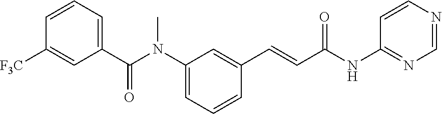 [SMILES: CN(C(=O)c1cccc(C(F)(F)F)c1)c1cccc(/C=C/C(=O)Nc2ccncn2)c1]

(E)-3-(3-(N-methyl-3-(trifluoromethyl)benzamido)phenyl)acrylic acid Int-7 was reacted with 4-aminopyrimidine using experimental conditions outlined in the General Procedure I to give Compound 92 as a light-yellow solid in 38% yield. ¹H-NMR (400 MHz, DMSO-d₆): δ 11.08 (s, 1H), 8.90 (d, J=1.3 Hz, 1H), 8.68 (d, J=5.9 Hz, 1H), 8.18 (dd, J=5.8, 1.3 Hz, 1H), 7.65 (d, J=7.8 Hz, 1H), 7.62-7.56 (m, 3H), 7.53-7.43 (m, 3H), 7.35 (t, J=7.8 Hz, 1H), 7.27 (d, J=7.3 Hz, 1H), 6.98 (d, J=15.8 Hz, 1H), 3.42 (s, 3H) ppm; MS m/z=427.0 [M+H]⁺.

### (E)-3-(3-Oxo-3-((2-(thiazol-2-yl)phenyl)amino)prop-1-en-1-yl)-N-(3-(trifluoromethyl)phenyl) benzamide (Compound 93)

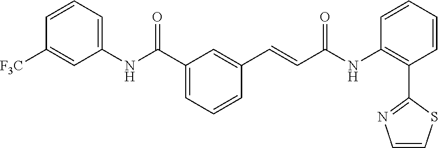 [SMILES: O=C(/C=C/c1cccc(C(=O)Nc2cccc(C(F)(F)F)c2)c1)Nc1ccccc1-c1nccs1]

(E)-3-(3-((3-(trifluoromethyl)phenyl)carbamoyl)phenyl)acrylic acid Int-12 was reacted with 2-(thiazol-2-yl)aniline Int-83 using experimental conditions outlined in the General Procedure I to give Compound 93 as a yellow solid in 55% yield. ¹H-NMR (400 MHz, DMSO-d₆): δ 12.18 (s, 1H), 10.66 (s, 1H), 8.59 (d, J=8.2 Hz, 1H), 8.32 (s, 1H), 8.28 (s, 1H), 8.12 (d, J=3.3 Hz, 1H), 8.09 (d, J=8.1 Hz, 1H), 8.04-7.95 (m, 3H), 7.92 (d, J=3.3 Hz, 1H), 7.76 (d, J=15.7 Hz, 1H), 7.69-7.60 (m, 2H), 7.56-7.44 (m, 2H), 7.27 (td, J=8.2, 1.0 Hz, 1H), 7.04 (d, J=15.7 Hz, 1H) ppm; MS m/z=494.1 [M+H]⁺.

### (E)-3-(3-((2-(1,3,4-Thiadiazol-2-yl)phenyl)amino)-3-oxoprop-1-en-1-yl)-N-(3-(trifluoromethyl)phenyl)benzamide (Compound 94)

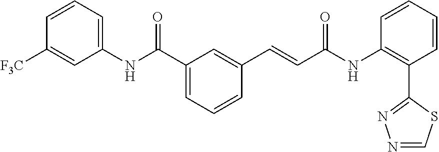 [SMILES: O=C(/C=C/c1cccc(C(=O)Nc2cccc(C(F)(F)F)c2)c1)Nc1ccccc1-c1nncs1]

(E)-3-(3-((3-(trifluoromethyl)phenyl)carbamoyl)phenyl)acrylic acid Int-12 was reacted with 2-(1,3,4-thiadiazol-2-yl)aniline Int-89 using experimental conditions outlined in the General Procedure I to give Compound 94 as an orange solid in 19% yield. ¹H-NMR (400 MHz, CDCl₃): δ 12.03 (s, 1H), 9.15 (s, 1H), 8.94 (d, J=8.5 Hz, 1H), 8.30 (s, 1H), 8.10 (s, 1H), 8.02 (s, 1H), 7.95 (d, J=7.8 Hz, 1H), 7.91 (d, J=7.8 Hz, 1H), 7.82 (d, J=15.6 Hz, 1H), 7.77 (d, J=7.8 Hz, 1H), 7.71 (dd, J=7.9, 1.4 Hz, 1H), 7.56-7.48 (m, 3H), 7.43 (d, J=7.9 Hz, 1H), 7.20 (td, J=7.6, 0.9 Hz, 1H), 6.82 (d, J=15.6 Hz, 1H) ppm; MS m/z=495.1 [M+H]⁺.

### (E)-N-Methyl-3-(3-oxo-3-(quinolin-8-ylamino)prop-1-en-1-yl)-N-(3-(trifluoromethyl)phenyl) benzamide (Compound 95)

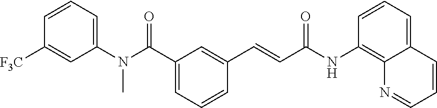 [SMILES: CN(C(=O)c1cccc(/C=C/C(=O)Nc2cccc3cccnc23)c1)c1cccc(C(F)(F)F)c1]

(E)-3-(3-(methyl(3-(trifluoromethyl)phenyl)carbamoyl)phenyl)acrylic acid Int-14 was reacted with 8-aminoquinoline using experimental conditions outlined in the General Procedure I to give Compound 95 as a yellow solid in 79% yield. ¹H-NMR (400 MHz, DMSO-d₆): δ 10.39 (s, 1H), 8.97 (dd, J=4.2, 1.6 Hz, 1H), 8.79 (dd, J=7.6, 1.0 Hz, 1H), 8.44 (dd, J=8.3, 1.5 Hz, 1H), 7.79-7.66 (m, 5H), 7.62 (t, J=8.0 Hz, 1H), 7.59-7.42 (m, 5H), 7.32 (t, J=7.5 Hz, 1H), 7.26 (d, J=7.4 Hz, 1H), 3.44 (s, 3H) ppm; MS m/z=476.1 [M+H]⁺.

### (E)-N-Methyl-N-(3-(3-oxo-3-(quinolin-8-ylamino)prop-1-en-1-yl)phenyl)-3-(trifluoromethyl) benzamide (Compound 96)

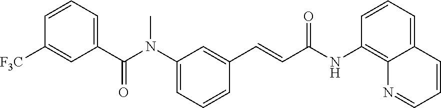 [SMILES: CN(C(=O)c1cccc(C(F)(F)F)c1)c1cccc(/C=C/C(=O)Nc2cccc3cccnc23)c1]

(E)-3-(3-(N-methyl-3-(trifluoromethyl)benzamido)phenyl)acrylic acid Int-7 was reacted with 8-aminoquinoline using experimental conditions outlined in the General Procedure I to give Compound 96 as a yellow solid in 68% yield. ¹H-NMR (400 MHz, DMSO-d₆): δ 10.36 (s, 1H), 8.97 (dd, J=4.2, 1.7 Hz, 1H), 8.79 (dd, J=7.7, 1.2 Hz, 1H), 8.44 (dd, J=8.3, 1.6 Hz, 1H), 7.78-7.39 (m, 11H), 7.33 (t, J=7.8 Hz, 1H), 7.23 (d, J=8.1 Hz, 1H), 3.45 (s, 3H) ppm; MS m/z=476.0 [M+H]⁺.

### (E)-3-(3-(5-Ethynylpyridin-2-ylamino)-3-oxoprop-1-enyl)-N-methyl-N-(3-(trifluoromethyl)phenyl)benzamide (Compound 97)

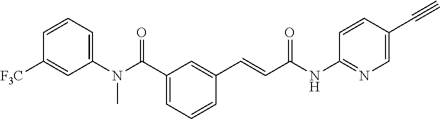 [SMILES: C#Cc1ccc(NC(=O)/C=C/c2cccc(C(=O)N(C)c3cccc(C(F)(F)F)c3)c2)nc1]

50% T₃P solution in EtOAc (15 mL) was added to a stirred solution of (E)-3-(3-(methyl(3-(trifluoromethyl)phenyl)carbamoyl)phenyl)acrylic acid Int-14 (1.0 g, 2.65 mmol) in pyridine (5.0 mL), and the reaction mixture was stirred for 15 min. Then, 5-ethynylpyridin-2-amine Int-106 (0.32 g, 2.65 mmol) was added at 0° C. and the reaction mixture was stirred at room temperature for 16 hrs. The solution was passed through a neutral alumina pad twice to remove the excess of T₃P. The crude product was purified by preparative HPLC (X SELECT C18 (19*250*5 um) using 10 mM ammonium bicarbonate in water and acetonitrile to afford compound 97 as an off-white solid in 51% yield (607.0 mg). ¹H-NMR (400 MHz, DMSO-d₆): δ 10.90 (bs, 1H), 8.47 (d, J=2.2 Hz, 1H), 8.24 (d, J=8.7 Hz, 1H), 7.92 (dd, J=8.7, 2.3 Hz, 1H), 7.61 (s, 1H), 7.59 (s, 1H), 7.55-7.51 (m, 5H), 7.32 (t, J=7.6 Hz, 1H), 7.25 (d, J=7.5 Hz, 1H), 6.99 (d, J=15.7 Hz, 1H), 4.35 (s, 1H), 3.43 (s, 3H) ppm; MS m/z=450.4 [M+H]⁺.

### (E)-N-(5-(3-(5-(4-(2-methoxyethyl)piperazin-1-yl)pyridin-2-ylamino)-3-oxoprop-1-enyl)pyridin-3-yl)-3-(trifluoromethyl)benzamide (Compound 98)

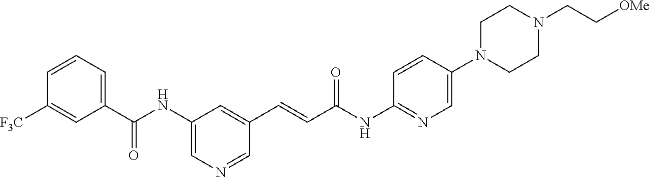 [SMILES: COCCN1CCN(c2ccc(NC(=O)/C=C/c3cncc(NC(=O)c4cccc(C(F)(F)F)c4)c3)nc2)CC1]

DIPEA (0.27 mL 1.49 mmol) was added at 0° C. to a stirred solution of (E)-ethyl 3-(5-(3-(trifluoromethyl)benzamido)pyridin-3-yl)acrylate Int-108 (0.10 g, 0.30 mmol) in DMF (2 mL), followed by HATU (0.17 g, 0.45 mmol) and the reaction mixture was stirred for 15 min. Then, 5-(4-(2-methoxyethyl)piperazin-1-yl)pyridin-2-amine Int-107 (0.085 g, 0.35 mmol) was added dropwise at 0° C. and the reaction mixture was stirred at room temperature for 36 hrs. Then the reaction mixture was diluted with water (10 mL) and extracted with Ethyl Acetate (2×25 mL). The combined organic layers were washed with water (3×10 mL), dried over anhydrous sodium sulfate and concentrated under reduced pressure. The crude product was purified by column chromatography (100-200 silica) using 10% Ethyl Acetate in Hexane as eluent to afford 0.048 g of (E)-N-(5-(3-(5-(4-(2-methoxyethyl)piperazin-1-yl)pyridin-2-ylamino)-3-oxoprop-1-enyl)pyridin-3-yl)-3-(trifluoromethyl)benzamide Compound 98 as a pale yellow solid in 30% yield. ¹H-NMR (400 MHz, DMSO-d₆): δ 10.82 (s, 1H), 10.62 (s, 1H), 8.88 (d, J=2.2 Hz, 1H), 8.62-8.53 (m, 2H), 8.35 (s, 1H), 8.31 (d, J=7.8 Hz, 1H), 8.11 (d, J=9.3 Hz, 1H), 8.08-8.00 (m, 2H), 7.84 (t, J=7.8 Hz, 1H), 7.64 (d, J=15.7 Hz, 1H), 7.44 (dd, J=9.1, 2.5 Hz, 1H), 7.15 (d, J=15.7 Hz, 1H), 3.48 (bs, 2H), 3.36 (bs, 2H), 3.25 (s, 3H), 3.14 (bs, 4H), 2.57 (bs, 4H) ppm; MS (ESI) m/z=555.55 [M+H]⁺.

### (E)-5-(3-(5-(4-(2-Methoxyethyl)piperazin-1-yl)pyridin-2-ylamino)-3-oxoprop-1-enyl)-N-(3-(trifluoromethyl)phenyl)nicotinamide (Compound 99)

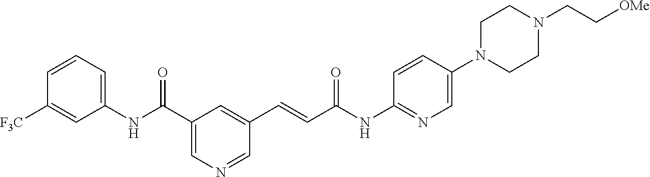 [SMILES: COCCN1CCN(c2ccc(NC(=O)/C=C/c3cncc(C(=O)Nc4cccc(C(F)(F)F)c4)c3)nc2)CC1]

DIPEA (0.27 mL 1.49 mmol) was added to a stirred solution of (E)-3-(5-(3-(trifluoromethyl)phenyl carbamoyl)pyridin-3-yl)acrylic acid (Int-104) (0.10 g, 0.30 mmol) in DMF (2 mL) at 0° C., followed by HATU (0.17 g, 0.45 mmol), and the reaction mixture was stirred for 15 min. Then, 5-(4-(2-methoxyethyl)piperazin-1-yl)pyridin-2-amine (Int-107) (0.085 g, 0.35 mmol) was added at 0° C. and the reaction mixture was stirred at room temperature for 36 hrs. Then the reaction mixture was diluted with water (10 mL) and extracted with Ethyl Acetate (2×25 mL). Combined organic layers were washed with water (3×10 mL), dried over anhydrous sodium sulfate and concentrated under reduced pressure. The crude product was purified by column chromatography (100-200 silica) using 10% Ethyl Acetate in Hexane as eluent to afford 0.016 g of (E)-5-(3-(5-(4-(2-methoxyethyl) piperazin-1-yl)pyridin-2-ylamino)-3-oxoprop-1-enyl)-N-(3-(trifluoromethyl) phenyl)nicotinamide Compound 99 as a pale-yellow solid in 10% yield. ¹H-NMR (400 MHz, DMSO-d₆): δ 10.85 (s, 1H), 10.58 (s, 1H), 9.10 (d, J=1.8 Hz, 1H), 8.99 (d, J=1.7 Hz, 1H), 8.49 (t, J=2.0 Hz, 1H), 8.24 (s, 1H), 8.11 (d, J=9.0 Hz, 1H), 8.07-8.03 (m, 2H), 7.72 (d, J=15.8 Hz, 1H), 7.65 (t, J=7.9 Hz, 1H), 7.51 (d, J=7.8 Hz, 1H), 7.44 (dd, J=9.1, 2.9 Hz, 1H), 7.23 (d, J=15.8 Hz, 1H), 3.48 (t, J=5.7 Hz, 2H), 3.25 (s, 3H), 3.15 (bs, 4H), 2.59 (bs, 6H); MS (ESI) m/z=555.1 [M+H]⁺.

### (E)-N-(3-(3-(5-(4-(2-Methoxyethyl)piperazin-1-yl)pyridin-2-ylamino)-3-oxoprop-1-enyl)phenyl)-3-(trifluoromethyl)benzamide (Compound 100)

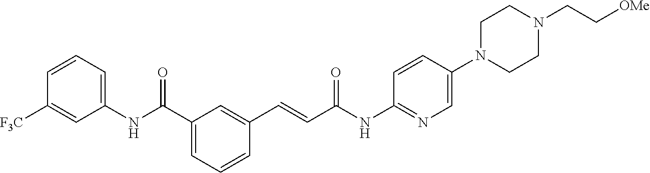 [SMILES: COCCN1CCN(c2ccc(NC(=O)/C=C/c3cccc(C(=O)Nc4cccc(C(F)(F)F)c4)c3)nc2)CC1]

DIPEA (0.27 mL, 1.49 mmol) was added to a stirred solution of (E)-3-(3-(3-(trifluoromethyl) benzamido)phenyl)acrylic acid Int-12 (0.10 g, 0.30 mmol) in DMF (2 mL) at 0° C., followed by HATU (0.17 g, 0.45 mmol) at 0° C., and the reaction mixture was stirred for 15 min. Then, 5-(4-(2-methoxyethyl)piperazin-1-yl)pyridin-2-amine (Int-107) (0.09 g, 0.35 mmol) was added dropwise at 0° C. and it was stirred at room temperature for 36 hrs. The reaction mixture was diluted with water (10 mL) and extracted with Ethyl Acetate (2×25 mL). Combined organic layers were washed with water (3×10 mL), dried over sodium sulfate and concentrated under reduced pressure. The crude product was purified by column chromatography (100-200 silica) using 10% Ethyl Acetate in hexane as eluent to afford 0.023 g of (E)-N-(3-(3-(5-(4-(2-methoxyethyl)piperazin-1-yl)pyridin-2-ylamino)-3-oxoprop-1-enyl)phenyl)-3-(trifluoromethyl)benzamide Compound 100 as a pale-yellow solid in 15% yield. ¹H-NMR (400 MHz, DMSO-d₆): δ 10.61 (s, 1H), 10.56 (s, 1H), 8.33 (s, 1H), 8.29 (d, J=7.8 Hz, 1H), 8.14-8.10 (m, 2H), 8.03 (d, J=2.9 Hz, 1H), 7.99 (d, J=7.8 Hz, 1H), 7.81 (t, J=7.8 Hz, 1H), 7.76 (d, J=8.0 Hz, 1H), 7.57 (d, J=15.6 Hz, 1H), 7.46 (t, J=7.1 Hz, 1H), 7.43 (dd, J=9.8, 2.3 Hz, 1H), 7.37 (d, J=7.8 Hz, 1H), 7.03 (d, J=15.7 Hz, 1H), 3.47 (t, J=5.8 Hz, 2H), 3.25 (s, 3H), 3.14 (t, J=4.9 Hz, 4H), 2.57 (t, J=4.9 Hz, 4H), 2.52 (t, J=5.8 Hz, 2H) ppm; MS m/z=554.6 [M+H]⁺.

### (E)-N-(3-(3-((5-(4-(2-Methoxyethyl)piperazin-1-yl)pyridin-2-yl)amino)-3-oxoprop-1-en-1-yl)phenyl)-3-(trifluoromethyl)benzamide (Compound 101)

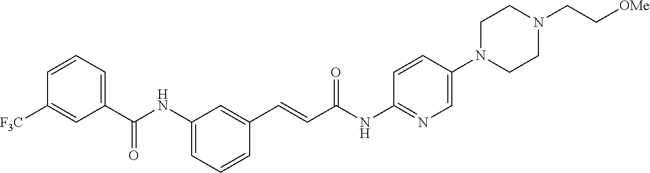 [SMILES: COCCN1CCN(c2ccc(NC(=O)/C=C/c3cccc(NC(=O)c4cccc(C(F)(F)F)c4)c3)nc2)CC1]

DIPEA (0.27 mL 1.49 mmol) was added to a stirred solution of (E)-3-(3-(3-(trifluoromethyl) phenylcarbamoyl)phenyl)acrylic acid Int-5 (0.10 g, 0.30 mmol) in DMF (2 mL) at 0° C., followed by HATU (0.17 g, 0.45 mmol), and the reaction mixture was stirred for 15 min. Then, 5-(4-(2-methoxyethyl)piperazin-1-yl)pyridin-2-amine (Int-107) (85.0 mg, 0.35 mmol) was added at 0° C. and the reaction mixture was stirred at room temperature for 36 hrs. Then the reaction mixture was diluted with water (10 mL) and extracted with Ethyl Acetate (2×25 mL). Combined organic layers were washed with water (3×10 mL), dried over anhydrous sodium sulfate and concentrated under reduced pressure. The crude product was purified by column chromatography (100-200 silica) using 10% Ethyl Acetate in Hexane as eluent to afford 0.05 g of (E)-3-(3-(5-(4-(2-methoxyethyl) piperazin-1-yl)pyridin-2-ylamino)-3-oxoprop-1-enyl)-N-(3-(trifluoromethyl) phenyl)benzamide Compound 101 as a pale yellow solid in 31% yield. ¹H-NMR (400 MHz, DMSO-d₆): δ 10.66 (s, 1H), 10.53 (s, 1H), 8.25 (s, 1H), 8.19 (s, 1H), 8.16-8.06 (m, 2H), 8.04 (d, J=2.7 Hz, 1H), 7.98 (d, J=7.7 Hz, 1H), 7.83 (d, J=8.1 Hz, 1H), 7.73-7.59 (m, 3H), 7.48 (d, J=7.6 Hz, 1H), 7.43 (dd, J=9.1, 2.9 Hz, 1H), 7.14 (d, J=15.8 Hz, 1H), 3.47 (t, J=5.7 Hz, 2H), 3.25 (s, 3H), 3.14 (t, J=4.6 Hz, 4H), 2.57 (t, J=4.6 Hz, 4H), 2.55-2.50 (m, 2H) ppm; MS m/z=554.6 [M+H]⁺.

### (E)-3-(3-Oxo-3-((2-(pyridin-2-yl)phenyl)amino)prop-1-en-1-yl)-N-(3-(trifluoromethyl) phenyl)benzamide (Compound 1

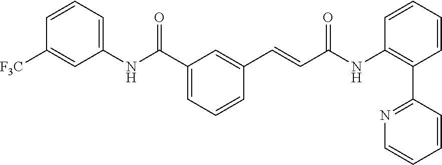 [SMILES: O=C(/C=C/c1cccc(C(=O)Nc2cccc(C(F)(F)F)c2)c1)Nc1ccccc1-c1ccccn1]

(E)-3-(3-((3-(trifluoromethyl)phenyl)carbamoyl)phenyl)acrylic acid Int-12 was reacted with 2-(pyridin-2-yl)aniline (Int-100) using experimental conditions outlined in the General Procedure I to give Compound 102 as a yellow solid in 15% yield. ¹H-NMR (400 MHz, CDCl₃): δ 12.68 (s, 1H), 9.03 (s, 1H), 8.66 (d, J=4.8 Hz, 1H), 8.56 (d, J=8.2 Hz, 1H), 8.09 (s, 1H), 8.01 (s, 1H), 7.98 (d, J=8.2 Hz, 1H), 7.83 (td, J=7.8, 1.8 Hz, 1H), 7.78 (d, J=7.8 Hz, 1H), 7.73 (d, J=8.1 Hz, 1H), 7.64 (dd, J=7.9, 1.3 Hz, 1H), 7.54 (d, J=15.7 Hz, 1H), 7.50-7.44 (m, 2H), 7.39 (d, J=7.7 Hz, 1H), 7.37-7.30 (m, 2H), 7.27-7.24 (m, 1H), 7.14 (td, J=7.6, 0.9 Hz, 1H), 6.49 (d, J=15.7 Hz, 1H) ppm; MS m/z=488.1 [M+H]⁺.

### (E)-N-(3-(3-((2-Morpholinopyrimidin-5-yl)amino)-3-oxoprop-1-en-1-yl)phenyl)-3-(trifluoromethyl)benzamide (Compound 103)

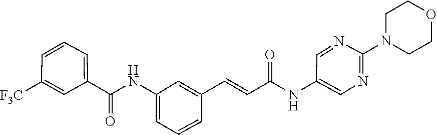 [SMILES: O=C(/C=C/c1cccc(NC(=O)c2cccc(C(F)(F)F)c2)c1)Nc1cnc(N2CCOCC2)nc1]

(E)-3-(3-(3-(trifluoromethyl)benzamido)phenyl)acrylic acid Int-5 was reacted with 2-morpholinopyrimidin-5-amine Int-109 using experimental conditions outlined in the General Procedure I to give Compound 103 as a yellow solid in 56% yield. ¹H-NMR (400 MHz, DMSO-d₆): δ 10.60 (s, 1H), 10.28 (s, 1H), 8.68 (s, 2H), 8.32 (s, 1H), 8.29 (d, J=7.9 Hz, 1H), 8.20 (s, 1H), 7.99 (d, J=7.9 Hz, 1H), 7.81 (t, J=7.8 Hz, 1H), 7.73 (d, J=8.1 Hz, 1H), 7.57 (d, J=15.7 Hz, 1H), 7.46 (t, J=7.8 Hz, 1H), 7.39 (d, J=7.7 Hz, 1H), 6.80 (d, J=15.7 Hz, 1H), 3.66 (s, 8H) ppm; MS m/z=498.0 [M+H]⁺.

### (E)-N-Methyl-N-(3-(3-oxo-3-((2-(5-(pyridin-4-yl)-1,3,4-oxadiazol-2-yl)phenyl)amino)prop-1-en-1-yl)phenyl)-3-(trifluoromethyl)benzamide (Compound 104)

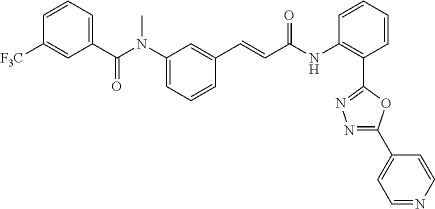 [SMILES: CN(C(=O)c1cccc(C(F)(F)F)c1)c1cccc(/C=C/C(=O)Nc2ccccc2-c2nnc(-c3ccncc3)o2)c1]

(E)-3-(3-(N-methyl-3-(trifluoromethyl)benzamido)phenyl) acrylic acid Int-7 was reacted with 2-(5-pyridin-4-yl-1,3,4-oxadiazol-2-yl)aniline using experimental conditions outlined in the General Procedure I to give Compound 104 as a brown solid in 15% yield. ¹H-NMR (400 MHz, DMSO-d₆): δ 10.76 (s, 1H), 8.93-8.83 (m, 2H), 8.42 (d, J=8.0 Hz, 1H), 8.19 (dd, J=7.9, 1.3 Hz, 1H), 8.14-8.04 (m, 2H), 7.76-7.46 (m, 8H), 7.41 (td, J=8.0, 0.9 Hz, 1H), 7.33 (t, J=7.7 Hz, 1H), 7.24 (d, J=7.3 Hz, 1H), 6.89 (d, J=15.7 Hz, 1H), 3.44 (s, 3H); MS m/z=570.1 [M+H]⁺.

### (E)-N-Methyl-3-(3-((5-morpholinopyridin-2-yl)amino)-3-oxoprop-1-en-1-yl)-N-(3-(trifluoromethyl)phenyl)benzamide (Compound 105)

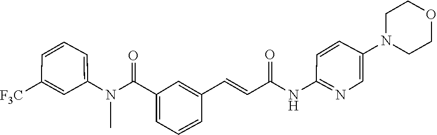

(E)-3-(3-(methyl(3-(trifluoromethyl)phenyl)carbamoyl)phenyl)acrylic acid Int-14 was reacted with 2-amino-5-morpholinopyridine using experimental conditions outlined in the General Procedure I to give Compound 105 as a yellow solid in 26% yield. ¹H-NMR (400 MHz, DMSO-d₆): δ 10.48 (s, 1H), 8.10 (d, J=9.1 Hz, 1H), 8.05 (d, J=2.9 Hz, 1H), 7.61 (s, 1H), 7.56-7.50 (m, 5H), 7.48-7.41 (m, 2H), 7.30 (t, J=7.7 Hz, 1H), 7.24 (d, J=7.7 Hz, 1H), 6.95 (d, J=15.8 Hz, 1H), 3.75 (t, J=4.7 Hz, 4H), 3.43 (s, 3H), 3.12 (t, J=4.7 Hz, 4H) ppm; MS m/z=511.7 [M+H]⁺.

### (E)-N-(3-(3-((5-(4-Ethylpiperazine-1-carbonyl)pyridin-2-yl)amino)-3-oxoprop-1-en-1-yl)phenyl)-3-(trifluoromethyl)benzamide (Compound 106)

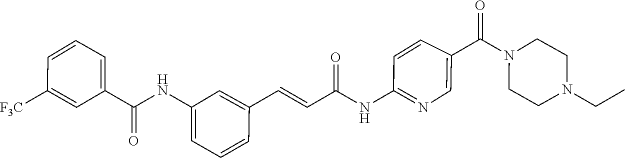 [SMILES: CCN1CCN(C(=O)c2ccc(NC(=O)/C=C/c3cccc(NC(=O)c4cccc(C(F)(F)F)c4)c3)nc2)CC1]

(E)-3-(3-(3-(trifluoromethyl)benzamido)phenyl)acrylic acid Int-5 was reacted with 5-(4-ethylpiperazine-1-carbonyl)pyridin-2-amine using experimental conditions outlined in the General Procedure I to give Compound 106 as an off-white solid in 51% yield. ¹H-NMR (400 MHz, DMSO-d₆): δ 10.71 (s, 1H), 10.62 (s, 1H), 8.84 (d, J=2.5 Hz, 1H), 8.33 (s, 1H), 8.29 (d, J=7.8 Hz, 1H), 8.27-8.23 (m, 2H), 8.00 (d, J=7.8 Hz, 1H), 7.82 (t, J=7.8 Hz, 1H), 7.73 (d, J=8.3 Hz, 1H), 7.65 (d, J=15.7 Hz, 1H), 7.61 (d, J=8.6 Hz, 1H), 7.48 (t, J=7.8 Hz, 1H), 7.42 (d, J=7.7 Hz, 1H), 6.88 (d, J=15.7 Hz, 1H), 3.63-3.59 (m, 2H), 3.49 (t, J=4.6 Hz, 2H), 2.42 (t, J=4.1 Hz, 2H), 2.39-2.27 (m, 4H), 1.00 (t, J=7.2 Hz, 3H); MS m/z=552.5 [M+H]⁺.

### (E)-N-(2-Morpholinoethyl)-6-(3-(3-((3-(trifluoromethyl)phenyl)carbamoyl)phenyl)acrylamido) nicotinamide (Compound 107)

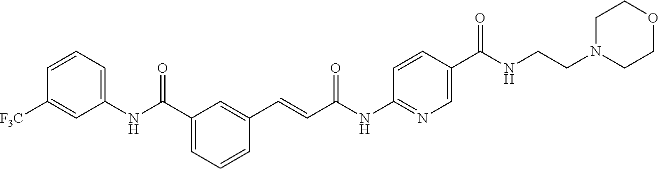 [SMILES: O=C(/C=C/c1cccc(C(=O)Nc2cccc(C(F)(F)F)c2)c1)Nc1ccc(C(=O)NCCN2CCOCC2)cn1]

(E)-6-(3-(3-((3-(trifluoromethyl)phenyl)carbamoyl)phenyl) acrylamido)nicotinic acid (Int-110) was reacted with 2-morpholinoethanamine using a variation of experimental conditions outlined in the General Procedure I with acetonitril used of DMF as a solvent to give Compound 107 as an off-white solid in 49% yield. ¹H-NMR (400 MHz, DMSO-d₆): δ 11.04 (s, 1H), 10.68 (s, 1H), 8.80 (dd, J=2.3, 0.6 Hz, 1H), 8.52 (t, J=5.5 Hz, 1H), 8.32 (d, J=8.7 Hz, 1H), 8.29-8.21 (m, 3H), 8.09 (d, J=8.5 Hz, 1H), 8.01 (d, J=7.8 Hz, 1H), 7.86 (d, J=7.8 Hz, 1H), 7.76 (d, J=15.8 Hz, 1H), 7.66 (d, J=7.8 Hz, 1H), 7.62 (d, J=8.7 Hz, 1H), 7.48 (d, J=7.7 Hz, 1H), 7.19 (d, J=15.8 Hz, 1H), 3.57 (t, J=4.6 Hz, 4H), 3.42-3.37 (m, 2H), 2.50-2.44 (m, 2H), 2.42 (s, 4H); MS m/z=568.1 [M+H]⁺.

### (E)-N-(3-(3-Oxo-3-((2-(5-(pyridin-4-yl)-1,3,4-oxadiazol-2-yl)phenyl)amino)prop-1-en-1-yl)phenyl)-3-(trifluoromethyl)benzamide (Compound 108)

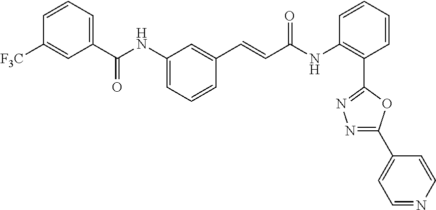 [SMILES: O=C(/C=C/c1cccc(NC(=O)c2cccc(C(F)(F)F)c2)c1)Nc1ccccc1-c1nnc(-c2ccncc2)o1]

(E)-3-(3-(3-(trifluoromethyl)benzamido)phenyl)acrylic acid Int-5 was reacted with 2-(5-pyridin-4-yl-1,3,4-oxadiazol-2-yl)aniline using experimental conditions outlined in the General Procedure I to give Compound 108 as an off-white solid in 11% yield. ¹H-NMR (400 MHz, DMSO-d₆): δ 10.80 (s, 1H), 10.63 (s, 1H), 8.86-8.84 (m, 2H), 8.42 (d, J=8.3 Hz, 1H), 8.34 (s, 1H), 8.30 (d, J=7.7 Hz, 1H), 8.19 (dd, J=8.0, 1.4 Hz, 1H), 8.17 (s, 1H), 8.10-8.04 (m, 2H), 8.00 (d, J=7.8 Hz, 1H), 7.88-7.78 (m, 2H), 7.73-7.69 (m, 1H), 7.67 (d, J=15.8 Hz, 1H), 7.48 (d, J=5.0 Hz, 2H), 7.42 (td, J=7.6, 0.8 Hz, 1H), 6.90 (d, J=15.7 Hz, 1H); MS m/z=556.1 [M+H]⁺.

### (E)-3-(3-Oxo-3-((2-(5-(pyridin-4-yl)-1,3,4-oxadiazol-2-yl)phenyl)amino)prop-1-en-1-yl)-N-(3-(trifluoromethyl)phenyl)benzamide (Compound 109)

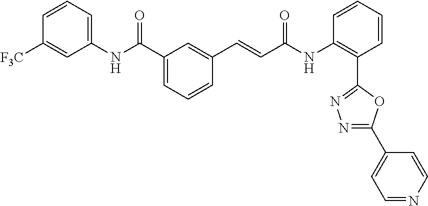 [SMILES: O=C(/C=C/c1cccc(C(=O)Nc2cccc(C(F)(F)F)c2)c1)Nc1ccccc1-c1nnc(-c2ccncc2)o1]

(E)-3-(3-((3-(trifluoromethyl)phenyl)carbamoyl)phenyl)acrylic acid Int-12 was reacted with 2-(5-pyridin-4-yl-1,3,4-oxadiazol-2-yl)aniline using experimental conditions outlined in the General Procedure I to give Compound 109 as a light brown solid in 35% yield. ¹H-NMR (400 MHz, DMSO-d₆): δ 10.82 (s, 1H), 10.67 (s, 1H), 8.88-8.84 (m, 2H), 8.44 (d, J=8.0 Hz, 1H), 8.31 (s, 1H), 8.26 (s, 1H), 8.20 (dd, J=7.9, 1.4 Hz, 1H), 8.12-8.05 (m, 3H), 8.01 (d, J=7.9 Hz, 1H), 7.96 (d, J=7.9 Hz, 1H), 7.77 (d, J=15.7 Hz, 1H), 7.71 (td, J=8.6, 1.5 Hz, 1H), 7.68-7.59 (m, 2H), 7.49 (d, J=7.8 Hz, 1H), 7.44-7.40 (td, J=7.9, 0.9 Hz, 1H), 7.09 (d, J=15.7 Hz, 1H) ppm; MS m/z=556.1 [M+H]⁺.

### (E)-3-(3-((3-(2-Methylpyrimidin-4-yl)phenyl)amino)-3-oxoprop-1-en-1-yl)-N-(3-(trifluoromethyl)phenyl)benzamide (Compound 110)

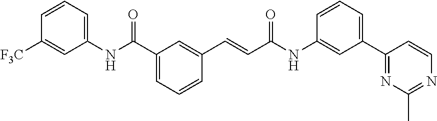 [SMILES: Cc1nccc(-c2cccc(NC(=O)/C=C/c3cccc(C(=O)Nc4cccc(C(F)(F)F)c4)c3)c2)n1]

(E)-3-(3-((3-(trifluoromethyl)phenyl)carbamoyl)phenyl)acrylic acid Int-12 was reacted with 3-(2-methylpyrimidin-4-yl)benzenamine using experimental conditions outlined in the General Procedure I to give Compound 110 as an off-white solid in 57% yield. ¹H-NMR (400 MHz, DMSO-d₆): δ 10.69 (s, 1H), 10.55 (s, 1H), 8.77 (d, J=5.4 Hz, 1H), 8.51 (t, J=1.9 Hz, 1H), 8.29-8.23 (m, 2H), 8.10 (d, J=8.1 Hz, 1H), 8.03-7.96 (m, 2H), 7.90-7.85 (m, 2H), 7.83 (d, J=5.4 Hz, 1H), 7.72 (d, J=15.7 Hz, 1H), 7.66 (d, J=7.7 Hz, 1H), 7.62 (d, J=7.9 Hz, 1H), 7.52 (t, J=8.0 Hz, 1H), 7.48 (d, J=7.7 Hz, 1H), 7.02 (d, J=15.7 Hz, 1H), 2.70 (s, 3H); MS m/z=503.0 [M+H]⁺.

### (E)-N-(4-(3-((5-Morpholinopyridin-2-yl)amino)-3-oxoprop-1-en-1-yl)phenyl)-3-(trifluoromethyl)benzamide (Compound 111)

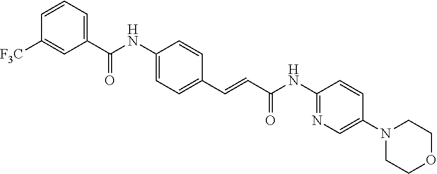 [SMILES: O=C(/C=C/c1ccc(NC(=O)c2cccc(C(F)(F)F)c2)cc1)Nc1ccc(N2CCOCC2)cn1]

(E)-3-(4-(3-(Trifluoromethyl)benzamido)phenyl)acrylic acid (Int-111) was reacted with 2-amino-5-morpholinopyridine using experimental conditions outlined in the General Procedure I to give Compound 111 as an orange solid in 20% yield. ¹H-NMR (400 MHz, DMSO-d₆): δ 10.64 (s, 1H), 10.46 (s, 1H), 8.30 (s, 1H), 8.28 (d, J=8.3 Hz, 1H), 8.13 (d, J=9.0 Hz, 1H), 8.05 (d, J=2.9 Hz, 1H), 7.99 (d, J=7.3 Hz, 1H), 7.87 (d, J=8.7 Hz, 2H), 7.81 (t, J=7.9 Hz, 1H), 7.62 (d, J=8.6 Hz, 2H), 7.57 (d, J=15.6 Hz, 1H), 7.44 (dd, J=9.2, 3.1 Hz, 1H), 6.95 (d, J=15.7 Hz, 1H), 3.75 (t, J=4.7 Hz, 4H), 3.12 (t, J=4.8 Hz, 4H); MS m/z=497.2 [M+H]⁺.

### General Procedure II.

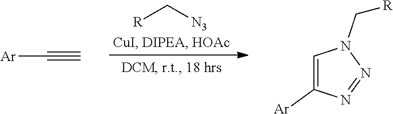

Copper(I) iodide (0.1 equiv.) was added to a solution of DIPEA (0.2 equiv.) and glacial acetic acid (0.2 equiv.) in DCM (1 M). Then the corresponding alkyne (1.0 equiv.) and azide (1.05 equiv.) were added. The reaction mixture was stirred at RT for 18 hours, then it was diluted with water and extracted with DCM (3×). Combined organic layers were washed with water (1×), brine (1×), dried over anhydrous sodium sulfate and concentrated in vacuo. The crude product was purified on reverse phase silica gel using a gradient of water and acetonitrile with 0.1% trifluoroacetic acid from 90:10 to 0:100. Fractions containing the desired product were combined, treated with saturated aqueous NaHCO₃ and extracted with DCM (3×). Combined organic phases were washed with brine (1×), dried over sodium sulfate, filtered and concentrated in vacuo to give the desired product.

### (E)-N-Methyl-3-(3-oxo-3-((5-(1-(2-(piperidin-1-yl)ethyl)-1H-1,2,3-triazol-4-yl)pyridin-2-yl)amino)prop-1-en-1-yl)-N-(3-(trifluoromethyl)phenyl)benzamide (Compound 112)

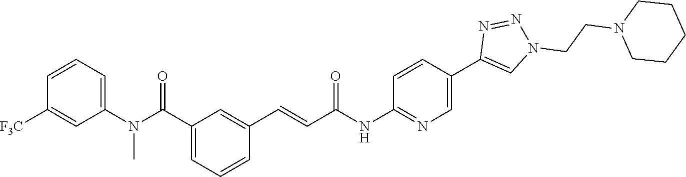 [SMILES: CN(C(=O)c1cccc(/C=C/C(=O)Nc2ccc(-c3cn(CCN4CCCCC4)nn3)cn2)c1)c1cccc(C(F)(F)F)c1]

(E)-3-(3-(5-Ethynylpyridin-2-ylamino)-3-oxoprop-1-enyl)-N-methyl-N-(3-(trifluoromethyl) phenyl)benzamide (Compound 97) was reacted with 1-(2-azidoethyl)piperidine (Int-112) using experimental conditions outlined in the General Procedure II to give Compound 112 as a white solid in 21% yield. ¹H-NMR (400 MHz, MeOD): δ 8.80 (dd, J=2.4, 0.8 Hz, 1H), 8.43 (s, 1H), 8.33 (dd, J=8.7, 0.7 Hz, 1H), 8.20 (dd, J=8.7, 2.4 Hz, 1H), 7.65-7.57 (m, 3H), 7.55-7.46 (m, 4H), 7.33-7.28 (m, 2H), 6.85 (d, J=15.7 Hz, 1H), 4.61 (t, J=6.7 Hz, 2H), 3.53 (s, 3H), 2.90 (t, J=6.7 Hz, 2H), 2.54-2.50 (m, 4H), 1.61 (dt, J=11.2, 5.6 Hz, 4H), 1.48 (dt, J=11.2, 5.9 Hz, 2H) ppm; MS m/z=604.1 [M+H]⁺.

### (E)-N-Methyl-3-(3-((5-(1-(3-(4-methylpiperazin-1-yl)propyl)-1H-1,2,3-triazol-4-yl)pyridin-2-yl)amino)-3-oxoprop-en-1-yl)-N-(3-(trifluormethyl)phenyl)benzamide (Compound 113)

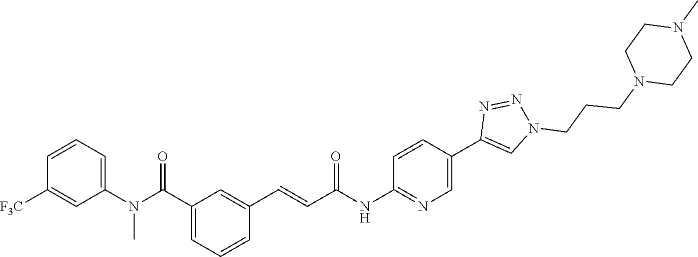 [SMILES: CN1CCN(CCCn2cc(-c3ccc(NC(=O)/C=C/c4cccc(C(=O)N(C)c5cccc(C(F)(F)F)c5)c4)nc3)nn2)CC1]

(E)-3-(3-(5-Ethynylpyridin-2-ylamino)-3-oxoprop-1-enyl)-N-methyl-N-(3-(trifluoromethyl) phenyl)benzamide (Compound 97) was reacted with 1-(3-azidopropyl)-4-methylpiperazine (Int-113) using experimental conditions outlined in the General Procedure II to give Compound 113 as a white solid in 51% yield. ¹H-NMR (400 MHz, DMSO-d₆): δ 10.83 (s, 1H), 8.82 (dd, J=2.3, 0.7 Hz, 1H), 8.63 (s, 1H), 8.31 (d, J=8.7 Hz, 1H), 8.23 (dd, J=8.6, 2.3 Hz, 1H), 7.62 (s, 1H), 7.59 (s, 1H), 7.56-7.49 (m, 5H), 7.32 (t, J=7.6 Hz, 1H), 7.25 (d, J=7.8 Hz, 1H), 7.01 (d, J=15.8 Hz, 1H), 4.43 (t, J=7.0 Hz, 2H), 3.43 (s, 3H), 2.37-2.27 (m, 10H), 2.13 (s, 3H), 2.02 (quint., J=7.0 Hz, 2H) ppm; MS m/z=633.2 [M+H]⁺.

### (E)-N-Methyl-3-(3-((5-(1-(3-morpholinopropyl)-1H-1,2,3-triazol-4-yl)pyridin-2-yl)amino)-3-oxoprop-1-en-1-yl)-N-(3-(trifluoromethyl)phenyl)benzamide (Compound 114)

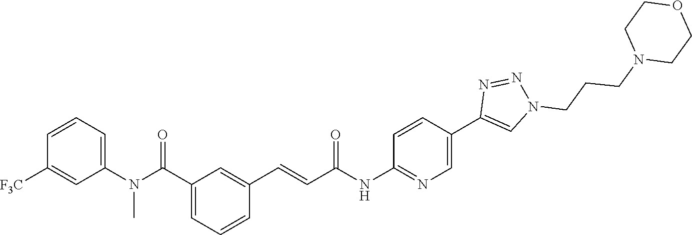 [SMILES: CN(C(=O)c1cccc(/C=C/C(=O)Nc2ccc(-c3cn(CCCN4CCOCC4)nn3)cn2)c1)c1cccc(C(F)(F)F)c1]

Sodium azide (6.8 mg, 0.11 mmol) was suspended in dry N,N-dimethylformamide (250 μL) and 4-(3-chloropropyl)morpholine (16.4 mg, 0.10 mmol) was added. The reaction mixture was stirred for 2 hours at room temperature, then (E)-3-(3-(5-Ethynylpyridin-2-ylamino)-3-oxoprop-1-enyl)-N-methyl-N-(3-(trifluoromethyl)phenyl)benzamide (Compound 97) (89.9 mg, 0.20 mmol) was added, followed by copper(I) iodide (3.8 mg, 0.02 mmol). The reaction mixture was stirred at RT for 18 hrs before it was diluted with water (5 mL) and extracted with EtOAC (3×5 mL). Combined organic phases were washed with brine (1×10 mL), dried over sodium sulfate and concentrated in vacuo. The crude product was purified on reversed-phase silica gel using a gradient (90:10 to 0:100) of water and acetonitrile with 0.1% trifluoroacetic acid. Fractions containing the desired product were combined and treated with saturated sodium bicarbonate solution. The mixture was extracted with dichloromethane (3×) and combined organic layers were washed with brine (1×), dried over sodium sulfate, filtered and concentrated in vacuo to give 11.5 mg of Compound 114 as a white solid in 19% yield. ¹H-NMR (400 MHz, DMSO-d₆): δ 8.80 (dd, J=2.3, 0.8 Hz, 1H), 8.43 (s, 1H), 8.34 (dd, J=8.7, 0.6 Hz, 1H), 8.20 (dd, J=8.7, 2.4 Hz, 1H), 7.63-7.58 (m, 3H), 7.55-7.46 (m, 4H), 7.31-7.28 (m, 2H), 6.86 (d, J=15.7 Hz, 1H), 4.55 (t, J=6.9 Hz, 2H), 3.69 (t, J=4.7 Hz, 4H), 3.53 (s, 3H), 2.46 (bs, 4H), 2.42 (t, J=7.1 Hz, 2H), 2.17 (quintet, J=7.1 Hz, 2H). MS m/z=620.2 [M+H]⁺.

### (E)-N-methyl-3-(3-oxo-3-((5-(1-(3-(piperidin-1-yl)propyl)-1H-1,2,3-triazol-4-yl)pyridin-2-yl)amino)prop-1-en-1-yl)-N-(3-(trifluoromethyl)phenyl)benzamide (Compound 115)

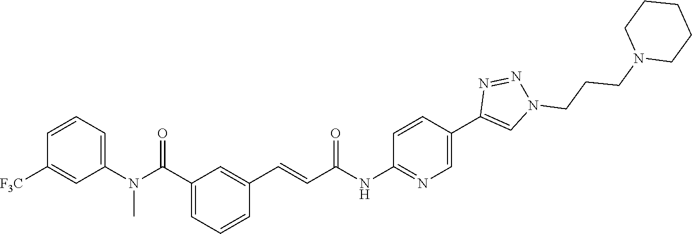 [SMILES: CN(C(=O)c1cccc(/C=C/C(=O)Nc2ccc(-c3cn(CCCN4CCCCC4)nn3)cn2)c1)c1cccc(C(F)(F)F)c1]

Sodium azide (6.8 mg, 0.105 mmol) was suspended in dry N,N-dimethylformamide (250 μL) and 1-(3-chloropropyl)piperidine (16.2 mg, 0.10 mmol) was added. The reaction mixture was stirred for 24 hours at RT. Then (E)-3-(3-(5-Ethynylpyridin-2-ylamino)-3-oxoprop-1-enyl)-N-methyl-N-(3-(trifluoromethyl)phenyl)benzamide (Compound 97) (44.9 mg, 0.10 mmol) was added, followed by copper(I) iodide (1.0 mg, 0.005 mmol), and the reaction mixture was stirred at RT for 48 hours. Then the reaction mixture was diluted with water (5 mL) and extracted with EtOAc (3×5 mL). Combined organic phases were washed with brine (1×10 mL), dried over sodium sulfate and concentrated in vacuo. The crude product was purified on reversed phase silica chromatography using a gradient (90:10 to 0:100) of water and acetonitrile with 0.1% trifluoroacetic acid. Fractions containing the desired product were combined, treated with saturated aqueous sodium bicarbonate solution and extracted with dichloromethane (3×). Combined organic phases were washed with brine (1×), dried over sodium sulfate and concentrated in vacuo to give 21.4 mg of Compound 115 as a yellow solid in 35% yield. ¹H-NMR (400 MHz, DMSO-d₆): δ 10.83 (s, 1H), 8.82 (dd, J=2.3, 0.7 Hz, 1H), 8.64 (s, 1H), 8.31 (d, J=8.7 Hz, 1H), 8.23 (dd, J=8.7, 2.3 Hz, 1H), 7.62 (s, 1H), 7.59 (s, 1H), 7.56-7.48 (m, 5H), 7.32 (t, J=7.6 Hz, 1H), 7.25 (d, J=7.7 Hz, 1H), 7.01 (d, J=15.7 Hz, 1H), 4.43 (t, J=7.0 Hz, 2H), 3.43 (s, 3H), 2.30 (bs, 6H), 2.08-1.96 (m, 2H), 1.50-1.43 (m, 4H), 1.37 (bs, 2H); MS m/z=618.1 [M+H]⁺.

### (E)-3-(3-((5-(1-(3-(Dimethylamino)propyl)-1H-1,2,3-triazol-4-yl)pyridin-2-yl)amino)-3-oxoprop-1-en-1-yl)-N-methyl-N-(3-trifluoromethyl)phenyl)benzamide (Compound 116)

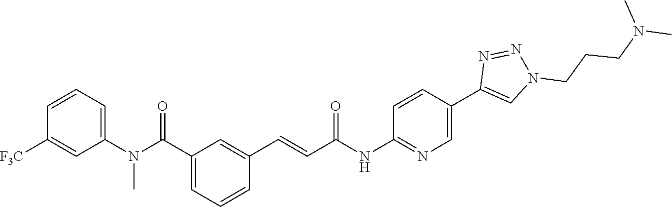 [SMILES: CN(C)CCCn1cc(-c2ccc(NC(=O)/C=C/c3cccc(C(=O)N(C)c4cccc(C(F)(F)F)c4)c3)nc2)nn1]

(E)-3-(3-(5-Ethynylpyridin-2-ylamino)-3-oxoprop-1-enyl)-N-methyl-N-(3-(trifluoromethyl) phenyl)benzamide (Compound 97) was reacted with 3-azidopropyldimethylamine (Int-115) using experimental conditions outlined in the General Procedure II to give Compound 116 as an off-white solid in 66% yield. ¹H-NMR (400 MHz, DMSO-d₆): δ 10.83 (s, 1H), 8.82 (dd, J=2.4, 0.8 Hz, 1H), 8.64 (s, 1H), 8.31 (d, J=8.7 Hz, 1H), 8.23 (dd, J=8.6, 2.3 Hz, 1H), 7.62 (s, 1H), 7.59 (s, 1H), 7.56-7.49 (m, 5H), 7.32 (t, J=7.7 Hz, 1H), 7.25 (d, J=7.8 Hz, 1H), 7.01 (d, J=15.8 Hz, 1H), 4.43 (t, J=7.1 Hz, 2H), 3.43 (s, 3H), 2.22 (t, J=6.9 Hz, 2H), 2.14 (s, 6H), 2.05-1.95 (m, 2H); MS m/z=578.1 [M+H]⁺.

### (E)-3-(3-((5-(1-(3-(Dimethylamino)propyl)-1H-1,2,3-triazol-4-yl)pyridin-2-yl)amino)-3-oxoprop-1-en-1-yl)-N-(trifluoromethyl)phenyl)benzamide (Compound 117)

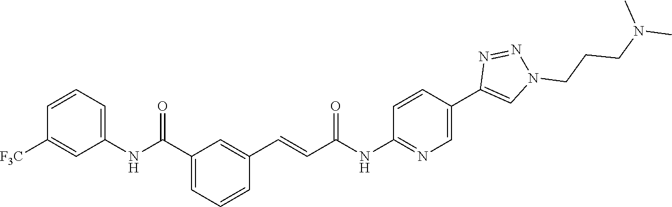 [SMILES: CN(C)CCCn1cc(-c2ccc(NC(=O)/C=C/c3cccc(C(=O)Nc4cccc(C(F)(F)F)c4)c3)nc2)nn1]

(E)-3-(3-((5-ethynylpyridin-2-yl)amino)-3-oxoprop-1-en-1-yl)-N-(3-(trifluoromethyl) phenyl)benzamide (Compound 65) was reacted with 3-azidopropyldimethylamine (Int-115) using experimental conditions outlined in the General Procedure II to give Compound 117 as a white solid in 74% yield. ¹H-NMR (400 MHz, DMSO-d₆): δ 10.90 (s, 1H), 10.69 (s, 1H), 8.83 (d, J=2.3 Hz, 1H), 8.65 (s, 1H), 8.35 (d, J=8.6 Hz, 1H), 8.28-8.22 (m, 3H), 8.09 (d, J=8.3 Hz, 1H), 8.01 (d, J=7.8 Hz, 1H), 7.86 (d, J=7.9 Hz, 1H), 7.75 (d, J=15.7 Hz, 1H), 7.66 (d, J=7.8 Hz, 1H), 7.62 (d, J=8.3 Hz, 1H), 7.48 (d, J=7.8 Hz, 1H), 7.20 (d, J=15.8 Hz, 1H), 4.44 (t, J=7.1 Hz, 2H), 2.24 (t, J=6.8 Hz, 2H), 2.15 (s, 6H), 2.01 (quintet, J=7.0 Hz, 2H) ppm; MS m/z=564.1 [M+H]⁺.

### (E)-3-(3-((5-(1-(3-Morpholinopropyl)-1H-1,2,3-triazol-4-yl)pyridin-2-yl)amino)-3-oxoprop-1-en-1-yl)-N-(3-(trifluoromethyl)phenyl)benzamide (Compound 118)

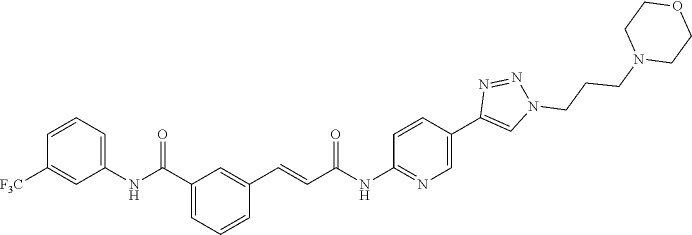 [SMILES: O=C(/C=C/c1cccc(C(=O)Nc2cccc(C(F)(F)F)c2)c1)Nc1ccc(-c2cn(CCCN3CCOCC3)nn2)cn1]

(E)-3-(3-((5-ethynylpyridin-2-yl)amino)-3-oxoprop-1-en-yl)-N-(3-(trifluoromethyl) phenyl)benzamide (Compound 65) was reacted with 1-(3-azidopropyl)-morpholine (Int-114) using experimental conditions outlined in the General Procedure II to give Compound 118 as an off-white solid in 66% yield. ¹H-NMR (400 MHz, DMSO-d₆): δ 10.90 (s, 1H), 10.67 (s, 1H), 8.83 (d, J=2.3 Hz, 1H), 8.65 (s, 1H), 8.35 (d, J=8.6 Hz, 1H), 8.30-8.19 (m, 3H), 8.09 (d, J=8.8 Hz, 1H), 8.00 (d, J=7.7 Hz, 1H), 7.86 (d, J=7.7 Hz, 1H), 7.75 (d, J=15.8 Hz, 1H), 7.66 (d, J=7.7 Hz, 1H), 7.62 (d, J=8.4 Hz, 1H), 7.48 (d, J=7.8 Hz, 1H), 7.19 (d, J=15.8 Hz, 1H), 4.45 (t, J=6.9 Hz, 2H), 3.57 (t, J=4.5 Hz, 4H), 2.34 (bs, 4H), 2.30 (t, J=7.3 Hz, 2H), 2.04 (quintet, J=6.9 Hz, 2H); MS m/z=606.0 [M+H]⁺.

### (E)-3-(3-Oxo-3-((5-(1-(3-(piperidin-1-yl)propyl)-1H-1,2,3-triazol-4-yl)pyridin-2-yl)amino)prop-1-en-1-yl)-N-(3-(trifluoromethyl)phenyl)benzamide (Compound 119)

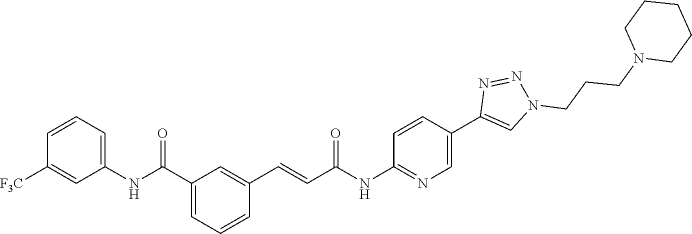 [SMILES: O=C(/C=C/c1cccc(C(=O)Nc2cccc(C(F)(F)F)c2)c1)Nc1ccc(-c2cn(CCCN3CCCCC3)nn2)cn1]

(E)-3-(3-((5-ethynylpyridin-2-yl)amino)-3-oxoprop-1-en-1-yl)-N-(3-(trifluoromethyl) phenyl)benzamide (Compound 65) was reacted with 1-(3-azidopropyl)piperidine (Int-116) using experimental conditions outlined in the General Procedure II to give Compound 119 as an off-white solid in 78% yield. ¹H-NMR (400 MHz, DMSO-d₆): δ 10.90 (s, 1H), 10.68 (s, 1H), 8.83 (dd, J=2.3, 0.8 Hz, 1H), 8.65 (s, 1H), 8.35 (d, J=8.7 Hz, 1H), 8.29-8.21 (m, 3H), 8.09 (d, J=8.0 Hz, 1H), 8.00 (d, J=7.7 Hz, 1H), 7.86 (d, J=7.8 Hz, 1H), 7.75 (d, J=15.7 Hz, 1H), 7.66 (d, J=7.8 Hz, 1H), 7.62 (d, J=8.4 Hz, 1H), 7.48 (d, J=7.8 Hz, 1H), 7.19 (d, J=15.8 Hz, 1H), 4.43 (t, J=7.0 Hz, 2H), 2.30 (bs, 4H), 2.25 (t, J=6.9 Hz, 2H), 2.02 (quintet, J=7.0 Hz, 2H), 1.53-1.44 (m, 4H), 1.41-1.32 (m, 2H). MS m/z=604.3 [M+H]⁺.

### (E)-N-Methyl-3-(3-((5-(1-(2-morpholinoethyl)-1H-1,2,3-triazol-4-yl)pyridin-2-yl)amino)-3-oxoprop-1-en-1-yl)-N-(3-(trifluoromethyl)phenyl)benzamide (Compound 120)

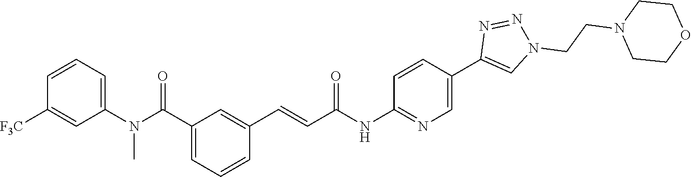 [SMILES: CN(C(=O)c1cccc(/C=C/C(=O)Nc2ccc(-c3cn(CCN4CCOCC4)nn3)cn2)c1)c1cccc(C(F)(F)F)c1]

(E)-3-(3-(5-Ethynylpyridin-2-ylamino)-3-oxoprop-1-enyl)-N-methyl-N-(3-(trifluoromethyl) phenyl)benzamide (Compound 97) was reacted with 1-(2-azidoethyl)morpholine (Int-117) using experimental conditions outlined in the General Procedure II to give Compound 120 as an off-white solid in 51% yield. ¹H-NMR (400 MHz, DMSO-d₆): δ 10.83 (s, 1H), 8.83 (dd, J=2.3, 0.7 Hz, 1H), 8.62 (s, 1H), 8.32 (d, J=8.7 Hz, 1H), 8.24 (dd, J=8.6, 2.3 Hz, 1H), 7.62 (s, 1H), 7.60 (s, 1H), 7.58-7.49 (m, 5H), 7.32 (t, J=7.7 Hz, 1H), 7.25 (d, J=7.7 Hz, 1H), 7.01 (d, J=15.8 Hz, 1H), 4.55 (t, J=6.3 Hz, 2H), 3.56 (t, J=4.6 Hz, 4H), 3.43 (s, 3H), 2.80 (t, J=6.3 Hz, 2H), 2.45 (bs, 4H); MS m/z=606.4 [M+H]⁺.

### (E)-N-Methyl-3-(3-oxo-3-((5-(1-(2-(pyrrolidin-1-yl)ethyl)-1H-1,2,3-triazol-4-yl)pyridin-2-yl)amino)prop-1-en-1-yl)-N-(3-(trifluoromethyl)phenyl)benzamide (Compound 121)

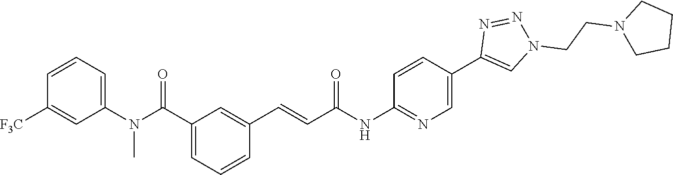 [SMILES: CN(C(=O)c1cccc(/C=C/C(=O)Nc2ccc(-c3cn(CCN4CCCC4)nn3)cn2)c1)c1cccc(C(F)(F)F)c1]

(E)-3-(3-(5-Ethynylpyridin-2-ylamino)-3-oxoprop-1-enyl)-N-methyl-N-(3-(trifluoromethyl) phenyl)benzamide (Compound 97) was reacted with 1-(2-azidoethyl)pyrrolidine (Int-18) using experimental conditions outlined in the General Procedure II to give Compound 121 as an off-white solid in 38% yield. ¹H-NMR (400 MHz, DMSO-d₆): δ 10.83 (s, 1H), 8.83 (dd, J=2.3, 0.8 Hz, 1H), 8.64 (s, 1H), 8.32 (d, J=8.7 Hz, 1H), 8.24 (dd, J=8.6, 2.3 Hz, 1H), 7.62 (s, 1H), 7.59 (s, 1H), 7.56-7.51 (m, 5H), 7.32 (t, J=7.6 Hz, 1H), 7.25 (d, J=7.5 Hz, 1H), 7.01 (d, J=15.7 Hz, 1H), 4.52 (t, J=6.3 Hz, 2H), 3.43 (s, 3H), 2.91 (t, J=6.3 Hz, 2H), 2.51-2.46 (m, 4H), 1.72-1.62 (m, 4H) ppm; MS m/z=590.2 [M+H]⁺.

### (E)-3-(3-Oxo-3-((5-(1-(2-(pyrrolidin-1-yl)ethyl)-1H-1,2,3-triazol-4-yl)pyridin-2-yl)amino)prop-1-en-1-yl)-N-(3-(trifluoromethyl)phenyl)benzamide (Compound 122)

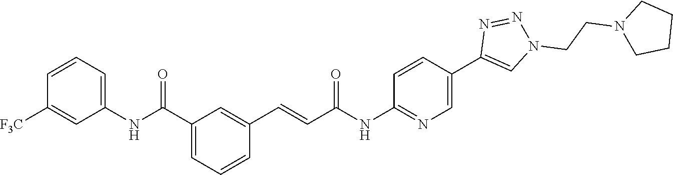 [SMILES: O=C(/C=C/c1cccc(C(=O)Nc2cccc(C(F)(F)F)c2)c1)Nc1ccc(-c2cn(CCN3CCCC3)nn2)cn1]

(E)-3-(3-((5-ethynylpyridin-2-yl)amino)-3-oxoprop-1-en-1-yl)-N-(3-(trifluoromethyl) phenyl)benzamide (Compound 65) was reacted with 1-(2-azidoethyl)pyrrolidine (Int-18) using experimental conditions outlined in the General Procedure II to give Compound 122 as an off-white solid in 54% yield. ¹H-NMR (400 MHz, DMSO-d₆): δ 10.90 (bs, 1H), 10.73 (bs, 1H), 8.83 (dd, J=2.3, 0.8 Hz, 1H), 8.65 (s, 1H), 8.35 (d, J=8.7 Hz, 1H), 8.29-8.22 (m, 3H), 8.10 (d, J=9.0 Hz, 1H), 8.02 (d, J=7.7 Hz, 1H), 7.86 (d, J=7.8 Hz, 1H), 7.75 (d, J=15.7 Hz, 1H), 7.65 (d, J=7.8 Hz, 1H), 7.62 (d, J=8.4 Hz, 1H), 7.48 (d, J=7.7 Hz, 1H), 7.21 (d, J=15.8 Hz, 1H), 4.53 (t, J=6.3 Hz, 2H), 2.91 (t, J=6.4 Hz, 2H), 2.53-2.44 (m, 4H), 1.72-1.61 (m, 4H); MS m/z=576.2 [M+H]⁺.

### (E)-3-(3-((5-(1-(2-Morpholinoethyl)-1H-1,2,3-triazol-4-yl)pyridin-2-yl)amino)-3-oxoprop-1-en-1-yl)-N-(3-(trifluormethyl)phenyl)benzamide (Compound 123)

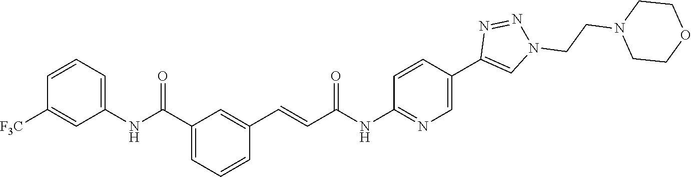 [SMILES: O=C(/C=C/c1cccc(C(=O)Nc2cccc(C(F)(F)F)c2)c1)Nc1ccc(-c2cn(CCN3CCOCC3)nn2)cn1]

(E)-3-(3-((5-ethynylpyridin-2-yl)amino)-3-oxoprop-1-en-1-yl)-N-(3-(trifluoromethyl) phenyl)benzamide (Compound 65) was reacted with 1-(2-azidoethyl)morpholine (Int-117) using experimental conditions outlined in the General Procedure II to give Compound 123 as an off-white solid in 23% yield. ¹H-NMR (400 MHz, DMSO-d₆): δ 10.90 (s, 1H), 10.67 (s, 1H), 8.84 (d, J=2.2 Hz, 1H), 8.63 (s, 1H), 8.35 (d, J=8.7 Hz, 1H), 8.29-8.22 (m, 2H), 8.22 (s, 1H), 8.09 (d, J=8.3 Hz, 1H), 8.00 (d, J=7.7 Hz, 1H), 7.86 (d, J=7.9 Hz, 1H), 7.75 (d, J=15.7 Hz, 1H), 7.66 (d, J=7.8 Hz, 1H), 7.62 (d, J=8.4 Hz, 1H), 7.48 (d, J=7.8 Hz, 1H), 7.20 (d, J=15.8 Hz, 1H), 4.55 (t, J=6.3 Hz, 2H), 3.56 (t, J=4.5 Hz, 4H), 2.80 (t, J=6.3 Hz, 2H), 2.45 (bs, 4H); MS m/z=592.1 [M+H]⁺.

### (E)-3-(3-Oxo-3-((5-(1-(2-(piperidin-1-yl)ethyl)-1H-1,2,3-triazol-4-yl)pyridin-2-yl)amino)prop-1-en-1-yl)-N-(3-(trifluoromethyl)phenyl)benzamide (Compound 124)

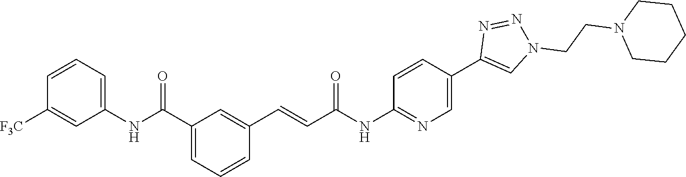 [SMILES: O=C(/C=C/c1cccc(C(=O)Nc2cccc(C(F)(F)F)c2)c1)Nc1ccc(-c2cn(CCN3CCCCC3)nn2)cn1]

(E)-3-(3-((5-ethynylpyridin-2-yl)amino)-3-oxoprop-1-en-1-yl)-N-(3-(trifluoromethyl) phenyl)benzamide (Compound 65) was reacted with 1-(2-azidoethyl)piperidine (Int-112) using experimental conditions outlined in the General Procedure II to give Compound 124 as a white solid in 40% yield. ¹H-NMR (400 MHz, DMSO-d₆): δ 10.90 (s, 1H), 10.67 (s, 1H), 8.83 (dd, J=2.4, 0.8 Hz, 1H), 8.60 (s, 1H), 8.35 (d, J=8.7 Hz, 1H), 8.29-8.22 (m, 3H), 8.09 (d, J=9.2 Hz, 1H), 8.00 (d, J=7.6 Hz, 1H), 7.86 (d, J=7.8 Hz, 1H), 7.75 (d, J=15.7 Hz, 1H), 7.66 (d, J=7.8 Hz, 1H), 7.62 (d, J=8.5 Hz, 1H), 7.48 (d, J=7.8 Hz, 1H), 7.19 (d, J=15.8 Hz, 1H), 4.52 (t, J=6.4 Hz, 2H), 2.75 (t, J=6.5 Hz, 2H), 2.40 (bs, 4H), 1.52-1.42 (m, 4H), 1.41-1.33 (m, 2H); MS m/z=590.1 [M+H]⁺.

### (E)-N-(3-(3-((2-(1-(3-morpholinopropyl)-1H-1,2,3-triazol-4-yl)phenyl)amino)-3-oxoprop-1-en-1-yl)phenyl)-3-(trifluoromethyl)benzamide (Compound 125)

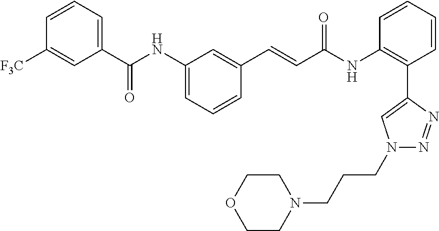 [SMILES: O=C(/C=C/c1cccc(NC(=O)c2cccc(C(F)(F)F)c2)c1)Nc1ccccc1-c1cn(CCCN2CCOCC2)nn1]

(E)-N-(3-(3-((2-Ethynylphenyl)amino)-3-oxoprop-1-en-1-yl)phenyl)-3-(trifluoromethyl)benzamide (Int-119) was reacted with 1-(3-azidopropyl)morpholine (Int-114) using experimental conditions outlined in the General Procedure II to give Compound 125 as an off-white solid in 60% yield. ¹H-NMR (400 MHz, DMSO-d₆): δ 11.20 (bs, 1H), 10.62 (s, 1H), 8.68 (s, 1H), 8.36-8.34 (m, 2H), 8.30 (d, J=7.8 Hz, 1H), 8.12 (s, 1H), 8.00 (d, J=8.2 Hz, 1H), 7.88-7.77 (m, 3H), 7.63 (d, J=15.6 Hz, 1H), 7.51-7.43 (m, 2H), 7.38 (td, J=7.5, 1.4 Hz, 1H), 7.24 (td, J=7.5, 1.0 Hz, 1H), 6.82 (d, J=15.7 Hz, 1H), 4.49 (t, J=6.9 Hz, 2H), 3.52 (t, J=4.6 Hz, 4H), 2.38-2.24 (m, 6H), 2.05 (quintet, J=6.9 Hz, 2H); MS m/z=605.0 [M+H]⁺.

### Cell Culture and Reporter Assay:

H1299 p53⁻/⁻ cells were maintained in Dulbecco's modified Eagle's medium (DMEM) with 10% (v/v) foetal calf serum (FCS) and 1% (v/v) penicillin/streptomycin. The cells were seeded at 1.0×10⁵ cells/well in 6-well plates, 24 hours prior to transfection. Cells were co-transfected with p53-pcDNA plasmid, p21-LacZ reporter plasmid and luciferase transfection efficiency plasmid using Lipofectamine® 2000 transfection reagent (Thermo Scientific) according to the manufacturer's instructions. In all cases, the total amount of plasmid DNA transfected per well was equilibrated by addition of the parental vector pcDNA3.1a (+).

### β-Galactosidase Assay and Western Blot Analysis:

Cells were harvested 24 hours after transfection and β-galactosidase activities were assessed using the Dual-light System (Applied Biosystems) according to the manufacturer's protocol. The β-galactosidase activity was normalized with luciferase activity for each sample. To check for expression levels of relevant proteins via western blot, around 5 μg of the cell lysates were probed for p53 with DO1 antibody followed by rabbit anti-mouse and for actin with horseradish peroxidise conjugated anti-actin C4 antibody.

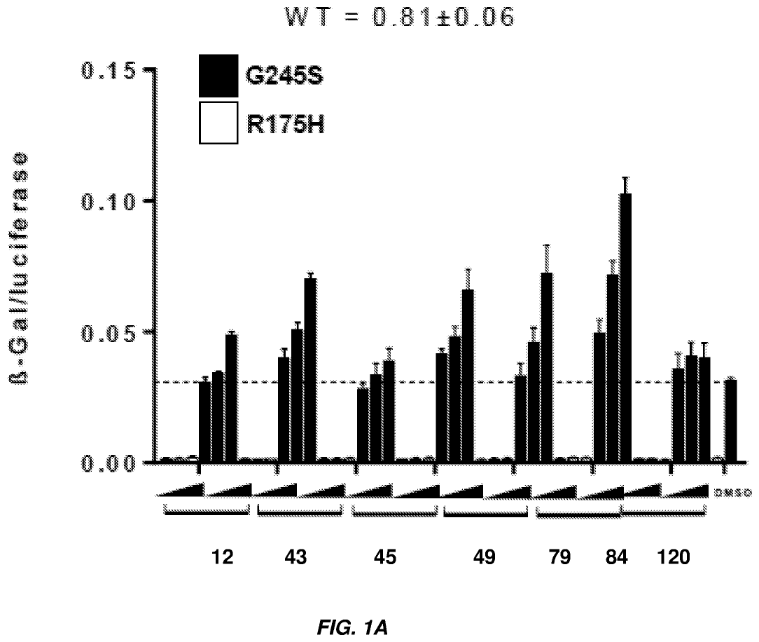

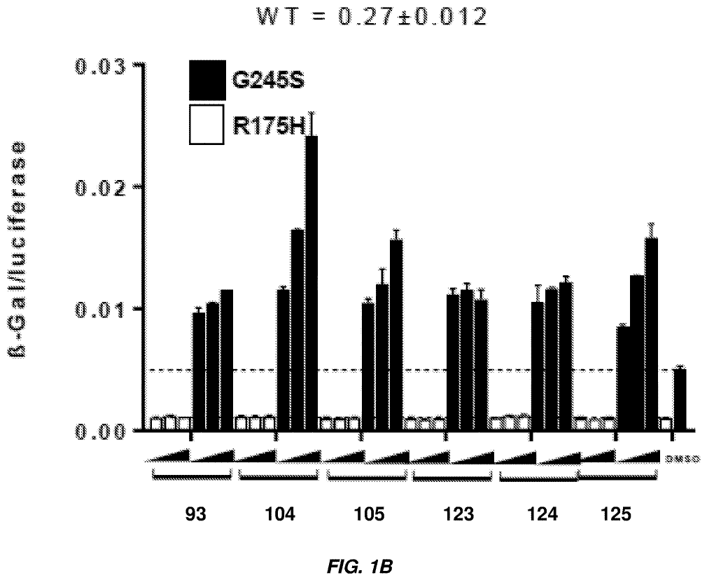

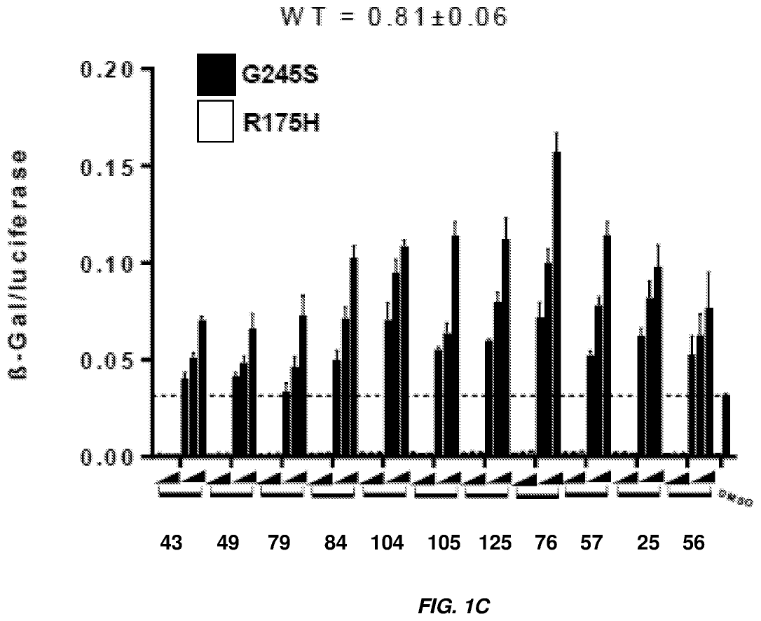

FIG. 1A-1C illustrate the p21 gene reporter assay results with H1299 p53-cells transfected with p53-R175H and p53-G245S plasmids for various compounds disclosed herein as compared to DMSO control at 2 μM, 5 μM and 10 μM respectively. Dose-dependent increase in p21 response was observed in cells carrying the p53-G245S mutant after treatment. Data shows average ±SD of two independent experiments. FIG. 1A is a bar chart illustrating the p21 gene reporter assay results with H1299 p53⁻/⁻ cells transfected with p53-R175H and p53-G245S plasmids for Compounds 12, 43, 45, 49, 79, 84 and 120, as compared to DMSO. FIG. 1B is a bar chart illustrating the p21 gene reporter assay results with H1299 p53⁻/⁻ cells transfected with p53-R175H and p53-G245S plasmids for Compounds 93, 104, 105, 123, 124, and 125, as compared to DMSO control. FIG. 1C is a bar chart illustrating the p21 gene reporter assay results with H1299 p53⁻/⁻ cells transfected with p53-R175H and p53-G245S plasmids for Compounds 43, 49, 79, 84, 104, 105, 125, 76, 57, 25, and 56, as compared to DMSO control.

What is claimed is:

1. A compound having the structure of Formula (I): 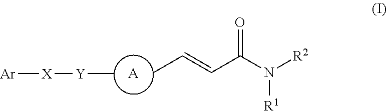
or a pharmaceutically acceptable salt thereof, wherein:
Ar is selected from the group consisting of C₆₋₁₀ aryl and 5 to 10 membered heteroaryl, each optionally substituted with one or more R¹⁰;
each of X and Y is independently selected from the group consisting of NR³, C(═O), and S(O)₂, provided that X and Y are not the same;
ring A is 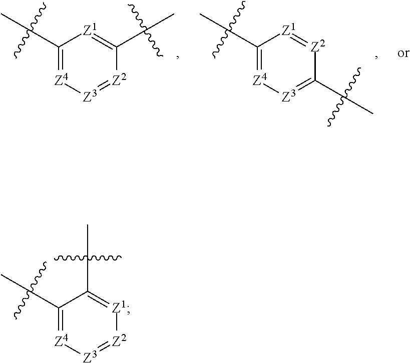
Z¹ is CR^{4a} or N;
Z² is CR^{4b} or N;
Z³ is CR^{4c} or N;
Z⁴ is CR^{4d} or N;
each of R¹ and R³ is independently selected from the group consisting of H, optionally substituted C₁₋₆ alkyl, optionally substituted C₆₋₁₀ aryl, optionally substituted 5 to 10 membered heteroaryl, optionally substituted 3 to 10 membered heterocyclyl, optionally substituted C₇₋₁₄ aralkyl, and optionally substituted C₃₋₇ carbocyclyl;
R² is selected from the group consisting of C₆₋₁₀ aryl and 5 to 10 membered heteroaryl, each optionally substituted with one or more R¹¹;
alternatively, R¹ and R² together with the nitrogen atom to which they are attached form a 3 to 10 membered heterocyclyl or a 5 to 10 membered heteroaryl, each optionally substituted with one or more R¹²;
each of R^{4a}, R^{4b}, R^{4c}, and R^{4d} is independently selected from the group consisting of H, optionally substituted C₁₋₆ alkyl, optionally substituted C₂₋₆ alkenyl, optionally substituted C₂₋₆ alkynyl, C₁₋₆ haloalkyl, C₁₋₆ alkoxy, C₁₋₆ haloalkoxy, (C₁₋₆ alkoxy)C₁₋₆ alkyl, C₁₋₆ alkylthiol, halo, —CN, —NO₂, —NR^{5a}R^{6a}, —OR^{7a}, —C(O)R^{8a}, —C(O)OR^{7b}, —C(O)NR^{5b}R^{6b}, —NR^{5c}C(O)R^{8b}, —NR^{5d}S(O)₂R^{9a} and —SO₂R^{9b};
each of R¹⁰, R¹¹ and R¹² is independently selected from the group consisting of C₁₋₆ alkyl, C₂₋₆ alkenyl, C₂₋₆ alkynyl, C₁₋₆ haloalkyl, C₁₋₆ alkoxy, C₁₋₆ haloalkoxy, (C₁₋₆ alkoxy)C₁₋₆ alkyl, C₁₋₆ alkylthiol, C₆₋₁₀ aryl, C₇₋₁₄ aralkyl, 5 to 10 membered heteroaryl, 3 to 7 membered heterocyclyl, C₃₋₇ carbocyclyl, halo, —CN, —NO₂, —NR^{5a}R^{6a}, —OR^{7a}, —C(O)R^{8a}, —C(O)OR^{7b}, —C(O)NR^{5b}R^{6b}, —NR^{5c}C(O)R^{8b}, —NR^{5d}S(O)₂R^{9a} and —S(O)₂R^{9b}, wherein each of C₁₋₆ alkyl, C₂₋₆ alkenyl, C₂₋₆ alkynyl, C₆₋₁₀ aryl, C₇₋₁₄ aralkyl, 5 to 10 membered heteroaryl, 3 to 7 membered heterocyclyl, and C₃₋₇ carbocyclyl is optionally substituted with one or more R¹³; or two geminal R¹² form oxo;
each R¹³ is independently selected from the group consisting of C₁₋₆ alkyl, C₂₋₆ alkenyl, C₂₋₆ alkynyl, C₁₋₆ haloalkyl, C₁₋₆ alkoxy, C₁₋₆ haloalkoxy, (C₆ alkoxy)C₁₋₆ alkyl, C₁₋₆ alkylthiol, optionally substituted phenyl, optionally substituted 5 or 6 membered heteroaryl, optionally substituted 3 to 7 membered heterocyclyl, optionally substituted C₃₋₇ carbocyclyl, halo, —CN, —NO₂, SEM, —(C₀₋₆ alkylene)NR^{5a}R^{6a}, —OR^{7a}, —C(O)R^{8a}, —C(O)OR^{7b}, —C(O)NR^{5b}R^{6b}, —NR^{5c}C(O)R^{8b}, —NR^{5c}S(O)₂R^{9a} and —S(O)₂R^{9b}, or two geminal R¹³ form oxo;
each of R^{5a}, R^{5b}, R^{5c}, R^{5d,} R⁶, and R^{6b} is independently selected from the group consisting of H, optionally substituted C₁₋₆ alkyl, optionally substituted (C₀₋₆ alkylene)C₆₋₁₀ aryl, optionally substituted (C₀₋₆ alkylene) 5 to 10 membered heteroaryl, optionally substituted (C₀₋₆ alkylene) 3 to 7 membered heterocyclyl, and optionally substituted (C04 alkylene)C₃₋₇ carbocyclyl; or R5a and R⁶ together with the nitrogen atom to which they are attached form an optionally substituted 3 to 7 membered heterocyclyl; or R^{5b} and R^{6b} together with the nitrogen atom to which they are attached form an optionally substituted 3 to 7 membered heterocyclyl; and
each of R^{7a}, R^{7b}, R^{8a}, R^{8b}, R^{9a}, and R^{9b} is independently selected from the group consisting of H, optionally substituted C₁₋₆ alkyl, optionally substituted C₂₋₆ alkenyl, optionally substituted C₂₋₆ alkynyl, optionally substituted C₆₋₁₀ aryl, optionally substituted C₇₋₁₄ aralkyl, and optionally substituted C₃₋₇ carbocyclyl.

2. The compound of claim 1, having the structure of Formula (II): 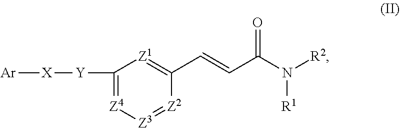
or a pharmaceutically acceptable salt thereof.

3. The compound of claim 2, having the structure of Formula (II-A), (II-B), (II-C) or (II-D): 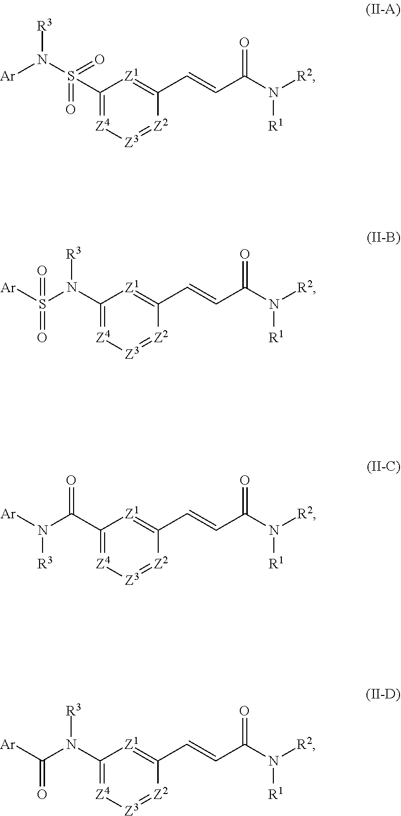
or a pharmaceutically acceptable salt thereof.

4. The compound of claim 3, wherein Z¹ is CR^{4a}, Z² is CR^{4b}, Z³ is CR^{4c} and Z⁴ is CR^{4d}.

5. The compound of claim 4, having the structure of Formula (III-A), (III-B), (III-C) or (III-D): 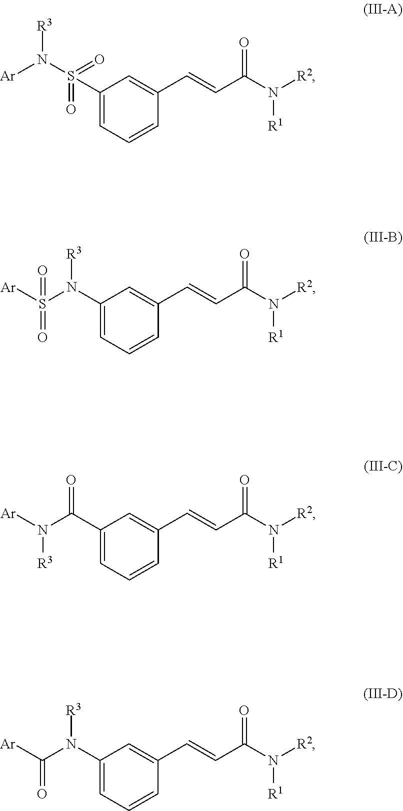
or a pharmaceutically acceptable salt thereof.

6. The compound of claim 3, wherein at least one of Z¹, Z², Z³, and Z⁴ is N.

7. The compound of claim 6, having the structure of Formula (IV-A), (IV-B), (IV-C) or (IV-D): 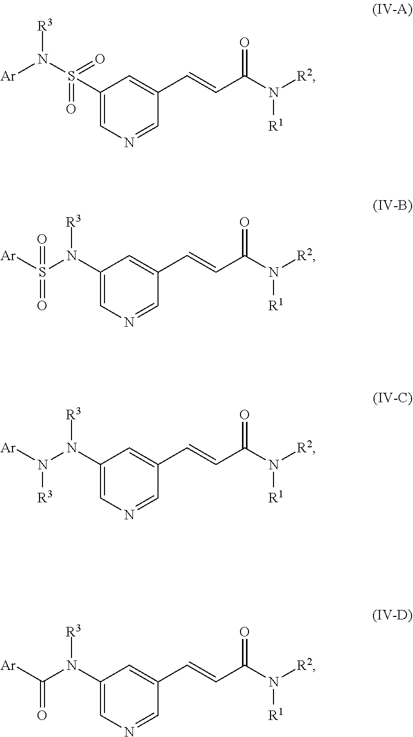
or a pharmaceutically acceptable salt thereof.

8. The compound of claim 1, having the structure of Formula (V): 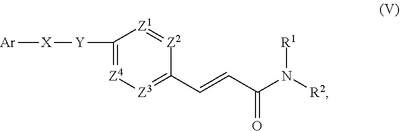
or a pharmaceutically acceptable salt thereof.

9. The compound of claim 8, having the structure of Formula (V-A), (V-B), (V-C) or (V-D): 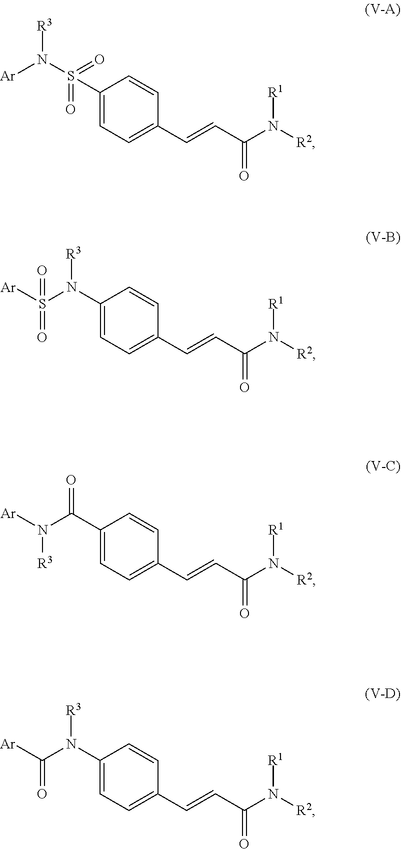
or a pharmaceutically acceptable salt thereof.

10. The compound of claim 1, wherein each of R¹ and R³ is independently H or C₁₋₆ alkyl.

11. The compound of claim 1, wherein Ar is a phenyl optionally substituted with one or more R¹⁰ and wherein each R¹⁰ is independently selected from the group consisting of halo, C₁₋₆ alkyl, C₁₋₆ haloalkyl, C₁₋₆ alkoxy, and C₁₋₆ haloalkoxy.

12. The compound of claim 1, wherein R² is selected from the group consisting of phenyl, oxadiazolyl, thiadiazolyl, thiazolyl, imidazolyl, oxazolyl, pyridyl, pyrimidyl, piridazinyl, benzothiazolyl, benzoxazolyl, and quinolyl, each optionally substituted with one or more R¹¹.

13. The compound of claim 12, wherein R₁₁ is selected from the group consisting of halo, —OH, —CN, C₁₋₆ alkyl, C₂₋₆ alkynyl, C₁₋₆ haloalkyl, C₁₋₆ haloalkoxy, C₁₋₆ alkoxy, —NR^{5a}R^{6a}, —C(O)OR^{7b}, —C(O)NR^{5b}R^{6b}, 5 membered heteroaryl, 6 membered heteroaryl, 9 membered heteroaryl, and 6 membered heterocyclyl, wherein each of the 5 membered heteroaryl, 6 membered heteroaryl, 9 membered heteroaryl, and 6 membered heterocyclyl is optionally substituted with one or more R¹³.

14. The compound of claim 13, wherein R¹¹ is —NH₂, —C(O)OR^{7b}, or —C(O)NR^{5b}R^{6b}; wherein R^{7b} is H or C₁₋₆ alkyl; and wherein R^{5b} is H and R^{6b} is optionally substituted (C₀₋₆ alkylene) 6 membered heterocyclyl, or R^{5b} and R^{6b} together with the nitrogen atom to which they are attached form an optionally substituted 6 membered heterocyclyl.

15. The compound of claim 13, wherein R¹¹ is selected from the group consisting of triazolyl, oxadiazolyl, thiadiazolyl, thiazolyl, imidazolyl, pyridyl, and benzoimidazolyl, each optionally substituted with one or more R¹³.

16. The compound of claim 1, wherein R¹ and R² together with the nitrogen atom to which they are attached form a 6 membered heterocyclyl optionally substituted with one or more R¹².

17. The compound of claim 16, wherein R¹² is C₂₋₆ alkynyl optionally substituted with one or more R³ or wherein two geminal R¹² form oxo.

18. The compound of claim 1, selected from the group consisting of Compounds 1-125 of Table 1, and pharmaceutically acceptable salts thereof.

19. A pharmaceutical composition comprising a compound of claim 1, or a pharmaceutically acceptable salt thereof, and a pharmaceutically acceptable excipient or carrier.

20. A method of treating cancer, comprising:
selecting a subject having a p53 mutation in the DNA-binding domain; and
administering a therapeutically effective amount of a compound of claim 1, or a pharmaceutically acceptable salt thereof, to the subject.
